(19) United States
(12) Patent Application Publication
(10) Pub. No.: US 2026/0137088 A1
(43) Pub. Date: May 21, 2026

(54) COMPOSITIONS OF ENDOPHYTES AND SYNTHETIC ENDOPHYTE CONSORTIA FOR IMPROVING NITROGEN AND PHOSPHORUS UPTAKE, NUTRITION, GROWTH, AND PERFORMANCE IN PLANTS AND METHODS OF MAKING AND USING THE SAME

(71) Applicants: INTRINSYX BIO INC., Sunnyvale, CA (US); University of Washington, Seattle, WA (US)

(72) Inventors: John L. Freeman III, Morgan Hill, CA (US); Douglas Baker, Davis, CA (US); Sharon L. Doty, Seattle, WA (US); Andrew Sher, Camano Island, WA (US); Michelle Lynn Levish, San Jose, CA (US); Twyla A. Belcher, Hayward, CA (US); Phuong Thuy PHAN, San Jose, CA (US)

(21) Appl. No.: 19/374,874

(22) Filed: Oct. 30, 2025

Related U.S. Application Data
(60) Provisional application No. 63/713,587, filed on Oct. 30, 2024.
(60) Provisional application No. 63/737,582, filed on Dec. 20, 2024.

(51) Int. Cl. A01N 63/20; A01P 21/00; C12N 1/20

(52) U.S. Cl. CPC A01N 63/20; A01P 21/00; C12N 1/20

(57) ABSTRACT

Endophyte inoculant compositions, methods of making such compositions, methods of using such compositions, and physiologically altered plants treating with such compositions are disclosed. The endophyte inoculant composition may include one or more of endophyte strains HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1 which promote plant mineral nutrient acquisition and uptake, vigor, health, growth, and yield when applied to non-native host plants.

### FIELD OF THE TECHNOLOGY

This technology relates to the manufacture, compositions, and methods that increase plant performance. The novel compositions include non-native endophytes applied to plants that incorporate the endophytes resulting in measurable plant benefits such as uptake from the atmosphere, soil, and water of nitrogen, phosphorus, other macro- and micronutrients, nutrient use efficiency, photosynthesis, growth, yield, carbon sequestration, tolerance to biotic and abiotic stressors and general plant health. The present technology has broad applications to plants generally, including applications in agricultural crop management, reducing of fertilizer and pesticide usage, reducing crop carbon footprints, improvement in crop mineral nutrition status which reduces plant pathogen loads and subsequently reduces pesticide usage, improvements in food quality and safety, increased plant health, biomass growth and yield rates for use in agriculture, landscaping and ornamental plants, and forestry.

### BACKGROUND

Endophytes are microorganisms (e.g., fungi and bacteria) that can have a symbiotic relationship with trees and plants, through which plant growth, fruit and seed yield, general health, and other characteristics can be improved. Salicaceae Endophytes for example Modulate Stomatal Behavior and Increase Water Use Efficiency in Rice. Frontiers in Plant Science, 9 doi10.3389/fpls.2018.00188. After inoculation, endophytes are incorporated into the plant tissues and can become an inheritable part of the plant. Endophytes can interpose between the cells and inside of the cells of a plant and thereby incorporate themselves into the tissue of the plant. Once incorporated into a plant and are also associated living on the plant root surface. The endophyte may improve plant nutrition by providing and augmenting the supply of macronutrients such as nitrogen, phosphorous, potassium, calcium, and sulfur, and micronutrients, such as iron, zinc, and magnesium, increasing photosynthesis, improving water use efficiency, and increased resistance to biotic and abiotic stress.

Despite the benefits provided by endophytes, there is very little practical utilization of endophytes in agriculture. Consumers and agricultural operations have become more aware of the damage that chemical fertilizers and pesticides cause to the environment and are now utilizing alternatives that are more natural and enhance the long-term sustainability of agriculture, livestock, and forest land production.

### SUMMARY OF THE INVENTION

The present disclosure provides for novel and inventive compositions comprising bacterial endophytes for application to non-native plant species, methods of making and using the same, and resulting novel plants. Specifically, one or more of the following endophyte strains may be included in an inoculant composition for application to a non-native host plant:

| Agricultural | | | |
|---|---|---|---|
| Research | | | |
| Culture | | | Tissue |
| Collection | | Genomic Species | isolated |
| (NRRL) | Strain | Identification | from |
| B-68480 | SD1 | Azotobacter beijerinckii | Populus |
| | | | trichocarpa |
| | | | root |
| B-68481 | SD2 | Azospirillum palustre | Populus |
| | | | trichocarpa |
| | | | root |
| DSM 35602 | 11R-B1 | Herbiconiux sp. | Populus |
| | | | trichocarpa |
| | | | root |
| B-68482 | 11R-B2 | Herbiconiux sp. | Populus |
| | | | trichocarpa |
| | | | root |
| B-68483 | 11R-BB | Sphingobium sp. | Populus |
| | | | trichocarpa |
| | | | root |
| B-68484 | 11R-BC | Herbiconiux sp. | Populus |
| | | | trichocarpa |
| | | | root |
| B-68485 | HT1-2 | Sphingobium sp. | Pluchea |
| | | | carolinsis |
| | | | stem |
| B-68486 | HT1-4 | Curtobacterium sp. | Pluchea |
| | | | carolinsis |
| | | | stem |
| B-68487 | HT1-7 | Curtobacterium sp. | Pluchea |
| | | | carolinsis |
| | | | stem |
| B-68488 | HT1-8 | Rhizobium wenxiniae | Pluchea |
| | | | carolinsis |
| | | | stem |
| B-68489 | HT1-9 | Azorhizobium sp. | Pluchea |
| | | | carolinsis |
| | | | stem |
| B-68490 | HT1-10 | Rhizobium sp. | Pluchea |
| | | | carolinsis |
| | | | stem |
| B-68491 | R10 | Rahnella aceris | Populus |
| | | | trichocarpa |
| | | | root |
| B-68492 | WP5 | Rahnella aceris | Populus |
| | | | trichocarpa |
| | | | stem |
| B-68493 | M2 | Pseudomonas sp. | Pinus |
| | | | muricata |
| | | | stem |
| B-68494 | M3 | Duffyella gerundensis | Pinus |
| | | | muricata |
| | | | stem |
| B-68496 | M5 | Pseudomonas sp. | Pinus |
| | | | muricata |
| | | | stem |
| B-68498 | TP SK3 | Paraburkholderia sp. | Thuja plicata |
| | | | stem |
| B-68499 | TP SK5 | Herbaspirillum sp. | Thuja plicata |
| | | | stem |
| B-68500 | TP SN7 | Paraburkholderia sp. | Thuja plicata |
| | | | stem |
| B-68501 | PM PF3 | Rahnella sp. | Pseudotsuga |
| | | | menziesii |
| | | | stem |
| B-68502 | PM SK6 | Rahnella sp. | Pseudotsuga |
| | | | menziesii |
| | | | stem |
| B-68503 | PS SN1 | Paraburkholderia sp. | Picea |
| | | | sitchensis |
| | | | stem |
| B-68504 | AWS1 | Priestia megaterium | Salicornia |
| | | | depressa |
| | | | Shoot |
| B-68506 | WP4-4-2 | Rhanella sp. | Populus |
| | | | trichocarpa |
| | | | stem |
| B-68508 | WP4-5-3 | Rhanella sp. | Populus |
| | | | trichocarpa |
| | | | stem |
| B-68509 | WP4-3-3 | Curtobacterium sp. | Populus |
| | | | trichocarpa |
| | | | stem |
| B-68510 | WP4-4-6 | Pseudomonas lutea | Populus |
| | | | trichocarpa |
| | | | stem |
| B-68511 | WP4-3-1 | Rhodotorula graminis | Populus |
| | | | trichocarpa |
| | | | stem |
| B-68512 | WP4-10-4 | Curtobacterium sp. | Populus |
| | | | trichocarpa |
| | | | leaf |
| B-68513 | WP1 | Rhodotorula graminis | Populus |
| | | | trichocarpa |
| | | | stem |
| B-68514 | WPL8-1 | Rahnella aceris | Populus |
| | | | trichocarpa |
| | | | stem |
| B-68515 | WP8 | Pseudomonas graminis | Populus |
| | | | trichocarpa |
| | | | stem |
| B-68081 | WW6 | Pseudomonas siliginis | Salix |
| | | | sitchensis |
| | | | stem |
| B-68078 | WW7 | Curtobacterium salicis | Salix |
| | | | sitchensis |
| | | | stem |
| B-68080 | PTD1 | Rhizobium sp. | Populus |
| | | | trichocarpa x |
| | | | Populus |
| | | | deltoides |
| B-68079 | WW5 | Sphingobium sp. | Salix |
| | | | sitchensis |
| | | | stem |

Pursuant to 37 C.F.R. §§ 1.831-1.835, a Sequence Listing in XML format is submitted herewith as a separate part of the disclosure and is hereby incorporated by reference in its entirety. The Sequence Listing XML file is named “1396007ARU01_sequence_listing.xml”, was created on Oct. 30, 2025, and has a file size of 77,975 bytes. The Sequence Listing XML file contains the nucleotide and/or amino acid sequence information for this application and is submitted in compliance with the requirements of 37 C.F.R. §§ 1.832-1.834.

The species identified in the table above were submitted to ARS Culture Collection (NRRL) located at 1815 N. University Street, Peoria, IL 61604. The 16S rDNA gene sequences for each of the above endophyte strains are listed in FIGS. 1A-1AO and found in the Sequence Listing XML file submitted with the present application. These strains were discovered in the tissues of a diverse set of wild tree and plant species found growing on rocky gravel substrata (granite and volcanic) with no soil or organic matter poplar trees, willow trees, bishop pines, Douglas firs, redcedars, spruce trees, pluchea bushes, and glasswort tidal marsh plants, which are all not nodule forming plants. Thus, the presence of potentially diazotrophic endophytes living inside all the branches and roots of these plant species was unexpected. Through a process of screening a large library of strains of endophytic bacteria, the above-identified set of endophytic bacterial strains were selected after the discovery that they have nitrogen use efficiency (NUE) traits and phosphorus use efficiency (PUE) that may be useful in improving crop plant health performance, including but not limited to the ability to grow on nitrogen free or nitrogen-limited media, the ability to enhance nitrogen (N) acquisition, and/or the ability to increase phosphorous (P) acquisition through increasing phosphate solubilization and increased root to shoot P uptake. These strains were further discovered to provide additional mineral nutrient uptake abilities in many crop plants inoculated with the strains. In strains where the nitrogenase genes are on plasmids, the plasmid can be lost and then regained from the microbial communities in crop plants. Inoculated plants showed colonization by the endophytes and increased mineral uptake from their roots and into their shoots. Subsequently, these unique strains were identified and isolated for this purpose among others. Furthermore, through optimization of the most efficient atmospheric nitrogen fixing strains along with a nitrogenase booster or helper strains, plus strains that can oxidize, hydrolyze, bio-transform and assimilate different nitrogen species from air, soil and water (e.g, N₂O, NH₃/NH₄ nitrate, nitrite, C—N), paired together with specific strains that mobilize insoluble forms of phosphorus and macronutrients (e.g., potassium, calcium, magnesium) plus those that make iron siderophores for iron and other micro-nutrient metals, the resulting novel inoculum combination formulations were found to increase plant biomass under nutrient limitations and enhance acquisition of macro-nutrient ions and plant required micronutrient ions in a synergistic manner. The application of the presently disclosed, novel microbe inoculum to non-native plants furthermore, results in increased growth, biomass, and yield in crop plants. The novel inoculum formulation also increases crop plant yield and quality in deficient, sufficient, and highly optimized agronomic conditions alike. These endophyte strains and combinations thereof are generally absent from agricultural crop plant varieties and the soils used in agricultural production.

Many of the endophyte strains discussed above have been demonstrated to be newly discovered endophyte species: Azorhizobium sp. HT1-9; Rhizobium sp. HT1-10, Sphingobium sp, HT1-2,, Curtobacterium sp. HT1-4, Curtobacterium sp. HT1-7, Herbiconiux sp. 11R-BC, Herbiconiux sp. 11RB-1, Herbiconiux sp. 11RB-2 Sphingobium sp. 11R-BB, Rahnella sp. WP4-4-2, Rahnella sp. WP4-5-3, Curtobacterium sp. WP4-3-3, Curtobacterium sp. WP4-10-4, Sphingobium sp. (WW5), Curtobacterium salicis (WW7), Pseudomonas sp. M2, Pseudomonas sp. M5, Rhizobium sp. PTD1, and Pseudomonas sp. WP8.

Many of the endophyte strains discussed above have been demonstrated to be newly discovered endophyte strains from pioneer tree and plant species: Rhizobium wenxinide. HT1-8, Azospirillum palustre SD2; Azotobacter beijerinckii SD1, Rahnella aceris R10, Rahnella aceris WPL8-1, Rahnella aceris WP5, Pseudomonas lutea WP4-4-6, and Rhodotorula graminis WP4-3-1, Duffyella gerundensis M3.

### Full Genome Sequence and Safety Analyses

The genomes of these endophyte strains were fully sequenced and analyzed for genes encoding proteins and/or phenotypes known to be harmful to plants or animals. The method of Varghese et al. was used to analyze the whole genome sequences of the endophyte strains. See https://www.ncbi.nlm.nih.gov/pmc/articles/PMC4538840/. The method reliably classifies strains as safe or potential pathogens through comparisons of the genome sequence to known harmful and pathogenic microbes for similarities in the genome. If the organisms fall within a cluster represented by human or plant pathogens, then the presence of virulence factors and antibiotic resistance genes are then assessed. No such pathogen species relatedness was found for any of these strains. To further confirm that the strains were safe for agricultural food crops, the genomes of the endophyte strains were analyzed for genes known to be associated with plant and human pathogens. No pathogen-related genes were found in any of the selected endophyte strains. Finally the genomes of the endophyte strains were analyzed using the program pathogen finder v 1.0 https://www.ncbi.nlm.nih.gov/biosample/docs/packages/Pathogen.cl.1_0/, and all the endophytes were found to be likely non-pathogenic FIG. 2.

### Biochemical Analyses for Safety

Additionally, the endophyte strains were analyzed to determine whether they exhibited any biochemical similarities with known pathological biochemistry in mammalian bacterial pathogens. The endophyte strains were grown on various media for comparison against common bacteria that are associated with known pathogens in mammals. The media chosen were MacConkey Agar (MAC), Mannitol Salt Agar (MSA), and Sheep's Blood Agar (SBA) (Hardy Diagnostics, Santa Maria, CA). These media are often used in the isolation and differentiation of mammalian pathogens, but it is important to note that commensal and mutualistic plant endophytic bacteria also produce a variety of results on these different plate mediums.

MAC is used for isolating and differentiating between Gram negative bacilli based on their ability to survive the crystal violet dye and utilize lactose. Strains that survive the medium will typically exhibit growth within 24-48 hours in the normal use case. The breakdown of lactose is detected via the formation of acidic byproducts, which turns the medium bright pink due to the neutral red pH indicator. Strains that grow and turn the medium pink are considered positive for this test. Strains that exhibit growth but do not turn the medium pink, and strains that do not grow, are all considered to be negative for this test.

MSA is typically used to isolate and differentiate between Gram positive cocci. The selective agent of this medium is 7.5% sodium chloride allowing halotolerant microbes to survive and grow on the medium. MSA contains a high concentration of the sugar mannitol relative to other carbon sources in the medium and phenol red as a pH indicator. Strains that survive the medium will typically exhibit growth within 24-48 hours in the normal use case. Halotolerant organisms that can utilize the mannitol will produce acidic metabolic byproducts. This turns the medium yellow and is considered a positive result. A negative result would include organisms that can survive the medium but do not turn the medium yellow, or organisms that do not survive the medium.

SBA is a differential medium used to encourage growth of fastidious clinical isolates and differentiate strains based on their hemolytic pattern (their ability to break down red blood cells). This medium does not contain any selective ingredient or pH indicator. Incubation for 24-48 hours at 37° C. is typically sufficient in the normal use case. A positive result is determined via the clearing of red blood cells in the medium in one of two different patterns. The first positive pattern, β-hemolysis, is the complete clearing of the red blood cells in the agar around the colonies with no red, green, or brown pigment remaining in the cleared area. The second positive pattern, α-hemolysis, is the partial clearing of the red blood cells around the colonies that results in a greenish-brown pigment in the medium. A negative result for this test would be growth without any clearing of the red blood cells in the medium, or growth of the microbe but no evidence of clearing the red blood cells. The latter negative result is traditionally called “γ-hemolysis”.

Each endophyte strain was inoculated onto MAC and MSA and grown at 25.5° C. for 24-48 hours. If the strains survived the medium, they were evaluated for their ability to utilize lactose on MAC and mannitol on MSA and were reported as positive (+) or negative (−) for the test. If the strains were unable to grow on the medium, they were reported as having no growth (NG). Each strain was also inoculated onto SBA and grown at 37° C., then evaluated at 24 and 48 hours for growth and any developed hemolytic patterns. Results are summarized in FIG. 2.

All strains that survived MAC were unable to utilize the lactose in the medium. Only one strain, Priestia megaterium (AWS1), a salt tolerant endophyte survived on the MSA and was able to utilize the mannitol in the medium. Many strains were unable to grow on SBA at 37° C. but were almost all non-hemolytic, of those that were able to grow, only AWS1 produced hemolytic activity, and it was a small partial cleared zone or beta-hemolysis immediately adjacent the colonies at 24 hrs under 37° C. conditions.

The strains were also analyzed for pathogenicity using PathogenFinder V1.0 web server that analyzes bacterial whole-genome sequence data to predict the potential for human pathogenicity. It uses protein families to determine if a bacterium is likely to be pathogenic or non-pathogenic to humans and leverages the growing database of sequenced bacterial genomes from both pathogenic and non-pathogenic strains to inform its likely pathogenic predictions.

The results cumulatively confirmed that no similarities to mammalian bacterial pathogens were found in the endophyte strains, and that the four strains of endophyte bacteria are safe for application to agricultural food crops and other applications. The results of the biochemical assays discussed above and the pathogen finder WGS safety analyses are provided in FIG. 2.

The screened, identified endophyte strains were developed into stocks through novel fermentation growth methods for use in novel inoculant formulations. The formulations are used to treat and improve non-native host plant species (those in which the endophytes do not naturally occur). The inoculant compositions of the present technology include additional constituents that promote long-term stability (long shelf life), delivery, colonization in the host plant, and efficacy.

The inoculant compositions of the present technology may include liquid seed treatments, seed coatings, freeze-dried powdered re-constitutable seed treatments, encapsulated dry beads, foliar sprays, in-furrow liquid products, and other formulations.

Compositions including the non-native endophytes may be “heterologously” applied to various plant species and agronomic crops, meaning that the applied endophyte strains are not naturally occurring in the treated host plant. Important agricultural, ornamental, and other host plant varieties and species can be heterologously treated with the endophyte strains of the present technology. A fundamental tenant of plant breeding, including genetically modified plant breeding, commences with the production of “clean” germplasm. This typically means free of microbes. As such, most propagation material for annual crops is a microbiological vacuum. For perennial plants, modern propagation methods adopt various clean up procedures that also result in germplasm free of microbes. In contravention of conventional practices, the technology disclosed herein provides bacterial endophytes to heterologous monocotyledonous and dicotlyledonous plants that result in the plants outperforming plants that do not contain these beneficial endophyte strains.

In some implementations, host plant treated with endophyte inoculant as disclosed herein may be plants that are cultivated by humans for food, feed, fiber, fuel, and/or industrial purposes, and may include, but are not limited to, wheat (e.g., Triticum aestivum, Triticum spelta, Triticum monococcum, Triticum dicoccum, Triticum durum, Triticum turgidum, and Triticum rigidum), corn (e.g., Zea mays including subspecies such as Zea mays indenata, Zea mays indurata, Zea mays amylacea, Zea mays saccharata, and Zea mays everta), soy (e.g., Glycine max), cotton (e.g., Gossypium arboretum, Gossypium herbaceum, Gossypium hirsutum, Gossypium barbadense), broccoli (e.g., Brassica oleracea italica), kale (e.g., Brassica oleracea acephala), tomatoes (e.g., Solanum lycopersicum), rice (e.g., Oryza sativa), barley (e.g., Hordeum vulgare), beets (e.g., Beta vulgaris), peas (e.g., Pisum sativum), potatoes (e.g., Solanum tuberosum), sugarcane (e.g., Saccharum officinarum), bananas (e.g., Musa acuminata and Musa balbisiana), spinach (e.g., Spinacia oleracea), lettuce (e.g., Lactuca sativa), zucchini (e.g., Cucurbita pepo), peppers (e.g., Capsicum annuum) rape seed (e.g., Brassica napus), alfalfa (e.g., Medicago sativa), conifers (e.g., Pseudotsuga menziesii, Pinus taeda, and Alnus rubra), salicaceae (e.g., Salix sitchensis, Salix nigra), Populus (e.g. Populus trichocarpa, Populus nigra, Populus deltoides and all Hybrid Populus crosses DxT TxN DxNXT DxN etc.), eucalyptus (e.g., Eucalyptus rostrata, Eucalyptus tereticornas, Eucalyptus cladocalyx and Eucalyptus globulus), rosaceae (e.g., Malus domestica, Pyrus communis, Prunus avium, Prunus dulcis, Prunus persica, Prunus armeniaca and Prunus americana). The endophyte strains may be applied in various settings, including to host plants grown under greenhouse or field conditions and in a variety of cultural methods. The endophytes strains may be applied mechanically, manually, through irrigation, through artificial inoculation, and generally by disposition onto or into a plant, plant element, plant tissue, seed, seedling, or onto or into a plant growth medium such that the treatment exists on or in the plant, plant element, plant tissue, seed, seedling, or plant growth medium in a manner not found in nature.

In various implementations, the heterologous application may be to a non-native host plant variety, to a plant at a stage in plant development in which the endophyte strain(s) are not naturally present or in a growth environment in which the same endophyte stains(s) are not naturally present. For example, an endophyte strain that is naturally found in stem tissue of a willow tree is considered heterologous to any tissue of a maize, spring wheat, cotton, soybean plant that naturally lacks such endophyte strain. In some embodiments, non-naturally occurring application may be the presence of the non-native endophyte in the host plant tissue or in the tissue of a different plant element, tissue, cell type, or other physical location in or on the plant than that which is naturally occurring. For example, if an endophyte species or strain is heterologously disposed where an endophyte is normally found in the root tissue of a plant element but not in the leaf tissue, is applied to the leaf.

A “host plant” includes any plant, particularly a plant of agronomic importance, to which a non-native endophyte can be heterologously applied. The detectable inclusion of endophyte strains in a host plant may result in improved growth characteristics, stress resistance, and/or other characteristics of the host plant. The endophyte strains also improve agricultural traits in crop plant varieties, such as yield and nutritional composition of harvested portions of the crop plants in comparison to untreated plants having no non-native endophyte strains. The non-native endophyte may colonize a host plant or element thereof when it can be stably detected within the plant or plant element over a period time, such as periods of days, weeks, months, or years.

Specific formulations of the heterologous endophyte strains provide unique inoculant benefits to plant hosts that enhance over all plant growth, health, yield, and quality, reduce plant resistance to biotic and abiotic stress, and prevent infection via induced plant resistance to plant/seed diseases and pests. Some of these endophyte strains have both NUE and phosphorus uptake efficiency (PUE) capabilities. These particular strains can serve dual purposes in treating crop plant to reduce both fertilizer and pesticide applications.

The ability of the endophyte strains to colonize non-native plant hosts has been experimentally demonstrated through various methods, including polymerase chain reaction (PCR) analyses on host plant tissues for specific genetic markers and 16s sequencing of each endophytic strains, detection of specifically selected diazotrophic colony-forming units (CFU) isolated from surface sterile host plant tissues, genetic mcherry, RFP, CFP, YFP or GFP fluorescent marker tagging for laser fluorescence confocal microscopy localization, and other appropriate methods. The presence of the genetic material of the endophyte strains in the host plant, CFU in the host plant tissue, and other physiological measures such as increased chlorophyll leaf content, enhanced root branching and growth, increased shoot biomass, enhanced leaf mineral nutrient ion content, have all demonstrated successful colonization of host plants by the endophyte strains.

Additionally, biochemical and molecular analyses have characterized the modes of action through which the endophyte strains improve the performance, yield, mineral nutrition and health of the host plants. These analyses include, but are not limited to, acetylene reduction assays, growth on nitrogen-free medium, measuring exogenous ammonia and ammonium production using a quantitative probe plus meter and chemical test kits, nifH and nifD nitrogenase gene specific PCR, ICPMS shoot ion concentration profiling of leaf tissues, quantifying exogenous insoluble phosphorus mobilization in liquid cultures using fluorescent dyes in a spectrophotometric plate reader, measuring Fe-siderophore production on CAS plates, and bioinformatics and genomics of genetic pathways responsible for these biochemical traits. Endophyte colonization in non-native heterologous host plants results in measurable improvements in the uptake of nitrogen, phosphorus, potassium, and other macro- and micronutrients, enhance nutrient use efficiency, photosynthesis, growth, yield, carbon sequestration, and general plant health.

A host plant comprising one or more endophyte strains in its tissues exhibits detectable changes in the content of at least one nutritional trait and this improvement may be passed on through asexual propagation (e.g., a cutting of stems, roots, or leaves, layering, division, separation, grafting, budding, and micropropagation.) or through seeds. The resulting offspring of the endophyte-associated host plant or a tissue therein may have one or more endophyte strains within their tissues and at least one increased nutritional quality trait when compared with untreated plants of the same species. The offspring of root stock, cuttings, or tissue culture produced cultivars may exhibit such phenotypic traits and enhanced performance because of the presence of the heterologously endophytic strain(s) in their tissue. The levels of a nutritional trait may be measured in an asexually propagated offspring, a seed, or an offspring grown from a seed of the host plant and compared with the levels of the nutritional quality trait in a comparable tissue from a reference agricultural plant not comprising the heterologous endophyte strain(s). The presence or improvement of a phenotypic trait in an asexually propagated or germinated offspring of a host plant may be measured by various methods, including, but not limited to increased height, overall biomass, root mass, shoot biomass, seed germination, seedling survival, photosynthetic efficiency, improved nitrogen use efficiency (NUE), improved phosphorus use efficiency (PUE), increased macronutrient content, increased micronutrient content, seed/fruit number or mass, fruit yield, leaf chlorophyll content, photosynthetic rate, root length, abiotic stress resistance, biotic stress resistance, disease resistance, wilt recovery, turgor pressure, or any combination thereof, as compared to an untreated control plant of the same species, grown under similar conditions.

The selected endophyte strains HT1-2, HT1-4, HT1-7, HT1-8, HT1-9, HT1-10, SD1, SD2, R10, 11R-BC, 11R-BB, 11RB-1, 11RB-2, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8, and WPL8-1, for use in treating host plants were developed into stocks through microbial fermentation processes. Microbial stock maintenance and fermentation methods may be used to increase the expression of nitrogenase genes and maintain the plasmids in their active forms. The endophyte strains may be grown in bacterial growth media having limited nitrogen and other specialized characteristics (e.g., chelated iron and/or magnesium) to enhance the atmospheric nitrogen fixation and provide other beneficial features of the endophyte strains. Nitrogenase upregulation may be induced by growing and fermenting the endophyte strains in nitrogen-limited media (NLM—microbiological media that contains multiple carbon sources, buffering salts, indispensable metals, acidification inhibitor and a limited nitrogen source from amino acids) or nitrogen-free growth media (NFM—microbiological media that contains multiple carbon sources, buffering salts, indispensable metals, acidification inhibitor and no provided nitrogen source), which primes the endophyte strains for increased atmospheric N₂ absorption and assimilation. See, e.g., the following reference regarding nitrogen-free examples: R. J. Rennie, A single medium for the isolation of acetylene reducing (dinitrogen-fixing) bacteria from soils, Canadian Journal of Microbiology, vol. 27, no. 1, pp. 8-14, 1981.

Additionally, nitrogenase enzyme activity and/or gene expression may be further enhanced through pairing at least one strong diazotrophic endophyte strain together with at least one compatible helper or synergistic nitrogenase booster strain. Such upregulation may result in higher levels of nitrogenase gene expression (e.g., nifH, nifD, nifK, nifE, nifN, nifB), and/or alternative nitrogenase like genes (e.g., Fe—S clusters) and is measurable through PCR analysis. Increased nitrogenase enzyme activity can also be measured using the acetylene reduction assay (ARA).

In other implementations, a rich media may be utilized for fermentation of the endophyte strains, which is a microbiological media that contains at least one carbon source at greater concentrations than in NFM or NLM media, buffering salts, and high concentrations of amino acids and nitrogen.

The fermentation broth utilized to ferment the endophyte strains may include various constituents to allow for growth and health of the endophyte strains during the fermentation process. The Nitrogen-Free or Nitrogen-Limited Medias used in the fermentation process may include one or more salts, such as sodium chloride, phosphate salts (e.g., monopotassium phosphate, dipotassium phosphate, and other phosphate salts), sulfate salts (e.g., MgSO₄), chloride salts (e.g., CaCl₂)), and other appropriate salts, but excluding nitrates, ammonium salts, and other sources of nitrogen. The fermentation solution may further include other appropriate constituents, such as yeast extract, agar, and other appropriate ingredients. The resulting composition may be utilized as a liquid composition for treating a host plant. See, e.g., the following reference regarding nitrogen-limited media examples: R. J. Rennie, A single medium for the isolation of acetylene reducing (dinitrogen-fixing) bacteria from soils, Canadian Journal of Microbiology, vol. 27, no. 1, pp. 8-14, 1981. In order to drive upregulation of microbial nitrogenase genes in the endophyte strains, the Nitrogen-Free or Nitrogen-Limited Medias may be virtually free from nitrogen. However, after fermentation in the NLM broth, the resulting novel composition includes one or more of nitrogen constituents in limited amounts such as physiological ranges of 30-100 mg NH3, or NH₄⁺/L, amino acids such as glutamate, glutamine, histidine, etc., and other nitrogen-containing compounds such as nitrate, nitrite, carbamic acid, and other nutritional constituents. FIG. 3A provides specific examples of other constituents that may be in the fermented composition.

Phosphorus (P) is a vital macronutrient required for plant growth, playing critical roles in processes such as energy transfer, photosynthesis, and nutrient uptake. However, the majority of phosphorus in soil exists in insoluble forms, such as calcium phosphate, iron phosphate, and aluminum phosphate, which are unavailable for plant absorption. Some of the endophyte strains are phosphate-solubilizing endophytes (“PSEs” such as AWS1, SD1, HT1-10, HT1-8, TP-SK5, TP-SN7, WW7, WP4-5-3, PM SK6, WP1, WP5, and others) make a series of organic acids or other insoluble phosphorus (P) mobilizing compounds that are able to mobilize these insoluble phosphate forms, improving plant P bioavailability and enhancing plant performance. The endophytes live extracellularly inside the apoplast, between plant cells of the vasculature inside roots, stalks, stems, and branches. The endophytes in stalks and stems exude these compounds to keep the phosphorus from binding to other metals. These strains can also assist in solubilizing potassium (K), and other macronutrient ions (SO₄²⁻ and Ca²⁺), converting them into soluble forms similarly to the way P is mobilized and acquired by endophytes and roots. The P and K root associated mobility are both affected by the inoculated bacterial endophytes through acidification, chelation, and ion exchange reactions.

In agricultural soils, insoluble phosphate predominantly exists as calcium phosphate in alkaline soils, iron phosphate in acidic soils, and aluminum phosphate under conditions with high aluminum content. These forms are tightly bound to soil minerals, rendering them inaccessible to plants. Phosphate-solubilizing endophytes can address this challenge through metabolic processes that solubilize and mobilize phosphate. PSEs produce organic acids such as gluconic acid, citric acid, malic acid, and oxalic acid, which chelate metal ions like calcium, iron, and aluminum, releasing phosphate ions into the soil solution. Additionally, PSEs produce phytosiderophores and enzymes like phosphatases which facilitate the mineralization of organic phosphorus compounds, further enhancing P availability.

An endophyte strain's ability to solubilize insoluble forms of phosphorus (e.g., Ca₃(PO₄)₂) from the soil through the production of various mobilizing compounds enhances the ability of crops to biogeochemically access free phosphate which is the plant root bioavailable form. For example, chelating agents and pH-reducing acidic compounds produced by the endophytes can facilitate better phosphate and other mineral access for plant roots and improve the uptake of and translocation of these nutrients from soil into roots and up into shoots. The mobilization of phosphorus by endophytes yields substantial benefits to host plants. Enhanced phosphorus uptake stimulates root development, increasing root length, biomass, and surface area. This improved root architecture allows plants to access water and other nutrients more effectively, particularly in nutrient-limited or saline soils. Furthermore, phosphorus is integral to ATP synthesis, enabling robust energy transfer for processes like photosynthesis, nutrient translocation, and stress tolerance. Endophyte-inoculated plants exhibit improved chlorophyll content, shoot biomass, and overall growth performance. This improvement demonstrates a synergistic relationship between plants and endophytes, which not only solubilize phosphate but also promote other plant growth traits and stress mitigation.

The mobilization of insoluble phosphate by endophyte strains, along with the absorption and uptake of solubilized phosphorus by the host crop plants from soil or rock, can be utilized to reduce P fertilizer crop input requirements. These phosphate acquisition mechanisms significantly improve the metabolic performance of the treated host plants, leading to increased biomass, stress tolerance, and other beneficial traits.

Some of the endophyte strains (e.g., SD1, WP4-4-2, WP4-5-3, WW7, WW5, and WW6) also make exogenous extracellular iron siderophore compounds that the plant then further exudes out from its roots, that are used to mobilize insoluble micronutrient mineral ions or metals such as iron, magnesium, zinc, copper, nickel, manganese, molybdenum and other divalent macro nutrient cations like calcium Ca₂₊ from the soil and to help keep them mobile once inside the root, which allows for better root to shoot transport and assimilation.

Combinations of endophyte strains, including co-fermented combinations of two or more endophyte strains disclosed herein may be applied to host plants to provide an increased benefit or additional benefits to the host plant, as compared to the benefits provided by application of a single endophyte. For example, one endophyte strain that induces a benefit in the host plant may induce such a benefit equally well in a plant that is also colonized with a different endophyte strain that also induces the same benefit or an additional benefit in the host plant. In some cases where two or more endophyte strains are heterologously applied to the same host plant, the host plant can experience a greater increase in a particular nutritional trait, growth trait, stress tolerance, and overall health of the host plant that exceeds an expected improvement in a trait, indicating a synergistic effect of the application of a plurality of endophyte strains to the host plant, including synergistically increasing nitrogen fixation in a combination of the endophyte strains to increase nitrogen assimilation in the host plant. Examples 1E-1F, 2E, 4C, and 44-54 provide data demonstrating synergistic effects in heterologous applications of multiple endophyte strains. The presently disclosed combinations of endophyte strains do not show incompatibility with each other or in the host plant, which can occur with endophyte strains other than those disclosed herein.

One or more additional constituents may be included in the inoculant compositions that improve the performance of the heterologous endophyte strains and to enhance effective application to and colonization in a range of host plants. The endophyte strain(s) of the present technology (e.g., HT1-9, HT1-2, 11R-BB, SD1, SD2, WP5, WP4-4-2, WP4-4-6, WP4-5-3, WW5, WW6, WW7, PTD1 etc.) are able to heterologously colonize a non-native host plant. Molecular and microbiological analyses performed on the tissues of treated host plants demonstrate that the endophyte strains applied heterologously to the non-native host plants via the inoculant compositions of the present technology have colonized the host plant and been established in the tissues of the host plant.

### Compositions

The inoculant compositions of the present technology are provided in liquid suspensions, seed treatments and coatings, foliar sprays, freeze-dried reconstitutable formulations, and solid forms (e.g., in-furrow, granular spray-dried/air-dried beads). The inoculant compositions of the present technology may include heterologous endophyte strains HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1, and combinations thereof. More specifically, the inoculant compositions may include an effective amount of one or more of the HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1, strains and one or more additional constituents to stabilize and improve the uptake and viability of the endophyte strains to enable practical use and application of the endophyte strain(s) to seeds, roots, stems, leaves, flowers, bulbs, and other structures of a non-native host plant. The inoculant compositions of the present technology provide for the promotion of plant vigor, health, growth, yield, and abiotic and biotic stress resistance. In some embodiments, the composition of the present technology may include additional endophyte or microbial species, such as additional beneficial Rhizobium strains, Curtobacterium species, Mycorrhizae species, Bacillus species, Priestia species, Azotobacter species, Azospirillum species, Sphingobium species, Herbiconjux species, Rhanella species, Pseudomonas species, or biocontrol bacterial species (e.g., Erwinia, Rhanella, Pseudomonas, Bacillus, and Paraburkholderia) endophytic yeast strains (Rhodotorula), and other beneficial microbial strains. In some embodiments, the inoculant composition may include endophyte Rhodotorula graminis yeast strain WP1.

Compositions may comprise one or more constituents that facilitate delivery, shelf-life, and/or efficacy of the applied endophyte strains and may include a surfactant, a buffer, a carrier, a tackifier, a microbial stabilizer, mineral or clay granule, a nutrient, an excipient, a wetting agent, and/or a salt. The additional constituents may exclude compounds that include amine, amides, and other nitrogen groups, to maintain a low nitrogen or substantially nitrogen-free environment for the endophyte strains.

The present compositions may be formulated to be shelf-stable, including liquid, suspension, and solid formulations. Shelf-stable formulations may include suspension formulations, dry formulations, powder formulations, and formulations comprising dried endophyte strains. The compositions may be shelf-stable for at least 3 weeks or longer under pre-determined conditions. For example, the composition may be stable for 10 weeks or longer at a variety of temperatures, including low temperatures (at or around freezing temperatures), at sustained high temperatures, or room temperature under standard temperature and pressure (STP) conditions. In some examples a liquid concentrate formulation of an endophyte fermentate or inoculum may be prepared using microbial filters, light heating to evaporate water from the liquid, or bulk centrifugation to make a semi-solid bacterial paste.

The formulations may include one or more dried endophyte strains that have a moisture content of the endophyte strains is reduced to 30% or less compared to undried endophyte strains. In some implementations, one or more endophyte strains included in the inoculant compositions may be freeze-dried. In other implementations, the endophyte strains may be dried using other methods, such as air drying, desiccation, and/or spray drying. The dried endophyte strains in the inoculant composition may enhance stability of the endophyte strains therein. In some embodiments, the formulation may contain dried endophyte strains and may be substantially stable at temperatures between about −20° C. and about 50° C. for at least about 4 weeks, and up to one or more years. In some embodiments the formulation contains a partially hydrated shell surrounding the endophytes in a carbohydrate carrier such as sodium alginate, calcium alginate, or magnesium alginate or other appropriate carbohydrate carrier (e.g., Scogin LDH), providing a hard, mostly dehydrated round or oval bead. The beads can range in sizes from about 400 nm to about 5 mm in average diameter, a range that includes nanobeads, microbeads, and capsule beads. The beads may additionally or alternatively contain thickeners, starch, carbohydrate, or mineral thickener, stabilizers and/or carriers.

In some embodiments, the dried endophytes may be used in various formulations deliverable to a host plant. In some embodiments, dried endophytes may be used in encapsulated granules. The dry endophyte formulation may be mixed with a dry granular solid or seeds and then the dry formulation may be encapsulated using binder such as hydroxypropyl cellulose to bind endophytes onto the dry granular solid (e.g., phosphate fertilizer, biochar, calcined clay, or seed). In other embodiments, the dried endophytes may be mixed with a dry carrier (e.g., insoluble, inert ingredients such as dolomite, kaolin clay, pyrophyllite, bentonite, montmorillonite, diatomaceous earth, acid white soil, vermiculite, and pearlite, and inorganic salts such as calcium carbonate) to form suspension granules. In other embodiments, the dried endophytes may be mixed with a dry carrier a fine solid carrier such as silicates or alumino silicates comprising single lattice or double lattice clays, and other appropriate carriers to form water-dispersible granules. In other embodiments, the dried endophytes may be milled to a fine power with the particle sizes from about 0.200 μm to about 500 μm and then mixed with a dry powder (e.g., graphite, talc, soybean protein powder) to form a dustable powder operable to lubricate and relieve static and friction of seeds or other substrates. In other embodiments, the dried endophytes may be milled to a fine power with the particle sizes from about 0.200 μm to about 500 μm and passed through an ion-rich region by applied voltage (0-20 kV) causing the endophyte formula to be charged which causes the individual particles of the formulation to repel each other to form an electrostatic powder operable to increase uniformity of coating on to solid a dry granular solid. In other embodiments, the dried endophytes may be milled to a fine power with the particle sizes from about 0.200 μm to about 500 μm and may be mixed with a wettable carrier, a surfactant, a wetting agent, a dispersant, and/or adjuvant as described herein to form a wettable powder formulation.

In some embodiments, the inoculant composition may include a stabilizer compatible with the endophyte strain(s) and that promotes the viability of the strains, and application to and colonization of the heterologous endophyte strains in a host plant. Examples of suitable stabilizers include guar gum, xantham gum, agarose, sucrose, glucose, ficoll, phytogel, sodium alginate, calcium alginate, magnesium alginate, glycine betaine, methyl cellulose, maltodextrin, molasses, and mixtures thereof. Additional usable stabilizers include one or more of trehalose, sucrose, glycerol, and methylene glycol, glucose, sucrose mineral oil, soy lecithin, peptone, monopotassium phosphate (KH₂PO₄), dipotassium phosphate (K₂HPO₄), hydroxypropyl-guar (HP-Guar), xantham gum polyvinylpyrrolidone, polyvinylpyrrolidone/vinyl acetate (PVP-VA), non-reducing sugars and sugar alcohols such as mannitol or sorbitol, and other suitable materials. The amount of the stabilizer in the composition may be in a range from about 5 wt % to about 50 wt % (e.g., between about 10 wt % to about 40 wt %, between about 15 wt % and about 35 wt %, between about 20 wt % and about 30 wt %, or any value or range of values therein).

In some embodiments, the composition may include a carrier such as an agriculturally acceptable carrier, which may be any material that can be added to a plant element without causing or having an adverse effect on the host plant or element thereof. The carrier can be a solid carrier or liquid carrier, and in various forms including microspheres, powders, emulsions, wide variety of polymers, dried powdered fertilizers such as potash, potassium phosphate, potassium nitrate, or other appropriate materials. The carrier may be any one or more of several carriers that confer a variety of properties, such as increased stability, wettability, flowability, and/or dispersibility.

In some embodiments, the agricultural carrier may be a solid, such as diatomaceous earth, loam, silica, magnesium silicate, alginate (e.g., sodium, calcium, or magnesium alginate), glycine betaine (natural or synthetic), clay, bentonite, biochar, vermiculite, seed cases, peat, wheat, bran, talc, lime, starch, cellulose (methylcellulose hemicellulose) fuller's earth, pasteurized soil, fertilizer powders or fertilizer salts (macro and micro nutrients), other plant, animal, or abiogenic products, or combinations thereof, including granules, pellets, or suspensions. In some embodiments, the solid carriers of a treatment formulation include, for example, mineral carriers such as dolomite, kaolin clay, pyrophyllite, bentonite, montmorillonite, diatomaceous earth, acid white soil, vermiculite, and pearlite, and inorganic salts such as calcium carbonate. Also, organic fine powders such as wheat flour, wheat bran, and rice bran may be used solid carriers. Mixtures of any of the ingredients are also contemplated as carriers, such as but not limited to, pasta (flour and kaolin clay) or flour-based pellets in loam, sand, or clay, etc. In some embodiments, the agricultural carrier may be soil or a plant growth medium, and/or food sources for the cultured organisms. In a particular embodiment, the endophyte strain may be encapsulated in calcium alginate, magnesium alginate, agarose, or other appropriate material with or without one or more carbohydrate stabilizers, such as sucrose, glucose, or other appropriate sugars. The encapsulated endophyte strains may be included in a suspension liquid or solid formulation able to be used in seed treatments and coatings, foliar applications, in-furrow applications, as a powdered fertilizer coating for all fertilizers, macronutrients, and micronutrients, including but not limited to, granular urea, ammonium nitrate, potassium nitrate, potassium phosphate, calcium phosphate and other implementations.

In some embodiments, the agricultural carrier may be a liquid carrier that confers a variety of properties, such as increased stability, wettability, flowability, and/or dispersibility. Liquid carriers may include vegetable oils such as soybean oil, neem oil, cottonseed oil, and other compositions such as glycerol, ethylene glycol, polyethylene glycol, propylene glycol, polypropylene glycol, and other suitable liquids. In some embodiments, the carrier may be a combination of liquid constituents, such as a water-in-oil emulsion, or other appropriate formulations. For example, water-in-oil emulsions may be prepared to include wettable powders, granules, gels, agar, thickeners, biopolymers, microencapsulated particles, and the like. Other agricultural carriers that may be used include water, plant-based oils, humectants, or combinations thereof. The composition may include wetting agents such as natural or synthetic surfactants, which can be nonionic or ionic surfactants, or a combination thereof. When such formulations are used as wettable powders, biologically compatible dispersing agents such as non-ionic, anionic, amphoteric, or cationic dispersing and emulsifying agents can be used.

In some embodiments, the surfactants that can be included in the composition may include nonionic and/or anionic surfactants. Examples of nonionic surfactants include alkylphenol alkoxylates, alcohol alkoxylates, polyoxyethylene glycerol fatty acid esters, castor oil alkoxylates, fatty acid alkoxylates, fatty amide alkoxylates, fatty polydiethanolamides, lanolin ethoxylates, fatty acid polyglycol esters, isotridecyl alcohol, fatty amides, methylcellulose/hemicellulose, fatty acid esters, alkyl polyglycosides, glycerol fatty acid esters, polyethylene glycol, polypropylene glycol, polyethylene glycol/polypropylene glycol block copolymers, polyethylene glycol alkyl ethers, polypropylene glycol alkyl ethers, polyethylene glycol/polypropylene glycol ether block copolymers, polyethylene oxide/polypropylene oxide block copolymers, and mixtures thereof. Examples of anionic surfactants include alkylaryl sulfonates, phenyl sulfonates, alkyl sulfates, alkyl sulfonates, aryl alkyl sulfonates, alkyl ether sulfates, alkylaryl ether sulfates, alkyl-polyglycol ether phosphates, polyaryl phenyl ether phosphates, alkyl-sulfosuccinates, olefin sulfonates, paraffin sulfonates, petroleum sulfonates, taurides, sarcosides, salts of fatty acids, alkyl-naphthalene sulfonic acids, naphthalene sulfonic acids and ligno sulfonic acids, condensates of sulfonated naphthalenes with formaldehyde or with formaldehyde and phenol and, if appropriate, urea, and also condensates of phenol sulfonic acid, formaldehyde and urea, lignosulfite waste liquors and lignosulfonates, alkyl phosphates, alkylaryl phosphates, for example tristyryl phosphates, and also polycarboxylates, such as, for example, polyacrylates, maleican hydride/olefin copolymers, including the alkali metal, alkaline earth metal, and mixtures thereof.

In some embodiments, the inoculant composition can include a tackifier or adherent for aiding in combining the endophyte strains with carriers that can contain other compounds that are not biologic. Such compositions help create coatings around the plant or plant element (e.g., for use in a seed coating) to maintain contact between the heterologous endophyte(s) and other materials with the plant or plant element. In some embodiments, adherents may include one or more of alginate, gums, starches, maltodextrin, lecithins, formononetin, polyvinyl alcohol, alkali formononetinate, hesperetin, polyvinyl acetate, cephalins, Gum Arabic, Xanthan Gum, carrageenan, PGA, other biopolymers, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Hemi Cellulose, Gum Ghatti, polyoxyethylene-polyoxybutylene block copolymers, and other suitable agents.

In some embodiments, one or more adjuvants may be used in the inoculant composition to help improve the delivery and performance of the endophyte strain(s). The composition can be combined with adjuvants to the creation of a particular product form or mixture, such as a liquid mixture for foliar applications. The composition can further comprise other agronomically suitable excipients such as solvents, pH modifiers, viscosity modifiers (rheology modifiers), crystallization inhibitor, antifoam agents, dispersing agents, wetting agents, humectants, anticaking agent, suspending agents, spray droplet modifiers, pigments, antioxidants, UV protectants, compatibilizing agents, sequestering agents, neutralizing agents, corrosion inhibitors, dyes, odorants, spreading agents, penetration aids, lubricants, sticking agents, thickening agents, freezing point depressants, antimicrobial agents, and the like. The composition content of these auxiliary excipients is not particularly limiting and may be determined by a skilled technician in the art according to the conventional protocols.

In some embodiments, suitable pH modifiers that can be included in the inoculant composition include buffers, such as alkali metal salts of weak inorganic or organic acids, such as, for example, phosphoric acid, phosphorous acid, boric acid, acetic acid, propionic acid, citric acid, fumaric acid, tartaric acid, oxalic acid, malic acid, oxalacetic acid, and succinic acid.

The inoculant composition may further include food sources for the cultured organisms, such as barley, rice, wheat, or other biological materials such as seed, plant elements, sugar cane bagasse, hulls or stalks from grain processing, ground plant material or wood from building site refuse, sawdust or small fibers from recycling of paper, fabric, or wood.

The inoculant composition may further include additional components used to enhance plant health and microbial survival, such as biostimulants, prebiotics, amino acids, fatty acids, plant proteins, fungicides, insecticides, nematicides, plant microbial boosters (prebiotic), plant hormones and elicitors, mineral macronutrients and micronutrients (liquid and dry), seed treatment polymers, commonly used dyes, carbohydrates and gels (alginate, mucilage, agarose, guar, xantham gum, etc.) powdered carriers (soy protein, talc, lime, starch biochar, cellulose/hemicellulose, silica, clay, nanotechnology structures including mineral nutrients, such as carbon dots buckyballs, carbon cages or other forms of nanotech, etc.). Exemplary biostimulants may include amino acid combinations (e.g., one or more of L-glutamine, L-lysine, L-methionine, L-arginine, and L-threonine), fatty acids, vitamins, plant proteins, phosphorous source, betaines, plant growth factors, and other appropriate constituents. Formulations of the endophyte strains discussed herein were combined with commercial biocide and nutrient products to test the viability of the endophytes under such conditions. The endophytes were found to be compatible with several such commercial products in laboratory and field settings. Several novel formulations were developed in view of the compatibility testing results.

The endophyte strains described herein can be combined with one or more of the agents described above to yield a composition suitable for application to a plant or tissue thereof, seedling, seed, or other plant element. The endophyte populations can be obtained from selection and growth in culture as described herein and added to the composition. Endophytes at different growth phases can be used. For example, endophytes at lag phase, early-log phase, mid-log phase, late-log phase, or stationary phase can be used.

The foregoing constituents may be combined in a liquid composition that may include effective amounts of one or more of endophyte strains HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1, For example, the inoculant compositions disclosed herein may include endophyte strains in an amount between about 0.1 to 90% by weight, for example, between about 1% and 80%, between about 5% and 70%, between about 10% and 60%, between about 15% and 50% in wet weight of the composition. The inoculant composition may include at least about 10³ CFU per mL to about, for example, at least about 10⁴ CFU per mL, at least about 10⁵ CFU per mL, at least about 10⁶ CFU per mL, at least about 10⁷ CFU per mL, at least about 10⁸ CFU per mL, at least about 10⁹ CFU per mL, at least about 10¹⁰ CFU per mL, or any value or range of values therein. In some embodiments, the inoculant composition may include 10¹ CFU/mL to 10¹⁰ CFU/mL, from 10² CFU/mL to 10¹⁰ CFU/mL, from 10³ CFU/mL to 10¹⁰ CFU/mL, from 10³ CFU/mL to 10¹⁰ CFU/mL, from 10³ CFU/mL to 10⁹ CFU/mL, from 10⁵ CFU/mL to 10⁸ CFU/mL. from 10⁶ CFU/mL to 10⁹ CFU/mL. from 10⁷ CFU/mL to 10⁹ CFU/mL. or from 10⁸ CFU/mL to 10⁹ CFU/mL.

An exemplary liquid formulation according the present invention may include two or more dried or undried endophyte strains (e.g., HT1-9 and 11R-BB prepared by through co-fermentation) at a concentration of about 10⁸ CFU/mL to about 10⁹ CFU/mL of each endophyte strain, one or more mono- or disaccharides (e.g., sucrose) in an amount of about 1 wt % to about 10 wt %, mannitol in an amount of about 1 wt % to about 10 wt %, sodium lactate in an amount of about 0.01% v/v to about 0.1% v/v, potassium phosphate salts (e.g., K₂HPO₄ and KH₂PO₄) in an amount of about 0.05 wt % to about 0.5 wt %, sodium molybdate in an amount of about 0.001 wt % to about 0.01 wt %, NaCl in an amount of about 0.005 wt % to about 0.05 wt %, CaCl₂) in an amount of about 0.005 wt % to about 0.05 wt %, Na₂FeEDTA in an amount of about 0.001 wt % to about 0.01 wt %, magnesium sulfate in an amount of about 0.01 wt % to about 0.1 wt %, yeast extract in an amount of about 0.005 wt % to about 0.05 wt %, and agar in an amount of about 1 wt % to about 10 wt %, all in distilled water. A further exemplary liquid formulation may include one or more dried or undried endophyte strains at a concentration range of about 10⁴-10¹⁰ CFU/mL of each strain alone or combined with one or more of the following: a low viscosity alginate (e.g., sodium alginate, magnesium alginate, calcium alginate, Scogin® LDH (Dupont), or other high purity alginate) in an amount of about 0.1% v/v to about 5% wt %, glycerol in an amount of about 0.1% v/v to about 5% v/v, and mono- and disaccharides (e.g., glucose and lactose) in an amount of about 1 wt % to about 10 wt %. A still further exemplary liquid formulation may include one or more dried or undried endophyte strains at a concentration range of about 10⁴-10¹⁰ CFU/mL of each strain alone or combined with one or more of the following: a low viscosity alginate (e.g., sodium alginate, magnesium alginate, calcium alginate, Scogin® LDH, (Dupont), or other low viscosity high purity alginate) 0.5-50% w/v, gelatin in an amount of about 1% v/v to about 5% wt %, PEG in an amount of about 1% v/v to about 10% v/v, and mono- and disaccharides (e.g., glucose and lactose) in an amount of about 1 wt % to about 10 wt %.

In some embodiments, the composition may be a suspension formulation including the foregoing constituents in the proportions described above. In such embodiments, the composition may further comprise one or more solid carriers, thickening agents, or bulking agents. Such constituents may include inorganic mineral earths, such as silica gels, silicates, talc, kaolin, Atta clay, limestone, lime, chalk, loess, clay, dolomite, diatomaceous earth, calcium sulfate and magnesium sulfate, magnesium oxide, attapulgite, montmorillonite, mica, vermiculite, synthetic silicic acids, amorphous silicic acids and synthetic calcium silicates, or mixtures thereof; and/or organic carriers, such as hydrocolloids, polymers, cellulose, methyl-cellulose, and/or hemicellulose powders and combinations thereof. The suspension composition may further comprise humectants, emulsifiers, anticaking agent, suspending agents, freezing point depressants, and the like. In some embodiments, the suspension formulation may include one or more of endophyte strains HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1, in the concentrations disclosed in the foregoing paragraph. An exemplary suspension formulation according to the present invention may include one or more dried endophyte strains at a concentration of about 10⁸ CFU/mL to about 10⁹ CFU/mL of each endophyte strain that are microencapsulated in sodium, calcium, or magnesium alginate (e.g., through a spray-drying process). The formulation may further include one or more mono- or disaccharides (e.g., sucrose) in an amount of about 0.1 wt % to about 10 wt %, and glycerol in an amount of about 0.1 wt % to about 20 wt %, all in distilled water.

In some embodiments, the composition may be a solid composition. The solid composition may be a dry, granulated, or flowing composition intended for dispersion or suspension in aqueous solution prior to delivery to plants. Dry fertilizer compositions may form a thoroughly dispersed suspension. In other contexts, dry fertilizer compositions may provide for slow release (as by low water-solubility or by encapsulation, e.g., sodium alginate), such as when the steady or controlled delivery of nutrients over time is desired. The solid composition may include an amount of endophyte strains HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8, and WPL8-1, in a range of about 10³ CFU per mL to at least about 10¹⁰ CFU per mL, or any value or range of values therein. For example, the solid composition may include one or more of the endophyte strains HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1, in an amount at a concentration of about 10⁸ CFU/mL to about 10⁹ CFU/mL of each endophyte strain. The solid formulation may comprise one or more solid carriers in an amount in a range of about 30 wt % to about 60 wt % (e.g., an amount in a range of about 40 wt % to about 55 wt %, an amount in a range of about 45 wt % to about 99.9 wt %, or any value or range of values therein). An exemplary solid formulation according to the present invention may include one or more dried endophyte strains at a concentration of about 10⁴ CFU/mL to about 10¹⁰ CFU/mL of each endophyte strain that are microencapsulated in alginate beads (sodium, calcium or magnesium 0.1-10% w/v) and a solid (starch 0.1-10% w/v) and dripped through a proprietary slurry formulation, batch drip, ion exchange and fluid bed drying process. The formulation may further include one or more mono- or disaccharides (e.g., sucrose) in an amount of about 1 wt % to about 10 wt %, and a clay (e.g., zeolite, bentonite, and/or other clay materials) in an amount of about 30 wt % to about 50 wt %.

### Manufacturing

The endophyte strains of the presently disclosed methods and formulations (HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1) may be prepared for inclusion in various formulations, including liquid, and dry formulations that may be applied to foliar tissues, stems, seeds, roots, and/or the soil in proximity to a seed or plant. Specifically, the inoculant compositions of the present technology may include liquid seed treatments, seed coatings, freeze-dried wettable powdered that can be re-constituted as a seed treatment or other application, spray-dried wettable powdered that can be re-constituted as a seed treatment or other application, encapsulated dry beads, foliar sprays, in-furrow liquid products, and other formulations. The endophyte strains may be fermented individually or may be co-fermented in various combinations of the endophyte strains. The endophyte strains were developed into stocks through novel fermentation growth methods for use in inoculant formulations. To stabilize the endophyte strains during fermentation, sufficient mineral nutrients, vitamins, carbon sources, aeration, temperature and duration may be utilized. Additionally, constituents that promote long-term stability (long shelf life), delivery, colonization in the host plant, and efficacy may be included in the inoculant compositions of the present technology.

In some implementations, the selected HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1 cells may be grown for application purposes in nitrogen free or nitrogen-limited media for 1-3 days individually until a target concentration of the endophyte strain(s) is present in a range of about 10⁷ CFU/mL to about 10¹⁰ CFU/mL. In some implementations, two or more endophyte strains may be combined and co-fermented to produce a fermentate having combined concentrations in a range of about 10³ CFU per mL to about 10⁹ CFU per mL, e.g., at least about 10⁴ CFU per mL, at least about 10⁵ CFU per mL, at least about 10⁶ CFU per mL, at least about 10⁷ CFU per mL, at least about 10⁸ CFU per mL, at least about 10⁹ CFU per mL, or any value or range of values therein. The fermentation process conditions may include a pre-determined incubation temperature in a range of about 20° C. to about 30° C. (e.g., about 23° C. to about 26° C., about 25° C., or any value or range of values therein), shaking the fermentation vessels at a rate in a range of about 25 rpm to about 300 rpm (e.g., about 75 rpm to about 250 rpm, about 125 rpm to about 225 rpm, about 200 rpm, or any value or range of values therein), and fermentation of volumes of about 1 L to about 10 L (e.g., about 2 L to about 8 L, about 4 L to about 6 L, about 4 L, about 2 L, or any value or range of values therein).

In order to drive upregulation of microbial nitrogenase genes in the endophyte strains, the fermentation media may be a Nitrogen Free (NF) or Nitrogen-Limited Media (NLM) that is virtually free from nitrogen, but may include one or more sugars, such as mannitol, mannose sucrose, glucose, fructose, lactose, and other appropriate sugars. The NF/NLM may also include one or more salts, such as sodium chloride, phosphate salts (e.g., monopotassium phosphate, dipotassium phosphate, and other phosphate salts), sulfate salts (e.g., MgSO₄), chloride salts (e.g., CaCl₂)), and other appropriate salts, but excluding nitrates, ammonium salts, and other sources of nitrogen. The fermentation solution may further include other appropriate constituents, such as yeast extract, agar, and other appropriate ingredients. The resulting composition may be utilized as a liquid composition for treating a host plant. See, e.g., the following reference regarding nitrogen-limited media examples: R. J. Rennie, A single medium for the isolation of acetylene reducing (dinitrogen-fixing) bacteria from soils, Canadian Journal of Microbiology, vol. 27, no. 1, pp. 8-14, 1981.

The fermentation process may end when the endophyte strains reach a stationary phase in which the microbial growth slows, and the microbes enter a semi-dormant state. Sterile water may be added to the fermentate in order to dilute the remaining sugars and nutrients to increase microbial shelf life, placing microbes in a dormant stationary phase, and reduces CO₂ formation.

Methods of producing the inoculants of the present technology, including for foliar and stem applications, seed applications, root applications, and soil and in furrow applications are discussed in detail below in the Examples, particularly Examples 2A-2D and 65-75.

### Applications

The compositions described herein comprising one or more endophyte strains may be applied to plants to increase the growth characteristics, health, stress resistance, and improve other characteristics of the plant. The compositions disclosed herein may be advantageously applied mechanically or manually or artificially inoculated to a plant or element thereof by any one of a number of means, such as, and without limitation, seed treatment, root wash, seedling soak, soil inoculant, in-furrow application, foliar spraying, foliar coating, side-dress application, wound inoculation, irrigating, fertigating, immersion, injecting, osmo-priming, hydroponics, aquaponics, aeroponics, or any combination thereof. In some embodiments, the compositions can also be applied directly to the plant or part of the plant, for example, a leaf, a root, a foliar, foliage, a tiller, a flower, a plant cell, a plant tissue, or a combination thereof. The compositions can be applied to seeds (e.g., as a coating or by treatment of the seed by spraying or immersion, etc.), and/or applied pre-emergent (before the seedlings emerge or appear above ground). The compositions can also be applied to other propagation materials of plants, such as a grain, some fruit, a tuber, a spore, a cutting, a slip, a meristem tissue, a plant cell, nut, or an embryo. In some examples, the composition may be applied as part a dip for the roots and/or other tissues of the host plant, as a seed coating, as a coating applied to the leaves and/or other elements of the host plant, as a powder to the surface of the leaves and/or other elements of the host plant, as a spray to the leaves and/or other elements of the host plant, as part of a drip to the soil and/or roots of the host plant, or other appropriate methods. The compositions can also be applied to the growth medium (e.g., by applying to the soil around the plants).

Application methods developed may be specific to different uses due to the specific inoculum formulations, and for successful crop plant synergistic combinations and due to the specific rates and methods of application by crop type, these methods used are summarized and provided in the table provided in FIG. 3B.

The inoculant compositions presently disclosed can improve phenotypic traits measured by various methods, including, but not limited to, increased height, overall biomass, total carbon, root mass, shoot biomass, seed germination, seedling survival, photosynthetic efficiency, seed/fruit number or mass, fruit yield, leaf chlorophyll content, photosynthetic rate, root length, abiotic stress resistance, biotic stress resistance, disease resistance, wilt recovery, turgor pressure, or any combination thereof, as compared to an untreated control plant of the same species, grown under similar conditions. Root stock, cuttings, or tissue cultures of the host plants may be used to produce cultivars that exhibit such phenotypic traits and enhanced performance as well. Also, the application of the inoculant composition may increase carbon fixation of the treated host plants. This is an economically attractive benefit, since it results in the removal of carbon dioxide from the atmosphere, as well as increased biomass. Indicators of greater carbon acquisition by the plants include greater CO₂ fixation activity, greater dry weight to fresh weight ratio, and overall biomass.

Treatment with the compositions disclosed herein may result in an increased macro- and micronutrient uptake from the soil and atmosphere. Treatment with the compositions may result in an increased rate of nitrogen uptake. The faster rate of nitrogen uptake also facilitates significant increases in utilization efficiency. Host plants heterologously treated with the compositions of the present show greater total nitrogen taken up, assimilated with a high level of nitrogen utilization efficiency, resulting in more protein production facilitating increased biomass production. The application of the compositions disclosed herein also results in greater nutrient uptake and utilization for other macro- and micronutrients. Experimental results have demonstrated increases in macronutrients nitrogen, potassium, phosphorus, calcium, and magnesium, as well as increased in micronutrients boron, copper, iron, manganese, molybdenum, nickel, sulfur, and zinc uptake in host plants treated with inoculant composition-see, e.g., Examples 8-11 below. The heterologous endophytes can take fixed or recalcitrant forms of certain nutrients in the soil, including phosphorous, and convert them to soluble forms which can be more efficiently utilized by a host plant. The endophyte strains are also able to generate iron siderophores that benefit the host plant, which chelate Fe in the plant tissues, aiding in Fe uptake in the host plant.

The increased macro- and micronutrient uptake is accompanied by increased catabolism carbon uptake, and carbon sequestration. Host plants heterologously treated with the compositions of the present show greater total carbon uptake and associated increases in RuBisCo carboxylation activity, carbon mass, and biomass. The host plants may also exhibit increased production of aromatic amino acids via the shikimic acid pathway. These aromatic amino acids serve as precursors for a wide range of secondary metabolites that are important for plant resistance to biotic and abiotic stress (e.g., oxidative, drought, and/or salt stress).

The application of the composition may thus result in the elevation of host plant adaptive tolerance to abiotic and biotic stress such as disease, cold, salinity, etc. In exemplary embodiments, the level of innate and adaptive tolerance to stress is clearly elevated. Abiotic stressors include temperature extremes, high salinity, drought, and other causes. Abiotic stressors can reduce yields and biomass dramatically and often kill plants. Lower growing temperatures are often encountered in agricultural production of grains, especially during the early parts of the growing season and can stress plants in several ways, beginning with poor germination and followed by stunting of seedling growth, yellowing of leaves, reduced leaf expansion, wilting and tissue death. Cold stress severely inhibits development of reproductive parts of the plant. Crop yield is reduced in response to cold stress in proportion to the extent of the damage to the plants. High salt concentrations in either soil or water are an increasing problem as salt accumulates in irrigated soils and irrigation water with higher salt concentration must be used. The effect of high salt concentrations can be referred to as osmotic stress because the high salt concentrations in soil and water interfere with transport of ions and water within a plant. Symptoms of high salt stress include inhibition of growth, wilting, yellowing, leaf drop, senescence, and death. The improved nutrient uptake and use efficiency resulting from the presence of the endophyte strain(s) in a host plant protects the host plant in the stress-inducing environment, and the host plant exhibit greater growth and biomass, even in abiotic stress conditions.

The application of the composition to the host plant also elevates adaptive tolerance through advanced mineral nutrition, resulting in biotic stress tolerance which inhibits viruses, bacteria, and fungus. The endophyte strains disclosed herein provide resistance in the host plants, which may be the result of activating induced systemic resistance (ISR) in the plants and/or other metabolic mechanisms. Thus, endophyte strains may be applied to a host plant or seed thereof as a nutritional treatment to help protect against pathogenic fungi, viruses, and bacteria.

Other aspects, objects and advantages of the presently disclosed technology are apparent from the detailed description that follows.

### BRIEF DESCRIPTION OF THE DRAWINGS

FIG. 1A shows SEQ ID NO. 1.

FIG. 1B shows SEQ ID NO. 2.

FIG. 1C shows SEQ ID NO. 3.

FIG. 1D shows SEQ ID NO. 4.

FIG. 1E shows SEQ ID NO. 5.

FIG. 1F shows SEQ ID NO. 6.

FIG. 1G shows SEQ ID NO. 7.

FIG. 1H shows SEQ ID NO. 8.

FIG. 1I shows SEQ ID NO. 9.

FIG. 1J shows SEQ ID NO. 10.

FIG. 1K shows SEQ ID NO. 11.

FIG. 1L shows SEQ ID NO. 12.

FIG. 1M shows SEQ ID NO. 13.

FIG. 1N shows SEQ ID NO. 14.

FIG. 1O shows SEQ ID NO. 15.

FIG. 1P shows SEQ ID NO. 16.

FIG. 1Q shows SEQ ID NO. 17.

FIG. 1R shows SEQ ID NO. 18.

FIG. 1S shows SEQ ID NO. 19.

FIG. 1T shows SEQ ID NO. 20.

FIG. 1U shows SEQ ID NO. 21.

FIG. 1V shows SEQ ID NO. 22.

FIG. 1W shows SEQ ID NO. 23.

FIG. 1X shows SEQ ID NO. 24.

FIG. 1Y shows SEQ ID NO. 25.

FIG. 1Z shows SEQ ID NO. 26.

FIG. 1AA shows SEQ ID NO. 27.

FIG. 1AB shows SEQ ID NO. 28.

FIG. 1AC shows SEQ ID NO. 29.

FIG. 1AD shows SEQ ID NO. 30.

FIG. 1AE shows SEQ ID NO. 31.

FIG. 1AF shows SEQ ID NO. 32.

FIG. 1AG shows SEQ ID NO. 33.

FIG. 1AH shows SEQ ID NO. 34.

FIG. 1AI shows SEQ ID NO. 35.

FIG. 1AJ shows SEQ ID NO. 36.

FIG. 1AK shows SEQ ID NO. 37.

FIG. 2 is a table providing exemplary nutritional constituents of a fermented composition.

FIG. 3A is a table identifying exemplary ingredients for culture broth.

FIG. 3B is a table identifying exemplary methods of application and rates of application.

FIG. 4A provides a table associated with the experiments of Example 1A.

FIG. 4B provides a table associated with the experiments of Example 1A.

FIG. 4C provides a table associated with the experiments of Example 1A.

FIG. 4D provides images associated with the experiments of Example 1A.

FIG. 4E provides a table associated with the experiments of Example 1B.

FIG. 4F provides a table associated with the experiments of Example 1C.

FIG. 4G provides a table associated with the experiments of Example 1D.

FIG. 4H provides a table associated with the experiments of Example 1D.

FIG. 4I provides a table associated with the experiments of Example 1D.

FIG. 4J provides a table associated with the experiments of Example 1E.

FIG. 4K provides a table associated with the experiments of Example 1F.

FIG. 4L provides a table associated with the experiments of Example 1G.

FIG. 4M provides a table associated with the experiments of Example 1H.

FIG. 4N provides a table associated with the experiments of Example 1H.

FIG. 4O provides images associated with the experiments of Example 1I.

FIG. 4P provides a table associated with the experiments of Example 1I.

FIG. 4Q provides a table associated with the experiments of Example 1I.

FIG. 4R provides a table associated with the experiments of Example 1J.

FIG. 4S provides a table associated with the experiments of Example 1J.

FIG. 4T provides a chart associated with the experiments of Example 1J.

FIG. 4U provides a table associated with the experiments of Example 1K.

FIG. 4V provides a chart associated with the experiments of Example 1L.

FIG. 4W provides a table associated with the experiments of Example 1L.

FIG. 5A provides a table associated with the experiments of Example 2A.

FIG. 5B provides a table associated with the experiments of Example 2B.

FIG. 5C provides a table associated with the experiments of Example 2C.

FIG. 5D provides a table associated with the experiments of Example 2D.

FIG. 5E provides a table associated with the experiments of Example 2E.

FIG. 5F provides a table associated with the experiments of Example 2F.

FIG. 6A provides a table associated with the experiments of Example 3A.

FIG. 6B provides a table associated with the experiments of Example 3A.

FIG. 6C provides a table associated with the experiments of Example 3B.

FIG. 6D provides a table associated with the experiments of Example 3C.

FIG. 6E provides a table associated with the experiments of Example 3D.

FIG. 6F provides a table associated with the experiments of Example 3E.

FIG. 6G provides a table associated with the experiments of Example 3F.

FIG. 6H provides a table associated with the experiments of Example 3G.

FIG. 6I provides a table associated with the experiments of Example 3H.

FIG. 7A provides a table associated with the experiments of Example 4A.

FIG. 7B is a table providing data associated with the experiments of Example 4B.

FIG. 7C is a table providing data associated with the experiments of Example 4C.

FIG. 7D is a table providing data associated with the experiments of Example 4D.

FIG. 7E is a table providing data associated with the experiments of Example 4E.

FIG. 8A is a table associated with the experiments of Example 5A.

FIG. 8B is a table associated with the experiments of Example 5B.

FIG. 8C is a table associated with the experiments of Example 5C.

FIG. 9A is a table associated with the experiments of Example 6A.

FIG. 9B is a table associated with the experiments of Example 6A.

FIG. 10A is provides gel data associated with the experiments of Example 7A.

FIG. 10B is a table associated with the experiments of Example 7B.

FIG. 10C is a table associated with the experiments of Example 7B.

FIG. 11 is a table associated with the experiments of Example 7C.

FIG. 12 is a table associated with the experiments of Example 7D.

FIG. 13A provides an enzyme pathway associated with the experiments of Example 8A.

FIG. 13B is a table associated with the experiments of Example 8A.

FIG. 13C is a graph associated with the experiments of Example 8A.

FIG. 13D is a graph associated with the experiments of Example 8B.

FIG. 13E is a graph associated with the experiments of Example 8C.

FIG. 13F is a graph associated with the experiments of Example 8D.

FIG. 13G provides an image associated with the experiments of Example 8E.

FIG. 13H provides an image associated with the experiments of Example 8E.

FIG. 13I provides an image associated with the experiments of Example 8E.

FIG. 13J provides an image associated with the experiments of Example 8E.

FIG. 13K provides an image associated with the experiments of Example 8E.

FIG. 13L provides an image associated with the experiments of Example 8E.

FIG. 13M is a graph associated with the experiments of Example 8E.

FIG. 14A provides an enzyme pathway associated with the experiments of Example 8F.

FIG. 14B provides images associated with the experiments of Example 8F.

FIG. 14C is a table associated with the experiments of Example 8F.

FIG. 14D is a graph associated with the experiments of Example 8F.

FIG. 14E is a graph associated with the experiments of Example 8F.

FIG. 15 is a graph associated with the experiments of Example 8G.

FIG. 16 provides images associated with the experiments of Example 9.

FIG. 17A provides images associated with the experiments of Example 10.

FIG. 17B provides images associated with the experiments of Example 10.

FIG. 17C provides images associated with the experiments of Example 10A.

FIG. 17D provides images associated with the experiments of Example 10A.

FIG. 17E provides images associated with the experiments of Example 10A.

FIG. 17F provides images associated with the experiments of Example 10A.

FIG. 17G provides images associated with the experiments of Example 10A.

FIG. 18A is a table associated with the experiments of Example 11.

FIG. 18B is a table associated with the experiments of Example 11.

FIG. 19 is a table associated with the experiments of Example 12.

FIG. 20 is a table associated with the experiments of Example 13.

FIG. 21 is a table associated with the experiments of Example 14.

FIG. 22 is a table associated with the experiments of Example 15.

FIG. 23 is a table associated with the experiments of Example 16.

FIG. 24 is a table associated with the experiments of Example 17.

FIG. 25A is a table associated with the experiments of Example 18.

FIG. 25B is a graph associated with the experiments of Example 18.

FIG. 25C is a graph associated with the experiments of Example 18.

FIG. 26A is a table associated with the experiments of Example 19.

FIG. 26B is a graph associated with the experiments of Example 19.

FIG. 27 is a graph associated with the experiments of Example 20.

FIG. 28A is a graph associated with the experiments of Example 21.

FIG. 28B is a graph associated with the experiments of Example 21.

FIG. 29 is a graph associated with the experiments of Example 22.

FIG. 30A is a table associated with the experiments of Example 23.

FIG. 30B is a table associated with the experiments of Example 23.

FIG. 31A is a table associated with the experiments of Example 24.

FIG. 31B is a graph associated with the experiments of Example 24.

FIG. 31C is a graph associated with the experiments of Example 24.

FIG. 32 is a table associated with the experiments of Example 25.

FIG. 33A is a table associated with the experiments of Example 26.

FIG. 33B is a graph associated with the experiments of Example 26.

FIG. 34A is a table associated with the experiments of Example 27.

FIG. 34B provides images associated with the experiments of Example 27.

FIG. 35 provides images associated with the experiments of Example 28.

FIG. 36A is a table associated with the experiments of Example 29.

FIG. 36B is a graph associated with the experiments of Example 29.

FIG. 36C provides images associated with the experiments of Example 29.

FIG. 36D is a graph associated with the experiments of Example 29.

FIG. 36E is a graph associated with the experiments of Example 29.

FIG. 37A is a table associated with the experiments of Example 30.

FIG. 37B is a graph associated with the experiments of Example 30.

FIG. 38 is a graph associated with the experiments of Example 31.

FIG. 39 is a graph associated with the experiments of Example 32.

FIG. 40A provides images associated with the experiments of Example 33.

FIG. 40B provides images associated with the experiments of Example 33.

FIG. 41 is a graph associated with the experiments of Example 34.

FIG. 42A is a graph associated with the experiments of Example 35.

FIG. 42B is a graph associated with the experiments of Example 35.

FIG. 43A is a graph associated with the experiments of Example 36.

FIG. 43B is a graph associated with the experiments of Example 36.

FIG. 44 is a graph associated with the experiments of Example 37.

FIG. 45A is a graph associated with the experiments of Example 38.

FIG. 45B provides images associated with the experiments of Example 38.

FIG. 45C provides images associated with the experiments of Example 38.

FIG. 45D provides images associated with the experiments of Example 38.

FIG. 46 is a table associated with the experiments of Example 39.

FIG. 47 is a table associated with the experiments of Example 40.

FIG. 48 is a table associated with the experiments of Example 41.

FIG. 49 is a graph associated with the experiments of Example 42.

FIG. 50 is a graph associated with the experiments of Example 43.

FIG. 51 is a graph associated with the experiments of Example 44.

FIG. 52 is a graph associated with the experiments of Example 45.

FIG. 53A is a graph associated with the experiments of Example 46.

FIG. 53B is a graph associated with the experiments of Example 46.

FIG. 54 is a graph associated with the experiments of Example 47.

FIG. 55 is a graph associated with the experiments of Example 48.

FIG. 56 is a graph associated with the experiments of Example 49.

FIG. 57 is a graph associated with the experiments of Example 50.

FIG. 58 is a graph associated with the experiments of Example 51.

FIG. 59 is a graph associated with the experiments of Example 52.

FIG. 60A is a graph associated with the experiments of Example 53.

FIG. 60B is a graph associated with the experiments of Example 53.

FIG. 60C is a graph associated with the experiments of Example 53.

FIG. 61 is a graph associated with the experiments of Example 54.

FIG. 62 is a graph associated with the experiments of Example 55.

FIG. 63 is a graph associated with the experiments of Example 56.

FIG. 64 is a graph associated with the experiments of Example 57.

FIG. 65 provides a graph associated with the experiments of Example 58.

FIG. 66 provides a graph associated with the experiments of Example 59.

FIG. 67A provides a table associated with the experiments of Example 60.

FIG. 67B provides a table associated with the experiments of Example 60.

FIG. 67C provides a table associated with the experiments of Example 60.

FIG. 67D provides a graph associated with the experiments of Example 60.

FIG. 68A provides a table associated with the experiments of Example 61.

FIG. 68B provides a table associated with the experiments of Example 61.

FIG. 69A provides a graph associated with the experiments of Example 62.

FIG. 69B provides a graph associated with the experiments of Example 62.

FIG. 70 provides a graph associated with the experiments of Example 63.

FIG. 71A provides a graph associated with the experiments of Example 64.

FIG. 71B provides a graph associated with the experiments of Example 64.

FIG. 72 provides a graph associated with the experiments of Example 65.

FIG. 73A provides images associated with the experiments of Example 66.

FIG. 73B provides images associated with the experiments of Example 66.

FIG. 73C provides a graph associated with the experiments of Example 66.

FIG. 73D provides a graph associated with the experiments of Example 66.

FIG. 73E provides a graph associated with the experiments of Example 66.

FIG. 73F provides a graph associated with the experiments of Example 66.

FIG. 73G provides a graph associated with the experiments of Example 66.

FIG. 74 provides a graph associated with the experiments of Example 67.

FIG. 75A is a table associated with the experiments of Example 73.

FIG. 75B is a table associated with the experiments of Example 73.

FIG. 76 is a table associated with the experiments of Example 74.

FIG. 77 is a table associated with the experiments of Example 75.

FIG. 78 provides graphs associated with the experiments of Example 78.

FIG. 79 provides gel data associated with the experiments of Example 79.

FIG. 80 is a table associated with the experiments of Example 80.

FIG. 81 provides gel data associated with the experiments of Example 81.

FIG. 82 is a table associated with the experiments of Example 82.

FIG. 83A is a table associated with the experiments of Example 83.

FIG. 83B is a table associated with the experiments of Example 83.

FIG. 83C is a table associated with the experiments of Example 83.

FIG. 83D is a table associated with the experiments of Example 83.

FIG. 83E is a table associated with the experiments of Example 83.

FIG. 84A is a table associated with the experiments of Example 84.

FIG. 84B is a table associated with the experiments of Example 84.

FIG. 84C is a table associated with the experiments of Example 84.

FIG. 84D is a table associated with the experiments of Example 84.

FIG. 84E is a table associated with the experiments of Example 84.

FIG. 85A is a table associated with the experiments of Example 85.

FIG. 85B is a table associated with the experiments of Example 85.

FIG. 85C is a table associated with the experiments of Example 85.

FIG. 85D is a table associated with the experiments of Example 85.

FIG. 85E is a table associated with the experiments of Example 85.

FIG. 86A is a table associated with the experiments of Example 86.

FIG. 86B is a table associated with the experiments of Example 86.

FIG. 86C is a table associated with the experiments of Example 86.

FIG. 87A is a table associated with the experiments of Example 87.

FIG. 87B is a table associated with the experiments of Example 87.

FIG. 87C is a table associated with the experiments of Example 87.

FIG. 87D is a table associated with the experiments of Example 87.

FIG. 87E is a table associated with the experiments of Example 87.

FIG. 87F is a table associated with the experiments of Example 87.

FIG. 88A is a table associated with the experiments of Example 88.

FIG. 88B is a table associated with the experiments of Example 88.

FIG. 88C is a table associated with the experiments of Example 88.

FIG. 89 is a table associated with the experiments of Example 89.

FIG. 90 is a table associated with the experiments of Example 90.

FIG. 91 is a table associated with the experiments of Example 91.

FIG. 92 is a table associated with the experiments of Example 92.

FIG. 93A is a table associated with the experiments of Example 93.

FIG. 93B is a table associated with the experiments of Example 93.

FIG. 94 is a table associated with the experiments of Example 94.

FIG. 95A is a table associated with the experiments of Example 95.

FIG. 95B is a table associated with the experiments of Example 95.

FIG. 95C is a table associated with the experiments of Example 95.

FIG. 95D is a table associated with the experiments of Example 95.

FIG. 95E is a table associated with the experiments of Example 95.

FIG. 95F is a table associated with the experiments of Example 95.

FIG. 95G is a table associated with the experiments of Example 95.

FIG. 95H is a table associated with the experiments of Example 95.

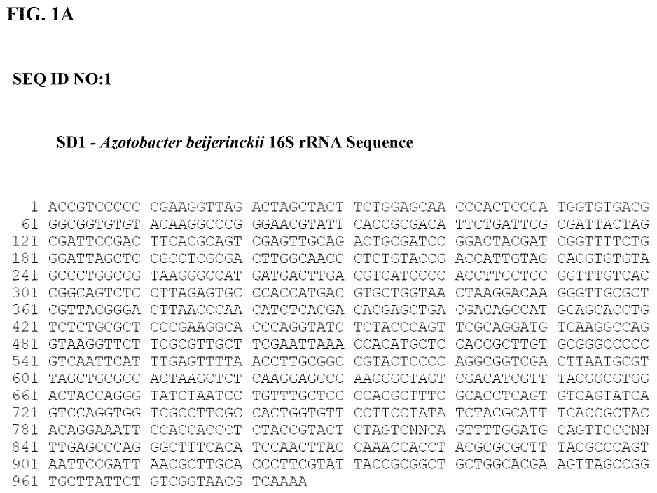

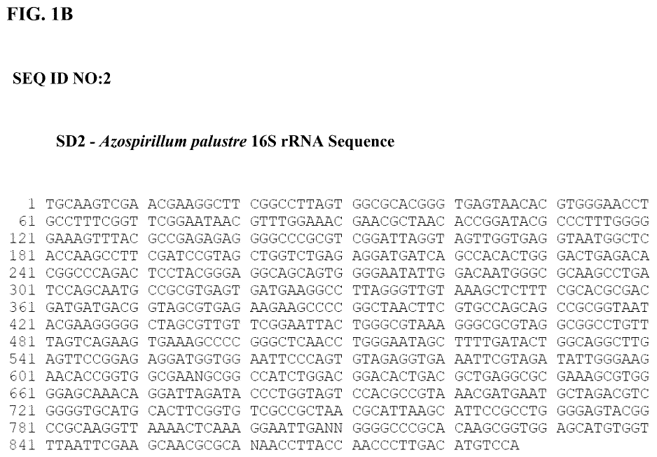

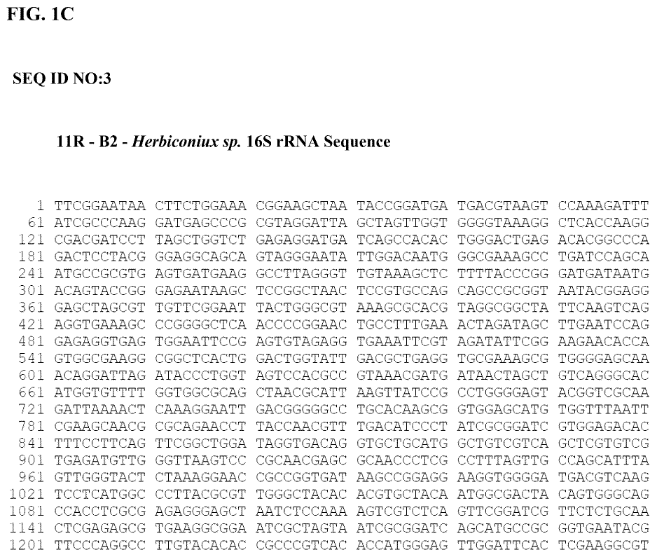

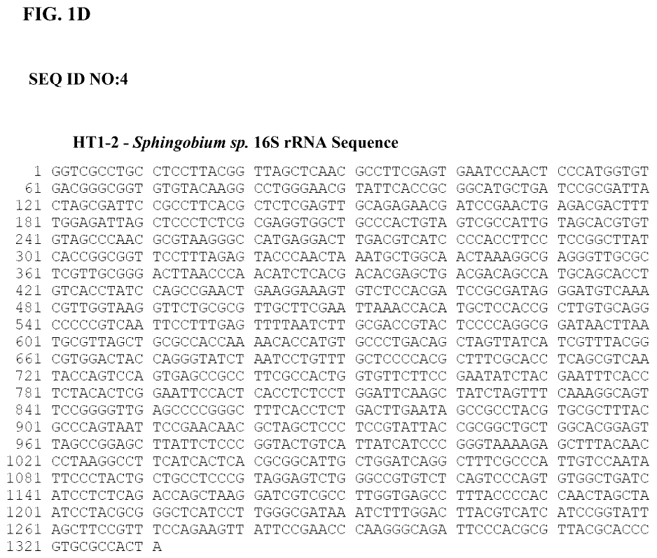

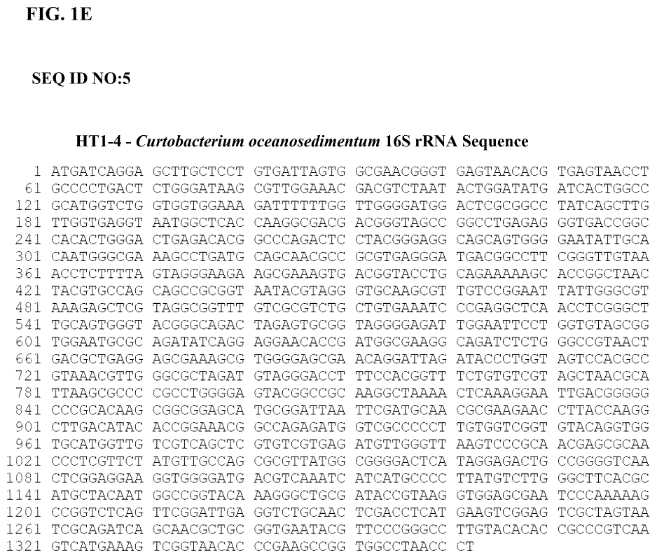

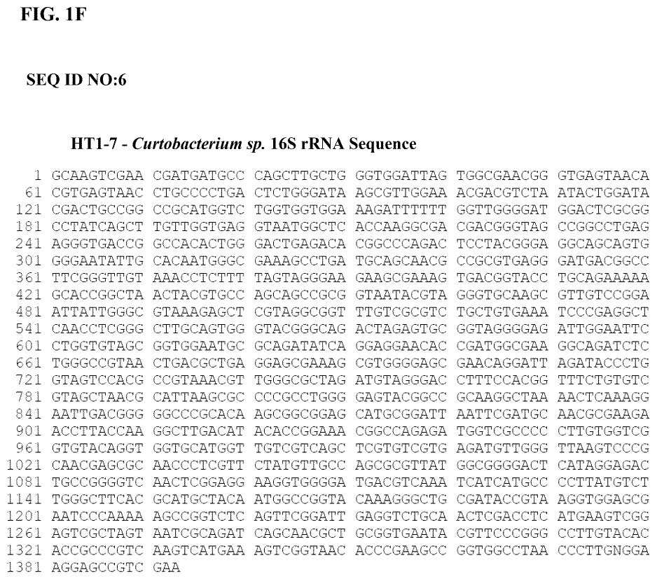

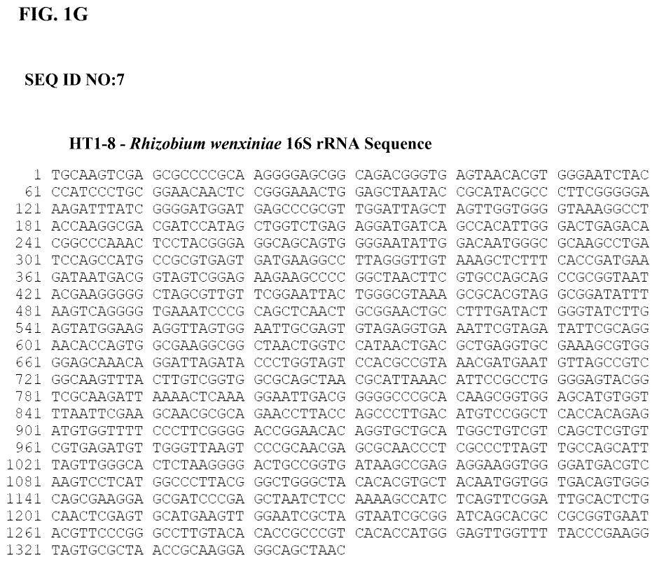

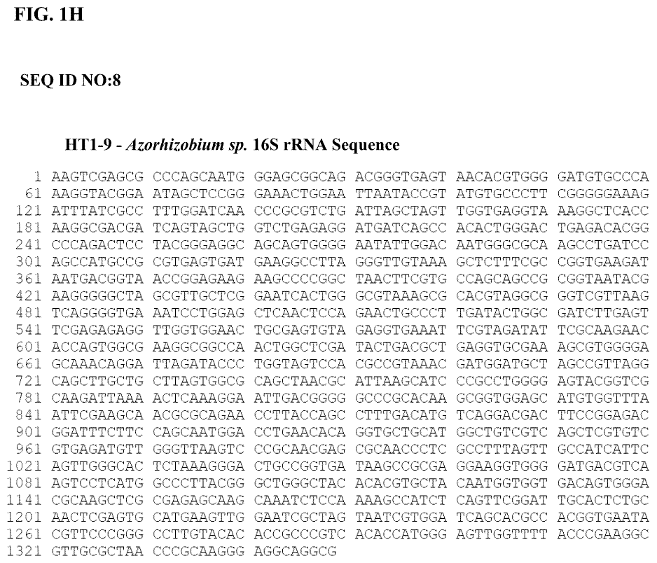

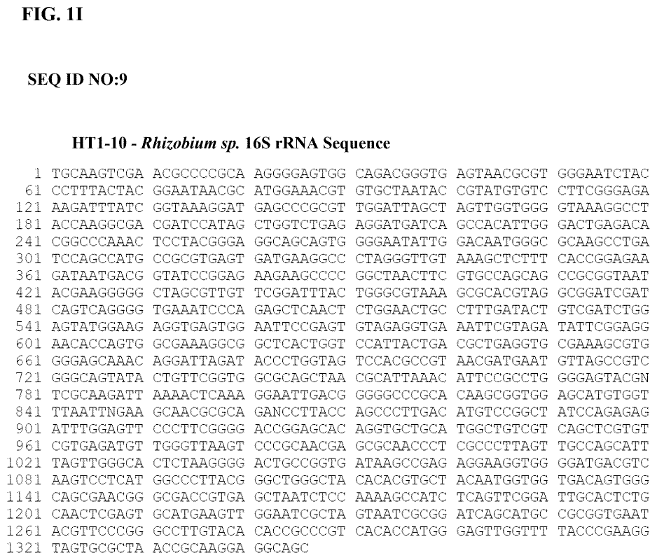

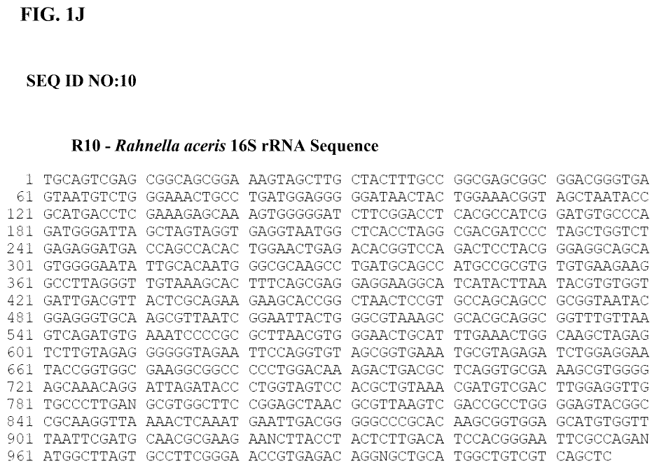

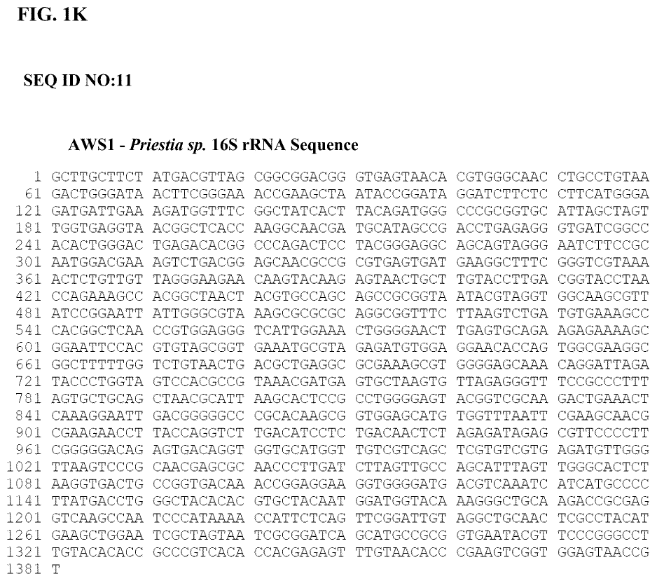

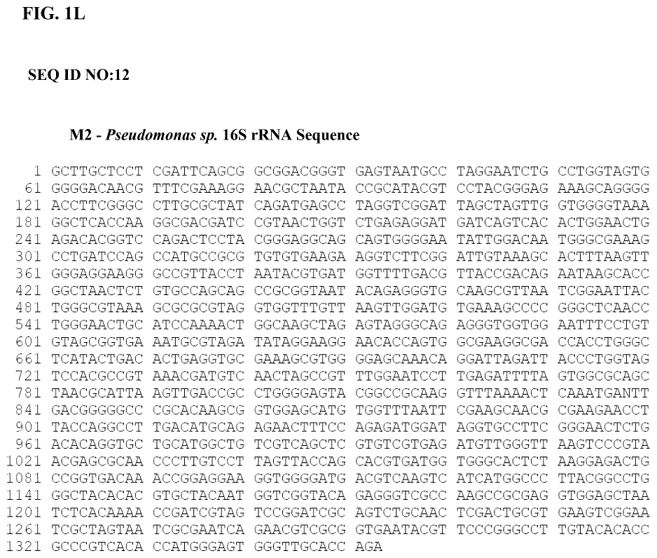

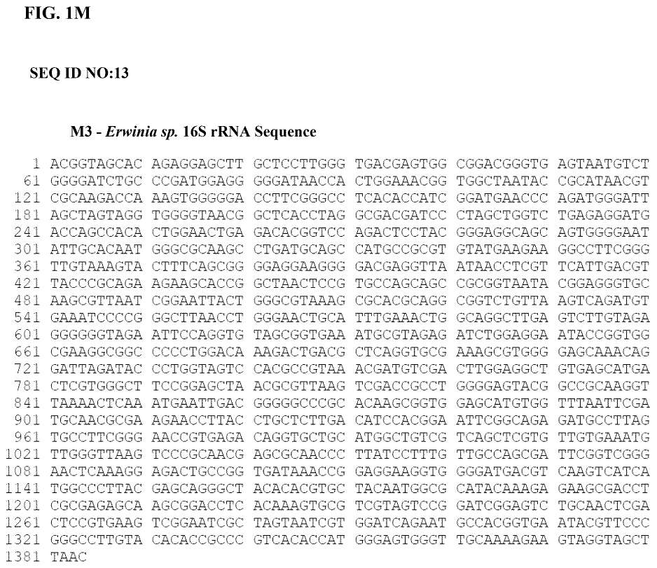

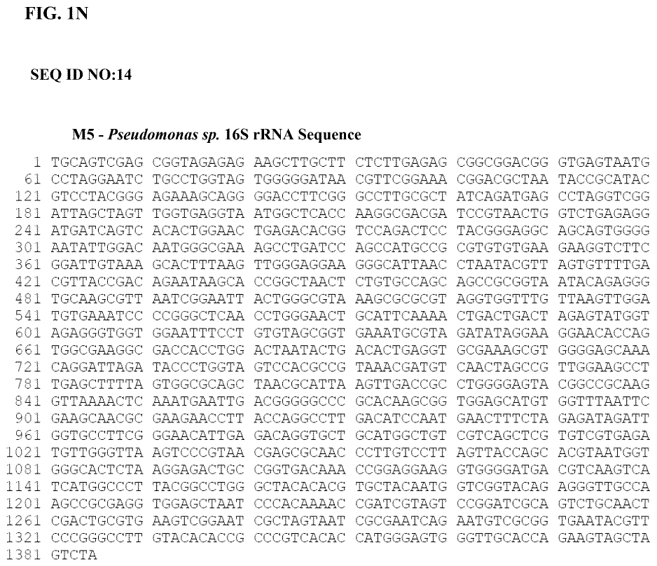

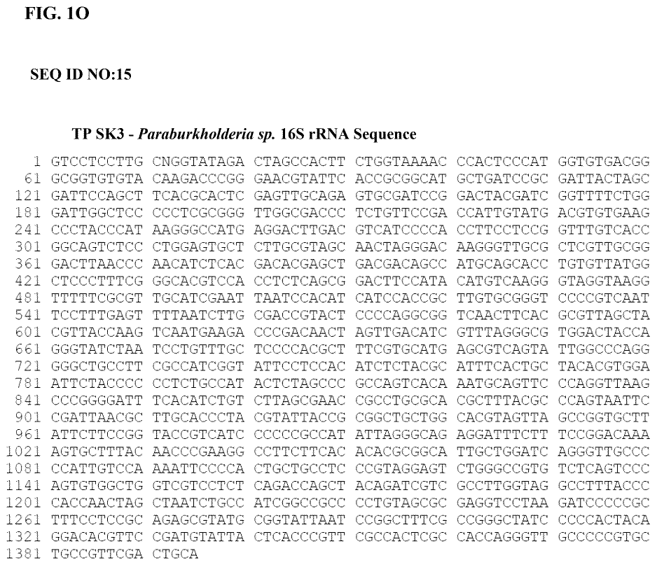

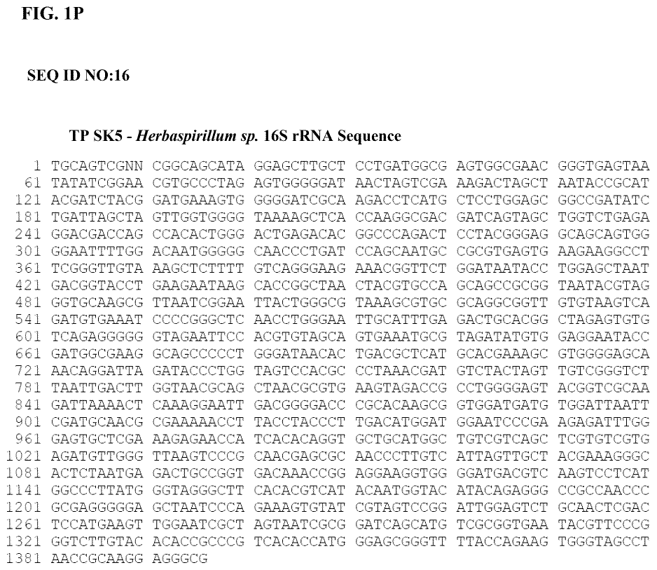

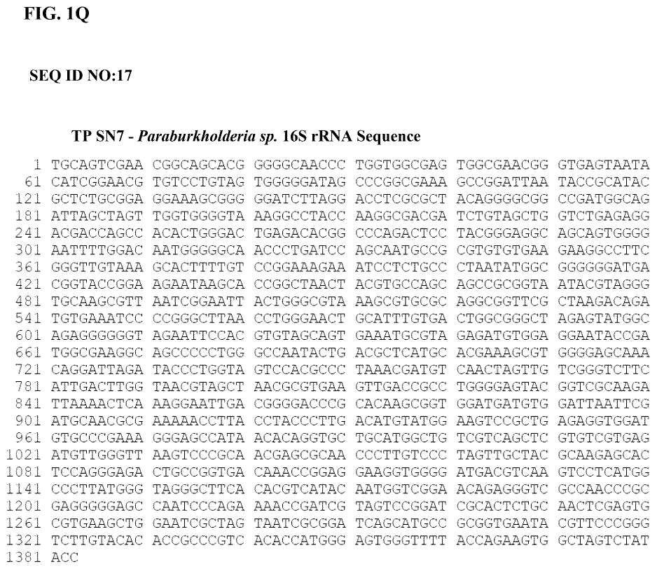

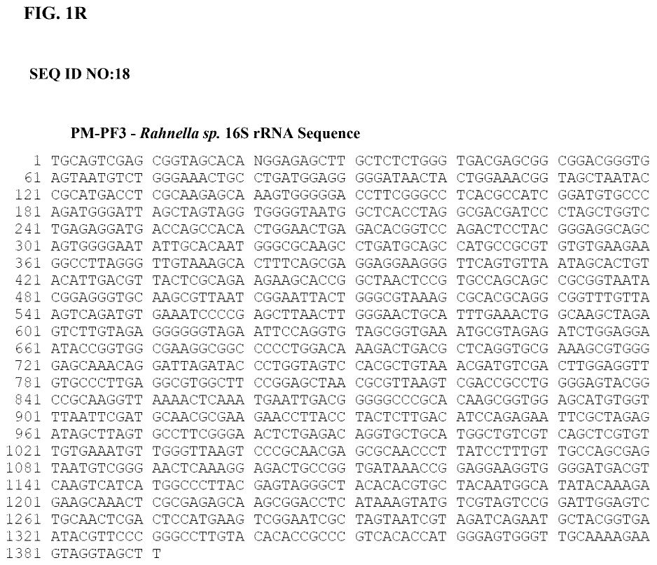

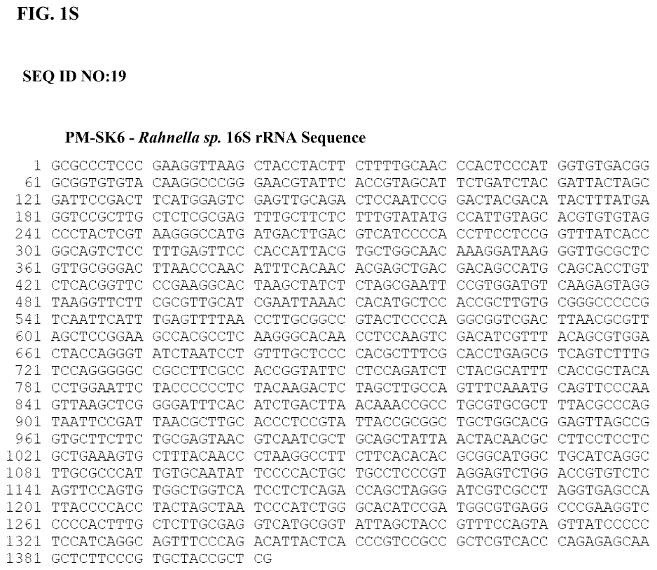

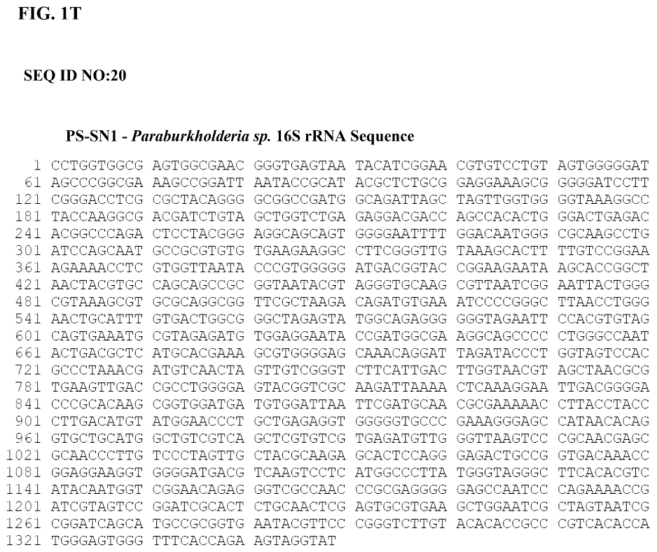

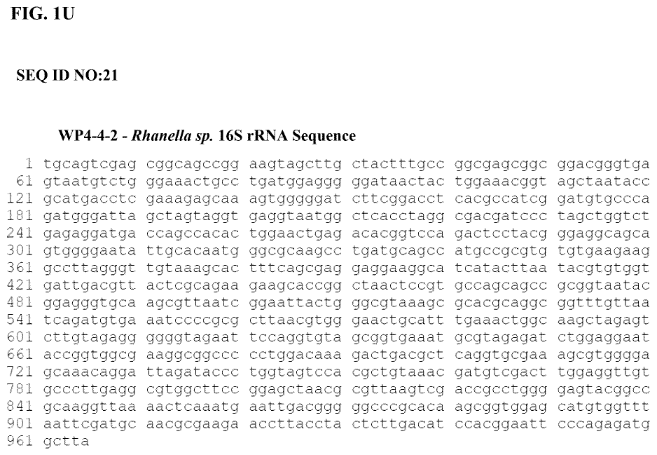

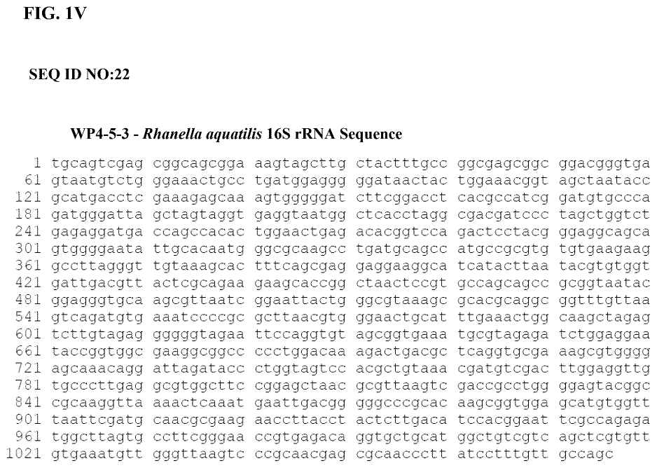

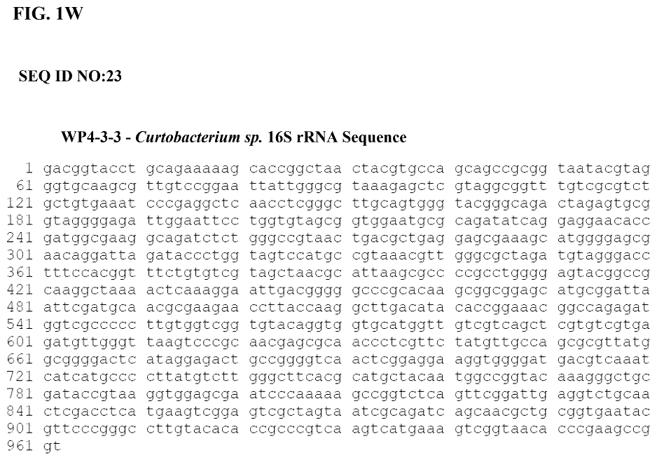

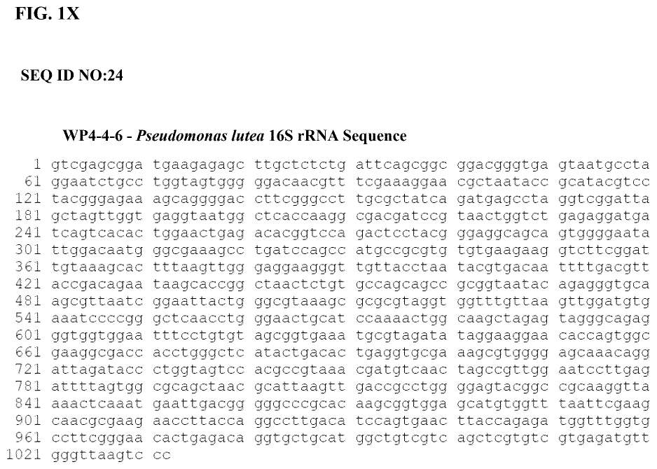

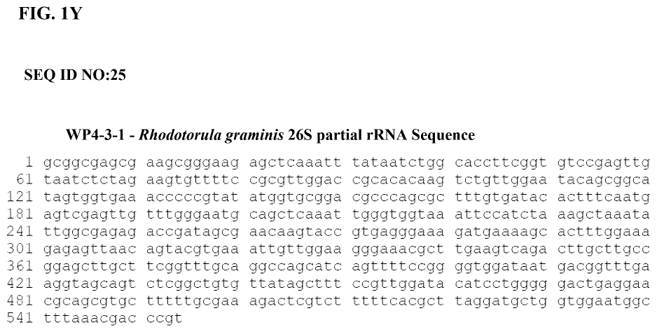

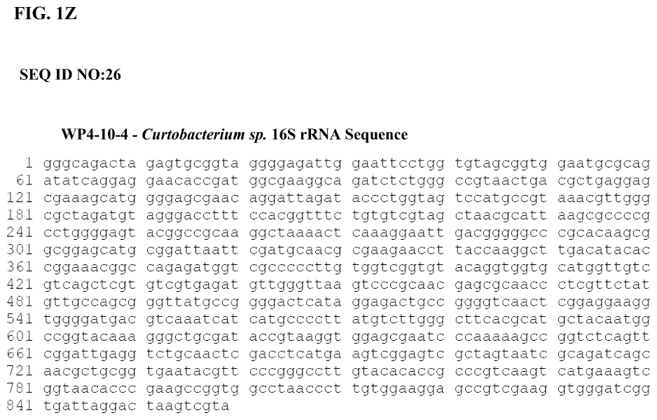

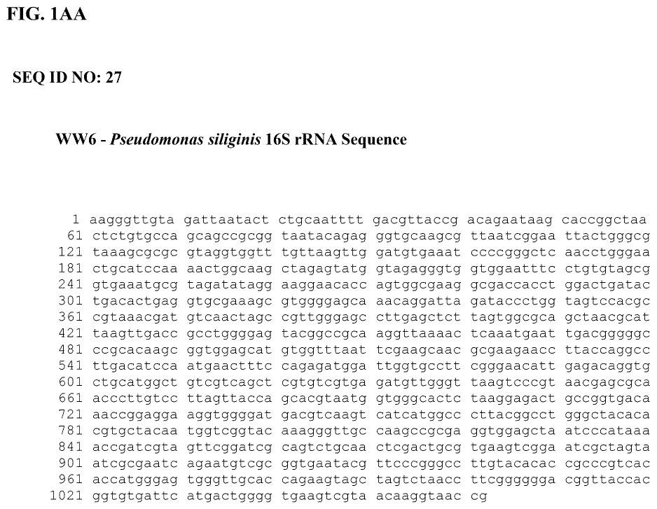

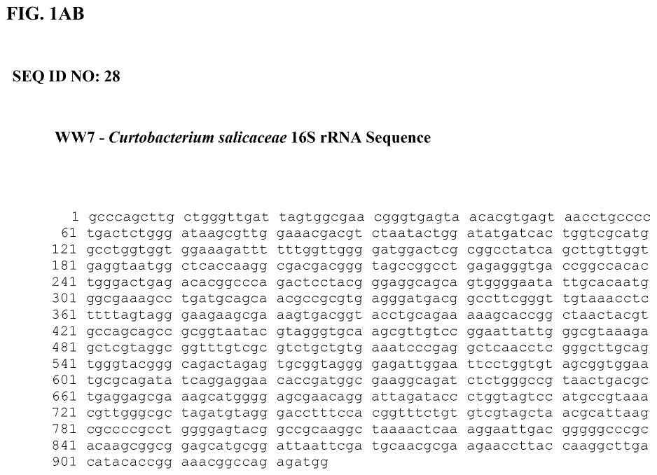

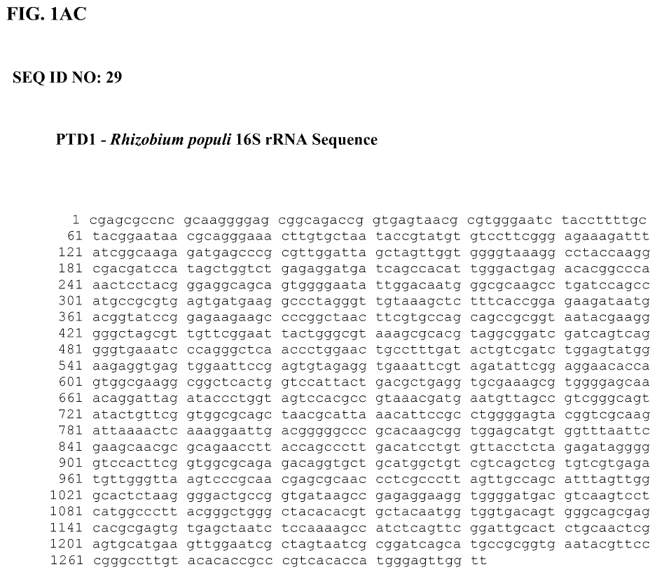

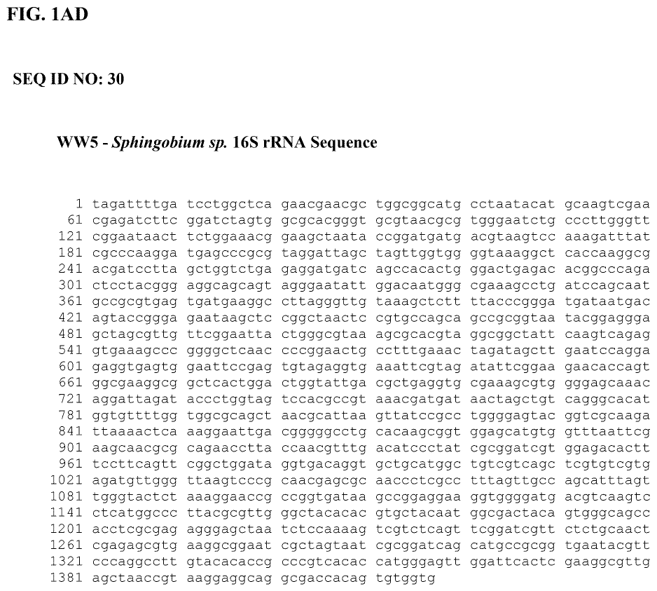

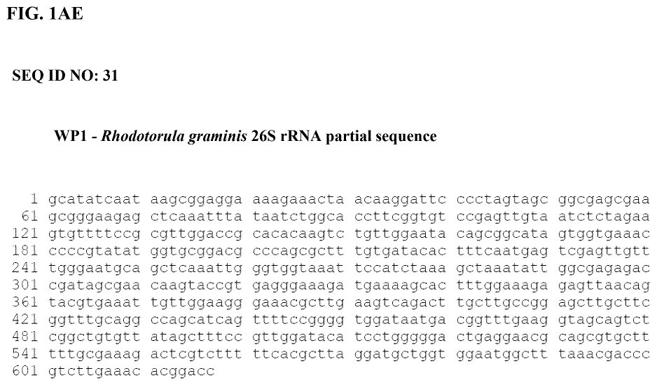

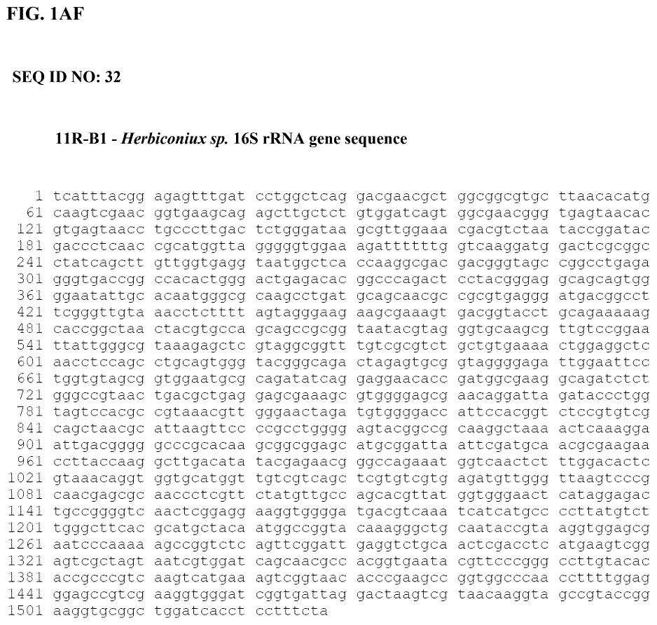

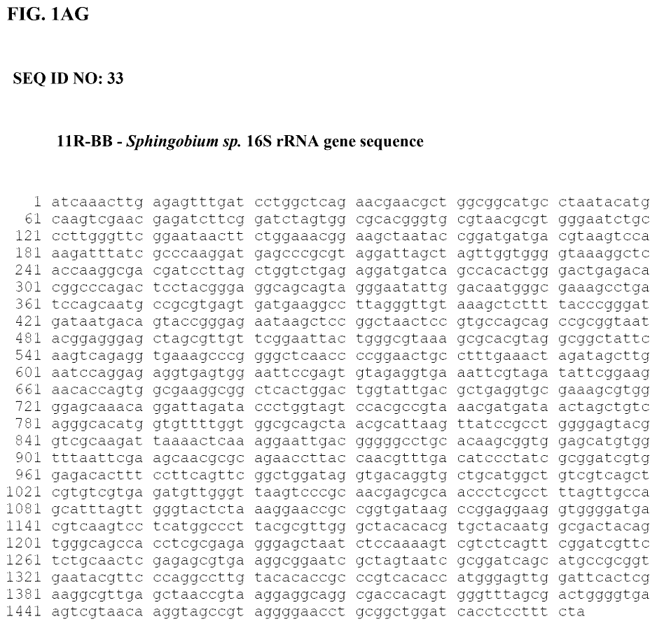

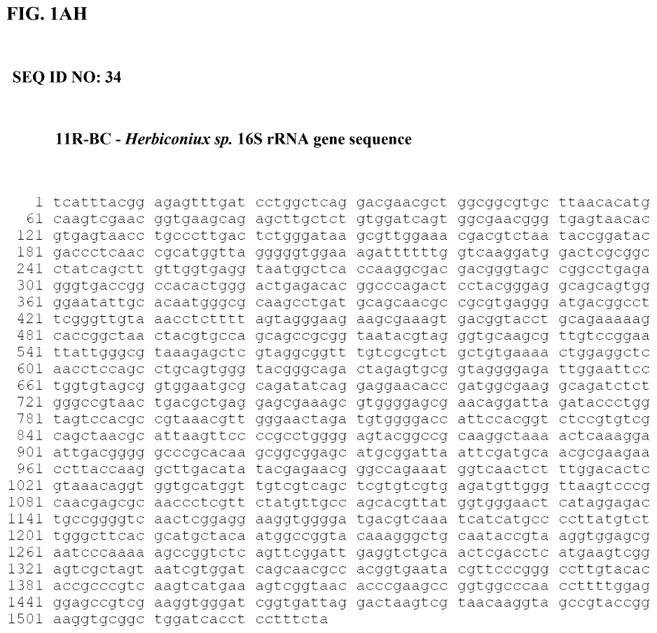

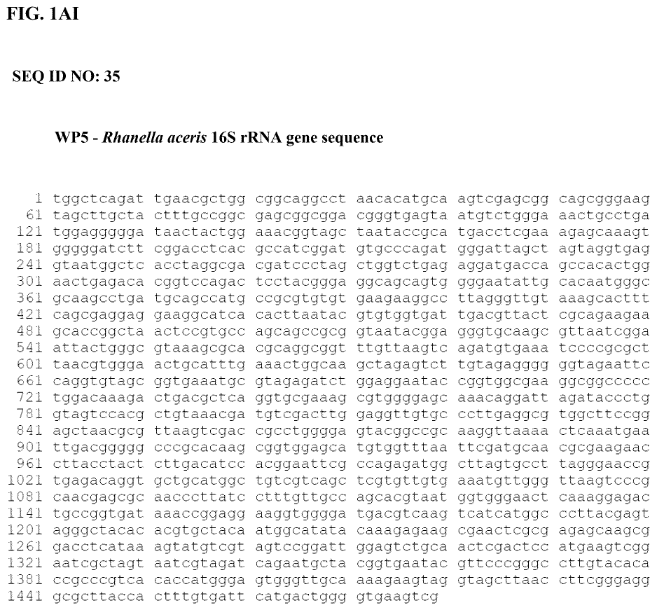

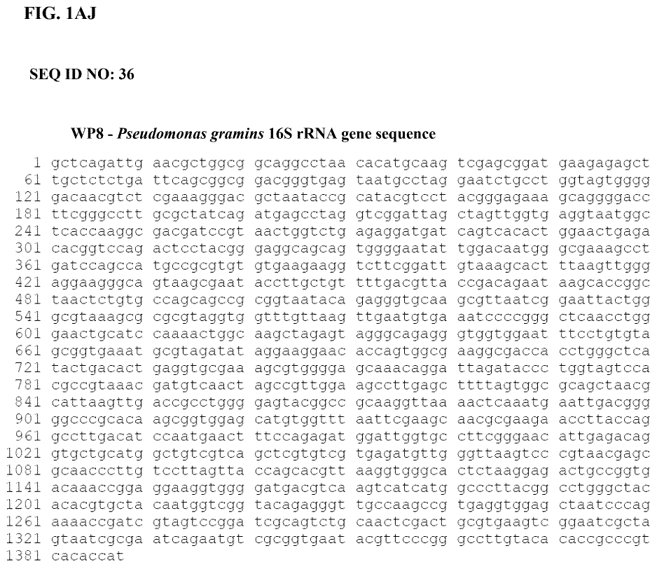

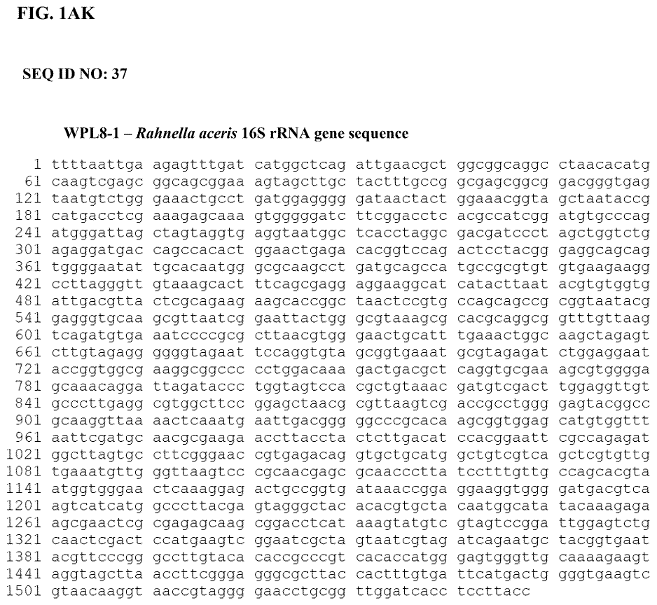

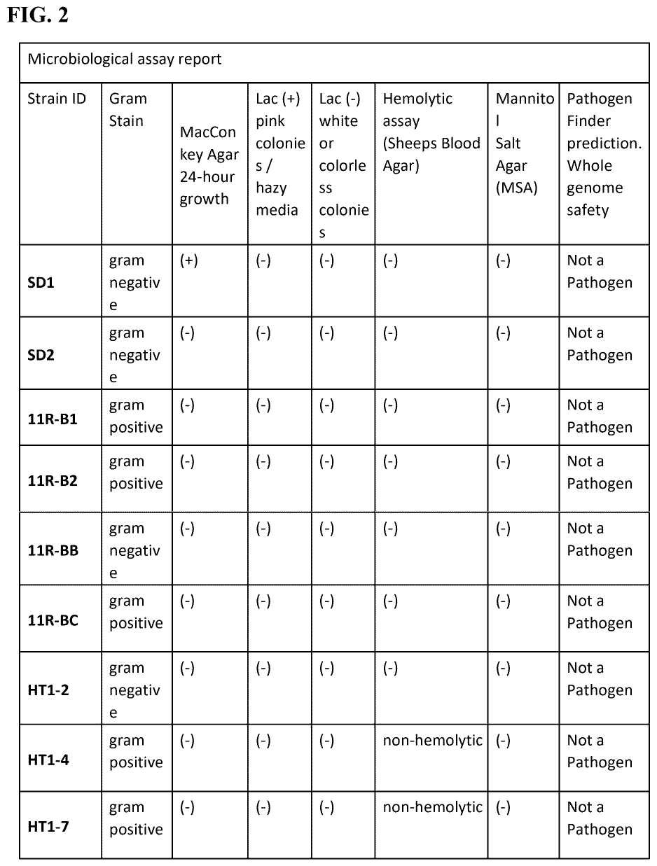

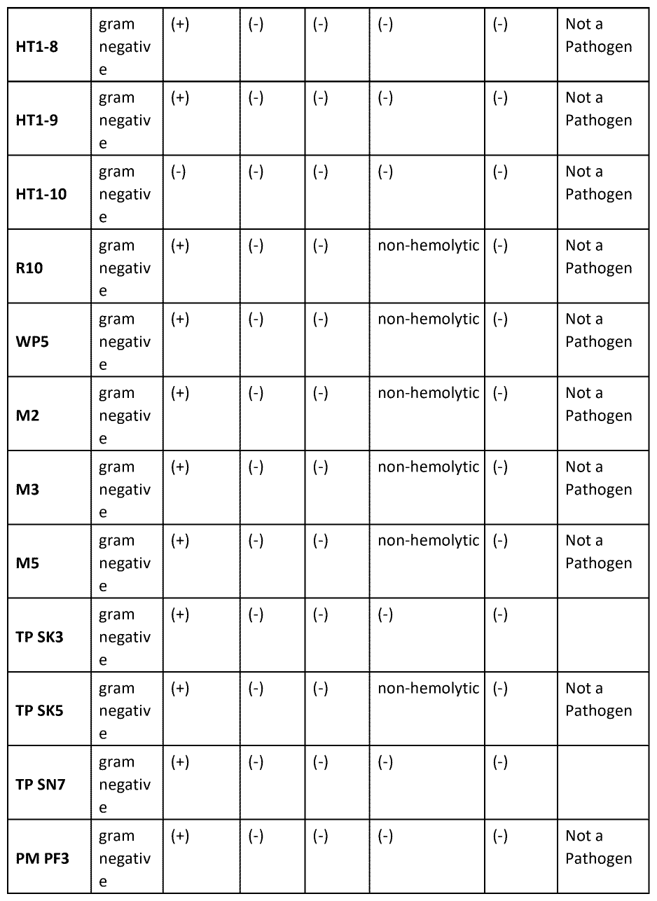

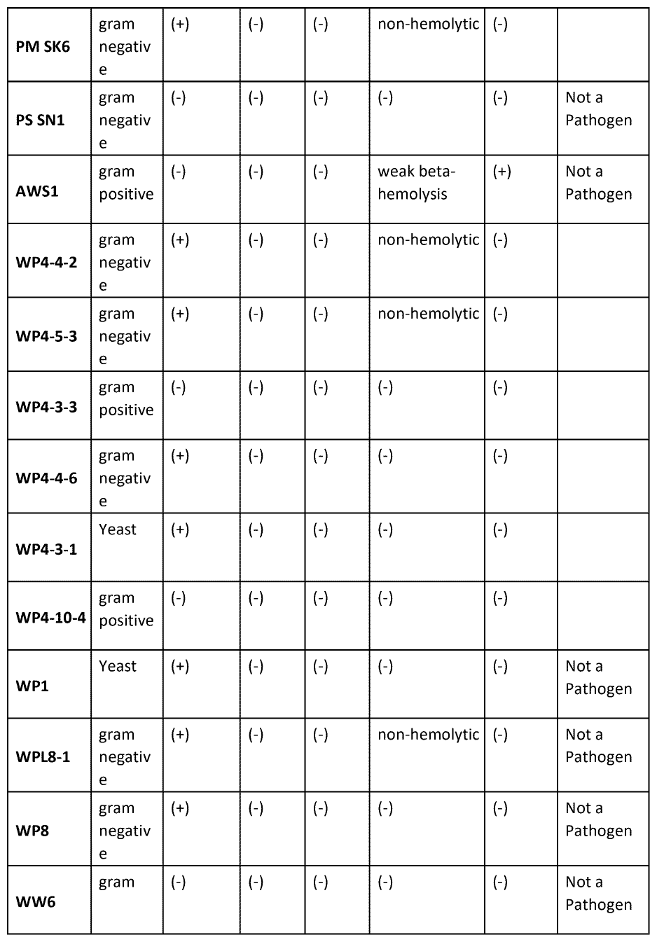

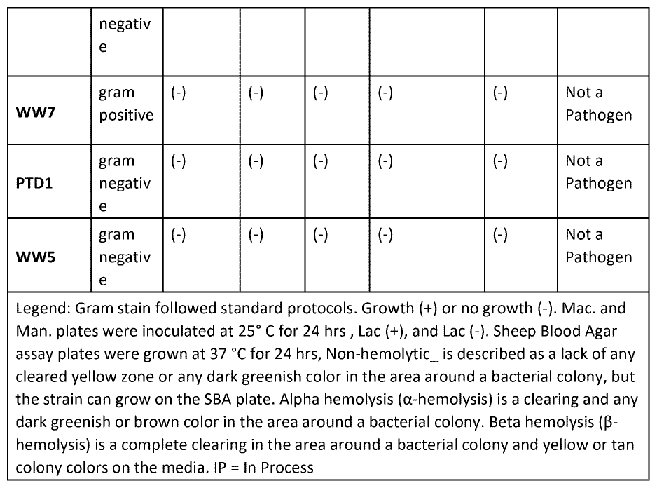

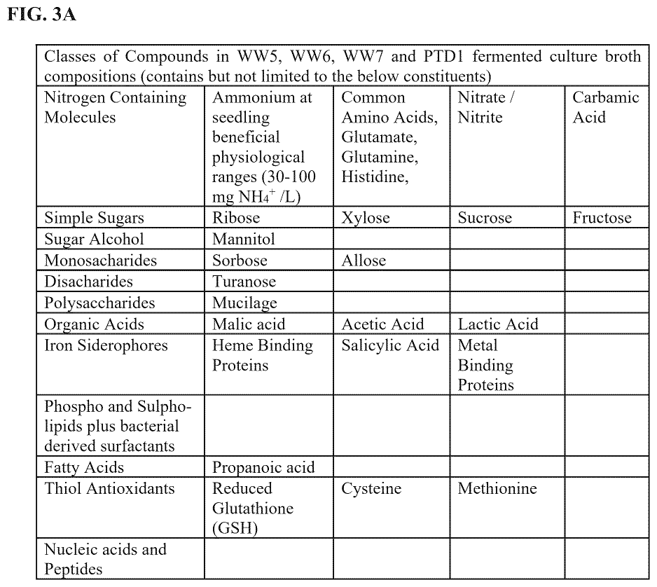

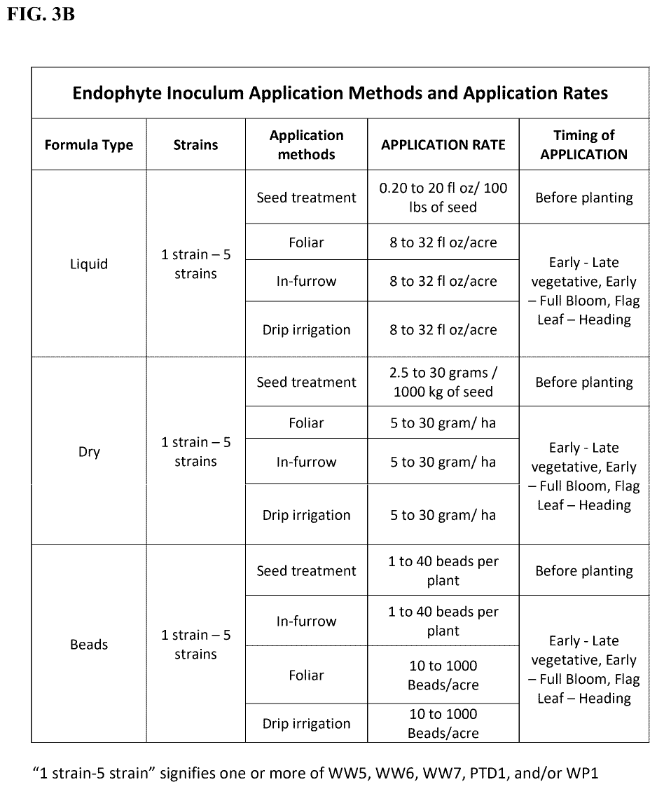

### DETAILED DESCRIPTION OF THE INVENTION

References will now be made in detail to certain embodiments of the invention, and example compositions and applications of such embodiments. While the invention will be described in reference to these embodiments, it will be understood that they are not intended to limit the invention. To the contrary, the invention is intended to cover alternatives, modifications, and equivalents that are included within the spirit and scope of the invention as defined by the claims. In the following disclosure, specific details are given to provide a thorough understanding of the invention. However, it will be apparent to one skilled in the art that the present invention may be practiced without these specific details.

### Methods

### Methods of Selection and Growth and Compositions

The present invention includes methods of growth and selection of diazotrophic endophyte strains. The methods include inoculating special nitrogen limited media, nitrogen-free growth media, or in some cases rich media (RM) and selecting colonies able to propagate on the specialized growth media. The ability of the endophyte strains to grow on nitrogen free and nitrogen limited medias were assessed.

### Example 1A

### Endophyte Selection and Testing

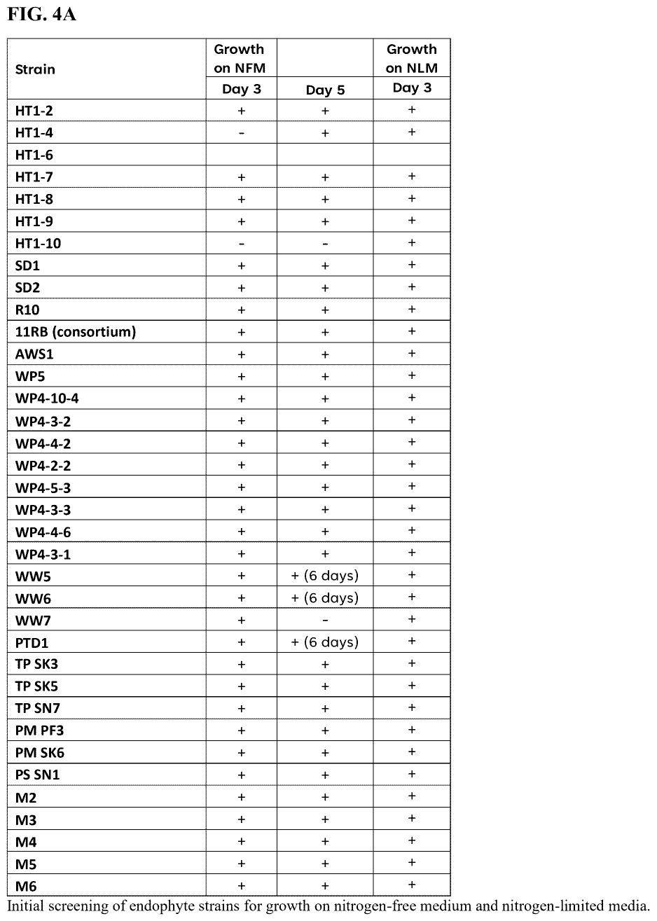

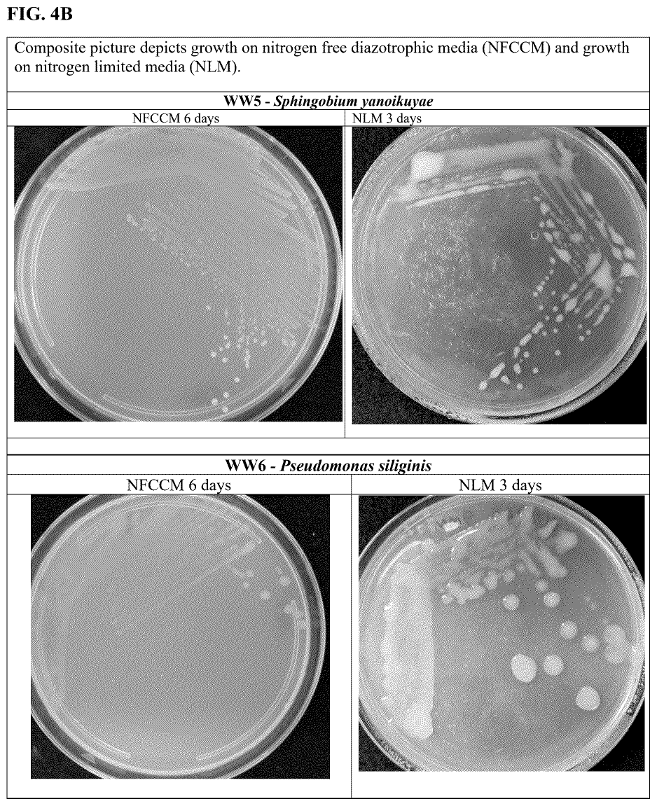

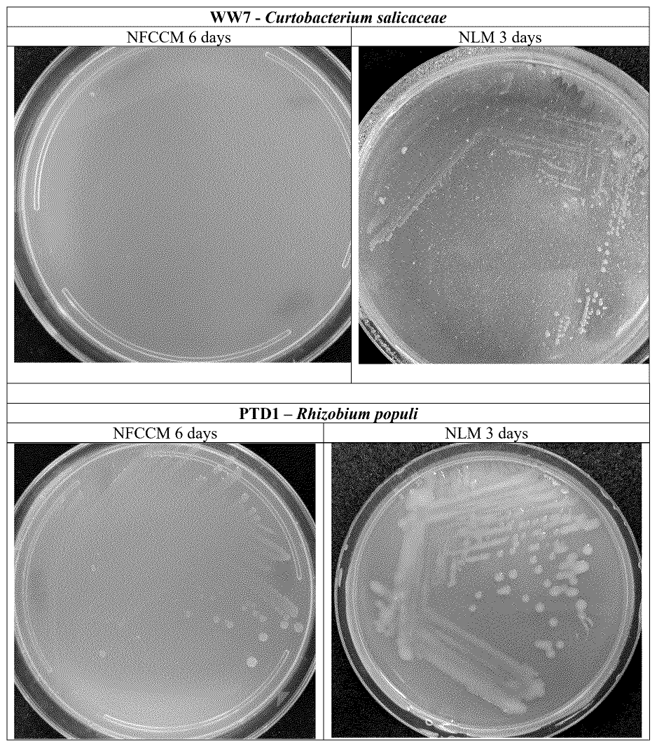

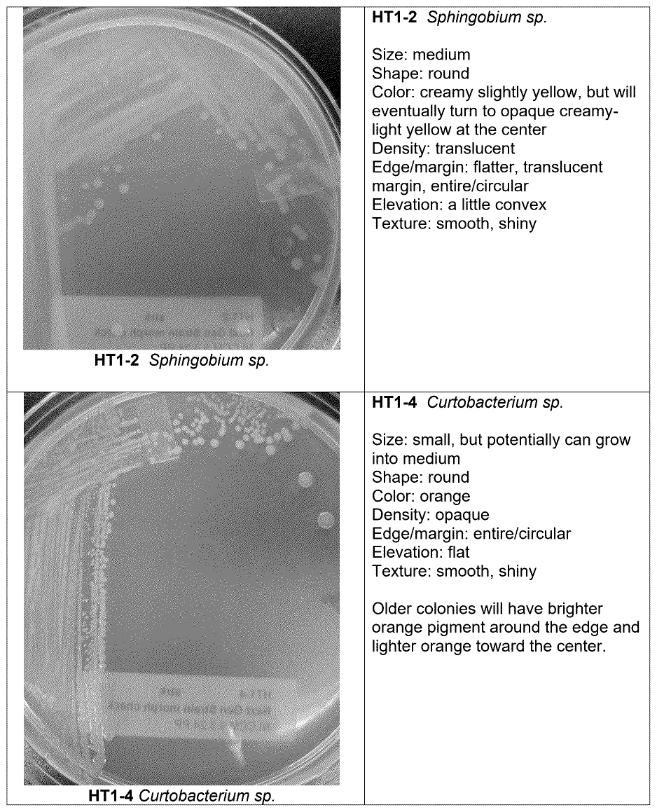

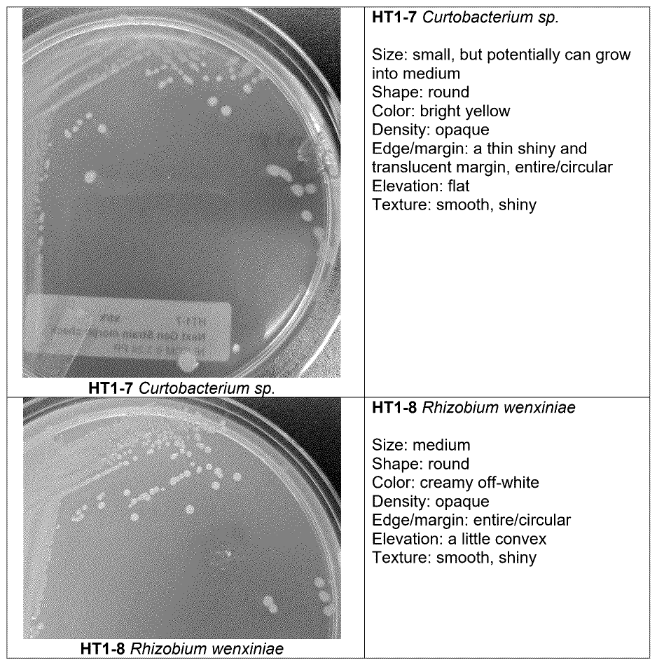

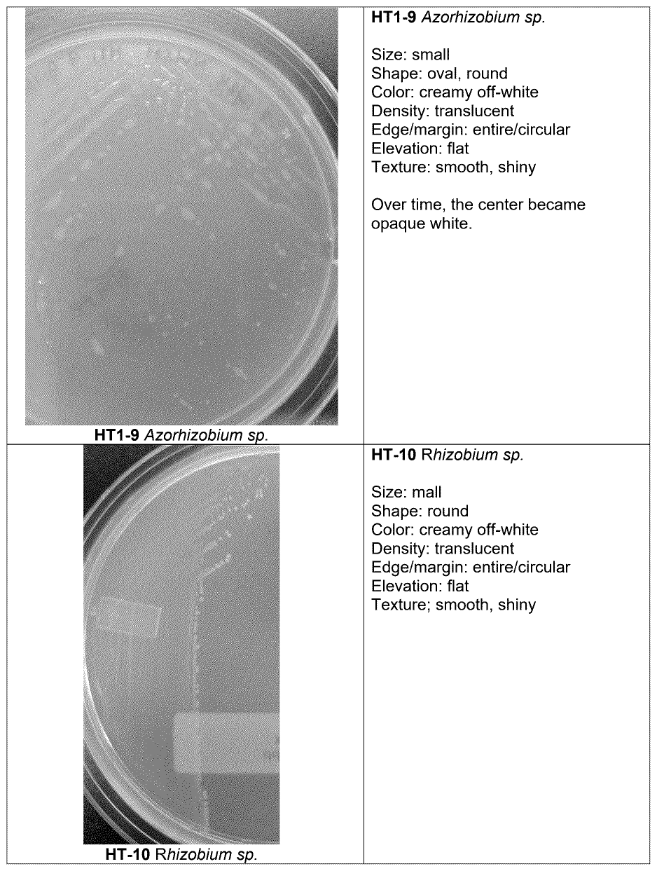

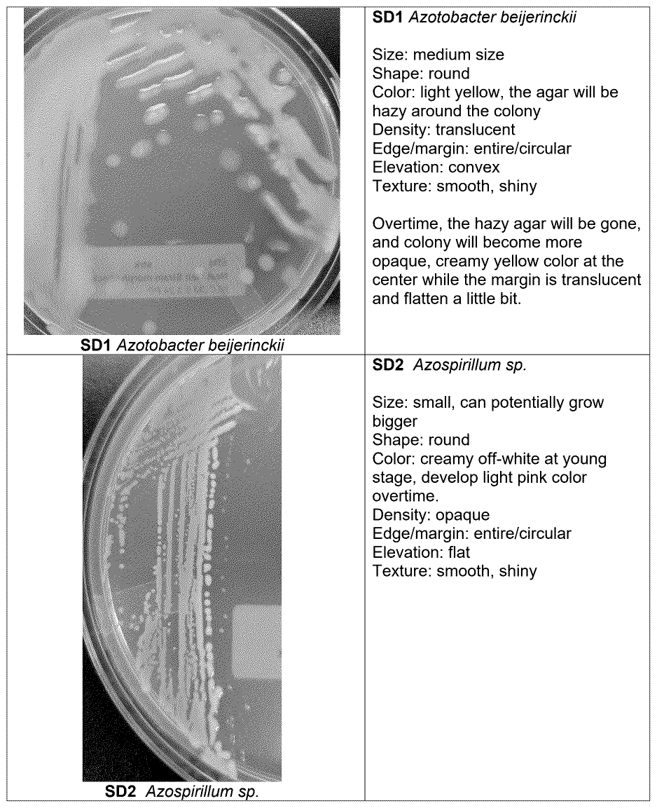

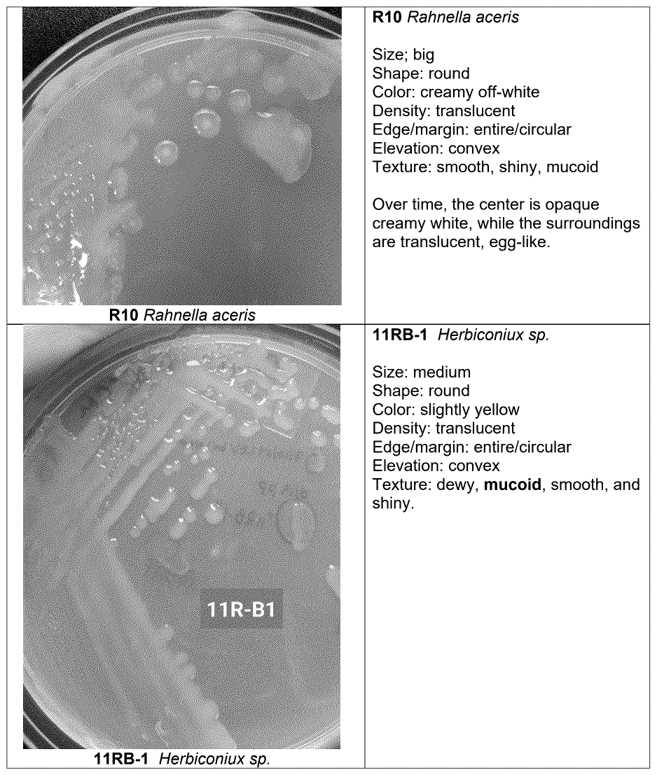

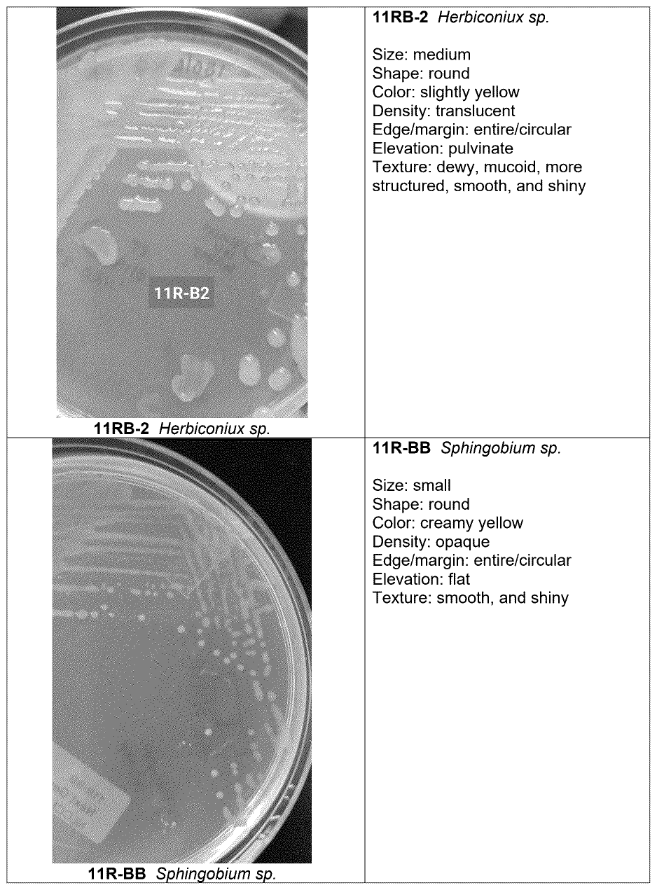

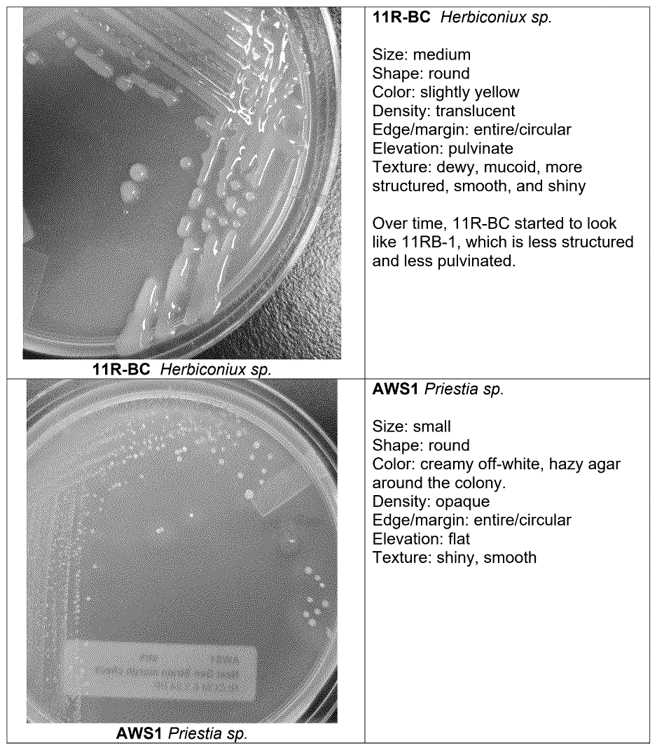

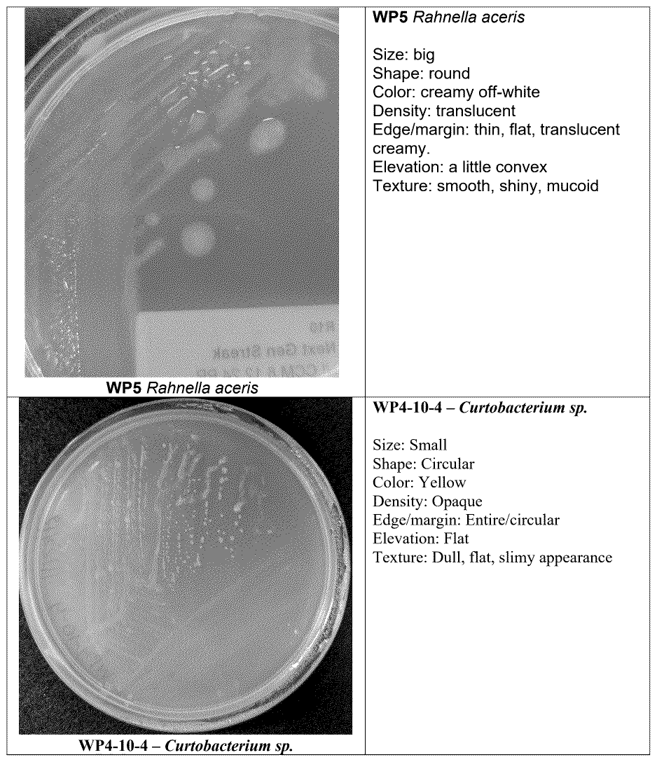

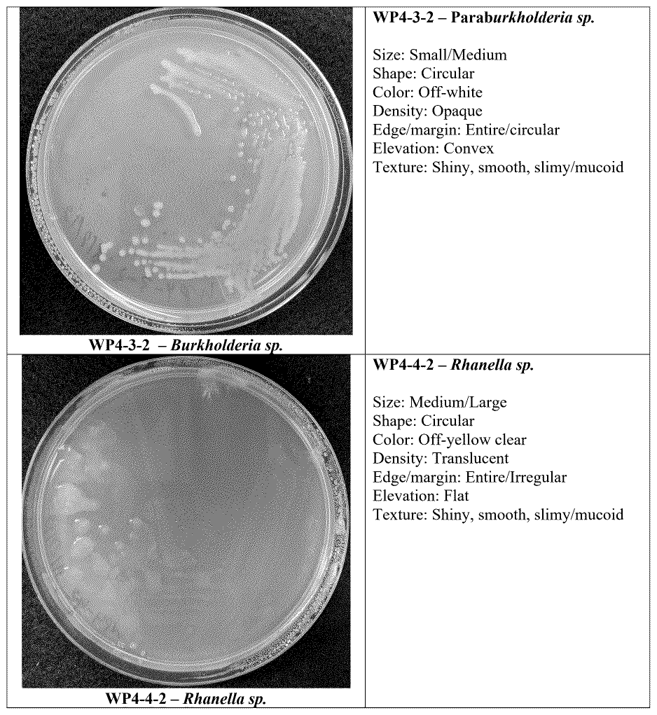

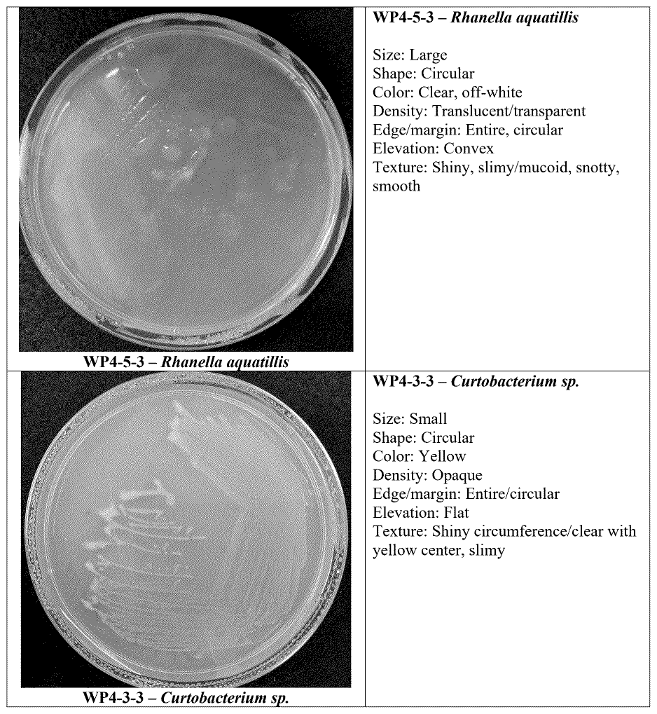

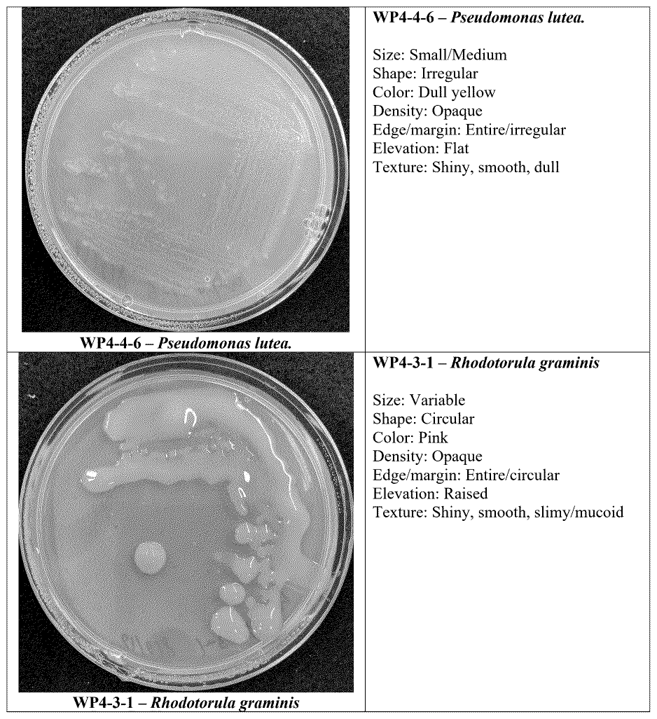

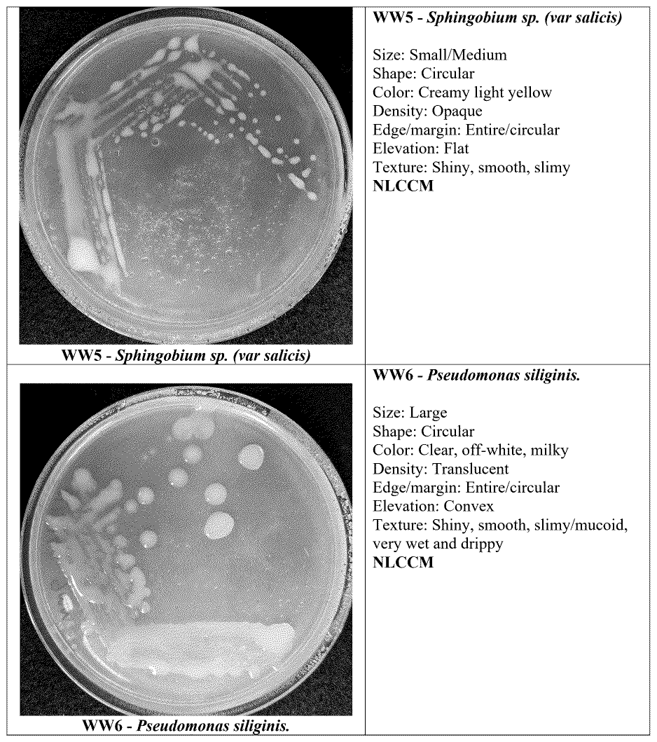

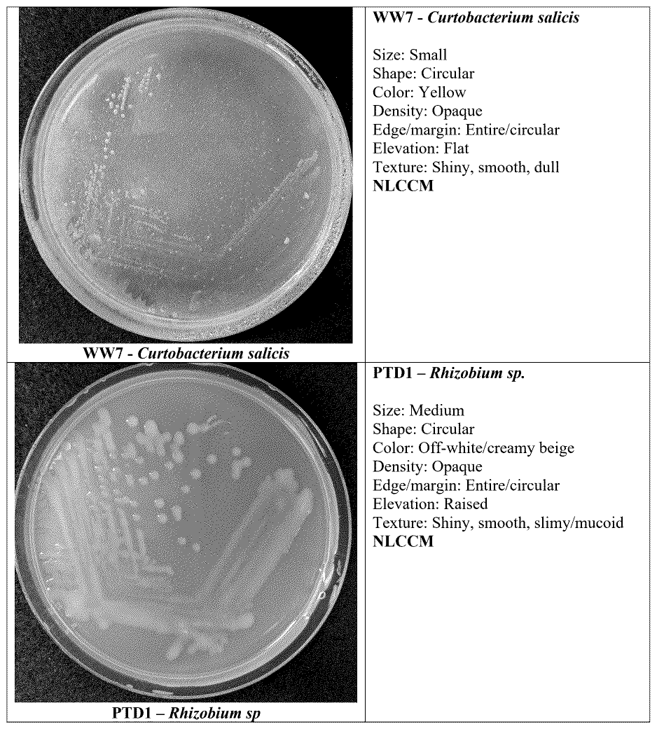

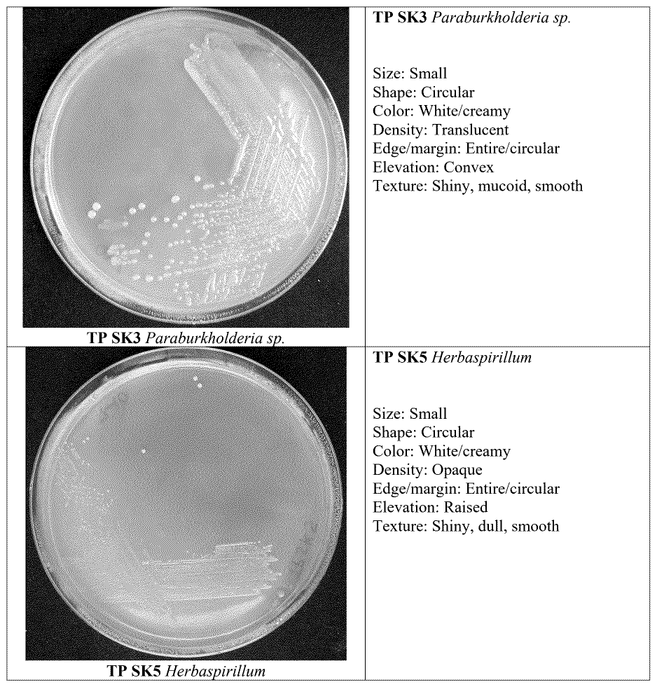

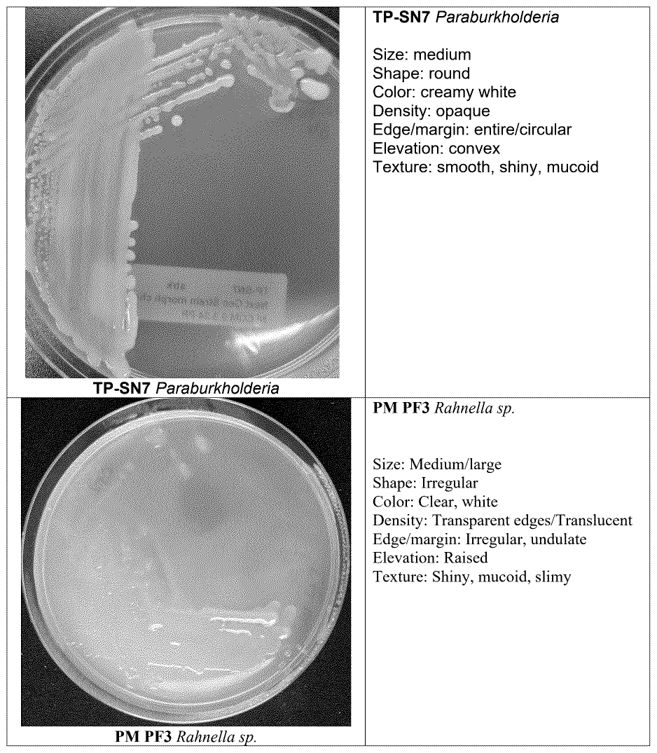

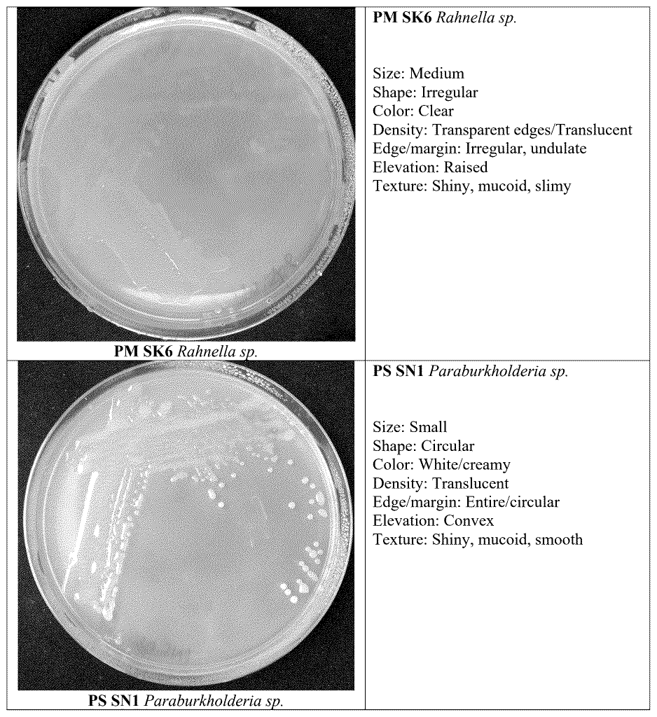

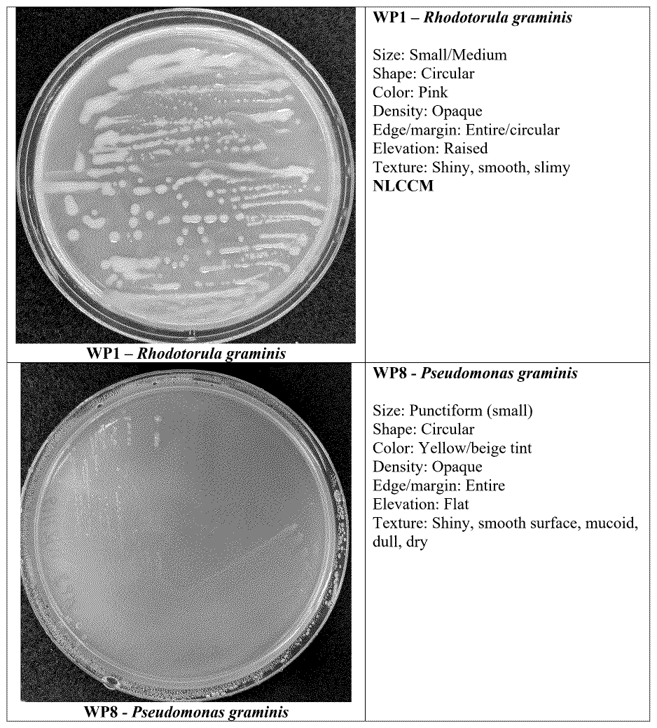

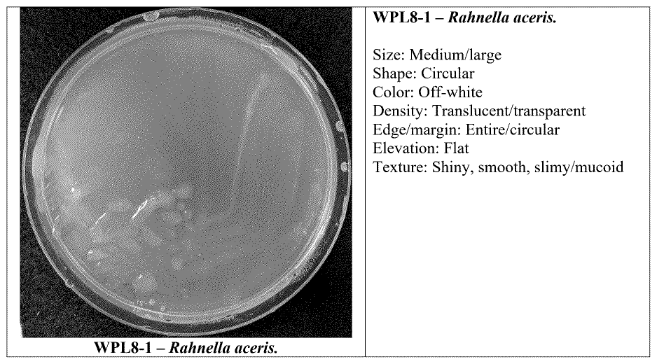

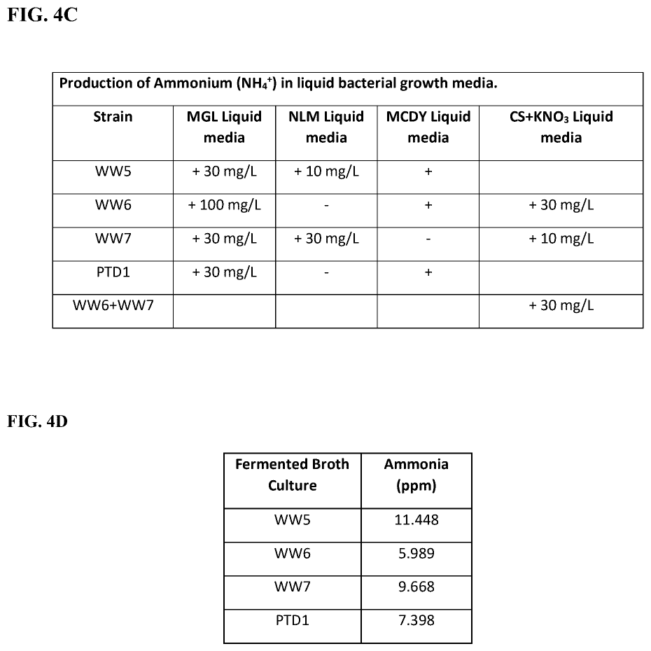

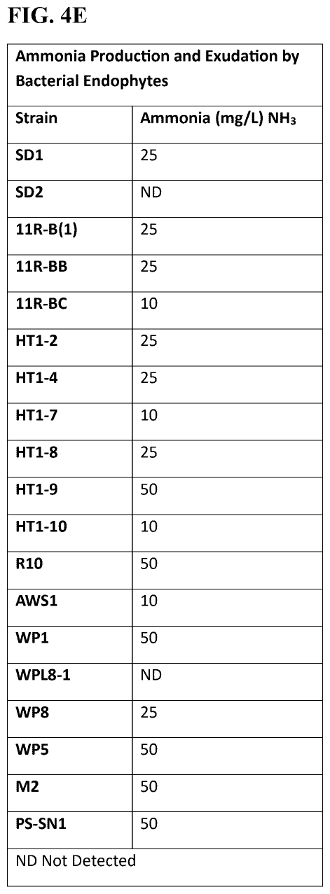

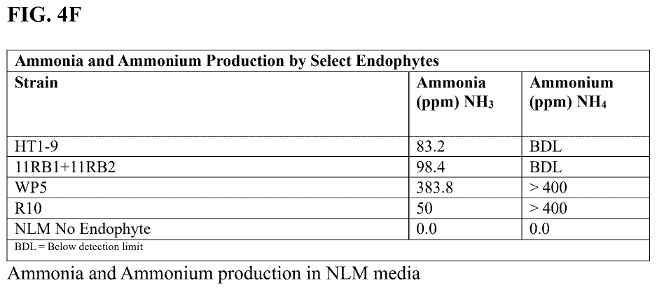

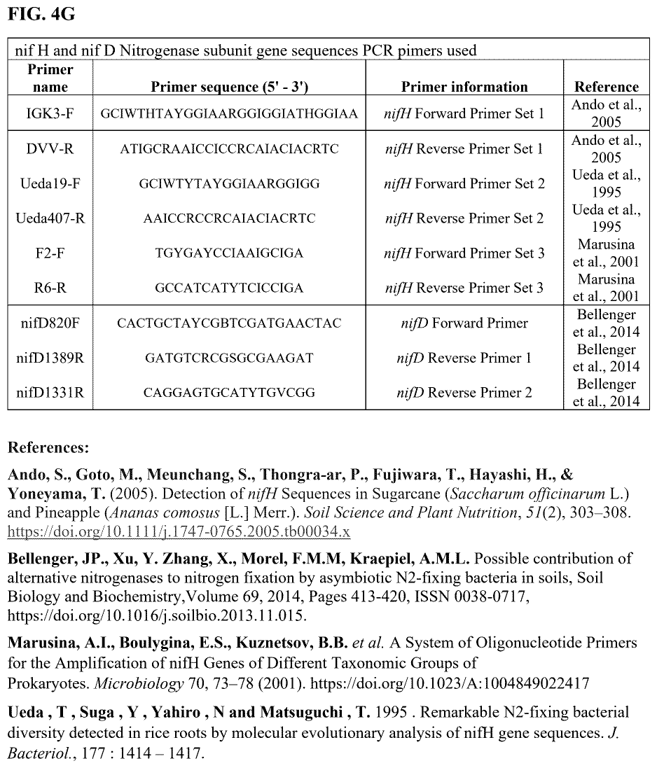

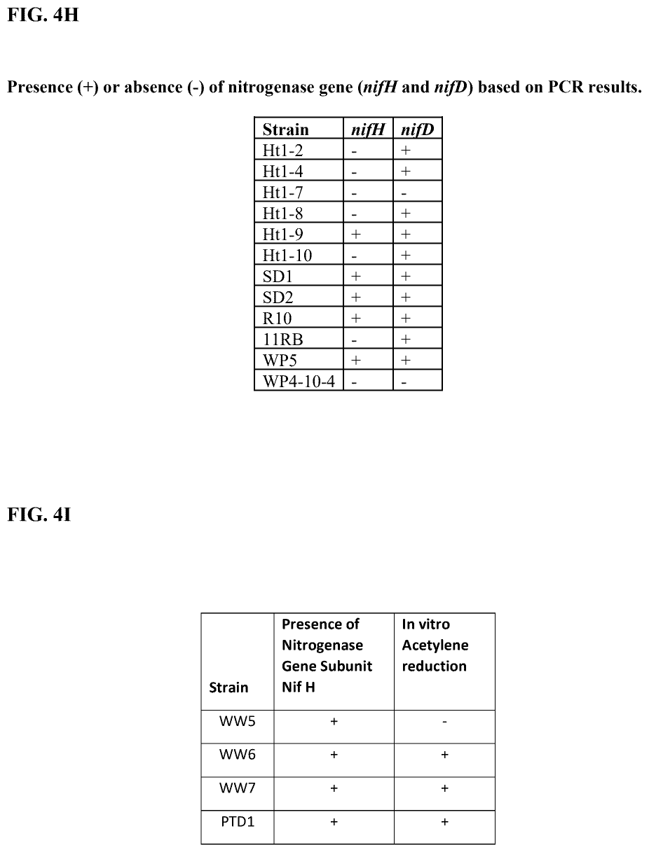

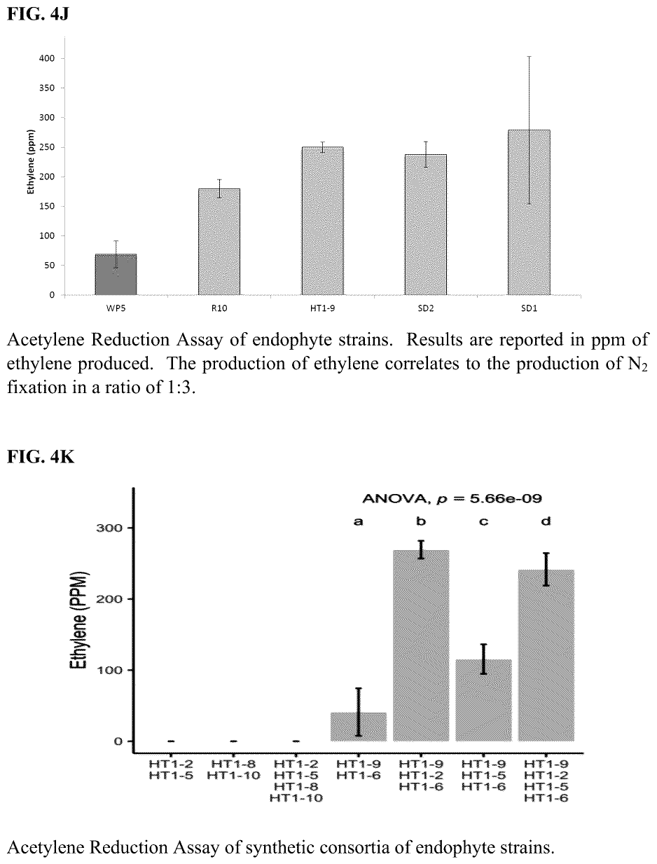

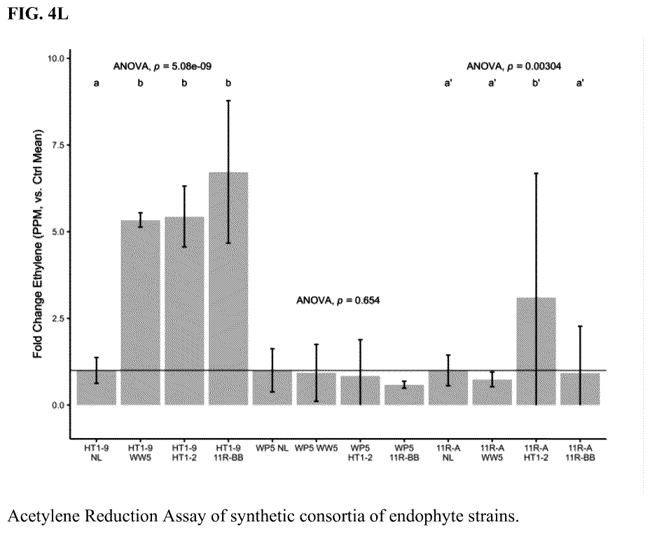

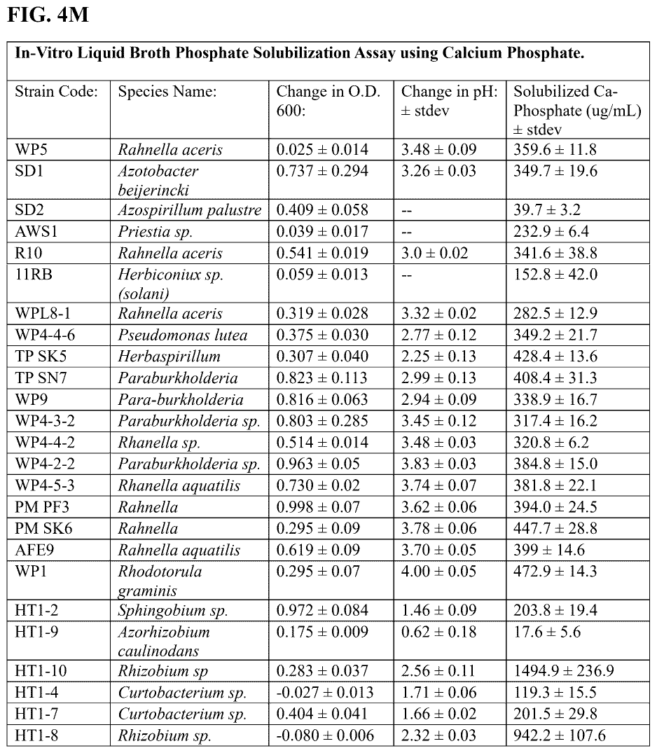

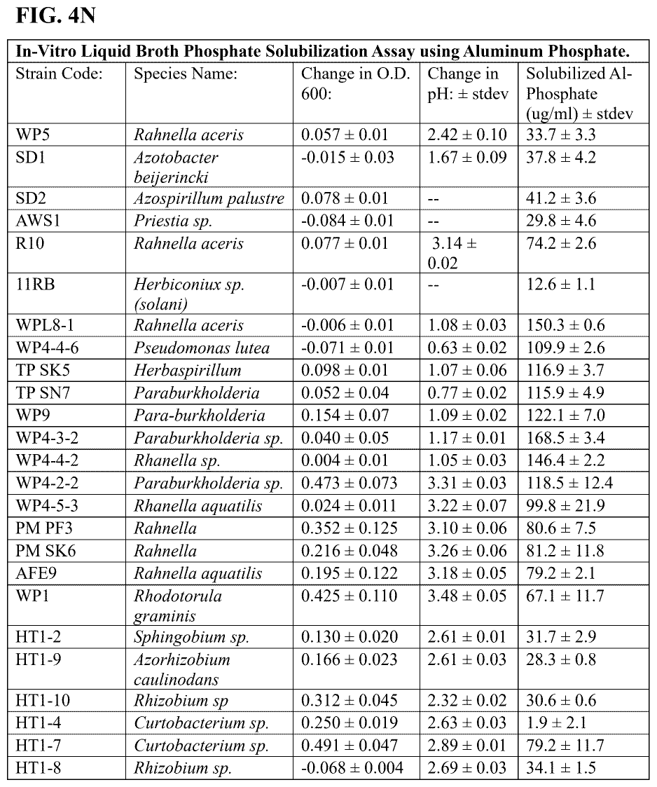

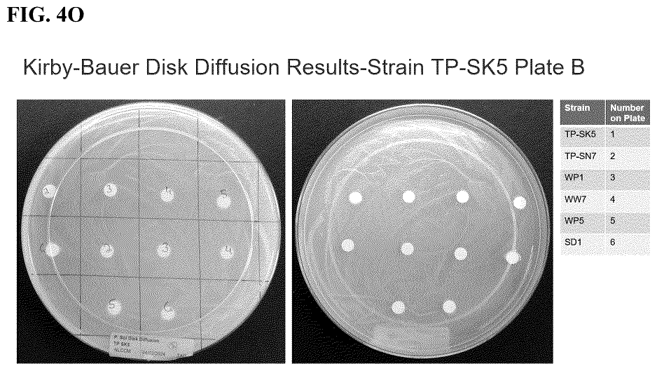

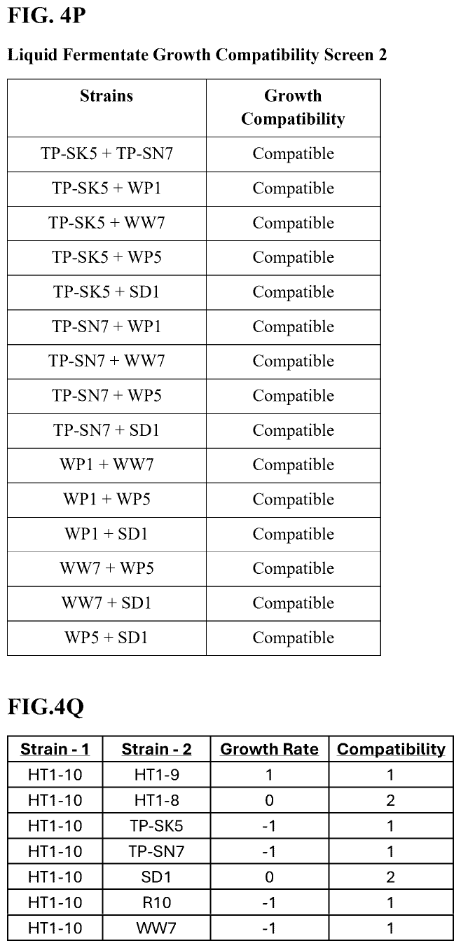

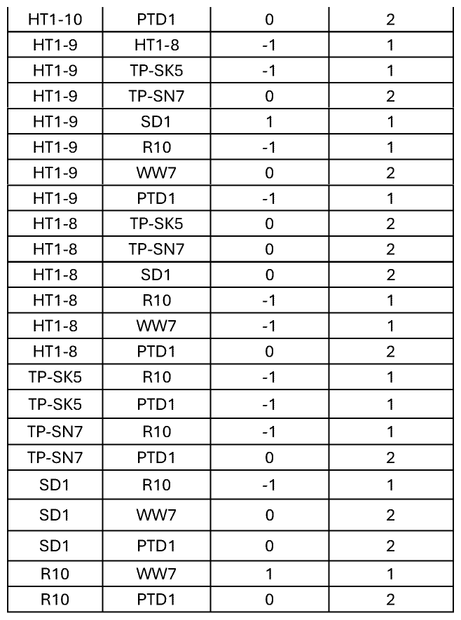

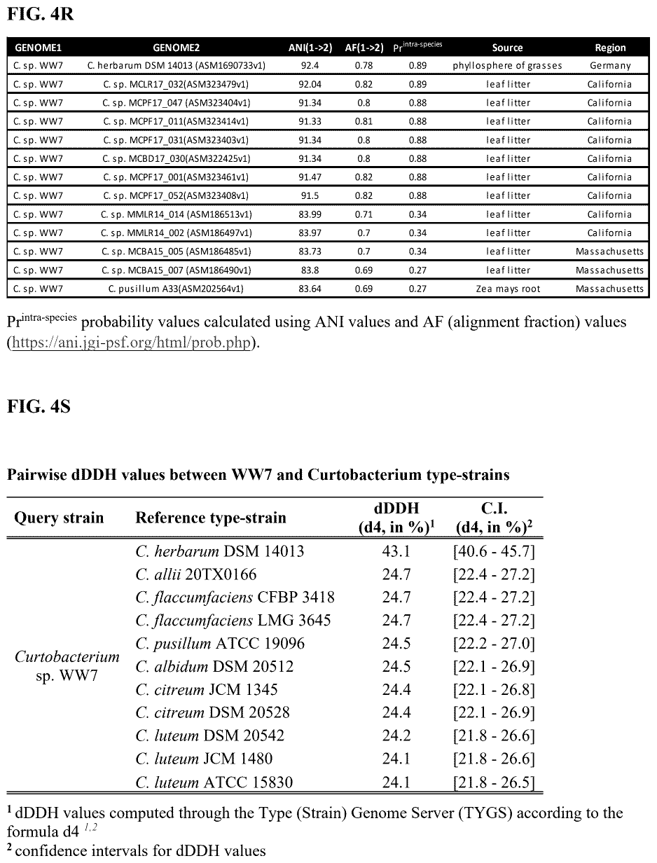

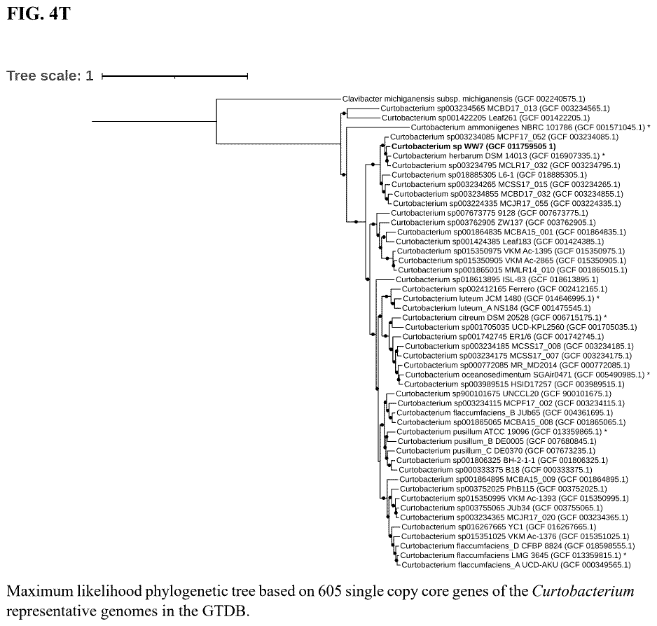

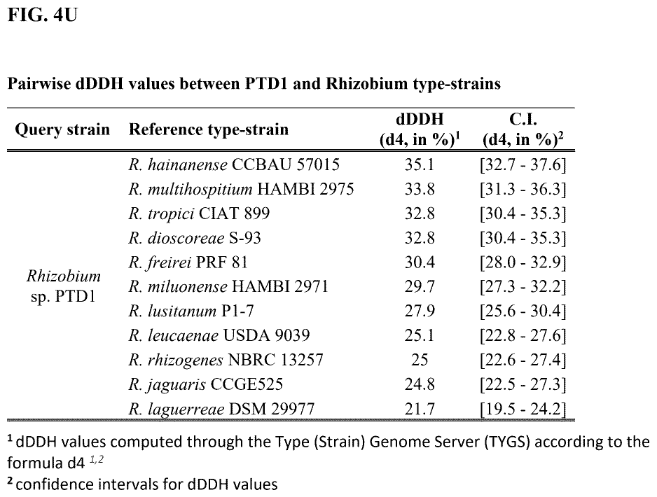

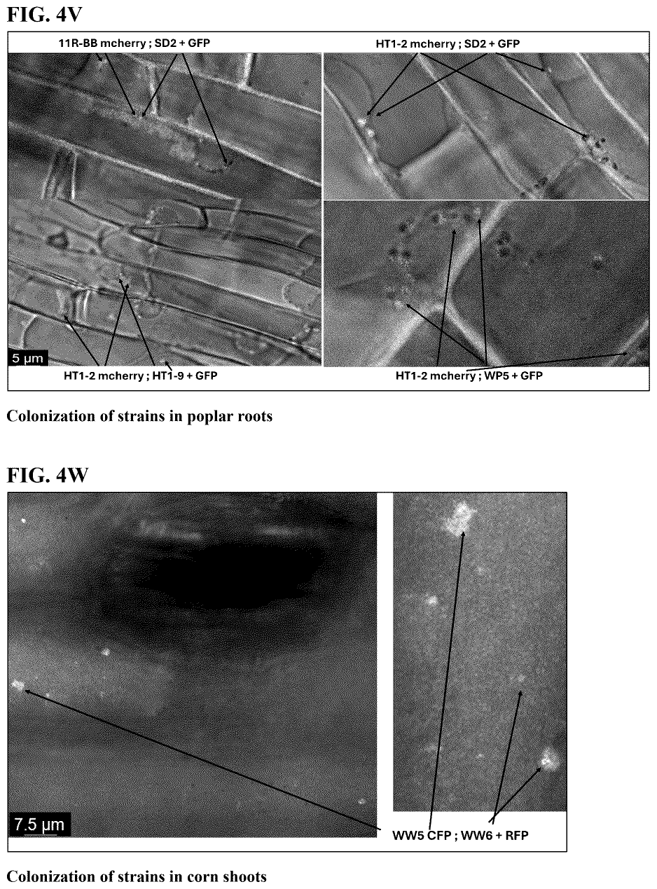

Each of the HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1 endophyte strains was tested and found positive for their ability to grow on nitrogen-limited media (NLM) and the strains each grew on nitrogen limited media to varying degrees as shown in FIGS. 4A-4D. Each endophyte strain was tested and found positive for their ability to grow on plant tissue culture grade agarose plates containing nitrogen limited media (NLM pH 7.6) and each grew on plant tissue culture grade agarose plates containing nitrogen free media (NFCCM pH 7.6) in FIG. 4A.

Each of the HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1 strains were further tested for their ability to grown on nitrogen-free media (NFM). All strains tested on NFM were able to grow colonies successfully, except the HT1-10 and WW7 strains. However, WW7 was able to grow on nitrogen-free Burke's media, which contains more vitamins and supplements than NFM but no nitrogen, suggesting this strain requires a series of cofactors for growth on N-free medium. Photographs of the colonies formed by each of the strains successfully tested on NLM and NFM media are provided in FIG. 4B.

A fermentation mixture comprising 10 mL broth cultures was prepared in 50 ml conical tubes. Each culture was inoculated with 100 μL with a normalized, QC broth culture of the endophyte strain(s). The conical tubes were set up on shaker in at a 45° angle and incubated at room temperature, shaking at 200 rpm for 72 hours. Optical densities (OD) of each culture were measured at 600 nm. 100 μL of 10-5 and a 10-6 dilution for each endophyte strain were plated on NLM agar plates. The plates were then incubated at 25° C. for 72 hours. CFUs formed on the plates were observed and recorded.

Select endophyte strains were also analyzed for whether they can produce ammonium in liquid fermentation under aerobic conditions. Separate assays for each of the WW5, WW6, WW7, and PTD1 endophyte strains showed that each of these endophyte strains was able to produce ammonium in such conditions.

Each strain was tested and found positive for their ability to produce ammonium (NH⁴⁺) exogenously in nitrogen limited medias, MGL (Mannitol-Glutamate/Luria-Bertani), NLM (Nitrogen Limited Media), MCDY (M series Yeast Media nitrogen base with amino acid supplements) and CS+KNO₃ (Corn Syrup+KNO₃), as shown in FIG. 4C. All sterile media tested negative for ammonium concentrations less than or equal to 0 mg/L.

In addition to the ability to produce ammonium NH₄⁺, the endophyte strains WW5, WW6, WW7, and PTD1 were evaluated to determine if they can also produce ammonia NH₃ in liquid fermentation under aerobic conditions. A fermentation mixture comprising 1 liter of nitrogen limited media (NLM) broth cultures was prepared in 2-liter flasks. Each culture was inoculated with 1 to 3 colonies from an NLM agar plate with a single endophyte strain. The flask was put on a shaker and incubated at room temperature, shaking at 125 rpm for 72 hours.

The endophyte strains were then analyzed for whether they can produce ammonia in liquid fermentation under aerobic conditions. Separate assays for each of the WW5, WW6, WW7, and PTD1 endophyte strains showed that all of the endophyte strains were able to produce ammonia in these conditions. The ammonia production data for each strain is provided in FIG. 4D.

### Example 1B

Some of the endophyte strains were tested for their ability to make Ammonia NH₃ the product of the nitrogenase enzyme that results from bacterial N₂ fixation and cellular exudation. The endophyte strains were prepared for the assay by growing starter cultures in large 96 well plates with 2 mL wells containing 1.5 mL NLM media in each and grown at 25° C. on a shaker table at 150 rpm for 72 hours. Bacteria were then prediluted to an OD of 0.1 and then 50 μL from the cultures were inoculated into large 96 well plates with 2 mL wells containing 1.5 mL NLM media and grown up at 25° C. on a shaker table at 150 rpm for 72 hours. The ammonia NH₃-concentration (mg/L) was then measured using water quality test strips UPC: 649910801545 SKU: PSS01V25 that detects ammonia in water for industrial applications and bioreactors. These strips are calibrated at 0, 10, 25, 50, and 100 ppm and gives colorimetric results that are compared to a standardized color indicator chart. These results are presented in FIG. 4E.

The presence of extracellular NH₃ in media suggests nitrogenase activity and the ability to fix N₂ and make and exude NH₃ into the media. The results in FIG. 4E demonstrate the use of diazotrophic N₂ fixing strains for making ammonia inside or at crop roots. The strains that produced exogenous NH₃ at approximately 50 mg/L included HT1-9, R10, WP1, WP5, M2, PS-SN1. The following strains produced about 25 mg/L exogenous NH₃: SD1, 11R-B1, 11R-BB, HT1-2, HT1-4, HT1-8, and WP8. The following strains produced about 10 mg/L exogenous NH₃: 11R-BC, HT1-7, HT1-10, and AWS1.

### Example 1C

### Ammonia and Ammonium Production in NLM Media.

A set of the below endophyte strains were tested for their ability to make ammonia NH₃ in synthetic liquid media. The cultures were grown in conical tubes with 10 mL NLM at 25° C. on a shake table (150 rpm) for 48-72 hours. Production of ammonia was measured with an Orion Standard Ammonia Ion Selective Electrode (Thermo Fisher Scientific, Chelmsford, MA) following the manufacturer's instructions. Ionic strength adjuster (ISA) was later added to each culture in a ratio of 200 μL per 10 mL culture, and vortexed. The resulting preparation was analyzed with the ammonia electrode using the Accumet XL 200 (Fisher Scientific, Singapore). Measured values were converted to ppm of ammonia using a standard curve then adjusted for background ammonia present in the NLM control.

Ammonium was also measured using Mouant Ammonium Test (Supelco) following the manufacturer's protocol. Briefly, 2 drops of the test reagent were added per 1 mL of endophyte culture then swirled to mix. A test strip was then used to quantify ammonium in the sample measured in ppm using quantitative chemical test kits.

The results are provided in FIG. 4F. WP5 produced the most exogenous NH₃ at 383.8 ppm. The 11RB1+11RB2 consortium produced 98.4 ppm NH₃. HT1-9 produced NH₃ at 83.2 ppm. The presence of extracellular NH₃ can represent high nitrogenase activity or the reduced ability to convert this precursor molecule into the N-containing amino acids glutamate and glutamine via the GOGAT pathway. From this result synthetic endophyte formulations can be made that preferentially supply multiple forms of N that are fixed, reduced, or incorporated into amino acids for direct use by all crop plants.

### Example 1D

### Endophyte Nitrogenase Genes.

The ability of the endophyte strains to fix nitrogen within the tissues of trees or plants is enabled by microbial nitrogenase genes within the endophyte bacteria. The nifH and nifD genes are often investigated to determine if a microbe is diazotrophic. The nifH gene encodes a nitrogenase gene subunit but is often difficult to find and define in different species even within strains having <3% sequence dissimilarity in their 16S rRNA genes can have up to 23% dissimilarity in nifH sequences meaning that often times the primers used for one species don't cross react with another species. The nifD gene however appears to be more consistent and encodes for the a subunit of nitrogenase, which contains the catalytic or active site for the reduction of nitrogen gas to ammonia and it binds the FeMo-cofactor, required for nitrogen fixation and participates in transfer of electrons from ferredoxin to nitrogenase, causing the reduction of N₂ to NH₃.

Each of the endophyte strains HT1-2, HT1-4, HT1-7, HT1-8, HT1-9, HT1-10, SD1, SD2, R10, 11R-B1, 11R-B2, 11R-BB, 11R-BC (consortia), AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, and WP1, WP8 and WPL8-1 were assayed for the presence of nitrogenase genes using specific primers for nifH and nifD through a PCR method. Some of the strains were found to include a copy of the nifD gene subunit and other strains were found to include a copy of the nifH gene subunit.

In the assay for the nifH subunit, a three phase PCR assay was conducted with three sets of primers. The primers are identified in their respective phases in FIG. 4G. The PCR method included preparing the required reagents were added to PCR tubes along with extracted DNA material from a selected endophyte strain. For nifH primer set 1, each PCR reaction tube contained 1× ExtremeTaq HiFi Red Mix (Azura Genomics, Raynham, MA), 320 nM of IGK3-F primer, 320 nM of DVV-R primer, 1.0-1.5 μL of the nifH DNA template, and molecular-grade water to a final volume of 20 μL per reaction. The tubes were mixed and centrifuged, then placed in a thermal cycler (Bio-Rad, Hercules. CA). The DNA products were amplified per the thermocycler program and primer parameters for 34 cycles plus 1 as follows: an initial denaturation was at 95° C. for 2 minutes; 35 cycles at 95° C. for 15 seconds; an annealing cycle at 55° C. for 15 seconds; and an elongation cycle at 72° C. for 45 seconds. A final elongation step was performed for 5 minutes at 72° C. before being held at 4° C. to stop the reaction. Results were evaluated with a 1.0-1.5% (w/v) agarose gel running at 100-120V for 30-90 minutes. A band at 400-500 bp was considered a positive hit. The method is described in Angel R, Nepel M, Panhölzl C, Schmidt H, Herbold C W, Eichorst S A, Woebken D. Evaluation of Primers Targeting the Diazotroph Functional Gene and Development of NifMAP—A Bioinformatics Pipeline for Analyzing nifH Amplicon Data. Front Microbiol. 2018 Apr. 30; 9:703.

The process for nifH primer sets 2 and 3 followed the same methodology used for primer set 1 with some modifications. Primer set 2 included the Ueda19-F and Ueda 407-R primers, and the elongation step was reduced from 45 seconds per cycle to 15 seconds per cycle, and the total cycle count increased from 35 to 40 cycles. Primer set 3 included the F2-F and R6-R primers and the elongation step was reduced from 45 seconds per cycle to 15 seconds per cycle.

The nifD gene encodes for the a subunit of nitrogenase, which contains the catalytic site for the reduction of nitrogen gas to ammonia and it binds the FeMo-cofactor, required for nitrogen fixation and participates in transfer of electrons from ferredoxin to nitrogenase, causing the reduction of N₂ to NH₃. Colony PCR analyses of the species and strains was performed and demonstrated nifD was present in several of the tested endophyte strains.

In the assay for the nifD subunit, a two-step nested PCR was used to screen the endophytes for the nifD gene. Both steps required the same master mix reagent ratios as nifH, but utilized different primers. The first round of PCR amplification was conducted using nifD820F: 5′-CAC TGC TAY CGB TCG ATG AAC TAC-3′ and nifD 1389R: 5′-GAT GTC RCG SGC GAA GAT-3′ primers followed by a second round of amplification using nifD820F and nifD1331R: 5′-CAG GAG TGC ATY TGV CGG-3′ primers. The PCR program consisted of an initial denaturation step at 95° C. for 2 minutes, followed by 31 cycles of 95° C. for 15 seconds, 55° C. for 15 seconds, and 72° C. for 30 seconds. A final elongation step was done for 5 minutes at 72° C. before the reaction mixture was held at 4° C. to stop the reaction. After the first PCR step was completed, PCR cleanup was performed by mixing 5 μL of the PCR product from step one 2 μL Exo-SAP-it (Thermo Fisher Scientific, Santa Clara, CA) in a PCR tube, then placing the mixture in the thermal cycler set to 37° C. for 15 minutes, followed by another 15 minutes at 80° C. The cleaned PCR product was used immediately in the second PCR step, which used the same thermal cycler PCR program with the nifD1331R reverse primer. Results were evaluated with a 1.0-1.5% (w/v) agarose gel running at 100-120V for 30-90 minutes. A band at approximately 500 bp was considered a positive hit. The results of the foregoing assays are shown in FIG. 4H. The method is described in Darcy L. McRose, Xinning Zhang, Anne M. L. Kraepiel, and François M. M. Morel, Diversity and Activity of Alternative Nitrogenases in Sequenced Genomes and Coastal Environments, Frontiers in Microbiology, 8; 2017. Additionally, whole genome sequencing an analysis showed that the SD1 and SD2 strains include the nifH nitrogenase gene, which is reflected in FIG. 4H.

Each of the WW5, WW6, WW7, and PTD1 endophyte strains were assayed for the presence of nitrogenase genes using specific primers for nifH genes through the PCR method. Each of the endophyte strains were found to include at least one copy of the nifH gene subunits.

The ability of the WW5, WW6, WW7, and PTD1 endophyte strains to fix nitrogen was further measured through the acetylene reduction assays. The acetylene reduction assay measures the ability of the nitrogenase enzyme to reduce acetylene gas to ethylene using gas chromatography to quantify the amount of ethylene produced. This is an indirect method of measuring N₂ fixation capacity, measuring the functional presence of the nitrogenase enzymes through its correlated ethylene production. The WW6, WW7, and PTD1 endophyte strains exhibited acetylene reduction activity, as shown in FIG. 4I.

### Example 1E

### Nitrogenase Enzyme Activity Using the Acetylene Reduction Assay (ARA).

An acetylene reduction assay (ARA) was used to demonstrate the ability of single endophyte strains to fix N₂. ARA is a widely used method for measuring biological nitrogen (N₂) fixation (BNF) in microbes. Nitrogenase is the enzyme responsible for the reduction of atmospheric nitrogen (N₂) to ammonia (NH₃) in nitrogen-fixing organisms. The ARA indirectly measures nitrogenase activity by exploiting its ability to reduce acetylene (C₂H₂) to ethylene (C₂H₄). This reduction can be quantified using gas chromatography (GC-MS), providing an indirect estimate of nitrogen-fixation capacity.

The amount of ethylene detected correlates directly with nitrogenase activity, as the enzyme reduces acetylene to ethylene in a reaction similar to nitrogen reduction to ammonia. The results were compared to negative controls to ensure that the ethylene detected is specifically due to nitrogenase activity. The quantification of nitrogenase activity is expressed as the rate of ethylene production. The biochemical conversion ratio (‘R ratio’) of N₂ fixation in relation to the reduction of acetylene to ethylene by the nitrogenase enzyme is 3-N₂ fixed to 1-acetylene reduced to ethylene.

Synthetic culture formulations of the WP5, R10, HT1-9, SD2, and SD1 endophyte strains were prepared for testing using ARA to quantify nitrogenase activity. The strains were cultured under nitrogen-limiting conditions as described herein to produce a fermentate and to induce nitrogenase expression. The cells were harvested from the fermentate and resuspended in a nitrogen-free medium. The bacterial suspension was then transferred to gas-tight, sealed vials or serum bottles, leaving a headspace for gas exchange.

Once the vials are prepared, acetylene gas is injected into the headspace at a concentration of approximately 10% (v/v). The strains were incubated in the sealed vials, allowing nitrogenase to reduce acetylene (C₂H₂) to ethylene (C₂H₄). After incubation, a gas-tight syringe was used to withdraw a sample of the headspace gas, which is then analyzed using a gas chromatograph equipped with a flame ionization detector (FID) to detect the amount of ethylene produced. The method was carried out as described in Hardy R W F, Holsten R D, Jackson E K, Burns R C (1968) The Acetylene-Ethylene Assay for N₂ fixation: laboratory and field evaluation. Plant Phys 43:1185-1207.

The ARA results are reported in FIG. 4J. Of those strains tested, SD1 exhibited the highest N₂ fixing strain at 280 ppm ethylene (C₂H₄). The HT1-9 strain exhibited 250 ppm C₂H₄. The SD2 strain product 240 ppm C₂H₄. The R10 strain produced 180 ppm C₂H₄. WP5 produced 60 ppm C₂H₄ Ethylene measured by GC-MS from the reduction of acetylene is an estimate of nitrogenase activity 3:1.

### EXAMPLE 1F

### Increased Nitrogenase Activity Through Synergistic Stacking of Different Strains Different Endophyte Species.

Acetylene reduction assay (ARA) as described in Example 1C was also used to demonstrate the ability of synthetic consortia of endophyte strains to fix N₂. Synthetic consortia formulations of two endophyte strains (HT1-9+HT1-6), three endophyte strains (HT1-9+HT1-6+HT1-2 and HT1-9+HT1-5+HT1-6), and four endophyte strains (HT1-9+HT1-2+HT1-5+HT1-6) were prepared for testing using ARA to quantify their collective nitrogenase activity. The ethylene production of these synthetic consortia are reported in FIG. 4K.

The synthetic consortia of HT1-6 and HT1-9 had a combined nitrogenase activity level of about 50 ppm of C₂H₄. The synthetic consortium of HT1-6, HT1-9, and with HT1-2 had higher nitrogenase activity compared to the consortium of HT1-6 and HT1-9 (F3,8=282.131, p<<0.001; Cohen's d=24.006). The synthetic consortium of HT1-6, HT1-9, and with HT1-5 also exhibited higher nitrogenase activity compared to the consortium of HT1-6 and HT1-9 (Cohen's d=7.831). No activity was detected in consortia that do not include the HT1-6 and HT1-9 strains. Data are mean±95% CI (n=3); one-way ANOVA. Different letters indicate significant differences (p<0.001).

### Example 1G

### Increased Nitrogenase Activity Through Synergistic Stacking of Different Strains Different Endophyte Species.

Acetylene reduction assay (ARA) as described in Example 1C was also used to demonstrate the compare single strain endophyte nitrogenase activity to synthetic consortia of endophyte strains to fix N₂. Synthetic consortia formulations of two endophyte strains HT1-9+WW5, HT1-9+HT1-2, and HT1-9+11R-BB were tested in comparison to HT1-9 alone. Synthetic consortia formulations of two endophyte strains WP5+WW5, WP5+HT1-2, and WP5+11R-BB were tested in comparison to WP5 alone. Synthetic consortia formulations of two endophyte strains 11R-A+WW5, 11R-A+HT1-2, and 11R-A+11R-BB were tested in comparison to 11R-A alone. The nitrogenase activity in diazotrophic co-cultures was enhanced by using each of three different synergistic strains WW5, 11R-BB and HT1-2 when applied synthetic consortia with another diazotrophic N₂ fixing strain The ethylene production of these synthetic consortia are reported in FIG. 4L.

Nitrogenase activity was measured in synthetic consortia of the three HT1-9, WP5, and 11R-A, each in combination with the synergistic strain HT1-2, WW5, and 11R-BB, respectively. The results demonstrated that the nitrogenase activity was 2.3-fold higher in the HT1-9+HT1-2 co-culture compared to HT1-9 alone (t(4) 393 −24.935, p<<0.001). Nitrogenase activity was 4.9 times higher in the WP5+WW5 co-culture compared to WP5 alone (t(4)=−394 54.861, p<<0.001). Nitrogenase activity was 1.4 times higher in the 11R-A+11R-BB co-culture compared to 11R-A alone (t(4)=−5.744, p=0.005). No nitrogenase activity was detected in controls with no bacteria (these data are not shown). Data are mean±95% CI (n=3); independent t-tests. Different letters indicate significant differences (p<0.01).

### Example 1H

### Quantification of the Insoluble Phosphate Solubilization Abilities for Tri Calcium Phosphate of the Synthetic Endophyte Single Strain Formulations.

To demonstrate the ability of endophyte strains to solubilize insoluble phosphate, a phosphate solubilization assays was conducted in a no phosphate liquid broth media that was amended with insoluble aluminum phosphate and tri-calcium phosphate at a neutral pH. This involved using a free phosphate-sensitive dye and a microplate reader to confirm the biochemical capabilities of the endophyte strains to solubilize two phosphate compounds while actively growing and replicating. This is a new physiological assay method, adapted from Varga et al., 2020. The endophyte strain cells were cultured in modified National Botanical Research Institute defined phosphate growth media (NBRIP) broth without phosphate to deplete any residual internal phosphate in the endophytes. This modified NBRIP media was specifically developed to screen phosphate solubilizing plant endophyte microorganisms. In terms of modifications from Varga et al., 2020, 1 ml NLM cultures as described herein were mixed with 4 mL of NBRIP at a pH in a range of 6.57-7.23, plus one of insoluble aluminum phosphate and insoluble tri-calcium phosphate. The resulting 5 mL culture tubes were incubated for 3 days at 25° C. with shaking at 220 rpm. After incubation, the following parameters were measured; O.D. 600, ending pH, and the specific absorbance at 650 nm (A650) of solubilized phosphate (mol/L) was measured using a Phosphate Colorimetric Assay Kit (Sigma-Aldrich, MAK030).

The mol/L phosphate solubility results are provided in FIGS. 4L and 4M. which show statistically significant increases (p<0.01) in phosphate solubilization for both insoluble aluminum phosphate and tri-calcium phosphate. This demonstrates a significant mobilization of insoluble phosphate by the selected endophyte strains and shows that they can be used to inoculate crops to enhance in-planta phosphate acquisition from insoluble forms of phosphate that are present in farm and agricultural soils. The change in pH results are also provided in FIGS. 4M and 4N, which show statistically significant decreases in pH (p<0.01) for strains in both insoluble aluminum phosphate and tri-calcium phosphate. This demonstrates a significant acidification of the media by the selected endophyte strains and that they can be used to inoculate crops to enhance phosphate solubility in soil caused by acidifying the rhizosphere.

### Example 1I

### Compatibility of Strains for Use as Crop Plant Treatments

A set of in vitro tests were conducted to confirm compatibility of endophyte strains and guide the selection of combinations of compatible endophyte strains for inclusion in various inoculum formulations for use as a foliar or seed treatment. A Kirby-Bauer disk diffusion assay and a dual streak plate assay were each used as tests for compatibility of the strains. All two-strain combinations of TP-SK5, TP-SN7, WP1, WW7, WP5, and SD1 were tested.

The Kirby-Bauer Disk Diffusion Assay was used to evaluate the potential antagonism or inhibitory activity between strains by analyzing the production of diffusible inhibitory metabolites. Mueller-Hinton agar plates were prepared at a consistent depth of about 3-5 mm to ensure uniform diffusion, and each endophyte strain was grown to a standardized inoculum density equivalent to the 0.5 McFarland Standard. Zones for each of endophyte strains TP-SN7, WP1, WW7, WP5, and SD1 were created in a grid pattern on the agar plates. A sterile swab was used to create a confluent lawn of the TP-SK5 strain on each agar plate. Sterile filter paper disks impregnated with a strain selected from TP-SN7, WP1, WW7, WP5, and SD1 and placed in their designates zones (zones 2-6) of the agar plate. This arrangement tests the compatibility of the TP-SK5 strain with the remaining endophyte strains. The plates were incubated at 28° C. for 48-72 hours, after which the plates were analyzed for zones of inhibition surrounding the disks. A lack of visible zones was found on the agar plates, indicating compatibility of the TP-SK5 strain with each of the other selected strains. FIG. 4O provides images of the Kirby-Bauer results, in which no zones of inhibition were observed.

The dual streak plate assay was performed as a complementary test to visually observe direct interactions between two endophyte strains on a single agar surface. All two-strain combinations of TP-SK5, TP-SN7, WP1, WW7, WP5, and SD1 were tested using this assay. Nutrient agar plates were prepared, and the two strains were streaked in parallel lines approximately 1 cm apart using sterile inoculation loops. Following incubation at 28° C. for 48-72 hours, the plates were assessed for growth patterns. Compatibility was indicated as all strains exhibited normal growth along their respective streak lines with no signs of inhibition or antagonistic interactions. Based on the results of the dual streak plate assay, all of the tested strains are compatible with each other. The results of the dual streak plate assays are reported in FIG. 4P.

Further dual streak plate assays were used to evaluate the combination of additional strains; HT1-10, HT1-9, HT1-8, TP-SK5, TP-SN7, SD1, R10, WW7, and PTD1. The plates were assessed for growth rates; (Slower growth: −1, Same growth rate: 0, Overgrowth: 1) and compatibility; (Not Compatible (−−): −2, Slightly Incompatible (−): −1, Slightly Compatible (+): 1, Very Compatible (++): 2). The results of the assay are reported in FIG. 4Q.

Together, the Kirby-Bauer Disk Diffusion Assay and the dual streak plate assay showed that the selected strains for inclusion in inoculum formulations exhibited no antagonistic interactions and are expected to be successfully fermented in multi-strain incubations. The results of these assays indicate that the proposed combinations of endophyte strains can be effectively used in foliar or seed treatments to enhance plant growth, nutrient uptake, and overall health without inhibitory interference.

### Example 1J

Genomic Analyses of Curtobacterium salicis (WW7)

Curtobacterium salicis (WW7) is a new nitrogen fixing diazotrophic bacterial species naturally found in willow trees, the grass phyllosphere (leaf), leaf litter/soil, and in corn roots. WW7 also produces organic acids malate and citrate that are able to solubilize insoluble forms of phosphate, and a Fe siderophore that solubilizes insoluble forms of iron. Curtobacterium salicis was isolated from Willow (Sitka sitchenses) tree stem vasculature.

WW7 was sequence by U.S. Department of Energy (DOE) Joint Genome Institute (JGI) using the Illumina MiSeq platform. The paired-end library was constructed from 376 ng of gDNA using the Nextera DNA Flex Library preparation kit and loaded in one flow cell. The library was barcoded in order to be mixed with 11 samples and sequenced using a 2×250-bp format. The MiSeq run was performed using the MiSeq Reagent Kit v3 (600 cycles) chemistry. The shotgun sequencing yielded 1,530,321 read. After trimming, quality filtering, and the removal of possible contamination using the BBMap package, 1,436,665 read pairs were used as input for SPAdes v3.13.0 genome assembler. The final assembly was generated using a multi-k-mer approach (k=77, 95 and 127).

The genome of Curtobacterium salicis (WW7) strain is represented by 18 scaffolds (N₅₀=329,216 bp) and 3,489,963 bp in length with a G+C content of 71.35%, corresponding to ˜84×coverage. WW7 genome completeness was calculated based on the presence of Actinomycetales lineage-specific single copy marker genes using CheckM v1.0.8. In this regard, a completeness of the 99% was achieved. Gene predictions for the draft assemblies were performed using Prokka v1.11 (7). Of the 3,363 predicted genes, 3286 were protein coding genes, 53 were tRNAs, 12 miscRNA, 1 tmRNA, and 11 rRNA. A total of 1,114 genes were assigned to Clusters of Orthologous Groups (COG), 1,082 were annotated with an enzyme commission (E.C.) number, and 1,722 were assigned to KEGG Orthology (KO).

In order to taxonomically classify WW7 down to species-level, two different strategies were used: (i) average nucleotide identity (ANI) analysis using the ANIm algorithm (see Seemann T. (2014). Prokka: rapid prokaryotic genome annotation. Bioinformatics 30:2068-2069. doi: 10.1093/bioinformatics/btu153). Prokka: rapid prokaryotic genome annotation. Bioinformatics 30:2068-2069. Doi: 10.1093/bioinformatics/btu153), and (ii) calculation of the intra-species probability (Printra-species) using the whole-genome based average nucleotide identity (gANI) strategy described in Varghese et al. (see Richter M and Rossello-Mora R. (2009) Shifting the genomic gold standard for the prokaryotic species definition. Proc Natl Acad Sci USA. 106:19126-19131). In this regard, WW7 genome was compared against all Curtobacterium genus genome assemblies publicly available in the NCBI GenBank database: 107 Curtobacterium genomes in total. By using the ANIm algorithm, all genomes were aligned against each other, and ANI values were used to build adjacency matrix. Such matrix was converted into a similarity matrix (FIG. 4R) and clusters of closely related genomes were extracted using a cutoff of 0.9 (which correspond to a 90% ANI). WW7 was closely related (ANI>90%) to Curtobacterium herbarum DSM 14013 (ASM1690733v1) and seven new Curtobacterium strains isolated from leaf litter in southern California (NCBI BioProject Accession Number: PRJNA391502): Curtobacterium sp. MCPF17_052 (NCBI Assembly ID: ASM323408v1), Curtobacterium sp. MCPF17_047 (NCBI Assembly ID: ASM323404v1), Curtobacterium sp. MCPF17_031 (NCBI Assembly ID: ASM323403v1), Curtobacterium sp. MCPF17_011 (NCBI Assembly ID: ASM323414v1), Curtobacterium sp. MCPF17_001 (NCBI Assembly ID: ASM323461v1), Curtobacterium sp. MCLR17_032 (NCBI Assembly ID: ASM323479v1), Curtobacterium sp. MCBD17_030 (NCBI Assembly ID: ASM322425v1)—see FIG. 4R. Additional WW7 was distantly related (84%<ANI) to two strains isolated from leaf litter in Massachusetts (MCBA15_007 (ASM186490v1), MCBA15_005 (ASM186485v1)) and a Curtobacterium pusillum (NCBI Assembly ID: ASM202564v1) isolated from corn roots.

To further evaluate the affiliation of the WW7 strain to Curtobacterium type strains, pairwise digital DNA-DNA hybridization values (dDDH) were calculated for the WW7 strain to determine its interspecies relatedness with the representatives (type-strains) of the Curtobacterium genus. Pairwise dDDH values between WW7 and Curtobacterium type-strains were lower than 70%, indicating that the WW7 strain is representative of a novel Curtobacterium species, as shown in FIG. 4S. See Kim M K, Kim Y J, Kim H B, Kim S Y, Yi T H, Yang D C 2008. Curtobacterium ginsengisoli sp. nov., isolated from soil of a ginseng field. Int J Syst Evol Microbiol. 58 (10): 2393-7. Similarly, the Genome Taxonomy database (GTDB) identifies WW7 as the only member of a novel species cluster i.e., Curtobacterium flaccumfacies (https://gtdb.ecogenomic.org/species?id=Curtobacterium%20flaccumfaciens), further supporting the lack of affiliation of this strain to any known Curtobacterium species.

Phylogenetic distances were also calculated according to the Anvi'o pan-genomic pipeline, using 605 concatenated single-copy protein coding genes assigned to clusters conserved in all GTDB Curtobacterium representative strains. The resulting phylogenetic tree shows a clear separation of strain WW7 from other Curtobacterium species, identifying C. herbarum as the closest type strain. See FIG. 4T. The Anvi'o pan-genome analysis pipeline was used to identify the 605 single copy core gene, and to generate a partition file. A partitioned analysis was then performed with IQ-TREE to calculate the best substitution model for each single-copy core gene. Bootstrap values were calculated based on 1000 replications and only nodes with a bootstrap value >80% are shown. Asterisks indicate Curtobacterium type-strains according to the List of Prokaryotic names with Standing in Nomenclature (LPSN) database. Clavibacter michiganensis was used as the outgroup.

### Example 1K

### Genomic Analysis of Rhizobium sp. (PTD1)

To evaluate the affiliation of the PTD1 strain to Rhizobium type strains, pairwise digital DNA-DNA hybridization values (dDDH) were calculated for the PTD1 strain to determine its interspecies relatedness with the closest representatives (type-strains) of the Rhizobium genus. Pairwise dDDH values between PTD1 and Rhizobium type-strains were lower than 70%, indicating that the PTD1 strain is representative of a novel Rhizobium species, as shown in FIG. 4U.

### Example 1L

### Colonization by 11R-BB, SD2, HT1-2, HT1-9, WP5, WW5, and WW6.

To demonstrate colonization of the endophytes in a mixed consortia formulations were made and used to treat poplar roots in a solution and sprayed on as a foliar to corn shoots. For poplar roots the small plexi glass Rhizochips were used to hold and help visualize fluorescently tagged strains in planta and imaged using an inverted confocal Leica STELLARIS 5 LIAchroic. Poplar roots were treated in solution with a mixed formulation of the endophytes was applied using the GFP tagged-strains (SD2, HT1-9, and WP5) together with their mCherry-tagged synergistic partners (11R-BB, HT1-2, and WW5). The poplar roots were examined seven days after treatment and showed successful colonization in root cells and vasculature. Images of the strains present in planta are shown in FIG. 4V.

Corn shoots were treated foliarly with a mixture of a CFP-tagged WW5 strain and an RFP-tagged WW6 strain. The corn shoots were examined fourteen days after treatment and showed successful colonization inside mesophyll cells and leaf vasculature. Images of the strains present in planta are shown in FIG. 4W.

### Example 2A

### Characterization of Inoculum Formulation CFU/ml for Use as a Foliar Spray and Seed Treatment.

To demonstrate survival of endophytes on seed synthetic culture formulations were used as a crop seed treatment. For this use the endophytes must survive on seed coats after a dry down dehydration step. The results demonstrate, through a series of seed dry down dehydration survival tests, clear abilities for some strains to be used in seed treatments.

To assess the nitrogen fixation of selected endophyte strains, the SD1, SD2, 11RB (11R-B1+11R-B2), HT1-2, HT1-4, HT1-7, HT1-8, HT1-9, HT1-10, R10, WP5, WP4-10-4, PTD1, WW5, WW6, WW7, 11R-B1, 11R-B2, 11R-BC, and certain combinations of the strains were fermented into a turbid suspension using novel methods. Cells were grown for application purposes in nitrogen-limited media for 1-3 days individually until they the endophyte strain was present in the media at a concentration in a range of about 10⁷ CFU/mL to about 10¹⁰ CFU/mL. In some implementations, two or more endophyte strains may be combined and co-fermented to produce a fermentate having combined concentrations in a range of about 10³ CFU per mL to about 10⁹ CFU per mL, e.g., at least about 10⁴ CFU per mL, at least about 10⁵ CFU per mL, at least about 10⁶ CFU per mL, at least about 10⁷ CFU per mL, at least about 10⁸ CFU per mL, and at least about 10⁹ CFU per mL, or any value or range of values therein. The fermentation process conditions may include a pre-determined incubation temperature in a range of about 20° C. to about 30° C. (e.g., about 23° C. to about 26° C., about 25° C., or any value or range of values therein), shaking the fermentation vessels at a rate in a range of about 25 rpm to about 300 rpm (e.g., about 75 rpm to about 250 rpm, about 125 rpm to about 225 rpm, about 200 rpm, or any value or range of values therein), and fermentation of volumes of about 1 L to about 10 L (e.g., about 2 L to about 8 L, about 4 L to about 6 L, about 4 L, about 2 L, or any value or range of values therein).

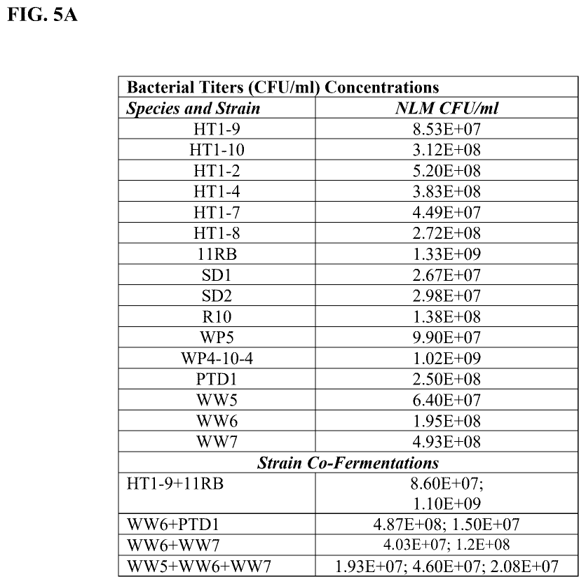

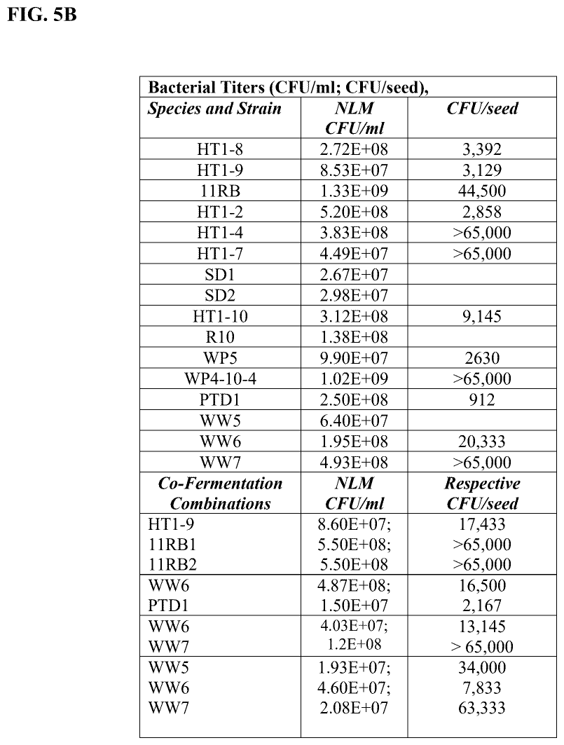

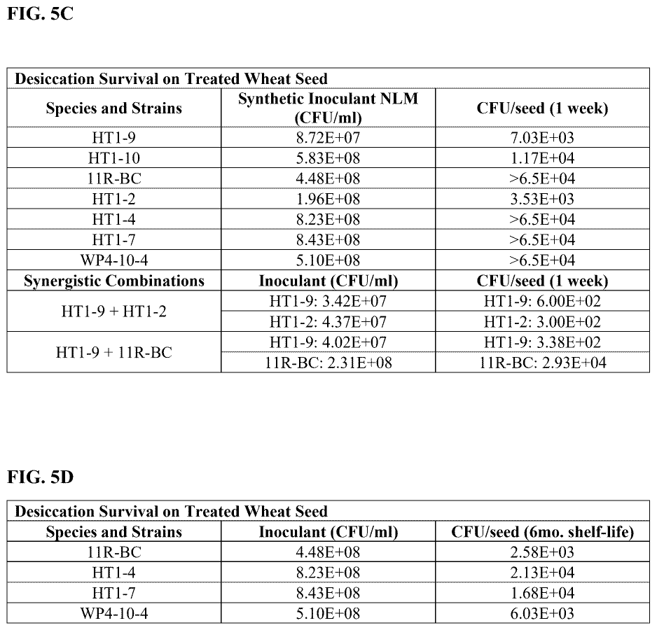

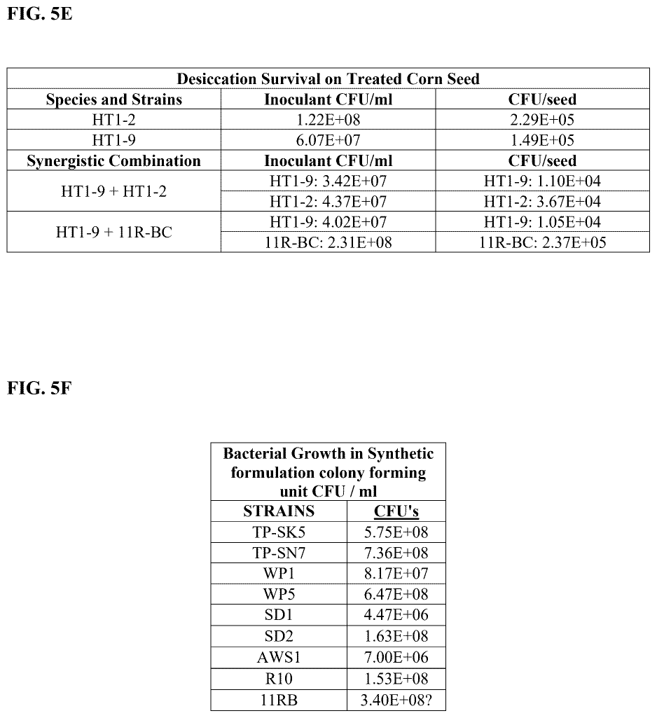

In order to drive upregulation of microbial nitrogenase genes in the endophyte strains, the Nitrogen-Limited Media may be virtually free from nitrogen, but may include one or more sugars, such as mannitol, mannose sucrose, glucose, fructose, lactose, and other appropriate sugars. The Nitrogen-Limited Media may also include one or more salts, such as sodium chloride, phosphate salts (e.g., monopotassium phosphate, dipotassium phosphate, and other phosphate salts), sulfate salts (e.g., MgSO₄), chloride salts (e.g., CaCl₂)), and other appropriate salts, but excluding nitrates, ammonium salts, and other sources of nitrogen. The fermentation solution may further include other appropriate constituents, such as yeast extract, agar, and other appropriate ingredients. The resulting composition may be utilized as a liquid composition for treating a host plant. See, e.g., the following reference regarding nitrogen-limited media examples: R. J. Rennie, A single medium for the isolation of acetylene reducing (dinitrogen-fixing) bacteria from soils, Canadian Journal of Microbiology, vol. 27, no. 1, pp. 8-14, 1981. The concentration of the tested endophyte strains after the fermentation process are provided in FIG. 5A.

### Example 2B

### Characterization of On-Seed Survival for Use as a Seed Treatment.

The resulting fermentation solution was subsequently applied to seeds for testing survival of the endophytes on the seeds. The strain formulations were mixed with a pure sodium alginate (0.5% w/v) a seed-compatible carbohydrate that dries to a hard partially hydrated protective shell. This blended formulated inoculum solution was then used to coat winter wheat seed (var. pro410 high yield soft winter wheat).

Winter wheat seeds (var. pro410 high yield soft winter wheat) were treated with a seed inoculant liquid composition comprising one or more heterologous endophyte strain from the following list: SD1, SD2, HT1-2, HT1-4, HT1-7, HT1-8, HT1-9, HT1-10, R10, WP5, WP4-10-4, PTD1, WW5, WW6, WW7, 11R-B1+11R-B2, and HT1-9+11R-B1+11R-B2. Seeds were treated at a rate 0.4 μL/seed. The fermentation cultures were prepared under conditions (containing endophytes at ˜10⁷ CFU/mL) in NLM media plus sodium alginate (0.5% w/v) as described herein and then refrigerated. The fermentation cultures were removed from the refrigeration, mixed well, and carefully pipetted onto the seeds at a rate of 3.4 mL/lb. of seed dispersed in 1 mL drops in a sterile laminar flow hood. The treated seeds were manually tumbled and massaged carefully after each 1 mL addition. Once the entire 3.4 mL/lb application was added, the seeds were massaged, shaken, and tumbled for 2-3 minutes until all seed appeared visibly wet in the bag. The bag was then opened for air-drying in the sterile laminar flow hood. After drying, the seeds were stored at room temperature for 3 weeks and then assayed for microbial survival.

For assessment of the bacterial seed, a seed coat wash and enumeration assay was conducted. The bacterial formulation dried coat was washed off of wheat seeds by using an enumeration and dilution plating methods as follows; for wheat 20 seed, or for corn 10 seeds, were first added separately to 10 mL of 1×PBS and the slurry solutions plus seeds were vortexed for 30 seconds to dissolve hard dried seed coats with bacteria. To ensure seed wash the tubes with seeds were then placed on the shaker at 150 RPM for 1 hour at 25° C. The seed wash solution plus seeds were then vortexed for an additional 30 seconds and then dilution plated using 1×PBS and plated on NLM high purity agar plates. The plates were then incubated for 3 days and colonies were counted by morphology type. The resulting bacterial titers are shown in FIG. 5B.

The results shown in FIG. 5B demonstrate positive survival on seed results for the top performing strains HT1-4, HT1-7, 11R-BC, HT1-9, Ht1-8, HT1-2, HT1-10, WP5, PTD1, WW5, WW5, WW6, and WW7. The eight surviving on seed dehydrated strains taken together as single strains, pairs of strains, three strains, four strains or five strains are useful to inoculate seeds and colonize subsequent germinated seedlings for long term shelf-life stable product survival on seed after treatments prior to planting seed.

### Example 2C

### On-Seed Survival Assessment of Single Stains and Combinations.

In further studies, winter wheat seeds (var. pro410 high yield soft winter wheat) were treated with a seed inoculant liquid composition comprising one or more heterologous endophyte strains as follows: 11R-BC, HT1-2, HT1-4, HT1-7, HT1-9, HT1-10, WP4-10-4, a synthetic consortium of HT1-9+11RB (11R-B1+11R-B2), and a synthetic consortium of HT1-9+11R-BC. The seed treatments were carried out as described in Example 2B. The endophyte formulations were mixed together with a low viscosity high purity sodium alginate solution (0.5% w/v) a bacterial endophyte seedling compatible carbohydrate that dries to a hard partially hydrated protective shell on seeds. This blended formulated inoculum solution was then used to coat seeds from crops at a rate of 40-80 ul/100-200 seeds. The treated seeds were stored 6 months.

After the 6-month period, the survival of the endophytes on the seeds was tested using a seed coat wash and enumeration assay as described in Example 2B. The results of the assay are provided in FIG. 5C. The results demonstrate on-seed survival of endophyte strains HT1-4, HT1-7, 11R-BC, HT1-9, HT1-10, HT1-2, and WP4-10-4. Synthetic consortia of HT1-9+HT1-2 and HT1-9+11R-BC demonstrated that multiple strains can be combined in synthetic consortia and successfully applied to seeds with long term survival, enabling long shelf-life.

### Example 2D

### On-Seed Survival Assessment of Single Stains and Combinations.

In further studies, winter wheat seeds (var. pro410 high yield soft winter wheat) were treated with a seed inoculant liquid composition comprising single, gram-positive heterologous endophyte strains as follows: 11R-BC, HT1-4, HT1-7, and WP4-10-4. The seed treatments were carried out as described in Example 2B. The endophyte formulations were mixed together with a low viscosity high purity sodium alginate solution (0.5% w/v) a bacterial endophyte seedling compatible carbohydrate that dries to a hard partially hydrated protective shell on seeds. This blended formulated inoculum solution was then used to coat seeds from crops at a rate of 40-80 ul/100-200 seeds. The treated seeds were stored 6 months.

After the 6-month period, the survival of the endophytes on the seeds was tested using a seed coat wash and enumeration assay as described in Example 2B. The results of the assay are provided in FIG. 5D. The results demonstrate on-seed survival of all tested endophyte strains 11R-BC, HT1-4, HT1-7, and WP4-10-4, demonstrating successful long-term survival, enabling long shelf-life.

### Example 2E

### On-Seed Survival Assessment of Single Stains and Combinations.

Further on-seed survival studies were conducted to test synthetic consortia that include synergistic enhanced pairs using two key nitrogenase booster strains (HT1-2 and 11R-BC) combined together with a strong diazotroph (HT1-9). These pairs were tested on winter wheat seeds (var. pro410 high yield soft winter wheat). The seed treatments were carried out as described in Example 2B.

A seed coat wash and enumeration assay was conducted on the seeds after dehydration at 25° C. and a 1-week storage period. The results shown in FIG. 5E. The results demonstrate on-seed survival of the consortia including a synergist booster strain and a diazotrophic strain in enhanced pairs. These combined strains were documented surviving well at CFU/seed rates greater than ˜10,500-˜237,500 CFU/seed for each dried and dormant wheat crop seed after one week.

### Example 2F

### P Solubilizing Synthesized Product Formulations Used for Foliar and Seed Treatments.

A series of inoculants were prepared for use in enhancing crop plant root-based phosphate solubilization from soil and to enhance root to shoot uptake of free soluble forms of phosphate in crop plants. Endophyte cells from strains TP-SK5, TP-SN7, WP1, WP5, SD1, SD2, AWS1, R10, and 11RB (11R-B1+11R-B2) were cultured in nitrogen-limited media.

The resulting composition may be utilized as a liquid composition for treating a host plant. The concentration of the tested endophyte strains after the fermentation process are provided in FIG. 5F. The results demonstrate that the species and strains listed below are useful for production of artificially synthesized inoculum cultures.

### Example 3A

### Nitrogen Use Efficiency Reduced N Fertilizer Crop Plant Growth Trials

Fermentates for SD1, SD2, 11RB (11R-B1+11R-B2), HT1-2, and WP4-10-4, and a co-fermentate of WW5+WW6+WW7 were prepared as described herein. The method includes a sequence of controlled steps designed to optimize seed germination and plant development. Initially, 15 mL of water is added to a germination box (“germ box”) containing seeds to ensure a moist environment conducive to germination. Once the water is added, the germ boxes with the treated seeds are placed in a refrigerator for a cold stratification period of 2 days, to vernalize and simulate winter conditions and break seed dormancy. After refrigeration, the seeds are exposed to light by placing the germ boxes under an LED light for 3 days at 25° C. in a grow room under sodium halide light (photon flux of 710 μmol m⁻² s⁻¹) on a 14 hr light/10 hr dark lighting cycle. From these, 5 to 6 germinated seeds are selected and carefully transplanted into individual pots. Each experimental treatment requires a total of 9 pots, with either 45 or 54 seeds being used across the various treatments to ensure robust statistical analysis of growth outcomes. Once germinated, plants were watered and fertilized in the trays with Hoagland's solution modified for reduced nitrogen at 50 ppm 2-3 times per week, as needed to maintain moist-slightly dry soil. Germinated seedlings are initially watered with 700-1000 mL 10 ppm Hoagland Solution and 200-500 ml of Hoagland solution is added every other day. The trays were also rotated on same day as watering. Controls were raised under the same conditions with no pre-treatment prior to planting. Plants were harvested individually at 24 days, dried, and weighed.

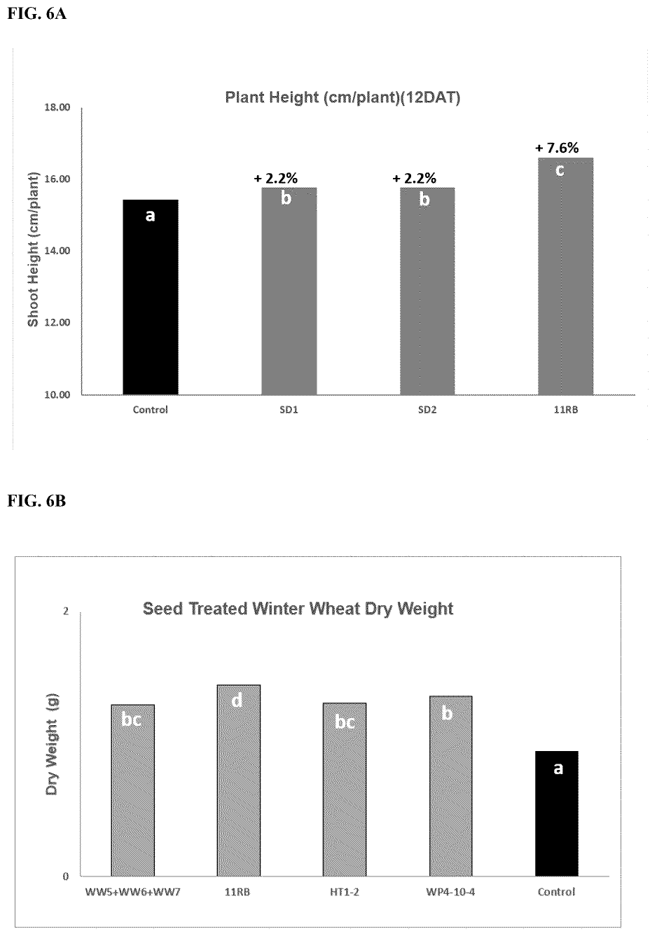

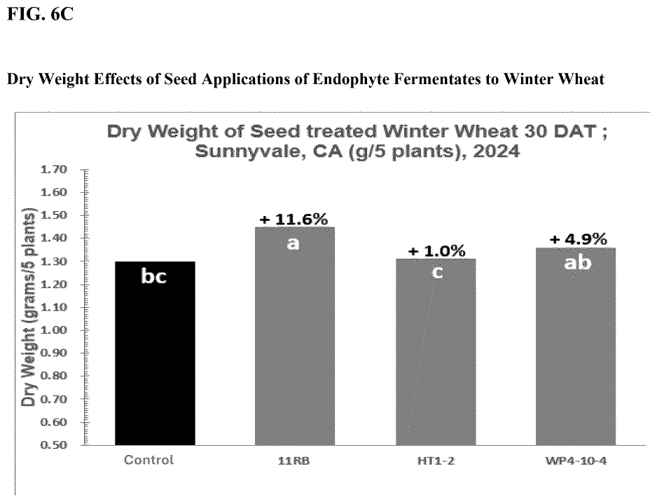

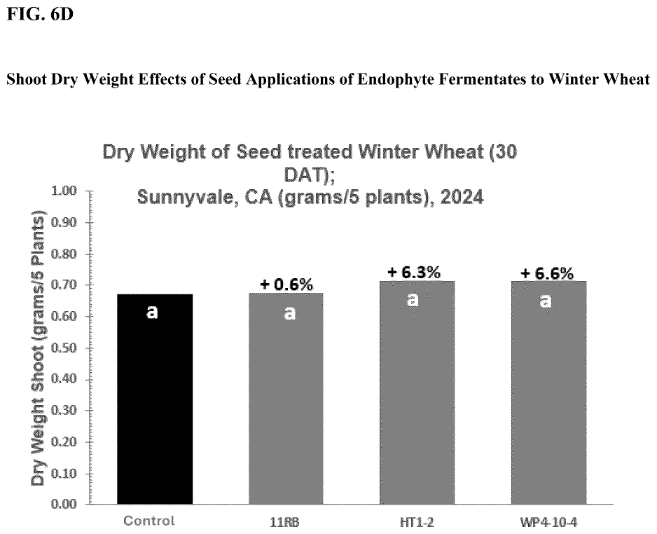

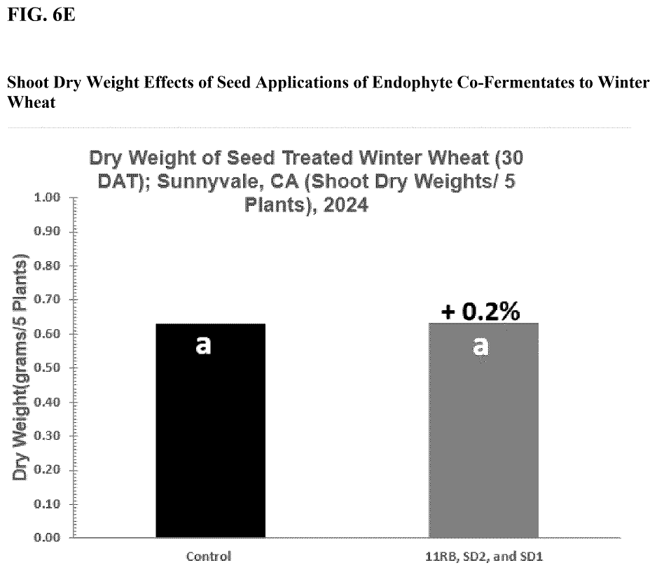

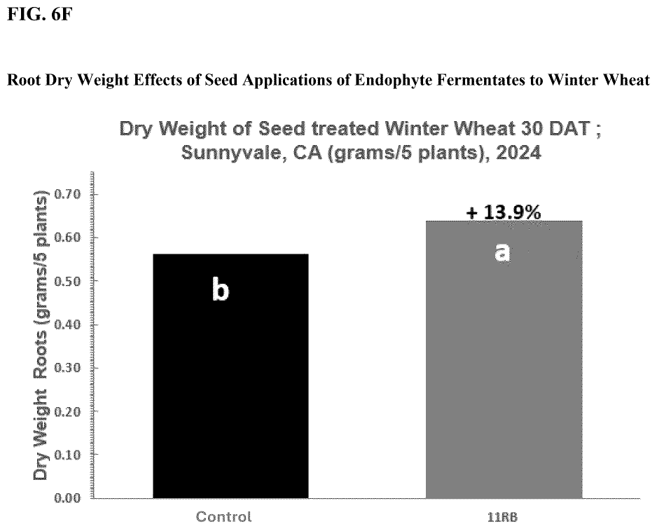

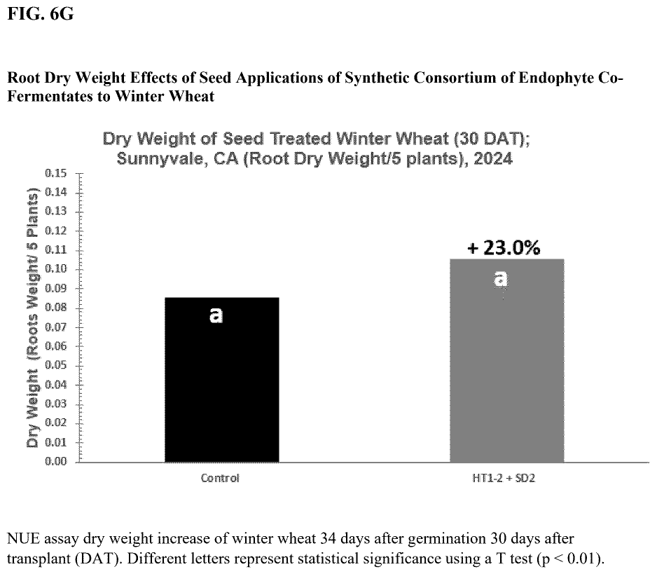

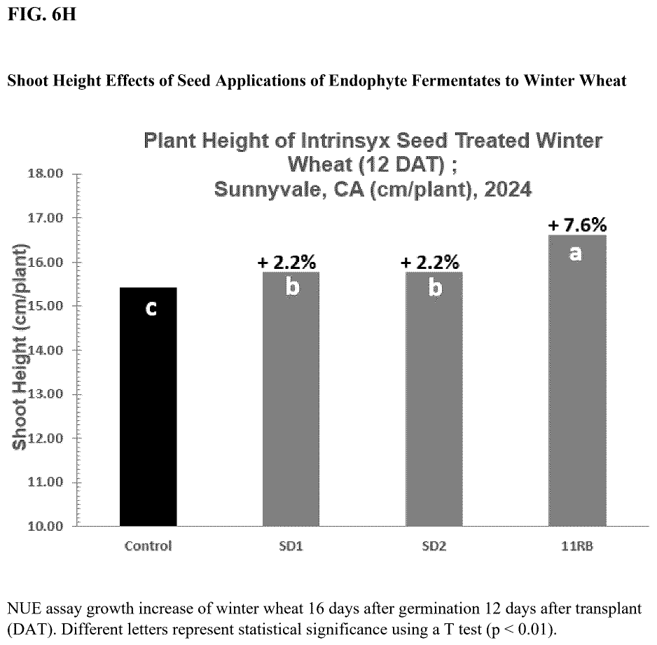

The statistical results of comparisons of the plants treated with SD1, SD2, and 11RB (11R-B1+11R-B2) to controls with respect to shoot height are reported in FIG. 6A. The statistical results of comparisons of the plants treated with 11RB (11R-B1+11R-B2), HT1-2, WP4-10-4, and the co-fermentate of WW5+WW6+WW7 to controls with respect to dry weight are reported in FIG. 6B.

### Example 3B

### Seed Application in GH Trials and Effects on Total Dry Weight

Fermentates for 11RB (11R-B1+11R-B2), HT1-2, and WP4-10-4 were prepared as described in Example 2A. Seed were treated and coated with one of the fermentates of 11RB(1), HT1-2, and WP4-10-4 as described in Example 2B. Control seeds were not treated with fermentate. The seeds were then germinated in a germ box as described in Example 3A. Five to six germinated seeds were selected and carefully transplanted into individual pots. Each experimental treatment requires a total of 9 pots, with either 45 or 54 seeds being used across the various treatments to ensure robust statistical analysis of growth outcomes. The control seeds were planted for comparison. The plants were harvested, dried, and weighed for comparison of total dry weight.

The statistical results of comparisons of the plants treated with 11RB (11R-B1+11R-B2), HT1-2, and WP4-10-4 to controls with respect to total dry weight are reported in FIG. 6C. The 11RB (11R-B1+11R-B2)-treated wheat showed a 11.6% increase in dry weight. The HT1-2-treated plants exhibited a smaller but positive increase of 1.0% compared to controls. The WP4-10-4 strain resulted in a 4.9% increase in dry weight.

### Example 3C

### Seed Application in GH Trials and Effects on Shoot Dry Weight

Fermentates for 11RB (11R-B1+11R-B2), HT1-2, and WP4-10-4 were prepared as described in Example 2A. Seed were treated and coated with one of the fermentates of 11RB (11R-B1+11R-B2), HT1-2, and WP4-10-4 as described in Example 2B. Control seeds were not treated with fermentate. The seeds were then germinated in a germ box as described in Example 3A. Five to six germinated seeds were selected and carefully transplanted into individual pots. Each experimental treatment requires a total of 9 pots, with either 45 or 54 seeds being used across the various treatments to ensure robust statistical analysis of growth outcomes. The control seeds were planted for comparison. The plants were harvested, dried, and weighed for comparison of shoot dry weight.

The statistical results of comparisons of the plants treated with 11RB (11R-B1+11R-B2), HT1-2, and WP4-10-4 to controls with respect to shoot dry weight are reported in FIG. 6D. The 11RB (11R-B1+11R-B2)-treated wheat showed a 0.6% increase in dry weight. The HT1-2-treated plants exhibited a greater positive increase of 6.3% compared to controls. The WP4-10-4 strain resulted in a 6.6% increase in dry weight.

### Example 3D

### Seed Application in GH Trials and Effects on Shoot Dry Weight

A fermentate for 11RB (11R-B1+11R-B2) was prepared as described in Example 2A. Seed were treated and coated with one of the fermentate of 11RB (11R-B1+11R-B2) as described in the method Example 2B. Control seeds were not treated with fermentate. The seeds were then germinated in a germ box as described in Example 3A. Five to six germinated seeds were selected and carefully transplanted into individual pots. Each experimental treatment requires a total of 9 pots, with either 45 or 54 seeds being used across the various treatments to ensure robust statistical analysis of growth outcomes. The control seeds were planted for comparison. The plants were harvested, dried, and weighed for comparison of root dry weight.

The statistical results of comparisons of the plants treated with 11RB (11R-B1+11R-B2) to controls with respect to shoot dry weight are reported in FIG. 6E. The 11RB-(11R-B1+11R-B2) treated wheat showed a 13.9% increase in dry weight.

### Example 3E

### Seed Application of Co-Fermentate in GH Trials and Effects on Root Dry Weight

A co-fermentate including endophyte strains HT1-2 and SD-2 was prepared as described in Example 2A. Seed were treated and coated with the co-fermentate of HT1-2 and SD-2 as described in the method of Example 2B. Control seeds were not treated with co-fermentate. The seeds were then germinated in a germ box as described in Example 3A. Five to six germinated seeds were selected and carefully transplanted into individual pots. Each experimental treatment requires a total of 9 pots, with either 45 or 54 seeds being used across the various treatments to ensure robust statistical analysis of growth outcomes. The plants were harvested, dried, and weighed for comparison of shoot dry weight 34 days after germination and 30 days after transplant.

The statistical results of comparisons of the plants treated with the co-fermentate of HT1-2 and SD-2 to controls with respect to shoot dry weight are reported in FIG. 6F. The treated wheat showed a 23.0% increase in dry shoot weight compared to controls.

### Example 3F

### Seed Application in GH Trials and Effects on Shoot Height

Fermentates for 11RB (11R-B1+11R-B2), SD-1, and SD-2 were prepared as described in Example 2A. Seed were treated and coated with one of the fermentates of 11RB (11R-B1+11R-B2), SD-1, and SD-2 as described in Example 2B. Control seeds were not treated with fermentate. The seeds were then germinated in a germ box as described in Example 3A. Five to six germinated seeds were selected and carefully transplanted into individual pots. Each experimental treatment requires a total of 9 pots, with either 45 or 54 seeds being used across the various treatments to ensure robust statistical analysis of growth outcomes. The plants were compared for shoot height 16 days after germination and twelve days after transplanting.

The statistical results of comparisons of the plants treated with 11RB (11R-B1+11R-B2), SD-1, and SD-2 to controls with respect to shoot height are reported in FIG. 6G. The 11RB (11R-B1+11R-B2)-treated wheat showed a 7.6% increase in shoot height. The SD-1-treated and SD-2-treated plants both showed an increase of 2.2% compared to controls.

### Example 3G

### Seed Application and Effects on Shoot Height

Fermentates for HT1-4, HT1-7, HT1-10, R10, and WP5 were prepared as described in Example 2A. Seed were treated and coated with one of the fermentates of HT1-4, HT1-7, HT1-10, R10, and WP5 as described in Example 2B. Control seeds were not treated with fermentate. The seeds were then germinated in a germ box as described in Example 3A. Five to six germinated seeds were selected and carefully transplanted into individual pots. Each experimental treatment requires a total of 9 pots, with either 45 or 54 seeds being used across the various treatments to ensure robust statistical analysis of growth outcomes. The plants were compared for shoot height sixteen days after planting and 12 days after transplant.

The statistical results of comparisons of the plants treated with 11RB (11R-B1+11R-B2), SD-1, and SD-2 to controls with respect to shoot height are reported in FIG. 6H. The HT1-4-treated wheat showed a 9.2% increase in shoot height. The HT1-7-treated wheat showed a 9.5% increase in shoot height. The HT1-10-treated wheat showed a 15.7% increase in shoot height. The R10-treated plants showed an increase of 12.9% compared to controls. The WP5-treated wheat showed a 10.1% increase in shoot height.

### Example 4A

### Foliar Application and Effects on Total Dry Weight

Fermentates for SD1, SD2, HT1-2, and HT1-9 were prepared as described in Example 2A. The fermentates of endophyte strains SD1, SD2, HT1-2, and HT1-9 were applied foliarly to winter wheat. After growing the winter wheat for one week, liquid fermentates of each of the endophyte strains was sprayed individually on the wheat plants. The spray was applied at a rate of 32 ounces per acre, diluted in a 20-gallon solution to ensure even distribution across the treated plants. Plants were harvested, dried, and weighed after 30 days.

A pot trial design was conducted on 9 pots for each treatment that were then thinned after 2 weeks to 4 tallest plants in each pot. Four-inch standard plastic green pots were exposed to LED lights at ˜700 μEinsteins at 25° C. in a controlled grow room. Plants were watered and fertilized 2-3 times per week as needed to maintain moist-slightly dry soil. The solution used for watering and fertilization was ¼ X Hoagland's solution modified for reduced nitrogen at 10 or 25 ppm N. Controls were raised under the same conditions with no endophyte formulation treatment prior to planting. After growth, plants were measured periodically for height and then at 30 days harvested individually, pooled dried and weighed.

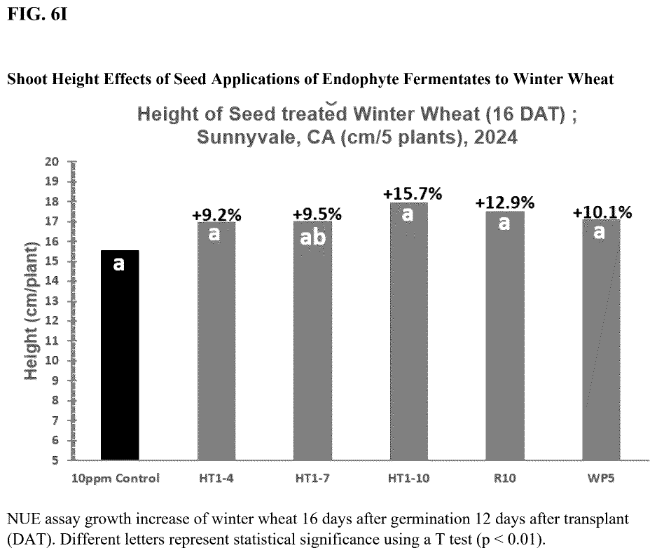

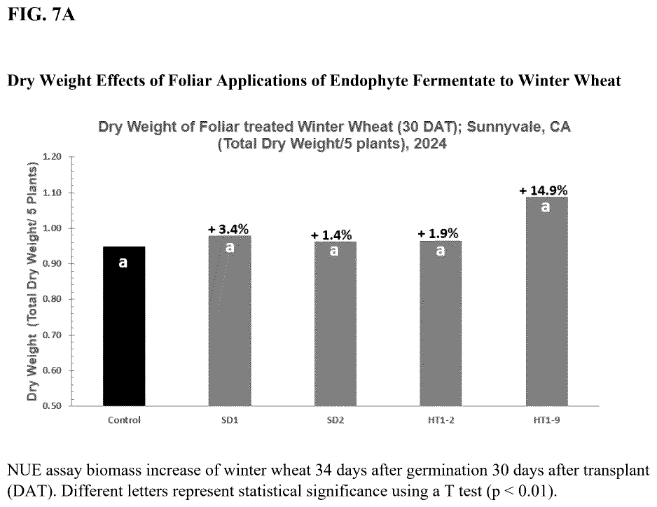

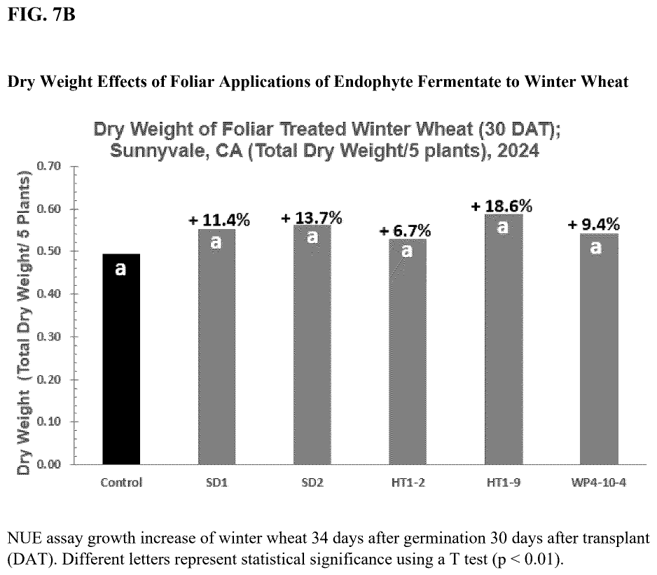

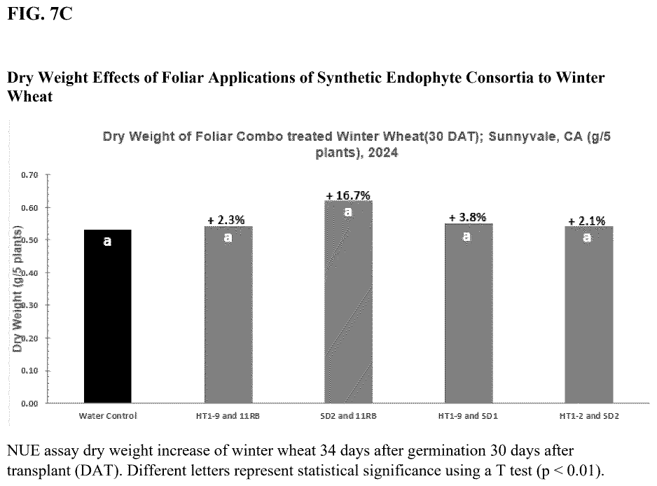

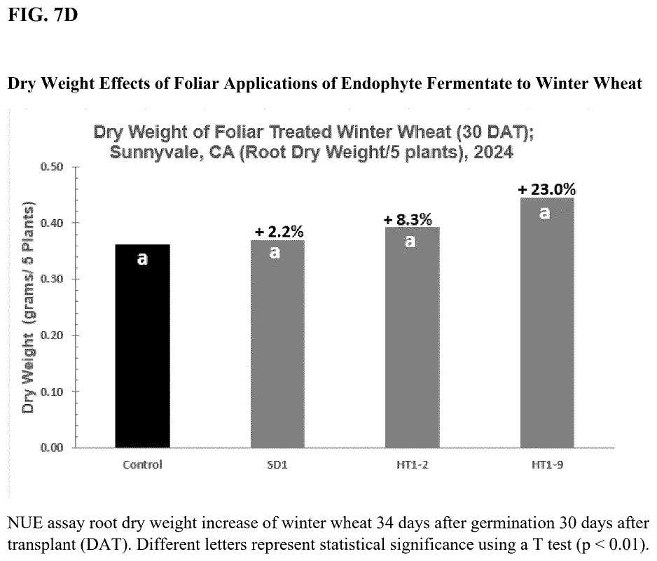

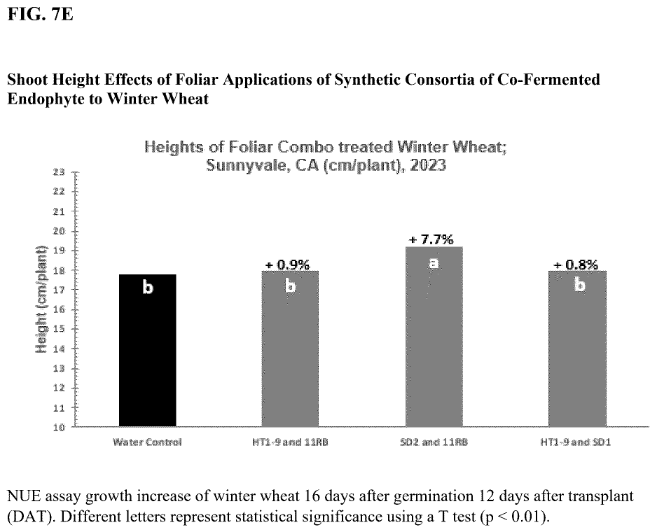

The results are provided in FIG. 7A and demonstrate single strain endophyte formulation foliar applications caused average whole plant (shoot+root) increases in dry weight under moderate N deficiency 30 days after transplant and 34 days after germination. The strains tested demonstrated varying effects on the dry weight of the winter wheat compared to the control group. The SD1-treated wheat showed a 3.4% increase in dry weight, while the SD2-treated plants exhibited a smaller but positive increase of 1.4%. The HT1-2 strain resulted in a 1.9% increase in dry weight, with HT1-9 showing a notably higher increase of 14.9%, as shown in FIG. 7A.

This method highlights the differential effects of various endophyte strains on the biomass accumulation of winter wheat, with HT1-9 emerging as a particularly effective treatment in promoting dry weight gain under the tested conditions. These findings show the tested endophyte strains enhance plant growth and biomass production.

### Example 4B

### Foliar Application and Effects on Dry Weight

Fermentates for SD1, SD-2, HT1-2, HT1-9, and WP4-10-4 were prepared as described in Example 2A. The fermentates of endophyte strains SD1, SD2, HT1-2, HT1-9, and WP4-10-4 were applied foliarly to winter wheat as described in Example 4A. Results are shown in FIG. 7B.

The strains tested demonstrated varying effects on the dry weight of the winter wheat compared to the control group. The SD1-treated wheat showed a 11.4% increase in dry weight. The SD2-treated wheat showed a 13.7% increase in dry weight. The treatment with the HT1-2 strain resulted in a 6.7% increase in dry weight. The treatment with the HT1-9 showing a notably higher increase of 18.6%. The treatment with the WP4-10-4 showing a notably higher increase of 9.4%. The results demonstrate single strain endophyte formulation foliar applications caused average whole plant (shoot+root) increases in dry weight per 5 wheat plants pooled (n=45 plants from 9 pots).

### Example 4C

### Foliar Application and Effects on Dry Weight in Field Test

Co-fermentates for HT1-9 and 11RB (11R-B1+11R-B2), SD-2 and 11RB (11R-B1+11R-B2), HT1-9 and SD-1, and HT1-2 and SD-2 were prepared as described in Example 2B, where the strain pairs were co-fermented under the described conditions. The synthetic consortia of co-fermented endophyte strains HT1-9 and 11RB (11R-B1+11R-B2), SD-2 and 11RB (11R-B1+11R-B2), HT1-9 and SD-1, and HT1-2 and SD2 were applied using a foliar spray to winter wheat according to the method described in Example 4A. The results are shown in FIG. 7C.

The strains tested demonstrated varying effects on the dry weight of the winter wheat compared to the control group, which was treated with water and no endophyte strains. The HT1-9 and 11RB (11R-B1+11R-B2) co-fermentate treatment resulted in the wheat showed a 2.3% increase in dry weight. The SD-2 and 11RB (11R-B1+11R-B2) co-fermentate treatment showed a 16.7% increase in dry weight. The HT1-9 and SD-1 co-fermentate treatment showed a 3.8 increase in dry weight. The HT1-2 and SD-2 co-fermentate showed a 2.1% increase in dry weight.

The results demonstrate a synergistic effect resulting from treatment with the SD-2 and 11RB (11R-B1+11R-B2) synthetic consortium, as the 16.7% increase in total dry weight is more than ten times greater than the increase in dry weight shown by treatment with SD-2 fermentate shown in Examples 4A and 4B, and is significantly higher than the increase in dry weight resulting from treatment with 11RB (11R-B1+11R-B2) fermentate alone, as shown in Example 3E.

### Example 4D

### Foliar Application and Effects on Root Dry Weight in Field Test

Fermentates for SD1, HT1-2, and HT1-9 were prepared as described in Example 2B. The fermentates of endophyte strains SD1, HT1-2, HT1-9, and WP4-10-4 were applied to winter wheat by foliar spray according to the method described in Example 4A. The results are shown in FIG. 7D.

The strains tested demonstrated varying effects on the root dry weight of the winter wheat compared to the control group. The SD1-treated wheat showed a 2.2% increase in root dry weight. The HT1-2 strain resulted in an 8.3% increase in root dry weight, with HT1-9 showing a notably higher increase of 23.0%, as shown in FIG. 7D.

HT1-9 emerging as a particularly effective treatment in promoting dry weight gain under the tested conditions. These findings show the tested endophyte strains enhance plant growth and biomass production.

### Example 4E

### Foliar Application of Synthetic Consortia and Effects on Shoot Height

Synthetic consortia of co-fermented HT1-9 and 11RB (11R-B1+11R-B2), SD-2 and 11RB (11R-B1+11R-B2), and HT1-9 and SD-1 were prepared as described in Example 2A. The co-fermentates of HT1-9 and 11RB (11R-B1+11R-B2), SD-2 and 11RB (11R-B1+11R-B2), and HT1-9 and SD-1 were applied using a foliar spray to winter wheat, as described in Example 2B with some modification. The plants were assayed at 16 days after germination 12 days after transplant. The results are shown in FIG. 7E.

The statistical results of comparisons of the plants treated with HT1-9 and 11RB (11R-B1+11R-B2), SD-2 and 11RB (11R-B1+11R-B2), and HT1-9 and SD-1 to controls with respect to shoot height are reported in FIG. 7E. The plants treated with HT1-9 and 11RB (11R-B1+11R-B2) showed a 0.9% increase in shoot height. The HT1-7-treated wheat showed a 9.5% increase in shoot height. The plants treated with SD-2 and 11RB showed a 7.7% increase in shoot height. The plants treated with HT1-9 and SD-1 showed an increase of 0.8% compared to controls.

### Example 5A

### Root Soak Application and Effects on Rice Dry Weight

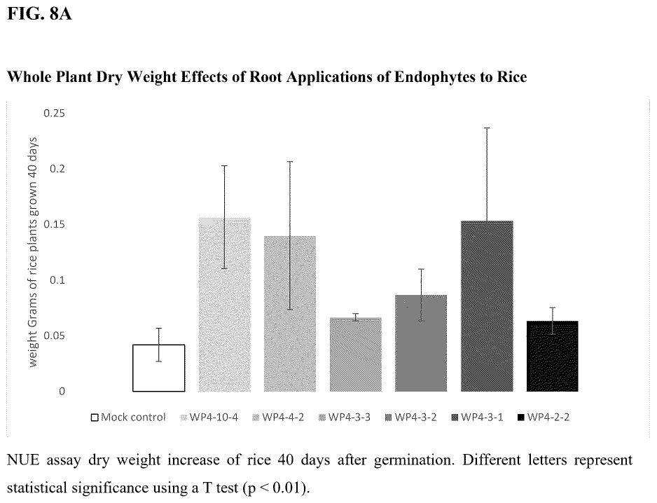

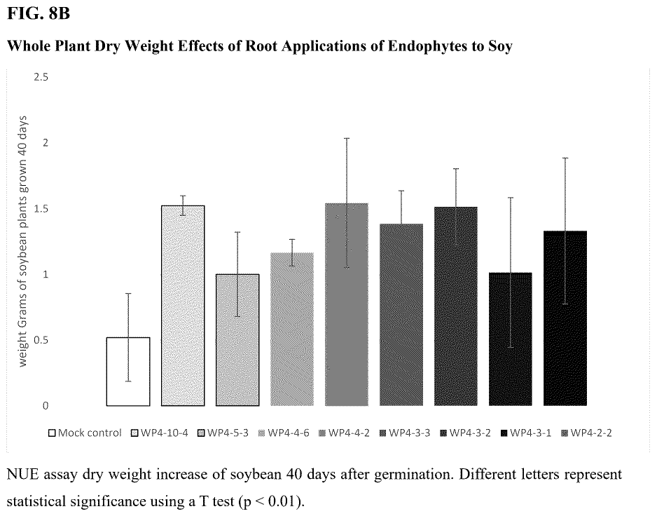

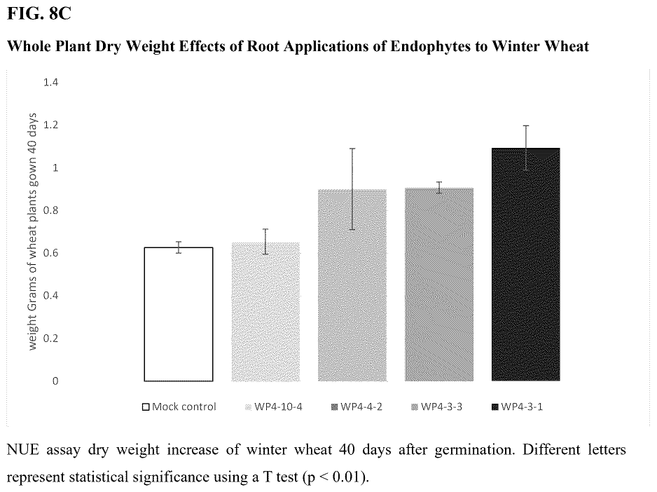

To assess the growth promoting effects of NUE tree endophytic bacterial strains, rice seeds were germinated on agar with Murashige and Skoog salts. Seedlings were inoculated with each endophyte grown in NLM media using a seedling in endophyte formulation soak or root drench method. Endophytes were prepared according to the method described in Example 2A. Six seedlings were then placed with their roots in a small amount of 1/10× diluted inoculum formulations in Magenta vessels and allowed to incubate overnight. Seedlings were then rinsed with sterile water several times before being planted in soil. The plants were grown in Sunshine mix #4 in 4-inch square pots that were 3 inches deep. Plants were grown under T5 blue light bulbs (6500K 54WATT) during a 16 hours light, 8 hours dark-light cycle. Lights were set about 3 feet from the plants and light intensity was measured at 200 μmole m-2s-1. The plants were watered freely when the soil was slightly dry and were not given any additional fertilizer. Plants were grown for 40 days. Above ground tissue was then removed, dried, and weighed. The results of dried weights of the treated plants versus controls are report in FIG. 8A.

Single inoculum formulations of endophyte strains used as a root drench on rice grown under NUE low N conditions resulted an average whole plant (shoot+root) biomass increases in dry weight per plant (n=6 plants) 40 days after germination and subsequent growth under N deficiency without applying nitrogen fertilizer. Treatments with WP4-10-4, WP4-3-1, and WP4-4-2 each resulted in an approximate 75% increase in dry weight. WP4-3-3 applications resulted in an ˜10% increase.

### Example 5B

### Root Soak Application and Effects on Soy Dry Weight

To assess the growth promoting effects of NUE tree endophytic bacterial strains, soy seeds were germinated on agar with Murashige and Skoog salts. Seedlings were inoculated with each endophyte grown in NLM media using a seedling in endophyte formulation, soak or root drench method. Endophytes were prepared according to the method described in Example 2A. Six seedlings were then placed with their roots in a small amount of 1/10× diluted inoculum formulations in Magenta vessels and allowed to incubate overnight. Seedlings were then rinsed with sterile water several times before being planted in soil. The plants were grown in Sunshine mix #4 in 4-inch square pots that were 3 inches deep. Plants were grown under T5 blue light bulbs (6500K 54WATT) during a 16 hours light, 8 hours dark-light cycle. Lights were set about 3 feet from the plants and light intensity was measured at 200 μmole m-2s-1. The plants were watered freely when the soil was slightly dry and were not given any additional fertilizer. Stakes were placed to prop up the soybeans when needed. Plants were grown for 40 days. Above ground tissue was then removed, dried, and weighed. The results of dried weights of the treated plants versus controls are report in FIG. 8B.

Single inoculum formulations of endophyte strains used as a root drench on soy plants grown under NUE low N conditions resulted an average whole plant (shoot+root) biomass increases in dry weight per plant (n=6 plants) 40 days after germination and subsequent growth under N deficiency with No applied N fertilizer. Treatments with WP4-10-4, and WP4-4-2, resulted in approximate 75% increase in dry weight. WP4-3-3 application resulted in a ˜65% increase in dry weight. Applications of WP4-4-6, WP4-3-1 and WP4-5-3 each resulted in 50-55% increases, respectively.

### Example 5C

### Root Soak Application and Effects on Winter Wheat Dry Weight

To assess the growth promoting effects of NUE tree endophytic bacterial strains, winter wheat seeds were germinated on agar with Murashige and Skoog salts. Seedlings were inoculated with each endophyte grown in NLM media using a seedling in endophyte formulation soak or root drench method. Endophytes were prepared according to the method described in Example 2A. Six seedlings were then placed with their roots in a small amount of 1/10× diluted inoculum formulations in Magenta vessels and allowed to incubate overnight. Seedlings were then rinsed with sterile water several times before being planted in soil. The plants were grown in Sunshine mix #4 in 4-inch square pots that were 3 inches deep. Plants were grown under T5 blue light bulbs (6500K 54WATT) during a 16 hours light, 8 hours dark-light cycle. Lights were set about 3 feet from the plants and light intensity was measured at 200 μmole m-2s-1. The plants were watered freely when the soil was slightly dry and were not given any additional fertilizer. Plants were grown for 40 days. Above ground tissue was then removed, dried, and weighed. The results of dried weights of the treated plants versus controls are report in FIG. 8C.

Single inoculum formulations of endophyte strains used as a root drench on winter wheat plants grown under NUE low N conditions resulted an average whole plant (shoot+root) biomass increases in dry weight per plant (n=6 plants) 40 days after germination and subsequent growth under N deficiency with No applied N fertilizer. Treatments with WP4-3-1 resulted in an approximate 75% increase in dry weight. Application of WP4-3-3 resulted in ˜45% increase in dry weight, respectively. Treatment with WP4-10-4 resulted in a ˜5% increase in dry weight.

### Example 6A

### Root Soak Application and Effects on Rice Dry Weight

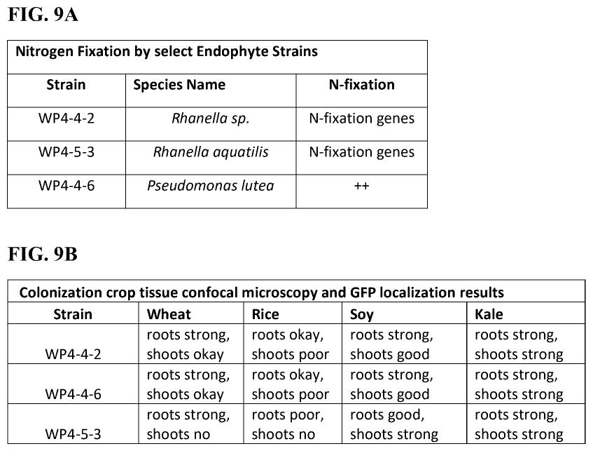

Five special NUE-enhancing endophyte strains provide enhanced nitrogen fixation and uptake. The strains were able to effectively colonize crop plant roots and shoots and positively affect growth and biomass in plants grown under full nitrogen deficiency in a greenhouse pot study. Nitrogen fixation effects of the strains are provided in FIG. 9A.

Localization studies using green fluorescent protein (GFP) expression and laser confocal microscope observation demonstrated single strain endophyte formulations applied by root soaking led to a colonization of crops. The plants treated with the endophytes using the root soak technique described in Example 5A. The endophytes demonstrated strong colonization in winter wheat, good colonization in rice, and strong or good colonization of both soy and kale. These strains are listed and their crop colonization activities are shown in FIG. 9B.

### Example 7A

### Two-Strain Product Co-Fermentation and Formulation

Co-fermentation or co-cultivation, of two endophyte strain(s) WW6 and WW7 was achieved using nitrogen limited media with tightly controlled fermentation conditions. Each strain was first plated on nitrogen limiting semi-solid medium (NLM). Three to four colonies may be selected and used to inoculate 2-liter seed starter cultures with fresh sterile NLM medium. The 2-liter flasks may be grown at about 25° C. to about 30° C. under constant agitation ranging from 200 rpm to about 500 rpm for 72 hours. Upon completion, two stains may be pooled into a single carboy and stored at 4° C. prior to inoculating 4500 liters of NLM media (pH 7.6). The fermentation media may be sterilized in a steam jacketed fermenter having a size in a range of about 1000 to about 30,000 liters. The co-fermentation/co-cultivation may be conducted for 3-4 days at an aeration rate of about 10 to about 30 PSI. The resulting colony forming unit of the two strain(s) ranged from 2.2×10⁸ CFU/mL to 1.23×10⁹ CFU/mL WW6 and 1.03×10⁸ CFU/mL to 1.4×10⁹ CFU/mL WW7 when plated on Tryptic Soy Broth Agar (TSBA).

The co-fermentation methods were applied to the WW6 and WW7 strains to demonstrate efficacy of the co-fermentation methods. The foregoing large-scale co-fermentation method was used to prepare a fermentate comprising both the WW6 and WW7 strains. A sample from the fermentation tank was tested in triplicate run sterile dilution series using a buffer solution diluent at a neutral pH ˜7.0. The serial dilutions of 10-4, to 10-9 were plated using the standard microbiology spread plate method. The media spread plates use includes NFM (Nitrogen free media), NLM (Nitrogen limited free media), TSA (Tryptic Soy Agar), PDA (Potato Dextrose Agar), and/or other media. The colonies were grown for 48-72 hours at about 75° F. to about 86° F. The colonies on the plates were then counted based on the morphology of each colony to arrive at a CFU/ml.

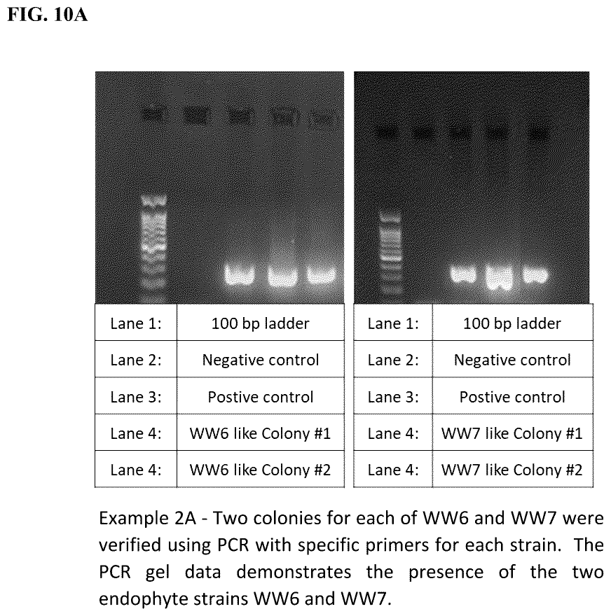

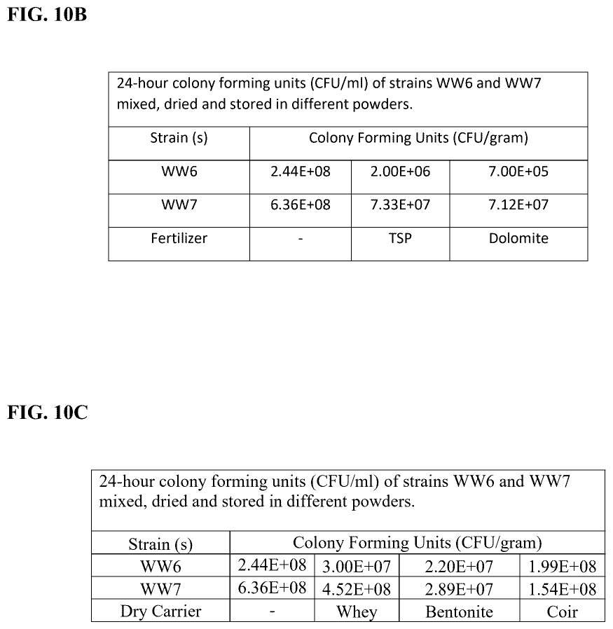

Additionally, two colonies for each morphology were verified using PCR with specific primers for each strain. FIG. 10A demonstrates via a PCR gel the presence of the two endophyte strains WW6 and WW7.

The foregoing co-fermentation procedure is applicable to other combinations of endophyte strains, such as any combination of WW5, WW6, WW7, and PTD1.

### Example 7B

Endophyte Compositions Made from Mixing Dry Fertilizers and Dry Powders for Combinatorial Use in Agriculture.

Two new compositions were made that contained the WW6 and WW7 endophyte strains plus Sodium Alginate (DuPont Nutrition USA, In) plus a dry fertilizer; triple super phosphate 0-28-0 (OCP group), and a separate one plus Dolomite Lime (Down To Earth, Inc). 3 mL of a mixture containing WW6+WW7+0.5% of alginate was separately added to 5 grams of the dry fertilizer. The compositions were dried at room temperature for 24 hours and stored. The bacterial enumeration of the colony forming units (CFU/gram) of the two strains were then determined by plating on NLM semi-solid medium.

The results given in FIG. 10B showed a minor reduction in survivability of both endophyte strains when mixed with different fertilizers in new compositions. These results demonstrate the liquid endophyte compositions can be used to make new compositions for delivery into commercial agricultural practices as NUE endophyte enhanced agricultural fertilizers.

Additionally, the liquid fermentate composition that contains WW6 and WW7 endophyte strains plus sodium alginate (DuPont Nutrition USA, Inc.) was used to generate new compositions comprising three different powdered dry carriers when combined with whey protein (Chemital técnicas alimentarias), sodium bentonite (Specialty Minerals, Inc), and coconut coir (W. Atlee Burpee & Co). The compositions were made through adding 3 ml of the liquid composition containing a mixture of WW6+WW7+0.5% of alginate and added to 5 grams of the different dry powdered carriers. The powdered carrier endophyte compositions were separately dried at room temperature for 24 hours and then stored prior to the enumeration of colony forming units (CFU/gram) by plating on NLM semi-solid medium.

The results are provided in FIG. 10C and show a minor reduction in survivability of both endophyte strains when mixed with the different dry powdered carriers. This demonstrates the endophyte composition can be made and used for blending, coating and delivery into commercial agricultural practices when used together with the different dry carriers.

### Example 7C

Survival of Endophyte Strains (WW6 and WW7) were Assayed after Freeze Drying and Combined with Different Powdered Carriers Along with a Mycorrhizae Powder.

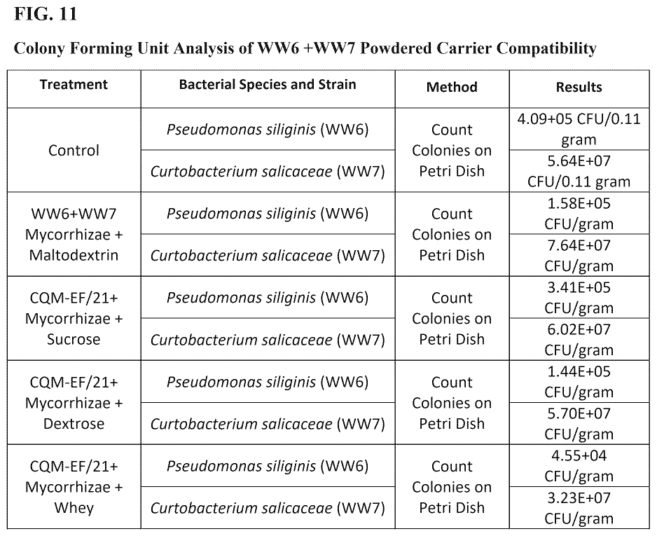

A variety of powdered carrier mixtures (Maltodextrin, Sucrose, Dextrose, Whey) were assayed for compatibility with a FD powder mixture of WW6+WW7. Rates of mixtures tested were as follows: powdered carrier 2.09 g (˜95% by weight), freeze-dried, powdered WW6+WW7 0.11 g (˜5% by weight), Mycorrhizae 0.0022 g (˜0.1% by weight). The results of the compatibility testing demonstrated the following results shown in FIG. 11.

The compatibility testing of freeze dried WW6+WW7 mixed with four possible fillers (maltodextrin, sucrose, dextrose & whey) along with Mycorrhizae yielded positive results. One powdered carrier that reduced the CFU the most was the whey product and it only slightly inhibited WW6. Maltodextrin and dextrose marginally reduced the CFU of the WW6 strain. The other powdered products did not reduce the CFU much at all. The sucrose had no CFU reductions of either strain in the assay and was the best powdered carrier.

Example results demonstrate the ability to mix freeze-dried endophyte powders into a variety of carriers that can dilute the freeze-dried inoculum to lower levels and result in the ability to then be used in either a fertilizer coat or be used as a re-constituted powder for subsequent resuspension and a variety of aqueous foliar applications.

### Example 7D

Short- and Long-Term Survival of Endophyte Strains (WW5, WW6, WW7, PTD1 and WP1) Over 1-2 Days and after Two Weeks when Combined with the Powdered Carrier Biochar Alone, with Biochar Plus a Carbohydrate Sugar Molasses.

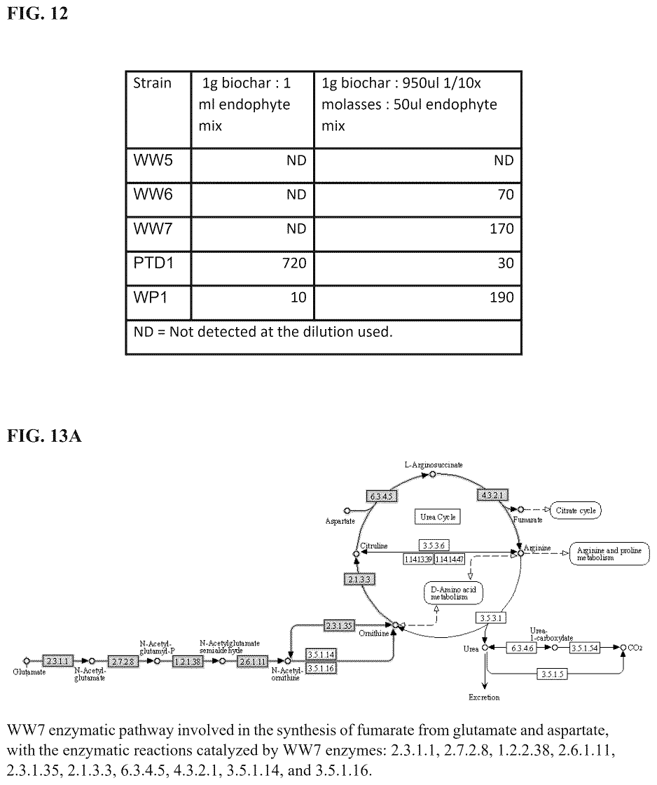

Biochar was treated with an inoculant composition of co-fermented WW5, WW6, WW7, PTD1 and WP1 alone and in conjunction with a 1/10× molasses solution. The powdered biochar material was dried at room temperature in open baggies and stored at 25° C. After two weeks the dried biochar (0.1 g) was resuspended in 1 mL of a potassium phosphate buffer and the survival of the endophytes by strain was assayed and the results CFU/ml. The data is provided in FIG. 12.

The results demonstrated that 50 μL of the co-fermented endophyte mix with 950 μL of 1/10× molasses applied per 1 g of biochar is an effective rate for survivability and stability of four of the five tested endophyte strains on the biochar powdered carrier for at least 2 weeks.

### Example 8A

### Endophyte Strain WW7 Solubilizing Insoluble Forms of Phosphate.

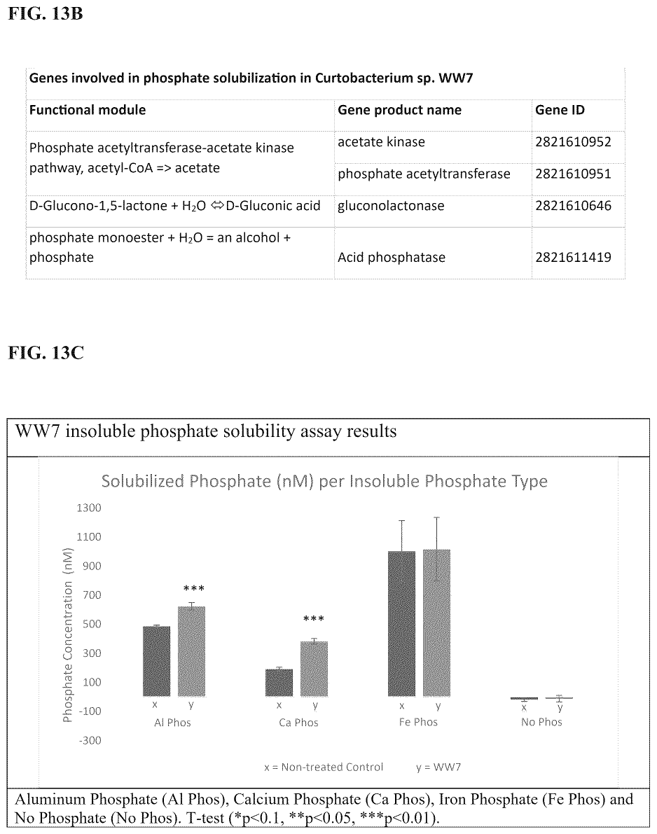

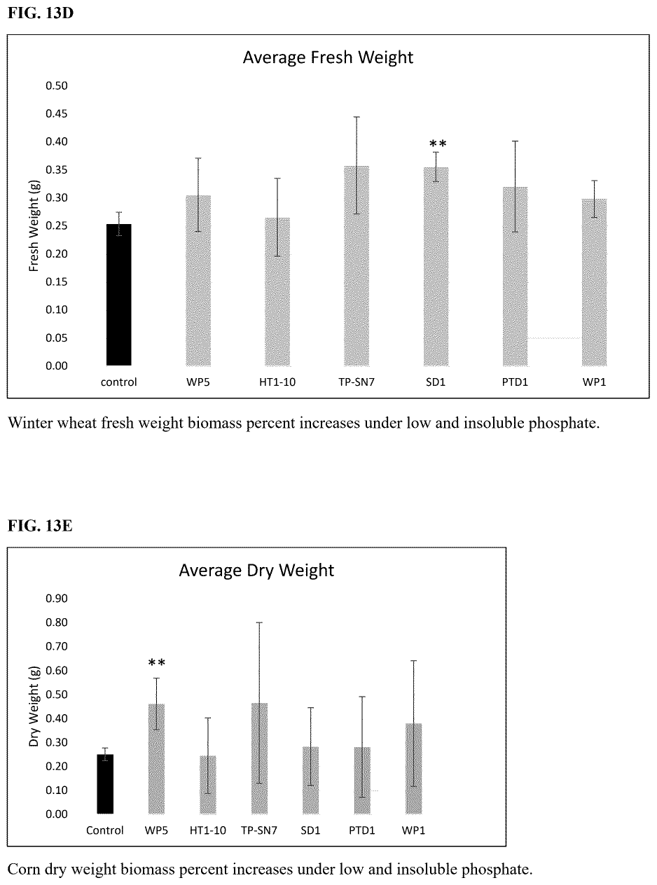

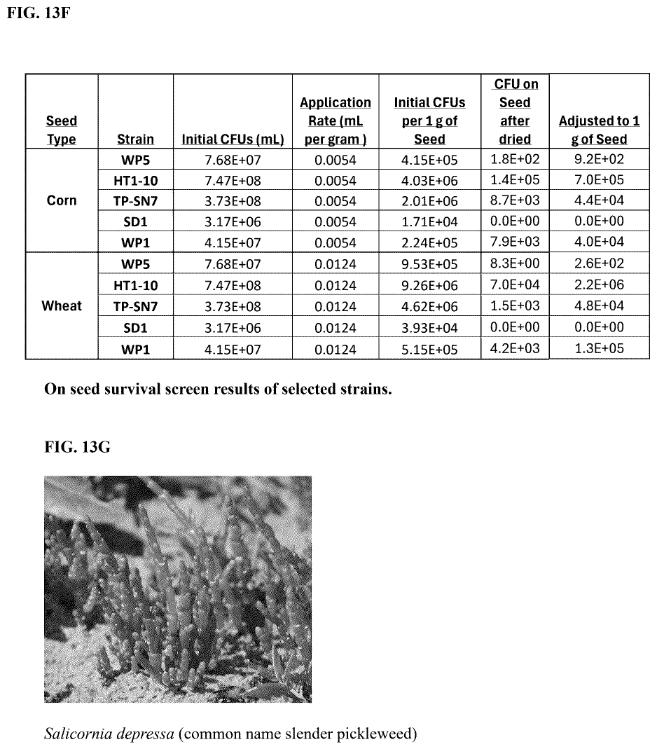

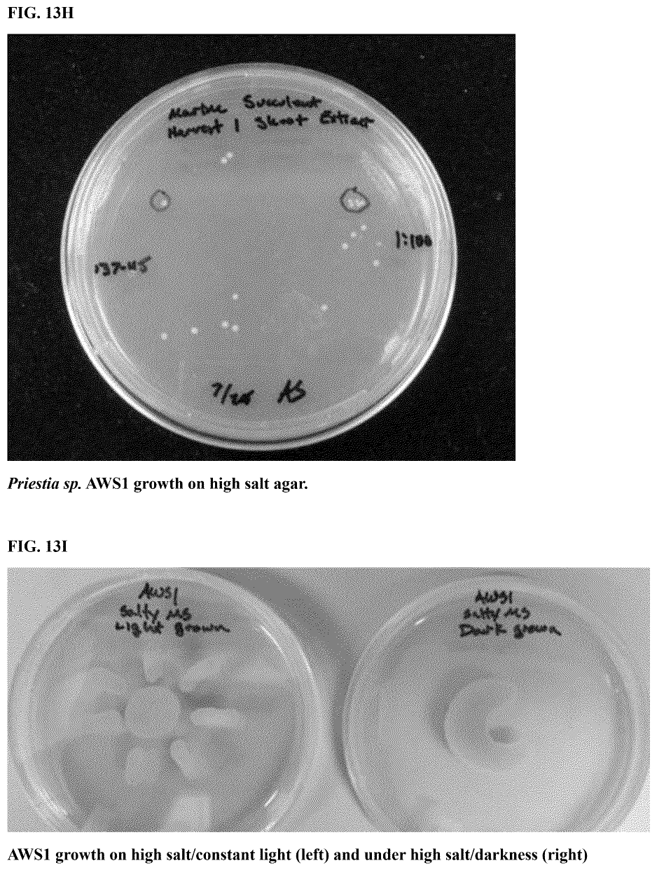

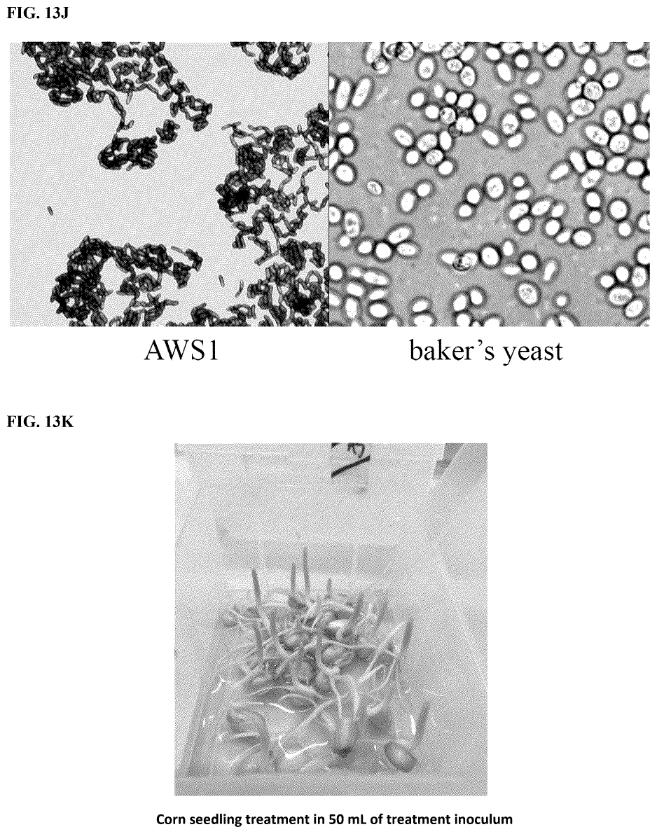

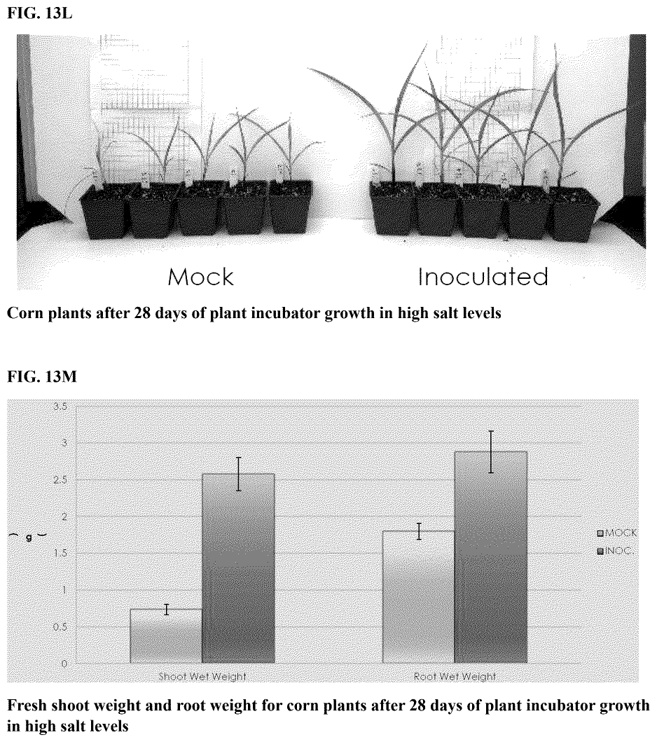

The ability of an endophyte strain(s) to augment the endophyte's metabolism to allow it to utilize insoluble forms of phosphorus (P) from soil or a soil solution and translocate P in planta and enhance P uptake relative to other soil particles or internal metal ions. The heterologous application of endophyte strain WW7 has been shown to have an enhancing effect on P uptake in host plants. Heterologous WW7 is apparently able to solubilize different insoluble P forms that are insoluble in a media solution mixture. The genome analyses of WW7 point to a potential genetic mechanism for the biosynthesis of Krebs cycle intermediates such as organic oxyacids malate and citrate that may be responsible for solubilizing forms of insoluble phosphate from soil allowing better plant uptake. Additionally, the endophyte may have exudates that help keep the phosphate ligand free once inside plants by out competing other metals that might tightly bind phosphate making it again insoluble and unavailable for assimilation. WW7 genomic data were used for the identification of protein-coding genes involved in the reactions of interest, the predicted proteome of WW7 was functionally annotated through the Kyoto Encyclopedia of Genes and Genomes (KEGG) database using KofamKOALA genome jp tools (https://www.genome.jp/tools/kofamkoala/). The Kyoto Encyclopedia of Genes and Genomes (KEGG) database was used as a functional database to query WW7 proteome to enzymatic reactions and pathways catalyzing the synthesis of malate and citrate, which are exuded by the roots and may solubilize insoluble P. A total of 1602 proteins in WW7 were mapped against the KEGG database. The enzymes involved in the synthesis of malate and citrate were the citrate synthase (gene id: 2821609409) and the fumarate hydratase (gene id: 2821609475), respectively. Malate can also be synthesized by the assimilation and conversion of aspartate and glutamate. In this respect, WW7 possesses a complete set of enzymes catalyzing the conversion of glutamate and aspartate into L-arginosuccinate, which is further converted in arginine with the consequent release of a molecule of fumarate for use in the citrate cycle pathway (see FIG. 13A). Glutamate is the first amino acid product in the GS-GOGAT pathway that produces 1 mole of glutamate from 1 mole each of NH₃ GOGAT pathway responsible for atmospheric and gaseous soil NH₃/NH₄ assimilation in bacteria. FIG. 13A shows the WW7 enzymatic pathway involved in the synthesis of fumarate from glutamate and aspartate, with the enzymatic reactions catalyzed by WW7 enzymes (Reaction Nos. 2.3.1.1, 2.7.2.8, 1.21.38, 2.6.1.11, 2.3.1.35, 2.1.3.3, 6.3.4.5, and 4.3.2.1).

### Phosphate Solubilization Genes

According to KEGG annotation, genes that are involved in the solubilization of inorganic and organic phosphate in other species were also detected in WW7. See FIG. 13B. The results indicate the presence of the acid phosphatase (AcPase) gene and genes involved in the synthesis of acetate and gluconic acid. The AcPases have been shown in other species to be involved in the solubilization of phosphate from phosphomonoesters, and acetate and gluconic acids have been shown in other species to be involved in the solubilization of phosphate from inorganic forms.

The ability of an endophyte strain to solubilize insoluble phosphorus (P) from soil or the soil solution by exogenous production of a variety of mobilizing compounds is important for helping plants to acquire required P, other nutrients such as potassium K, and micronutrient ions more efficiently from the soil. These chelating, or pH reducing acidic compounds, made by the endophytes, can apparently help plant roots to better access these minerals from soil and help with uptake and translocation from root to shoot in plants.

To demonstrate the ability of WW7 to solubilize insoluble forms of phosphate through secretion of exogenous compounds, an insoluble phosphate solubilization assay was conducted in liquid media using a phosphate sensitive dye and a microplate reader method to further confirm biochemical capability of WW7 to solubilize different phosphate species. A physiological assay method was also developed and modified from a previous method performed by Varga et al., Endophyte-Promoted Phosphorus Solubilization in Populus, Frontiers in Plant Science, 11; 2020; 1585 (“Varga”). WW7 cells were grown on modified National Botanical Research Institute (NBRIP) broth with no phosphate to deplete residual internal phosphate. Five milliliters of the modified NBRIP broth cultures with added phosphates or no phosphate as previous described by Varga were added to 10 mL of WW7 cell culture. These 10 mL tubes were then incubated for 3 days at 25° C. and shaking at 220 rpm. The 10 mL tubes were then removed from the shaker allowed to settle for 90 min. One mL of the culture was spun down 5,000 rpm for 5 min at 25° C. and the supernatant was used to measure solubilized phosphate. Solubilized phosphate absorbance was measured at 650 nm (A650) using a Phosphate Colorimetric Assay Kit (Sigma-Aldrich, MAK030). The supernatant from each sample was added to a 96 well plate at different dilution rates. Additionally, the phosphate standards provided in the kit were prepared to calculate the linear equation used to determine solubilized phosphate in each sample. Four to five technical replications were used to determine statistical differences between the samples.

The results given in FIG. 13C demonstrate that the growth and molecular activities of endophyte strain WW7 result in statistically significant (p<0.01) increases in phosphate solubilization for both insoluble suspended aluminum phosphate and tri-calcium phosphate, but not for iron phosphate. The strain WW7 increased phosphate solubilization from insoluble aluminum phosphate on average 29% and increased phosphate solubilization from insoluble tri-calcium phosphate on average 100%. These results demonstrate a substantial mobilization of insoluble phosphate by WW7.

WW7 may be mixed with other diazotrophic microbes to mobilize P such that the conversion of insoluble phosphate by WW7 and potential absorption of the solubilized P by a host plant obtained from soil or rock may be combined with enhanced nitrogen acquisition (e.g., from the atmosphere). Together these mechanisms may greatly enhance treated host plant metabolic performance, which may translate into enhanced biomass, stress tolerance, and other favorable characteristics.

### Example 8B

### Seed Coat Formulations and Treatment Effects

To assess the survivability of endophyte strains capable of solubilization of insoluble phosphorus for use in a seed treatment, the SD1, HT1-10, TP-SN7, WP5, and WP-1 endophytes were fermented into a turbid suspension as described in Example 2A. The resulting strain formulations were mixed with a pure sodium alginate (0.3-0.5% w/v), which is a bacterial compatible carbohydrate that allowed the microbes to enter a dried stasis in a partially hydrated shell on the surface of the dried seed. The resulting fermentation solution was subsequently applied to seeds for testing the survival of the endophytes after dehydration on corn and winter wheat seeds at rates of 40-80 ul of solution/100-200 seeds.

The fermentation cultures were prepared under conditions (containing endophytes at ˜107 CFU/mL) in NLM media and dry sodium alginate was added at 0.3-0.5% w/v. The fermentation cultures were carefully pipetted onto the seeds in a sterile laminar flow hood. The treated seeds were massaged, shaken, and tumbled for 2-3 minutes in a bag after application, until seed appeared visibly wet in the bag. The bag was then opened for air-drying in the sterile laminar flow hood. After drying, the seeds were stored at 25° C. for 3 weeks and then assayed for microbial survival.

For assessment of the bacterial seed survival, a seed coat wash and enumeration plating assay was conducted. The bacterial formulation dried coat was washed off of the wheat seeds by using an enumeration and dilution plating methods as follows; for wheat 20 seed, or for corn 10 seeds, were first added separately to 10 mL of 1×PBS and the slurry solutions plus seeds were vortexed for 30 seconds to dissolve hard dried seed coats with bacteria. To ensure seed wash the tubes with seeds were then placed on the shaker at 150 RPM for 1 hour at 25° C. The seed wash solution plus seeds were then vortexed for an additional 30 seconds and then dilution plated using 1×PBS and plated on agar plates. The plates were then incubated for 3 days and colonies were counted by morphology type. The resulting bacterial titers are shown in FIG. 13D.

The results shown in FIG. 6J demonstrate positive survival on seed results for the top performing strains SD1, HT1-10, TP-SN7, WP5, and WP-1. The results show that the tested strains are useful to inoculate seeds and colonize subsequent germinated seedlings for long term shelf-life stable product survival on seed after treatments prior to planting seed.

### Example 8C

### Seed Coat Phosphorus Solubilization Treatment Effects on Winter Wheat

The treated winter wheat seeds of Example 8B were tested for in planta effects on biomass under low phosphorus conditions to demonstrate the ability of the endophyte strains' abilities to mobilize insoluble phosphorus in the form of calcium phosphate in soil for uptake by the treated seeds and resulting wheat plants. Control seeds were not treated with fermentate. The ability of single-strain phosphate (P) solubilizing endophyte inoculum formulations to affect P nutrient use efficiency and growth in planta were assessed under field-relevant reduced P fertilizer conditions. Plants were germinated and grown in a controlled environment pot study with reduced levels of insoluble Calcium Phosphate (Ca₃(PO₄)₂) mixed in the soil equivalent to 20 lbs P/ac (0.04 mg P/g soil). The optimum P per acre rate is about 30 lbs P/ac. Plants were grown in a controlled plant-grow room at 25° C. under full spectrum white LED at about 450 μmol m⁻²S⁻¹, on a 14 hr day and 10 hr night cycle. The plants were supplemented with Hoagland's phosphate free fertilizer solution (P drop out). It was used at 0.25× without any P and contained all the other normal Hoagland's macro and micro nutrients in a well-defined plant growth fertilization solution.

Thirty days after germination the fresh weight biomass of each plant was measured to assess the endophyte inoculum seed treatment formulation effect on plant growth when in low phosphate fertilizer conditions, in which enhanced solubilization of insoluble calcium phosphate would provide an advantage. The results of the fresh weight biomass measurements of the different P-solubilizing endophyte treatment groups are provided in FIG. 13E.

Mean fresh weight biomass percentage increases in growth under low phosphate fertilizer conditions in the treated group were higher than that of controls that were not treated with P-solubilizing endophyte inoculum. Specifically, the percentage differences between the treatment groups and controls were: +44% for SD1, +44% for TP-SN7, +28% for PTD1, +25% for WP5, +20% for WP1 and +8% for HT1-10. These results demonstrate the ability of different endophyte seed coat formulations to increase total wheat plant biomass when growth under limited P and when fertilized in soil that includes insoluble forms of phosphorus.

### Example 8D

### Seed Coat Phosphorus Solubilization Treatment Effects on Corn

The treated corn seeds of Example 8B were tested for in planta effects on biomass under low phosphorus conditions to demonstrate the ability of the endophyte strains' abilities to mobilize insoluble phosphorus in the form of calcium phosphate in soil for uptake by the treated seeds and resulting corn plants. Control seeds were not treated with fermentate. The ability of single-strain phosphate (P) solubilizing endophyte inoculum formulations to affect P nutrient use efficiency and growth in planta were assessed under field-relevant reduced P fertilizer conditions. Plants were germinated and grown in a controlled environment pot study with reduced levels of insoluble Calcium Phosphate (Ca₃(PO₄)₂) mixed in the soil equivalent to 20 lbs P/ac (0.04 mg P/g soil). The optimum P per acre rate is about 55 lbs P/ac. Plants were grown in a controlled plant grow room at 25° C. under full spectrum white LED at about 450 μmol m⁻² S⁻¹, on a 14 hr day and 10 hr night cycle. The plants were supplemented with Hoagland's phosphate free fertilizer solution (P drop out). It was used at 0.25× without any P and contained all the other normal Hoagland's macro and micro nutrients in a well-defined plant growth fertilization solution.

Thirty days after germination the dry weight biomass of each plant was measured to assess the endophyte inoculum seed treatment formulation effect on plant growth when in low phosphate fertilizer conditions, in which enhanced solubilization of insoluble calcium phosphate would provide an advantage. The results of the dry weight biomass measurements of the different P-solubilizing endophyte treatment groups are provided in FIG. 13F.

Mean fresh weight biomass percent increases in growth under low phosphate fertilizer conditions in the treated group were higher than that of controls that were not treated with P-solubilizing endophyte inoculum. Specifically, the percentage differences between the treatment groups and controls were: +84% for WP5, +84% for TP-SN7, +52% for WP1, +12% for SD1, and +12% for PTD1. No difference from controls was observed for seeds treated with HT1-10. These results demonstrate the ability of different endophyte seed coat formulations to increase total corn plant biomass when growth under limited P and when fertilized in soil that includes insoluble forms of phosphorus.

### Example 8E

### Salt Tolerance in Corn Improvement by a P Mobilizing Endophyte.

In order to obtain endophytes potentially useful for providing both phosphate mobilization and salt and drought tolerance in crop plants, a novel endophyte isolation and screening approach was developed. This process utilized shoot cuttings from the halophytic plant Salicornia depressa (common name slender pickleweed) a widely dispersed salt tolerant estuary plant that grows in the ocean tidal flat of the Pacific Northwest, near Seattle, WA. Salicornia depressa thrives under extreme saline and osmotic stress, making it a suitable candidate for isolating endophytes with potential agricultural applications. FIG. 13G provides an image of a specimen.

The surface of the Salicornia depressa cuttings was sterilized and tissues were sampled to identify salt tolerant endophytes present in the plant. Tissue samples were macerated and inoculated on selection plates containing 1/10× minimal Murashigee and Skoog's MS media on selection plates which included an extremely high salt concentrations (30 g NaCl/1 L) to selectively culture halophilic species. The only bacterial isolate that grew under these conditions was endophyte strain AWS1, as shown in FIG. 13H.

AWS1 was subsequently tested for its ability to grow on agar plates under varied stress conditions, including high salt with either high light exposure or complete darkness. Under high salt, high light conditions, AWS1 grew well and produced a yellow pigment, as shown FIG. 13I. This pigment production may indicate the biosynthesis of carotenoids or other protective secondary metabolites that mitigate light-induced oxidative stress. Under high salt conditions with no light, AWS1 grew well, but produced no visible yellow pigment, indicating that pigment biosynthesis in AWS1 is light-dependent. AWS1 bacteria is a very large bacteria as seen in the microscopic image juxtaposed to an image of baker's yeast at the same magnification provided in FIG. 13J.

The genome of AWS1 was sequenced, and a detailed phylogenomic analysis was conducted using the Type Strain Genome Server (TYGS) to identify the taxonomic placement of AWS1. TYGS compares the whole genome sequence of AWS1 to a database of type strain genomes, performing a precise phylogenomic comparison through pairwise digital DNA-DNA hybridization (dDDH) and genome-based distance metrics. AWS1 was classified as a species Priestia megaterium, based on genomic similarity to existing Priestia strains.

To evaluate the functional in planta effects of AWS1 in crop systems under saline stress conditions, a seed treatment study was conducted using maize. Chlorine-sterilized maize seeds were germinated for 3 days in a sterile tip box wrapped in moist paper towels. The seedlings were separated into two groups: a control group treated with sterile media (MOCK) and a treatment group inoculated with AWS1. An AWS1 inoculum was prepared by fermenting AWS1 into a turbid suspension, as described in Example 2A.

The optical density of AWS1 after the fermentation was taken and culture was diluted to 0.1 OD at 600 nm visible light. As shown in FIG. 13K, the treatment group corn seedlings were treated with 50 ml was used to treat corn seedlings in tubs, which were shaken for 24 hours at 60 RPM, where the rotational direction was switched every 2 cycles. The inoculated treatment group and controls were subsequently grown in an incubator in salty soil. Corn plants were watered with a high salt solution at 15 g NaCl/1 L water for 28 days.

The corn plants were then assessed for shoot growth. The treatment group showed substantially more shoot growth as compared to controls—see FIG. 13L. As shown in FIG. 13M, the AWS1 inoculated plants also had +250% more fresh weight biomass and 3.33 times more shoot biomass than controls. The difference in root growth biomass was also pronounced; the treatment group roots had +65% greater fresh weight root biomass than the mock control plants. These results indicate that AWS1 confers salt tolerance in corn seedlings by enhancing shoot and root biomass production under high salinity conditions.

The results demonstrate the effectiveness of AWS1, a phosphate-solubilizing endophyte strain isolated from the native salt-tolerant halophyte Salicornia depressa, in enhancing salt tolerance in corn when irrigated with a saline solution. The ability of AWS1 to promote growth under these conditions highlights its potential for use as a plant inoculant to improve crop resilience in saline soils.

### Example 8F

### Iron Siderophore Production Assay of Genomic and Biochemistry

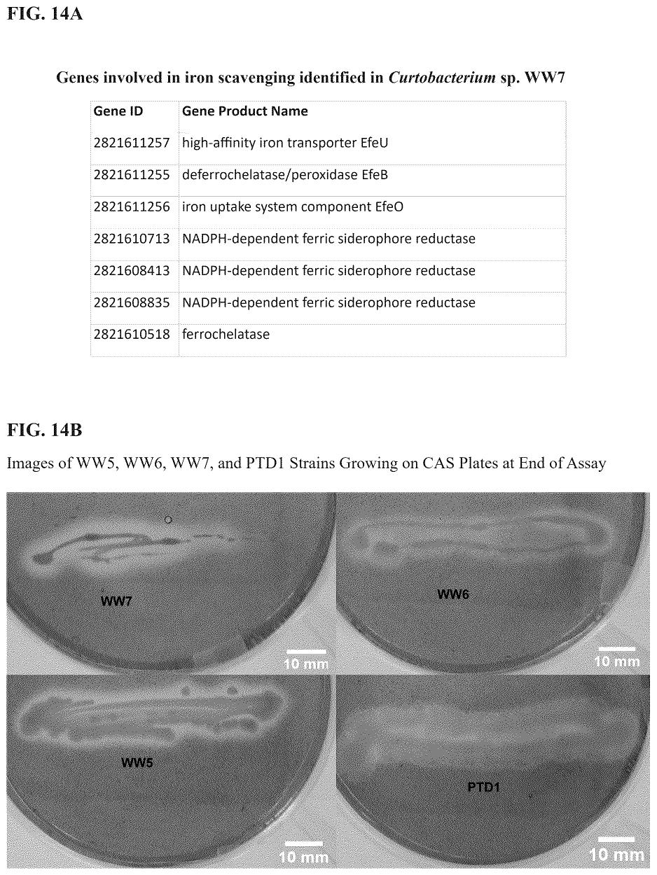

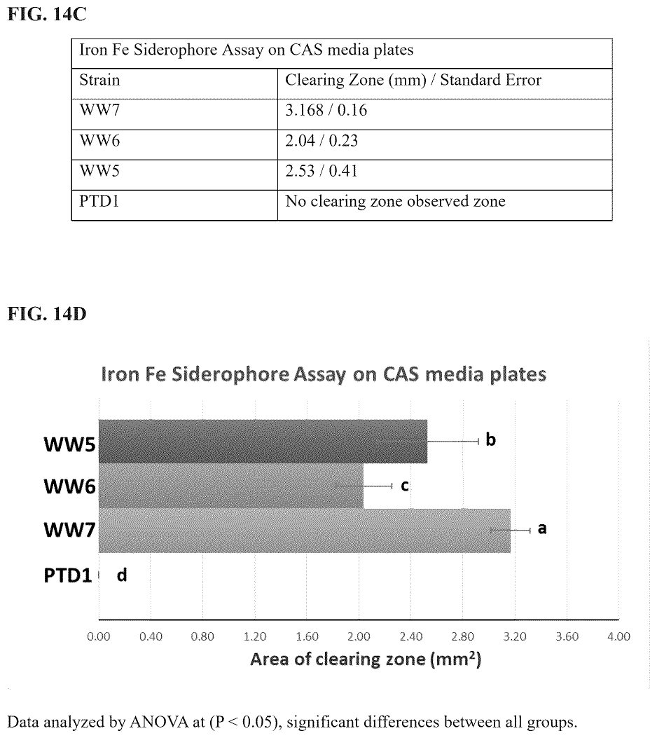

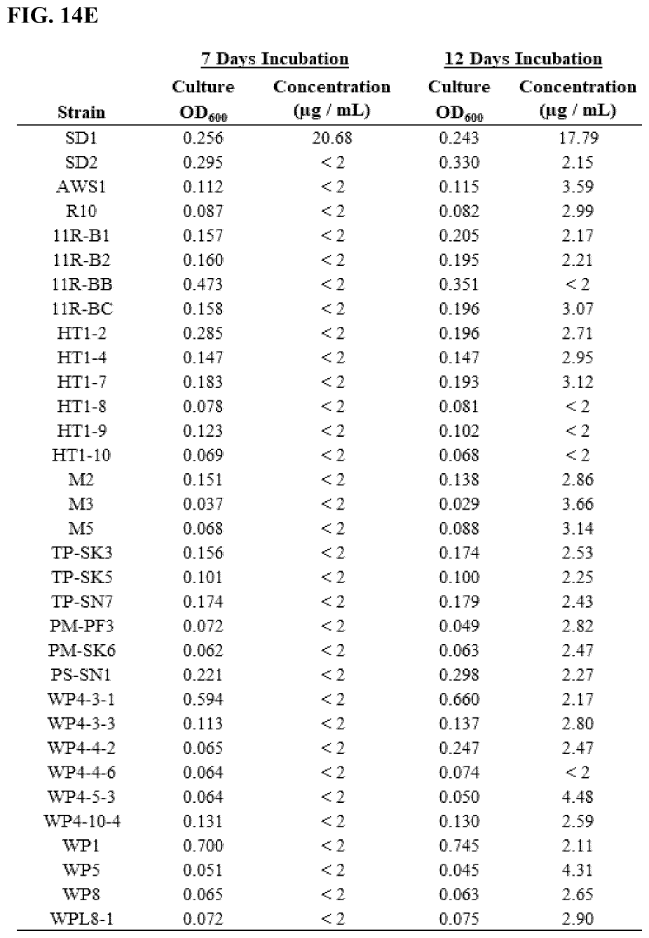

The endophyte strain(s) may be able to solubilize iron (Fe) from soil or the soil solution. This may be accomplished through production of a variety of solubilizing compounds and/or heme related binding factors. The endophyte strains may also augment host plants such that they have an improved ability to acquire required Fe, and to acquire other metals and micronutrient ions especially positively charged divalent cations from the soil. These Fe-siderophore chelating compounds made by some bacterial and yeast endophytes may help plants to better compete with the high cation exchange capacity of soil clay particles and may help uptake and translocate metal cations from root to shoot in plants. To ascertain the ability of strain WW7, a full genome analysis of WW7 genes were performed. WW7 was shown to have the genetic machinery required to make an Fe siderophore, which can transport or scavenge Fe ions. InterProscan results indicate the presence of the gene cluster efeUOB, which is involved in the recovery of ferrous and ferric iron from exogenous hemes, and three genes encoding the NADPH-dependent ferric siderophore reductase. See FIG. 14A. The latter catalyzes the reduction of ferric iron complexed with different siderophores such as ferric triscatecholates and ferric dicitrate. This enzymatic reaction releases bound ferrous iron.

Also, according to anti-smash analyses, the WW7 NADPH-dependent ferric siderophore reductase (Ga0372474_197) was found in a cluster with the gene coding for the enzyme non-ribosomal peptide synthetase-like protein (Ga0372474_207), which is typically found in a biosynthetic gene cluster (BGC) involved in the synthesis of Iron siderophores.

Assay for Fe siderophore production in all four endophyte strains

The endophyte strains WW5, WW6, WW7, and PTD1 for their Fe-siderophore production abilities. Fe-siderophore production was demonstrated in three out of the tested endophyte bacterial strains. The four strains were assayed using a microbial growth solid agar plate-based Fe-siderophore CAS media. Agar plates were prepared with CAS Agar plate preparation including Chromeazurol as a color indicator and FeCl₃. The endophyte strains were applied to individual plates.

Fe solubilization was demonstrated by the presence of discolored zones that developed in the CAS test plates, which appear as transparent white around the bacterial streaks growing on the plates. The zones are measured and quantified using image analysis software (e.g., ImageJ, a publicly available image analysis program provided by the National Institutes of Health-available at http://rsb.info.nih.gov/ij/). The capability of the strains to solubilize Fe were photographed, measured, and compared. See FIG. 14B.

The in vitro results in FIGS. 14B-14D, demonstrate that WW7 made an extracellular compound that scavenges insoluble Fe. WW7 showed the greatest Fe solubilization activity of all the strains. WW5 and WW6 also both exhibited significant Fe solubilization. However, the PTD1 Rhizobium sp. strain demonstrated little to no Fe solubilization activity and the insoluble Fe was observed immediately adjacent the colony streak. The test results demonstrate the production of Fe siderophores by WW5, WW6, and WW7. The test results demonstrate Fe solubilization by each of the WW5, WW6, and WW7 strains, and suggest the production of Fe siderophores was the greatest in WW7, followed by WW5 and WW6. The endophyte strains that produce these compounds can assist host plants in the solubilization and mobilization of Fe. The Fe solubilization activities of each strain are quantified in FIG. 14C and graphically represented in FIG. 14D.

### Assay for Fe Siderophore Production in Additional Endophyte Strains

The endophyte strains SD1, SD2, AWS1, R10, 11R-B1, 11R-B2, 11R-BB, 11R-BC, HT1-2, HT1-4, HT1-7, HT1-8, HT1-9, HT1-10, M2, M3, M5, TP-SK3, TP-SK5, TP-SN7, PM-PF3, PM-SK6, PS-SN1, WP4-3-1, WP4-3-3, WP4-4-2, WP4-4-6, WP4-5-3, WP4-10-4, WP1, WP5, WP8, and WPL8-1 were tested for their Fe-siderophore production abilities. Endophyte strains were grown on NLM agar plates at 25° C. for 72 hours. Colonies of each strain were transferred from the agar plate to a liquid nitrogen-limited medium without iron (NLM-Fe). Cultures were incubated at 25° C. on a shaker table at 150 rpm for up to 12 days, with samples collected at 7 and 12 days for evaluation. Cell density was determined for each sample by measuring absorbance at 600 nm with a spectrophotometer (SpectraMax Plus 384, Molecular Devices, Sunnyvale, USA). Fe-siderophore production of the strains were assayed using the SideroTec-Total Assay according to the manufacturer instructions (Accuplex Diagnostics, Kildare, Ireland).

The results provided in FIG. 14E indicate the strain SD1 had the highest Fe-siderophore production with 20.68 μg/ml at 7 days of incubation. Additional strains had less Fe-siderophore production however they required 12 days of incubation to go above detection limit of the assay.

### Example 8G

### Gas Chromatographic Analysis and Identification

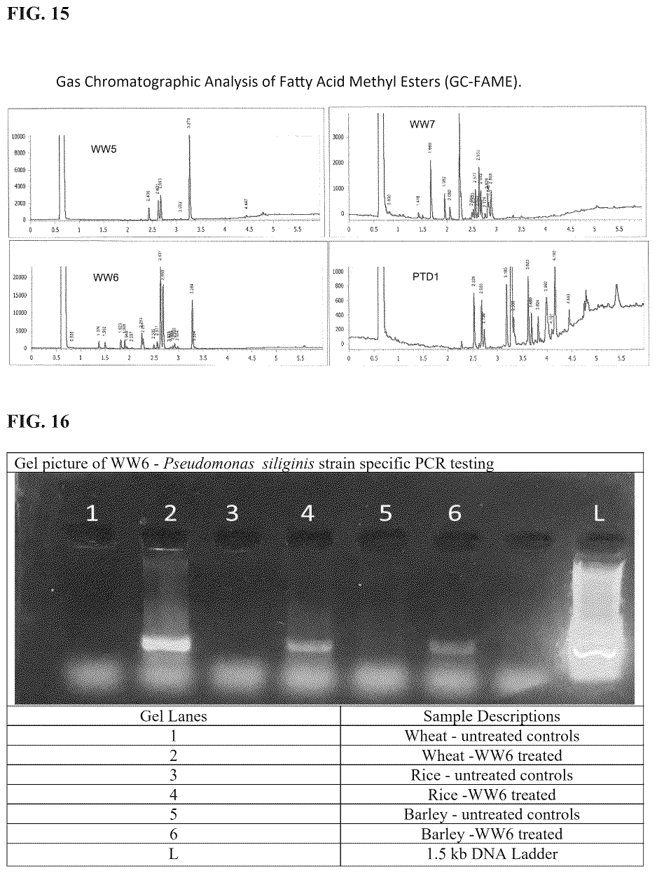

Bacterial Identification by Gas Chromatographic Analysis of Fatty Acid Methyl Esters (GC-FAME) was performed. The GC-FAME analysis provides a unique FAME ID chemical identification chromatogram that is highly specific for each endophyte strain. This allows us to track these microbial isolates individually and positively confirm them each based on their unique Fatty Acid Methyl Ester signature. The unique chromatograms of each strain are shown in FIG. 15.

### Methods of Use

The formulations disclosed herein may be advantageously applied to plants by several means, including and without limitation, spraying, irrigating, coating, immersion, injecting, in furrow, or any combination thereof. The compositions according to the invention can be applied to a leaf, a root, a foliar, foliage, a tiller, a flower, a plant cell, a plant tissue, seeds (e.g., as a coating or by treatment of the seed by spraying or immersion, etc.), as a pre-emergent (before the seedlings emerge or appear above ground), a grain, a fruit, a tuber, a spore, a cutting, a slip, a meristem tissue, a plant cell, nut, or an embryo. In some examples, the composition may be applied as part a dip for the roots and/or other tissues of the host plant, as a seed coating, as a coating applied to the leaves and/or other elements of the host plant, as a powder to the surface of the leaves and/or other elements of the host plant, as a spray to the leaves and/or other elements of the host plant, as part of a drip to the soil and/or roots of the host plant, as a dried alginate bead encapsulating the endophytes and delivering them to roots or other appropriate methods or inoculation.

The compositions according to the present invention are effective to improve the metabolism of a host plant (e.g., nutrient uptake, carbon uptake, growth, etc.). Thus, the compositions and methods of the present invention can be significantly economically advantageous, as the increase in growth characteristics may result in increased yield in harvestable crops and more robust plants. Exemplary methods are discussed below.

### Example 9

PCR Analysis for Endophyte Colonization of Host Plants after Root Soak Inoculation

The ability of the endophyte strain(s) to heterologously colonize host crop plants was tested using PCR techniques. The results categorically showed that the endophytes are inside the surface sterilized plant tissue. The in-planta PCR clearly demonstrated successful colonization in agriculturally important wheat, rice, and barley species that had been inoculated with the WW6 (Pseudomonas siliginis) endophyte strain by seed treatment.

Four sets of plants were grown after their seeds were treated as follows: An appropriate number of seeds was placed into bottom 11 cm by 11 cm seed germination box and 20 mL of inoculation solution was added (co-fermented endophytes WW6+WW7 both at ˜10⁷ CFU/ml in NLM media)). Seeds were germinated for 5 days before being transplanted to 3.5-inch pots containing a mixture of washed play sand, vermiculite, and perlite potting mixture. The plants were grown in a 25° C. grow room under sodium halide light with a 14 hr light/10 hr dark lighting cycle. Additionally, the plants were watered and fertilized with a Hoagland solution with reduced nitrogen at a concentration of 25 ppm in trays 2-3 times per week as needed to maintain moist-slightly dry soil. Fourteen days after transplanting, the plants were harvested individually. The plants were then removed from the soil and processed. DNA was isolated from the processed samples using a PureLink™ Microbiome DNA Purification Kit DNA isolation kit (Thermo Fisher Scientific).

Twenty microliter PCR reactions were set up using 2× HotStart PCR Master Mix (MCLAB), 1 μL of template DNA and 1 uM of PCR primers specific to a gene in WW6. PCR cycling conditions were as follows: 1 cycle of 95° C. for 10 minutes; 25 PCR cycles of 95° C. 30 for seconds, 65° C. 30 seconds, and 72° C. for 45 seconds; and ending 72° C. for 5 minutes. Ten uL of each of the PCR reactions were loaded on a 1.2% agarose gel and run at 120V through DNA QS710 Electrophoresis (IBISCI). FIG. 16 illustrates WW6 is present in the plants of all three tested host plants (wheat, rice, and barley) that were inoculated prior to germination, and is not present in controls for each of the three tested plants. The bands in lanes 2 (wheat treated with WW6), 4 (rice treated with WW6), and 6 (barley treated with WW6) the presence of genes specific to WW6. Thus, it is apparent that the WW6 endophyte strain is able to effectively colonize several host plants.

### Example 10

PCR Analysis for Endophyte Colonization of Host Plants after Seed Coating

The ability of the endophyte strain(s) to heterologously colonize host crop plants when applied with a seed coating was tested using PCR techniques. Shoots and roots of winter wheat (Triticum aestivum) and broccoli (Brassica oleracea) plants treated with WW6 (Pseudomonas siliginis), were evaluated for colonization and incorporation of the WW6 endophyte strain into the plant tissues. The plants were grown after seeds were treated with WW6 as follows: WW6 was fermented according to the method disclosed herein, then blended with 0.5% sodium alginate (Scogin™ LDH), and then used to coat the raw wheat seeds. The same seed coating material without the endophyte strains was applied to the control plants. The coated seeds were air dried at room temperature and stored for 1 month after coating treatment.

An appropriate number of seeds was placed into the bottom 11 cm by 11 cm seed germination box. Seeds were germinated for 5 days before being transplanted to 3.5-inch pots containing a mixture of washed play sand, vermiculite, and perlite potting mixture. The plants were grown in a 25° C. grow room under sodium halide light with a 14 hr light/10 hr dark lighting cycle. Additionally, the plants were watered and fertilized with a Hoagland solution with reduced nitrogen at a concentration of 25 ppm in trays 2-3 times per week as needed to maintain moist-slightly dry soil. Fourteen days after transplanting, the plants were then removed from the soil and processed. Root and shoot tissues from the plants were harvested. DNA was isolated from the processed samples using a PureLink™ Microbiome DNA Purification Kit DNA isolation kit (Thermo Fisher Scientific).

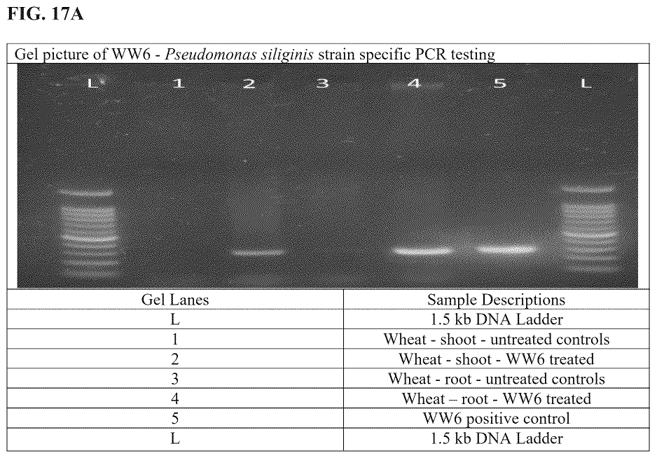

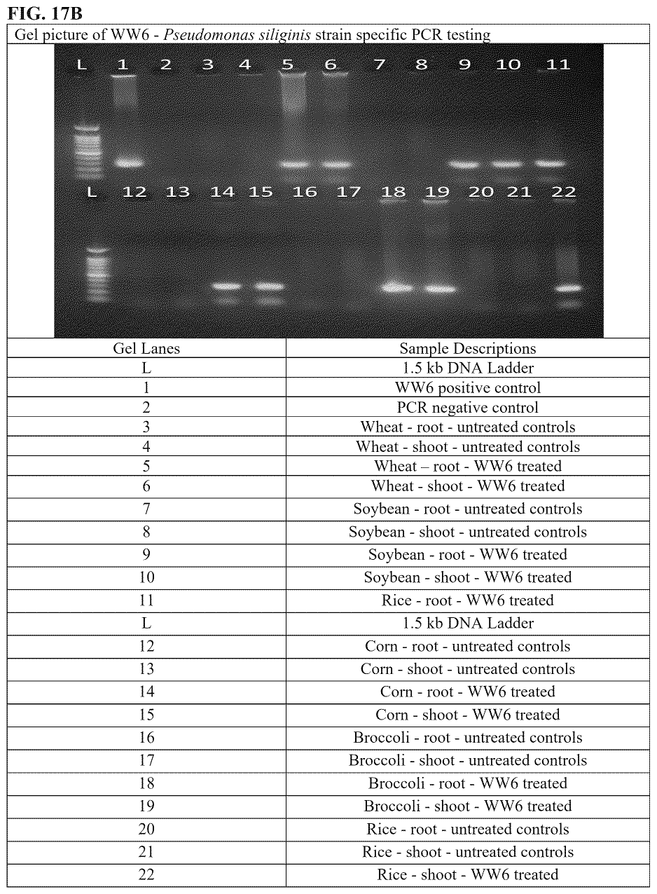

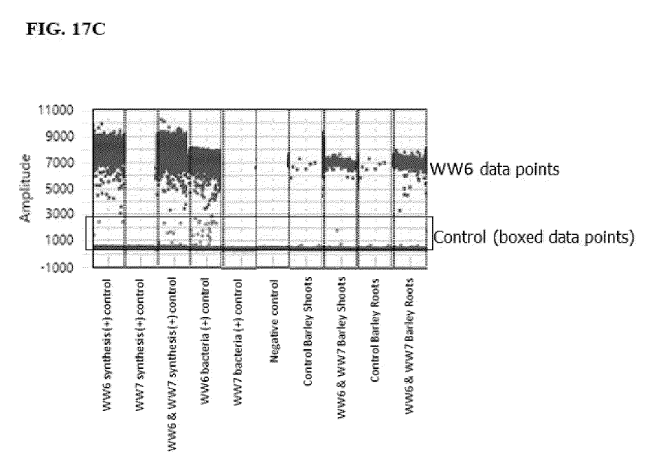

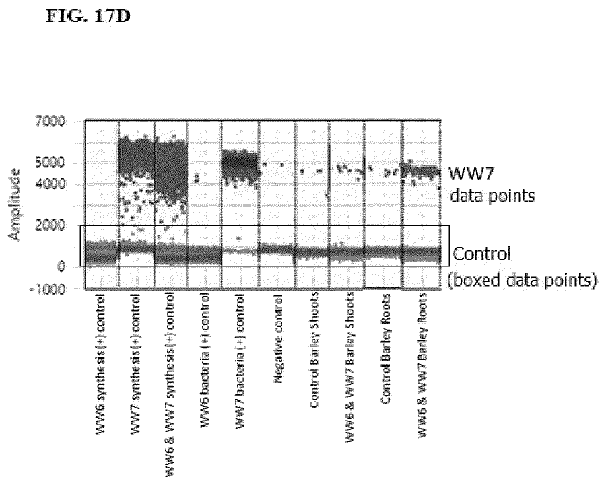

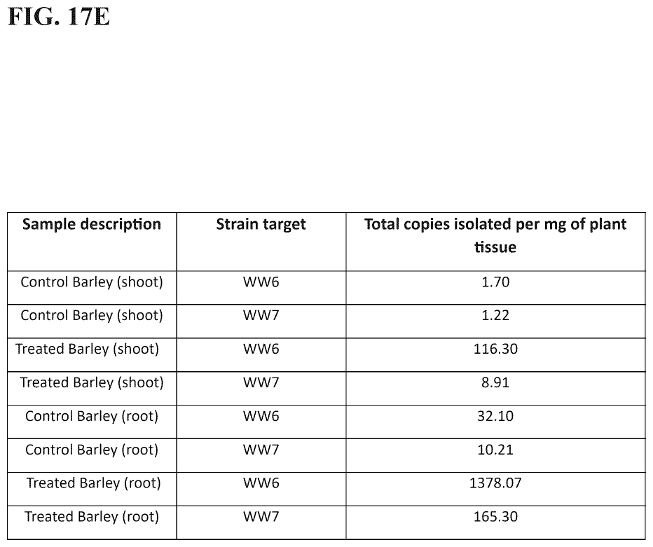

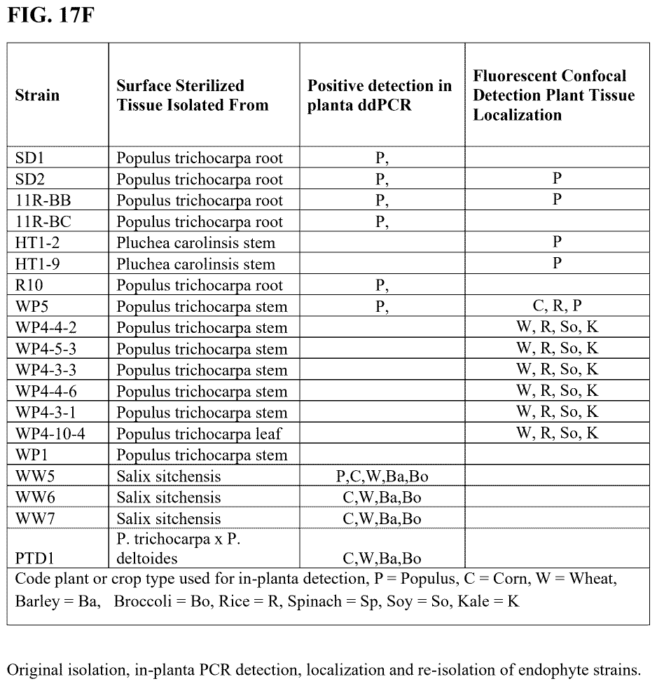

PCR were performed as described in Example 5 above. FIGS. 17A and 17B provide electrophoresis gel data for DNA encoding a protein only present in WW6. The gel demonstrates that WW6 specific DNA was present in the shoot and root tissues of winter wheat and broccoli host plants grown from treated seed, but not in the shoot and root tissues of controls. FIG. 17A provides electrophoresis gel data demonstrating that WW6 was present in the shoot and root tissues of winter wheat host plants grown from treated seed by virtue of the presence of the genomic specific PCR primers designed to a protein that produced DNA bands indicating the specific presence of the WW6 strain but not in the shoot and root tissues of controls. Thus, it is apparent that the WW6 endophyte strain is able to effectively colonize root and shoot tissues wheat host plants after seed inoculation.

FIG. 17B provides electrophoresis PCR gel data demonstrating that WW6 was present in the root tissues of the seedlings inoculated with the WW6 seed treatment after surface sterilization of the root tissues. Several treatment groups using WW6 endophyte strain were prepared: a first treatment of the WW6 liquid fermentate, a second treatment in which the broccoli seeds were treated with the liquid fermentate mixed was 0.5% sodium alginate (Scogin™ LDH from DuPont) and the seed coating material, and a third treatment in which the broccoli seeds were treated with the WW6 liquid fermentate mixed was 1% sodium alginate and the seed coating material. The three treatments were coated onto different groups of broccoli seeds. Plant growth, DNA isolation, and PCR were performed as described in Example 5 above.

FIG. 17B provides electrophoresis gel data demonstrating that WW6 was present in the shoot and root tissues of winter wheat, rice, soybean, broccoli, and corn host plants grown from treated seed by virtue of the presence of the genomic specific PCR primers designed to a protein that produced unique DNA bands indicating the presence of the WW6 strain, but not in the shoot and root tissues of controls. Thus, it is apparent that the WW6 endophyte strain is able to effectively colonize roots and shoot tissues in several host plants after seed inoculation.

### Example 10A—PCR Analysis for Endophyte Colonization of Host Plants after Seed Coating

### WW6 and WW7 in Barley

The ability of the heterologous endophyte strain(s) to colonize host crop plants when applied with a seed coating was tested using the quantitative ddPCR (digital droplet PCR) technique. Shoots and roots of barley (Hordeum vulgare) plants were evaluated for WW6 and WW7 colonization after a seed treatment composition was used to coat seed. The WW6 and WW7 fermentate was blended with 0.5% by weight sodium alginate (Scogin™ LDH) and this composition was used to coat raw barley seeds that were then air dried at room temperature and stored for a month. The same seed coat composition without the endophyte strains was applied to the control seeds.

An appropriate number of seeds were placed in a 11 cm×11 cm seed germination box with sterile filter paper. Seeds were germinated in deionized water for 5 days before being transplanted to 3.5-inch pots containing a mixture of washed play sand, vermiculite, and perlite potting mixture. The plants were grown in a 25° C. grow room under sodium halide light with a 14 hr light/10 hr dark lighting cycle. Additionally, the plants were watered and fertilized with a Hoagland's solution with reduced nitrogen ([25 ppm N]) applied in trays 2-3 times per week as needed to maintain moist soil. Twenty-one days after transplanting, the plants were then removed from the soil before roots and shoots were processed by surface sterilization using 2% bleach followed by a sterile water rinse. The shoots and roots were separated and then flash frozen with liquid nitrogen and then stored at −20° C. The plant material was then ground into a fine powder with a mortar and pestle using liquid nitrogen. 100 mg of each tissue sample was used to isolate DNA with DNeasy Plant Pro DNA isolation kit (QIAGEN, Inc).

A novel primer set was designed and targeted for multiplexing using our gene specific PCR primers to allow for analyses and differential quantification of both strains run at the same time. Strain specific primers were created with flourophores (FAM and Hex). The strain specific primers were validated for the WW6 and WW7 strains using gBlock, double stranded synthesis sequences of specific fragments from each strain. Bacterial genomic DNA were used as positive controls. The validated strain specific primers were used for PCR analysis of samples from the root and shoot tissue of the treated barley seeds for the presence of the WW6 and WW7 strains using a Droplet Digital PCR (ddPCR) machine Bio-Rad Laboratories, Inc.

The results shown in FIG. 17C shows a dot plot graph of the quantification of the WW6 strain PCR target using the FAM and Hex fluorescence signals for the assay of the WW6 strain. FIG. 17D shows a dot plot graph of the quantification of the WW7 strain PCR target using the FAM and Hex fluorescence signals. The experimental results were quantified using the QuantaSoft software (Bio-Rad Laboratories, Inc.).

The total copies of hybridized DNA isolated per mg of plant tissue, which was calculated from the copy number concentration provided by the QuantaSoft software (Bio-Rad Laboratories, Inc.) is tabulated in the table shown in FIG. 17E. The results demonstrated quantitative in-planta detection of both strains WW6 in root and shoot and also WW7 in the root and shoot. The highest detection level was quantified for WW6 in the barley shoot vs the control uninoculated plants.

The data demonstrate that the WW6 and WW7 strains were present in the shoot and root tissues of barley host plants grown from treated seed by virtue of the presence of the genomic specific primers designed to hybridize to unique DNA bands indicating the presence of the WW6 strain, but not in the shoot and root tissues of controls. Thus, it is apparent that the WW6 and WW7 endophyte strains are effectively able to colonize roots and shoot tissues in several crop plants after seed inoculation.

### In-Planta PCR Detection, Localization and Re-Isolation

The ability of various endophyte strains to colonize select plant seedlings (poplar, corn, wheat, barley, broccoli, rice, soy, and kale) through seed inoculation was evaluated using digital droplet PCR (ddPCR) as a sensitive detection method. In these experiments, endophytic bacterial strains listed in FIG. 17F (SD1, SD2, 11R-BB, 11R-BC, R10, WP5, PTD1, WW5, WW6, WW7, and PTD1), were tested for in-planta colonization in cottonwood. The strains were applied to surface sterilized seeds via an inoculation seed treatment prior to germination. Seeds were placed in sterile Magenta germination vessels containing the endophyte inoculum and incubated overnight to germinate. After germination, the seedlings were transplanted into potting soil and grown for approximately 21 days before harvest. The plants were grown in a 25° C. grow room under sodium halide light with a 14 hr light/10 hr dark lighting cycle. Additionally, the plants were watered and fertilized with a Hoagland's solution with reduced nitrogen ([25 ppm N]) applied in trays 2-3 times per week as needed to maintain moist soil. Twenty-one days after transplanting, the plants were then removed from the soil before roots and shoots were processed by surface sterilization using 2% bleach followed by a sterile water rinse. The shoots and roots were separated and then flash frozen with liquid nitrogen and then stored at −20° C. The plant material was then ground into a fine powder with a mortar and pestle using liquid nitrogen. Root and shoot tissues from the inoculated plants were harvested.

100 mg of each tissue sample was used to isolate DNA with DNeasy Plant Pro DNA isolation kit (QIAGEN, Inc). Digital droplet PCR was then performed on each DNA sample to test for the presence of endophyte-specific DNA. To specifically detect each endophyte strain, strain-specific PCR primer sets as listed in FIG. 17G were used for detecting the corresponding strains. Each primer set had been validated to amplify only its target strain's DNA and not any other strain or host plant DNA, ensuring the specificity of the ddPCR detection for each endophyte.

The ddPCR reactions were prepared with the validated strain-specific primers and the extracted plant DNA as template, then partitioned for individual PCR amplification. Thermal cycling was carried out to amplify the DNA fragments. All six inoculated endophyte strains were detected in the plant tissues, whereas no endophyte DNA was detected in the uninoculated control plants. In particular, strains SD1, SD2, 11R-BB, 11R-BC, R10, WP5, WW5, WW6, WW7, and PTD1 were found to colonize the plant tissues of inoculated seedlings (yielding positive ddPCR signals in root-derived DNA), consistent with its colonization of aboveground plant tissue. FIG. 17F provides electrophoresis gel data. Thus, the ddPCR analysis confirms that each of the tested endophyte strains successfully colonized the seedlings following seed inoculation, while no endophyte DNA was detectable in control seedlings.

Additional Fluorescent Confocal Detection for SD2, 11R-BB, HT1-2, HT1-9, WP5, WP4-4-2, WP4-5-3, WP4-3-3, WP4-4-6, WP4-3-1, and WP4-10-4 using GFP, RFP, CFP-tags were used to identify endophytes inside plant tissue and confirm localization. FIG. 17F also shows positive florescent identification in the plant tissue for tagged strains SD2, 11r-BB, HT1-2, HT1-9, WP5, WP4-4-2, WP4-5-3, WP4-3-3, WP4-4-6, WP4-3-1, and WP4-10-4.

### Example 11

Analysis for Endophyte Colonization of Host Plants after Foliar Application

The performance of spinach plants (Spinacia oleracea) in the field treated with WW6 and WW7 endophytes in a foliar spray was tested, and the ability of the endophyte strain(s) to heterologously colonize the host plant was also measured.

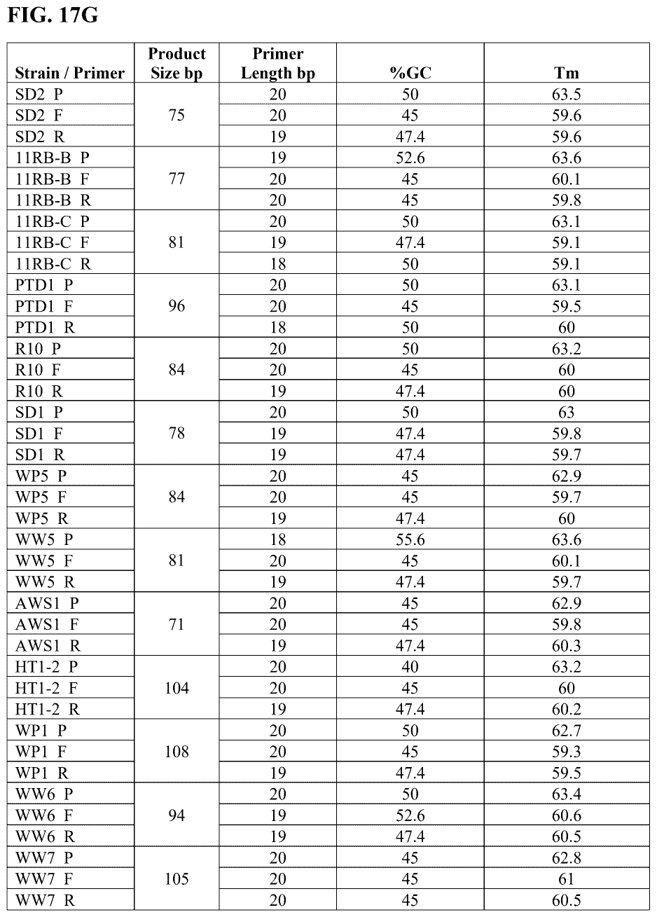

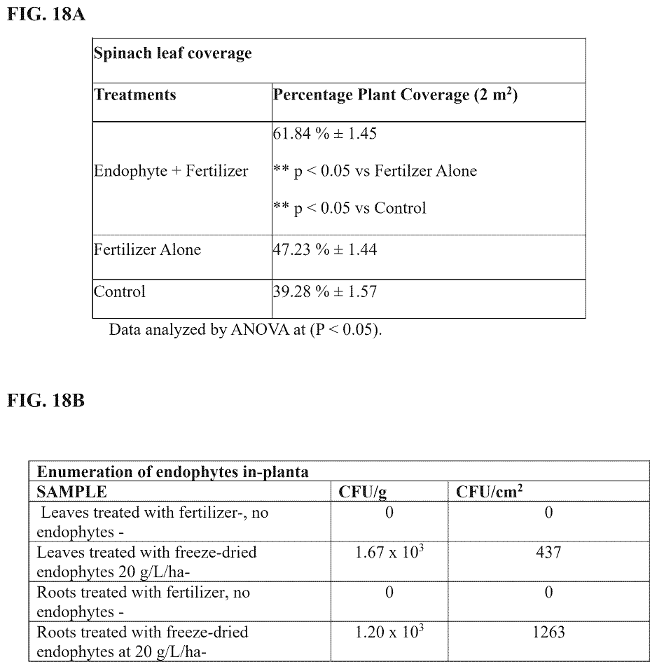

A foliar inoculant composition including co-fermented WW6 and WW7 freeze dried powder resuspended in water composition was applied to spinach plants together with a 10-5-3 CaO liquid fertilizer formulation Greenstim™ (a concentrated glycine betaine extracted from beetroot with 12% total nitrogen from Massó, S.A. Agro Department) following commercial rates. Spinach was cultivated using a full nutrient regimen applying 550 L of 10-5-3 3% CaO fertilizer (715 kg of fertilizer, 18 kg/day) through fertigation on daily basis to each plot. The foliar composition was applied at the four true leaf stage at the foliar rate of 1 L per hectare with an WW6+WW7 endophyte concentration of 20 g/L. The experimental plot was divided into three blocks of six beds each. Sampling was carried out in the central part of the four central beds of each block. Percent canopy coverage was assessed thirty-five days after application using a software tool (Canopeo™) for analyzing and measuring canopy cover in photographic pictures. At the same time leaves were harvested for nutrient analyses and for surface sterilization followed by in planta endophyte quantification. The results of percentage plant coverage on the plot bed data showed that the endophyte foliar treatment caused a 30.92%, statistically significant (p<0.05) increase in spinach leaf canopy cover when compared to the commercial fertilizer alone and a 58%, statistically significant increase over the control treatment (p<0.05). See FIG. 18A.

To assay and quantify the in-planta endophyte presence in spinach tissues, leaves and roots were harvested 35-days post-foliar inoculum treatment and washed, and surface sterilized for the detection of the strains WW6 and WW7 inside the plant tissues. All samples were weighed and photographed to make CFU calculations in relation to sample weights, and foliar or root area. The ends of the leaves and roots were separately sealed in plastic bags before the surface sterilization in a laminar flow hood. A surface wash was conducted with distilled water to remove dust, soil, and other contaminants. Subsequently, the samples were placed in a sterile flask and 70% ETOH alcohol was added and the flask was shaken for 2 minutes (150 rpm). After shaking, the alcohol solution was removed, and a 1% solution of sodium hypochlorite bleach was added. The mixture was shaken for an additional 2 minutes. Subsequently, the sodium hypochlorite solution was also removed, and the samples were cleaned 3 times by manual shaking for 1 minute in sterile water.

Leaves and roots were ground in 50 ml of saline solution 0.9%. The extract was set in a sterile tube and was left to stand for 1 hour to allow for the release of the endophytes from the ground tissue. Several serial dilutions were of the extract were made using sterile water. 100 μl of each dilution was applied to a Potato Dextrose Agar (PDA) plate and actinomycetes isolation agar with glycerol.

The agar plates were incubated until bacteria growth were visually identified. Bacterial concentration was carried out getting the results expressed in CFU/g and CFU/cm² of analyzed material.

FIG. 18B data demonstrated that the endophytes (WW6 and WW7) were present in the surface sterilized plant leaves and roots of the spinach host plants inoculated with the foliar inoculant composition. The endophyte strains were not present in the spinach plants that received the Greenstim liquid fertilizer treatment alone (see detailed discussion above).

WW6 and WW7 endophytes were detected both in the leaves and roots of the host plants 35 days after treatment with the foliar inoculant composition comprising the WW6 and WW7 endophytes. No endophytes were present, and no endophytes were detected in the standard treatment control plants. When endophytes concentration is expressed in CFU/g, more endophytes concentration has been found in leaves rather than roots, but the difference is slight, and it can be assumed that the concentration in leaves and roots is the same in both parts. The CFU/cm² surface area was visually analyzed using Adobe photoshop software and calculated from the measurement.

The foregoing data demonstrate that the endophytes have successfully colonized and improve the foliar growth and biomass of the treated host spinach plants 35 days after foliar application. The results demonstrate the efficacy of the composition and the foliar application method, which provided enhanced production through better establishment, growth and soil coverage of the spinach host plants.

### Example 12

### Effects of Single Endophyte Strain Seed Treatment on Total Nutrient Accumulation

Corn seeds (Zea mays) were treated with a seed inoculant composition comprising one heterologous endophyte strain selected from WW5, WW6, and WW7, and compared to control corn seeds that were treated with the inoculant composition without an endophyte included. Four sets of corn plants were grown as follows: an appropriate amount of corn seed was placed into bottom of a large gallon-sized Ziplock bag and sealed. Three groups of corn seeds were each treated with a specific endophyte culture (WW5, WW6, or WW7). The WW5, WW6, and WW7 cultures were prepared under conditions (containing endophytes at ˜10⁷ CFU/ml) in NLM media plus sodium alginate (0.5% w/v) and then refrigerated. The cultures were removed from the refrigeration, mixed well, and carefully pipetted onto the seeds at a rate of 3.4 mL/lb of seed dispersed in 1 mL drops in a sterile laminar flow hood. The bag and seed were manually tumbled and massaged carefully after each 1 mL addition. Once the entire 3.4 mL/lb application was added, the seeds were massaged, shaken, and tumbled for 2-3 minutes until all corn seed appeared visibly wet in the bag. The bag was then opened for air-drying in the sterile laminar flow hood to allow air flow in the hood to air dry the corn seed. After drying, the seeds were stored at room temperature for 3 weeks and then planted and germinated in a mixture of washed play sand, vermiculite, and perlite potting mixture, in 1 gallon felt smart pots at 25° C. in a grow room under sodium halide light (photon flux of 710 μmol m⁻² s⁻¹) on a 14 hr light/10 hr dark lighting cycle. Once germinated, plants were watered and fertilized in the trays with Hoagland's solution modified for reduced nitrogen at 50 ppm 2-3 times per week, as needed to maintain moist-slightly dry soil. Controls were raised under the same conditions with no pre-treatment prior to planting. Plants were harvested individually at 24 days, dried, and weighed. Tissues were sent out for inductively coupled plasma mass spectrometry (ICP-MS) analysis to determine the ion content of the tissues. Nutrient accumulation of shoot biomass was calculated by multiplying the total shoot dry weight by each sample shoot concentration.

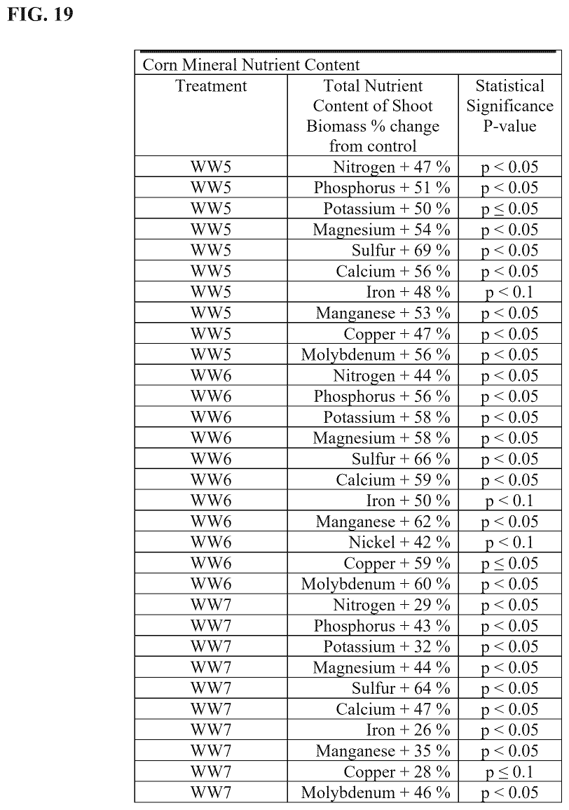

As shown in FIG. 19, corn plants inoculated with endophyte strains WW5, WW6, or WW7 accumulated significantly higher levels of macro and micro-mineral nutrients across the important mineral ion profile as measured by Total Nutrient Content of Shoot Biomass % change relative to untreated control plants when all were grown in a Hoagland's drop out N nutrient solution supplemented at 50 ppm bioavailable nitrogen.

The data in FIG. 19 demonstrate that the heterologous endophytes have successfully improved both macro- and micronutrient uptake and incorporation in host corn plants grown from treated seeds under reduced nitrogen. The results demonstrate the efficacy of the heterologous endophytes to enhance physiological performance of non-native host plants and source nitrogen from the air. Endophyte seed treatment compositions increased nitrogen in corn shoots as follows: WW5 47%, WW6 45% and WW7 29%.

### Example 13

Endophyte Screen Assay of Crop Plant Yield when Grown Under Limited Bioavailable Forms of both Nitrogen and Phosphorus.

The WW5, WW6, WW7, and PTD1 endophyte strains were further screened in greenhouse pot studies wherein plants were inoculated using endophyte strains encapsulated in alginate beads either individually or in a mix of all four strains. Alginate beads encapsulated endophytes inside calcium alginate were placed next to a seed using 1 bead per seed and pots were watered equally using controlled drip irrigation to germinate seeds. Plants were specifically grown under limited nutrient concentrations in pot media with purposefully reduced bioavailable soluble nitrogen and phosphorus forms, where the pot media included nitrate<13 ppm, ammoniacal N<6 ppm and phosphate<11 ppm.

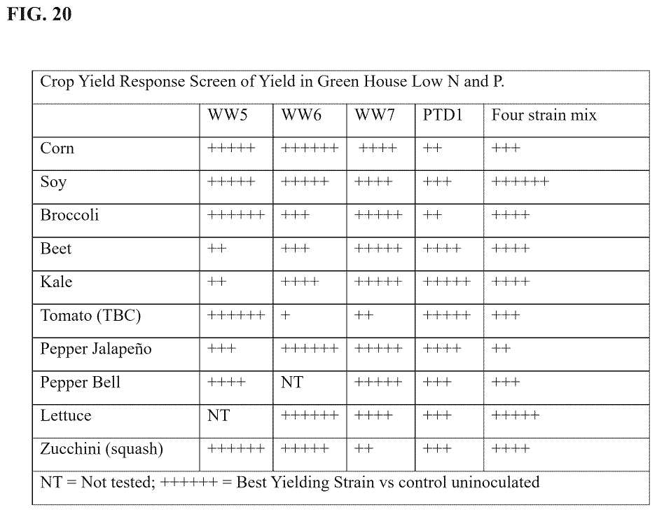

The results in FIG. 20 demonstrate that the four selected endophytes had a positive response in a wide variety of crop plants increasing yield under limited bioavailable nitrogen and phosphorus when using commercially available, agronomically relevant, commonly used seeds.

### Example 14

### Effects of Combined Endophyte Strain (WW6+WW7) Seed Treatments on Total Nutrient Accumulation and Shoot Biomass

Canola seeds (Brassica napus) treated with a seed inoculant composition comprising co-fermented WW6 and WW7 heterologous endophyte strains were grown and compared to control canola plants seeds that were treated with the inoculant composition without an endophyte included. Treated seeds were grown as follows: an appropriate amount of canola seed was commercially treated at a rate of 500 mL the mixed co-fermented WW6 and WW7 strains, 500 mL of 1% alginate per metric ton of seed together with Integral pro (BASF) and prebiotic UBS 016 (Unium Bioscience Ltd.) both following manufacturer's instructions to aid the endophytes in survival, colony growth, and colonization of the host plant. The pre-biotic includes a microbial nutrient package, plant biostimulants, osmoprotectants, buffers, and seed lubricants. Endophyte survival was confirmed on the seed by adding the seeds to a 0.2 M Phosphate resuspension solution and then plating on NLM semi-solid medium at the proper dilution. Control canola seeds received the same crop protection package. The seeds were then stored for 1 month under normal industry seed storage conditions (4° C. to 15° C.) and was commercially planted in Cuxwold, Lincolnshire, United Kingdom in the fall using a seed drill in a large scale replicated CRO field trial.

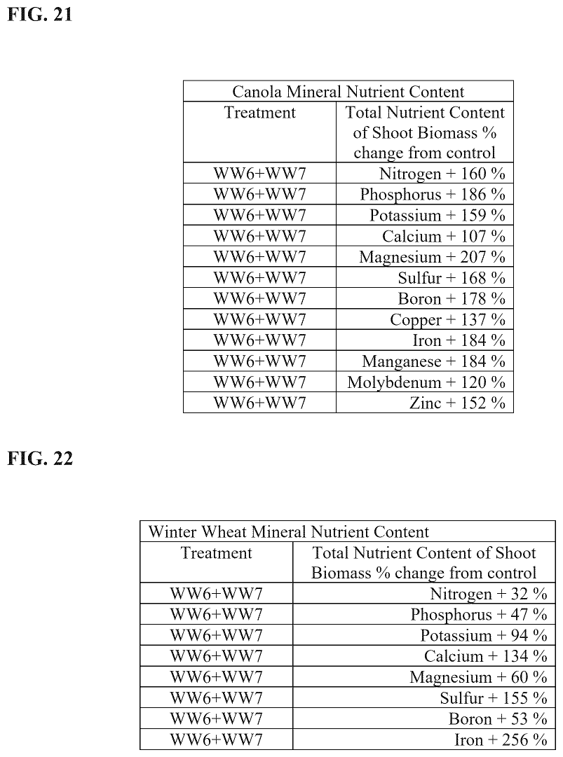

Shoots were harvested in early vegetative mid spring and sent off for agronomic mineral nutrient analyses. Nutrient accumulation of shoot biomass was calculated by multiplying the total shoot weight by shoot ion concentrations. As shown in FIG. 21, canola plants inoculated with the co-fermented WW6 and WW7 strains accumulated significantly higher levels of macro- and micronutrients and the increased nutrient accumulation versus controls is expressed as Total Nutrient Content of Shoot Biomass % change from control.

The foregoing data demonstrate that the co-fermented heterologous endophytes WW6 and WW7 successfully improved macro- and micronutrient uptake and incorporation in canola plants grown from treated seeds. The results demonstrate the efficacy of the co-fermented heterologous endophytes to enhance physiological performance of non-native host plants.

### Example 15

### Effects of Combined Strains WW6+WW7 on Total Nutrient Accumulation and Biomass

Winter wheat seeds (Triticum aestivum) treated with a seed inoculant composition comprising co-fermented WW6 and WW7 heterologous endophyte strains were grown and compared to control winter wheat plants seeds that were treated with the inoculant composition without any endophytes included. The inoculant composition was combined with a pre-biotic composition UBS 016 from Unium Bioscience Ltd. to aid the endophytes in survival, colony growth, and colonization of the host plant. Treated seeds were grown as follows: an appropriate amount of wheat seed was commercially treated at a rate of 500 mL of the mixed co-fermentate, 500 mL of 1% alginate, and 1000 ml of a 10% UBS 016 in water per 1 metric ton of seed. A crop protection package comprising fludioxonil and sedaxane to protect against seed-borne diseases was also added. Vibrance Duo® from Syngenta AG was used as the crop protection package, which contains 25 g/l sedaxane and 25 g/l fludioxonil. The Vibrance Duo® product was applied at 2 L per metric ton.

Control seed of the same variety received the same crop protection package and endophyte survival was confirmed on the seed by adding the seeds to a 0.2 M Phosphate resuspension solution and then plating on NLM semi-solid medium at the proper dilution. Seed was then stored for 1 month under normal industry conditions (4° C. to 15° C.) and was commercially planted in Cuxwold, Lincolnshire, United Kingdom in the fall using a seed drill in a large scale replicated CRO field trial at conventional rates.

Shoots were harvested in vegetative stage in late spring five months after planting and sent off for agronomic mineral nutrient analyses. Nutrient accumulation of shoot biomass was calculated by multiplying the total shoot weight by shoot ion concentrations. As shown in FIG. 22, winter wheat plants inoculated with the co-fermented WW6+WW7 accumulated significantly higher levels of macro- and micronutrients and the increased nutrient accumulation versus controls is expressed as Total Nutrient Content of Shoot Biomass % change from control.

The foregoing data demonstrate that the co-fermented heterologous endophytes WW6 and WW7 successfully improved macro- and micronutrient uptake and incorporation in winter wheat plants grown from inoculated seeds. The results demonstrate the efficacy of the co-fermented heterologous endophytes to enhance physiological performance of non-native host plants.

### Example 16

### Effects of Combined Strains WW6 and WW7 on Total Nutrient Accumulation Shoot Biomass

Spring oats seeds (Avena sativa var Elyann and SO1) treated with a seed inoculant composition incorporating co-fermented WW6 and WW7 fermentate at a 10⁷ CFU/ml heterologous endophyte strains were grown and compared to control seeds that were treated with the inoculant composition without an endophyte included. Treated seeds were grown as follows: an appropriate amount of oat seed was commercially treated at a rate of 500 mL the mixed co-fermentate, plus 500 mL of 1% alginate and 1000 ml of a 10% pre-biotic composition UBS 016 (from Unium Bioscience Ltd.) in water per metric ton of seed, and seed disease protectant Redigo (Bayer) following manufacturer instructions. Endophyte survival on the seed was confirmed by adding the seeds to a 0.2 M Phosphate resuspension solution and then plating on NLM semi-solid medium at the proper dilution. Control seed of the same variety received the same pre-biotic treatment but received no endophyte application. Oat seed was then stored for 1 month under normal industry seed storage conditions and was commercially planted in Suffolk, UK in May in a large scale replicated CRO field trial run at conventional fertilizer rates.

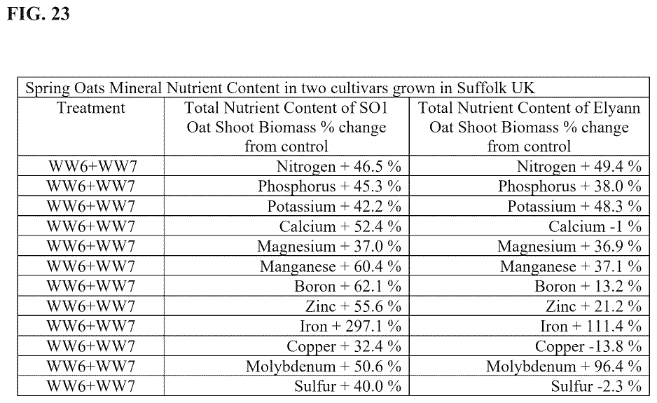

Shoots were harvested in early vegetative stage in mid-spring five months after planting and sent off for agronomic mineral nutrient analyses. Nutrient accumulation of shoot biomass was calculated by multiplying the total shoot weight by shoot ion concentrations. As shown in FIG. 23, spring oat plants inoculated with the co-fermented WW6+WW7 accumulated significantly higher levels of macro- and micronutrients and the increased nutrient accumulation versus controls is expressed as Total Nutrient Content of Shoot Biomass % change from control.

The foregoing data demonstrate that the co-fermented heterologous endophytes WW6 and WW7 successfully improved macro- and micronutrient uptake and incorporation in spring oat plants grown from inoculated seeds. The results demonstrate the efficacy of the co-fermented heterologous endophytes to enhance physiological performance of non-native host plants.

### Example 17

### Effects of Combined Strains WW5+WW6+WW7+PTD1 on Nutrient Concentrations

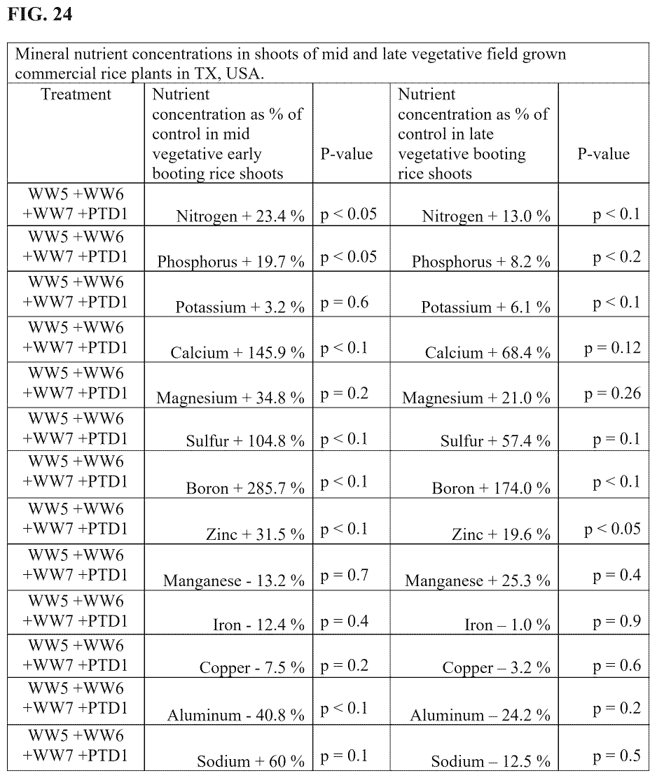

Asian rice (Oryza sativa) hybrid XP753 seeds were treated with a seed inoculant composition comprising a co-fermented WW5+WW6+WW7+PTD1 heterologous endophyte strains overlaid on seeds after the seeds were coated with a pre-treatment of two fungicide/insecticide products, GA3 (gibberellic acid), a dye, and a flowable zinc micronutrient coating. 500 mL endophyte fermentate and 1% alginate was added to 2,205 pounds of rice seed. Examination of the seed coat quality of the commercial treated seeds showed survival of the WW5+WW6+WW7+PTD1 endophyte strains, expressed as colony forming unit CFU per seed as follows: WW5 2.0×10⁶; WW6 8.0×10⁵; WW7 3.6×10⁶; PTD1 1.2×10⁶. Control seeds were treated with the pre-treatment, but not the seed inoculant composition. Seeds were then stored for 2 months under normal industry seed storage conditions and were commercially planted by a large grower in Clay County Arkansas, USA in spring 2020. Rice fields were fertilized with 400 lbs urea per acre, that is equivalent to 184 lbs of N per acre. Shoots including the leaves were pooled in mid vegetative (early booting) and separately again in late vegetative (booting), air dried and sent off for agronomic mineral nutrient content analyses. As can be seen FIG. 24, plants inoculated with (WW5+WW6+WW7+PTD1) accumulated higher levels of plant relevant macro and micro-nutrients and these differences are expressed as % change in leaf nutrient concentrations from control.

### Example 18

### Effects of (WW5, WW6, WW7 and PTD1) Applied as a Seed Treatment on Harvest Yield Under Reduced Nitrogen Fertilizer and Normal Nitrogen Fertilizer Rates in Agricultural Fields.

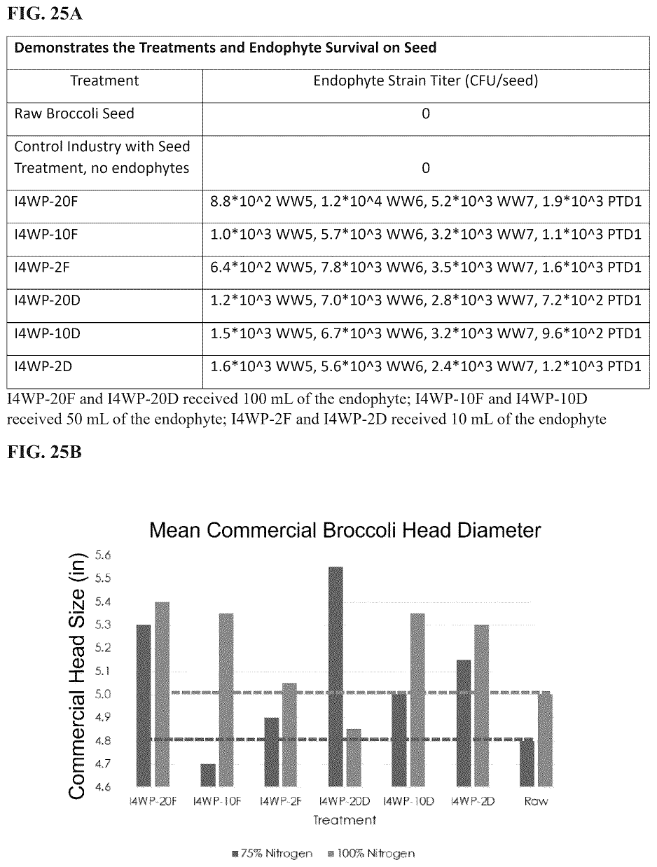

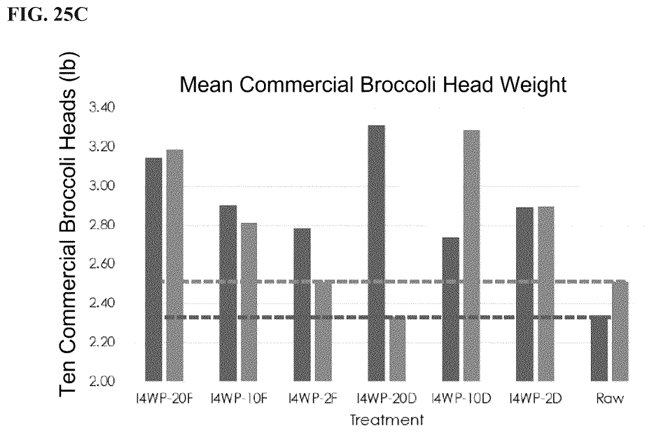

Broccoli seed was first commercially treated with a mixture of the endophyte fermentate plus 1% alginate and applied at different rates using two different crop protection packages and industry leading methods. Seed rates of endophytes applied were 10 mL, 50 mL and 100 mL of fermentate mixed into a commercial slurry and applied per 1 kg broccoli seed. Control seed of the same variety received the same crop protection packages minus the Endophytes. Endophyte survival was then assayed for the presence of each microbe. The microbe mix demonstrated survival of the strains (WW5, WW6, WW7, PTD1) on the seed after drying—the four-strain mix is denoted as “14WP” in FIG. 25A. The seed coat enumeration was done using 10 seeds washed in 10 ml of water to remove seed coat and then assayed. Dilution plating results showed clear survival with bacterial titers provided as Colony Forming Units/Seed (CFU/seed) of each strain that was still alive and dehydrated successfully then dormant on the seed FIG. 25A.

Broccoli seed was then stored for 2 months under normal industry conditions (<25° C. in dark packaging) and was commercially planted in Salinas, CA in the fall using commercial methods in a large scale replicated CRO field trial on a production farm fertilized at normal and 25% reduced nitrogen fertilizer rates compared to commercial Four nitrogen applications were applied through the drip. 12 gallons/acre of Calcium Ammonium Nitrate (17-0-0) was applied twice and 5 gallons/acre of (17-0-0) was applied an additional two times for the full rate of fertilizer were applied over the season. In the treatments with 25% reduced nitrogen fertilizer rates, the amount of nitrogen was reduced by 25% each time the fertilizer was applied. Additionally, all treatments received a total of 8 applications of 0-0-6-3% Ca fertilizer throughout the season. Irrigation was controlled at the discretion of the farm manager following commercial farming standards. After one month of growth there was a notable difference in plant size between treatments that received 100% nitrogen rates versus the treatments receiving 25% less nitrogen. There were no symptoms of any disease or damage from pests during the trial for any treatment group. At the time of harvest, there was still a marked difference in foliage between treatments that received 100% nitrogen rates versus the treatments receiving 25% less nitrogen. The difference in uniformity and commercial quality was then measured at harvest for all the treatments. In choosing commercial broccoli heads, the grower took into consideration different criteria such as head size (diameter in inches), smoothness of the head, dark green color, and firmness. The largest mean commercial head diameter at harvest was observed for the highest two endophyte treatment rates 100 mL (applied to the 14WP-20F and 14WP-20D groups) and 50 mL (applied to the 14WP-10F and 14WP-10D groups) per 1 kg for both seed crop protection packages under both 25% less N fertilizer and for the normal 100% fertilizer as seen in FIG. 25B denoted as 75% N and 100% N rates, respectively.

The largest mean commercial head weight at harvest was observed for the highest two endophyte treatment rates 100 mL and 50 mL per 1 kg seed for both crop protection packages under both 25% less N fertilizer and for the normal 100% fertilizer as seen in the FIG. 25C denoted as 75% N and 100% N rates, respectively.

### Example 19

### Effects of WW6 and WW7 Applied Individually as a Seed Treatment Under Reduced Nitrogen Fertilizer (32 ppm N) in a Hoagland's Drop Out Indoor Grow Room Pot Study.

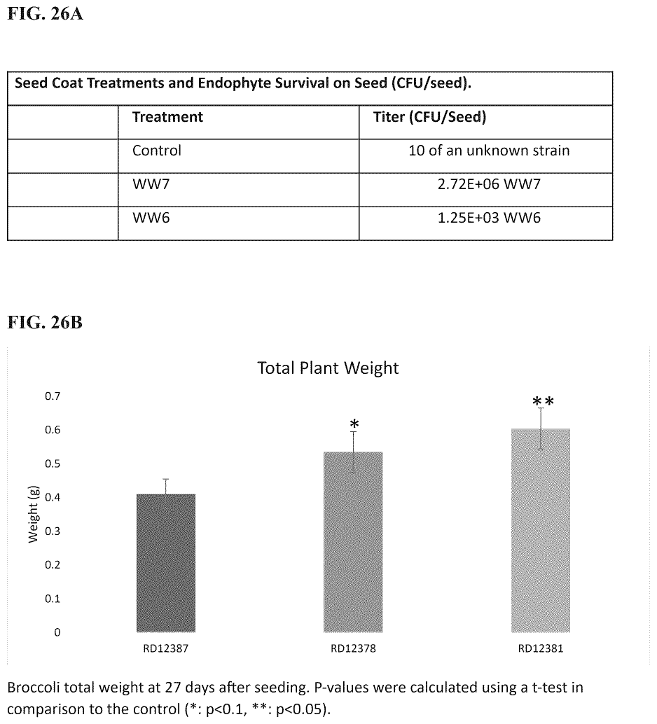

Early effects on shoot growth of the WW6 and WW7 endophytes applied individually to Brassica species under limited nitrogen in a controlled environment were tested. Endophyte strains WW6 and WW7 were applied individually in a fermentate inside a commercial seed coating process using both clay and dip coats onto broccoli seeds that were germinated and grown in flats with artificial soil-less media. Controls were the commercial coats applied alone without the endophyte fermentate mixture. Eight seeds per treatment were planted ½ inch deep in 2″ of inorganic planting media (all DI water washed; ⅓ play sand, ⅓ perlite, ⅓ vermiculite) in 10″×20″ plastic growing trays. Trays were watered using a Hoagland's N drop out solution modified with a 70% reduction in optimal nitrogen at 32 ppm total N pH 7 on Monday, Wednesday, and Friday for the term of the experiment. The study lasted twenty-seven-days and was performed in an indoor grow room under greenhouse lights applied for 14 hours per day with 710 μmol/m²s¹, an ambient temperature was 25° C., and 50% humidity. Seed coat survivability and dilution plating was assayed to determine the survival of the endophyte compositions together with crop protection products after dehydration on the commercially coated seeds. See FIG. 26A.

At harvest (27 day old), total plant seedling fresh weights were obtained for all treatments and the results are reported below. The WW7 treatment group (RD12378) showed a significant 30% increase in seedling weight over the control. The WW6 treatment group (RD12381) showed a highly significant 47% increase over the control. See FIG. 26B.

### Example 20

### Effects of WW5, WW6, WW7, and a Co-Fermented Mixture Applied as a Seed Treatment Under Reduced Nitrogen Fertilizer in a Controlled Environment Grow Room.

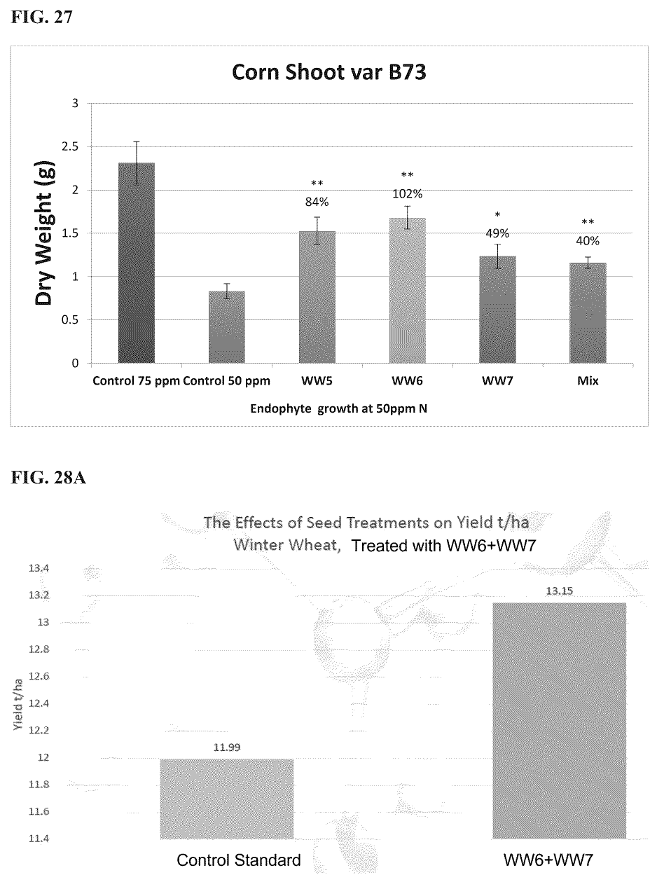

Effects on shoot growth of the WW5, WW6, and WW7 endophytes applied individually to corn seeds under limited nitrogen in a controlled environment were tested. Endophyte strain formulations were applied in combination with a 1% w/v sodium alginate (Scogin LDH) onto corn seeds that were germinated and grown in 1 gallon felt smart pots with artificial soil-less media. Eight seeds per treatment group were planted ½ inch deep in inorganic planting media (DI water washed; ⅓ play sand, ⅓ perlite, ⅓ vermiculite). Pots were watered using Hoagland's N drop out solution modified to include nitrogen at 50 ppm total N on Monday, Wednesday, and Friday for the term of the experiment. Two control groups were included, each treated with a modified Hoagland's solution and no endophytes. A first control group was treated with Hoagland's modified to include 75 ppm total N and a second control group and the endophyte treatment groups was treated with Hoagland's modified to include 50 ppm total N. The study was performed in an indoor grow room under greenhouse lights applied for 14 hours per day with 710 μmol/m²s¹, an ambient temperature was 25° C., and 50% humidity. At day 24 after emergence, the plants were harvested, washed, and dried at 45° C. in individual paper bags for 1 month. The dry weight of the shoots was then measured and recorded. The results are provided in FIG. 27. The endophyte seed coat formulations caused increased corn shoot dry weight biomass as follows; WW6+102% p>0.05, WW5 84% p>0.05 and WW7 49% p>0.01.

### Example 21

### Effects of WW6 and WW7 Applied as a Seed Treatment in the Field to Winter Wheat.

Winter wheat seeds were treated with co-fermented WW6+WW7 endophyte inoculant. The co-fermented WW6+WW7 endophyte mixture was fermented in low-nitrogen media and then freeze-dried into a powder. Five grams of the freeze-dried fermentate was mixed with 5 grams dried sodium alginate in 1 liter of water. The mixture was then combined with a commercially available pre-biotic composition UBS 016 (from Unium Bioscience Ltd.) in a ratio of about 3:1 to about 5:1 of the mixture to the pre-biotic composition. The combination resulted in a final seed slurry that was applied at a rate of about 4 L to about 6 L per metric ton of winter wheat seed. The control group was treated with the pre-biotic without fermentate. The seeds were planted commercially in the field in early November and the trial lasted until normal harvest in the middle of the following summer when the wheat grain was weighed, and final yield at harvest results were recorded. The treatment group showed a substantial 10% increase in crop yield, as shown in FIG. 28A.

Additionally, nitrogen accumulation was measured in the shoots of the winter wheat plants from February to June. The plants were harvested at each time point per 1 meter squared, dried and sent off for total Nitrogen measurements using the Kjeldahl method. The consistent increase of total nitrogen per hectare of wheat shoot was measured and is depicted in the results given in FIG. 28B which demonstrated a +30% increase in Kg of total nitrogen accumulation in wheat shoots/Ha when measured at the final sampling in June. The results clearly demonstrated the impact the diazotrophic N₂ fixing endophyte strains had on this wheat variety in the field. The endophyte treatment increased total plant shoot nitrogen per hectare throughout the vegetative growing season and demonstrated a +100% nitrogen shoot accumulation increase in early May and +30% nitrogen shoot accumulation per hectare increase in early June.

### Example 22

Carbon Accumulation in Plants Treated with Endophyte Strains.

The capability of the WW5, WW6, WW7, and PTD1 endophyte bacterial strains as a mixed consortia to increase total plant carbon accumulation when grown in field conditions was assayed using fast growing Populus trees planted in a Lower Mississippi Alluvial Valley field soil. Trees were inoculated using about 20 calcium alginate beads containing encapsulated endophytes and applied to the base of the Populus cuttings at planting. The replicate blocks on site were planted in (3 trees×5 trees) the field site contained five replicated blocks. Dormant, unrooted 22.86 cm long hybrid poplar cuttings were obtained from Greenwood Resources (Portland, Oregon, USA) and treated with (Admire® Pro, Bayer Corp., Whippany, NJ, USA).

For carbon sampling leaf samples were returned to the lab, dried in a 60° C. oven and ground to a fine powder, then placed into tin capsules. Samples were analyzed in an ECS 4010 CHNS-O Analyzer (Costech Analytical Technologies Inc. Valencia, CA, USA) to estimate total C and N concentrations.

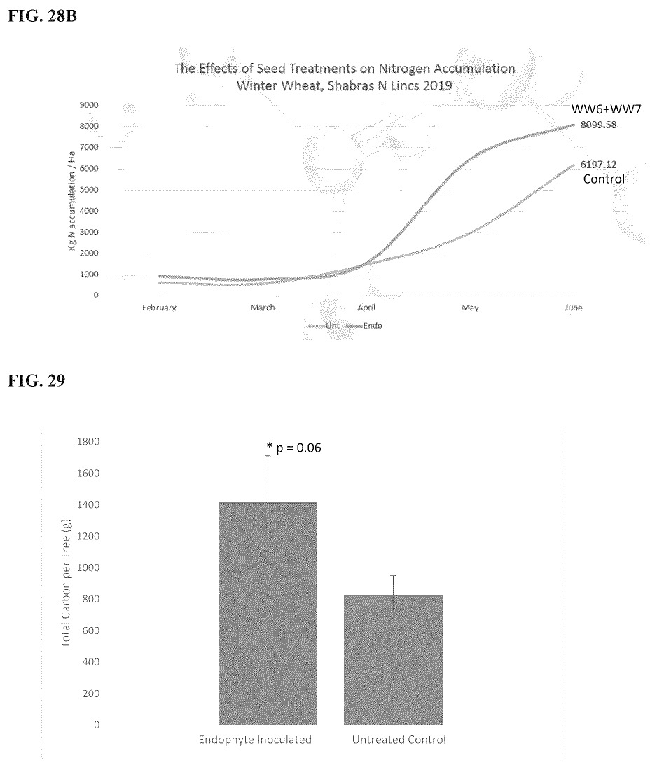

The statistically significant results demonstrated that endophyte inoculation increased total plant carbon content 71.01% at a p value=0.063 as shown in the below FIG. 29. Total carbon was calculated by multiplying the carbon percent by dry weight to the total biomass dry weight in n=12 trees for treated and n=12 trees for control. The results shown below FIG. 29 demonstrate significant increase in carbon accumulation in the treated group versus controls.

### Example 23

Compatibility with Crop Protection Chemistries Commonly Used for Seed Treatments.

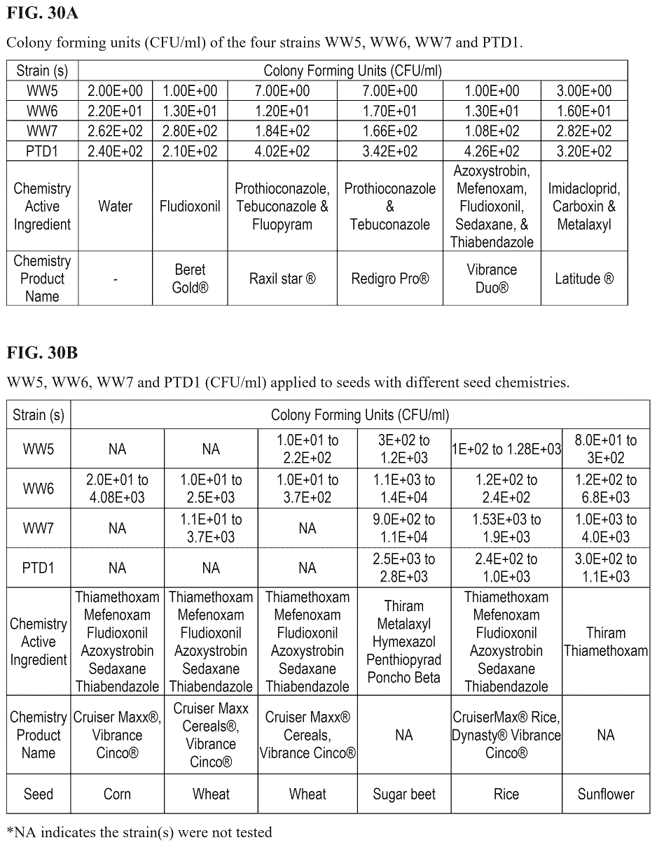

The endophyte strains WW5, WW6, WW7, and PTD1 were tested for their ability to survive when in combination with a variety of commonly used commercial seed crop protection chemistries. The endophyte strain(s) survivability was evaluated when added to five different seed crop protection chemistry solutions: Beret Gold® (Syngenta), Raxil Star® (Bayer CropScience), Redigro Pro® (Bayer CropScience), Vibrance Duo® (Syngenta) and Latitude® (Bayer CropScience). The solution mixes were prepared according to manufacture specifications. Five to six minutes after the mixtures were created the colony forming units (CFU/ml) of the strains were determined by thorough dilution and plating on NLM semi-solid media. The results given in FIG. 30A show all the strains can survive in the five different solution mixes.

WW5, WW6, WW7 and PTD1 survivability was evaluated when applied to the seeds of different crops and with different seed crop protection chemistry active ingredients: Mefenoxam, Fludioxonil, Azoxystrobin, Sedaxane, Thiabendazole Thiram, Metalaxyl, Hymexazol, Penthiopyrad, Poncho Beta and Thiamethoxam. The solution mixes were prepared according to manufacture specification and then the different strains were added to the solution before applying to the seeds by a commercial seed treater. The colony forming units (CFU/ml) of the strains that survived on the seed were evaluated by adding the seeds to a 0.2 M phosphate resuspension solution and then plating on NLM semi-solid medium at the proper dilution. The results given in FIG. 30B showed that the compositions of endophyte strains were resistant and survived when mixed in with a variety of commercial products and survived the temperatures and drying conditions found in commercial seed treatment processes.

### Example 24

Fertilizer, Micronutrient, & Herbicide Compatibility Commonly Used During in-Furrow and Foliar Applications.

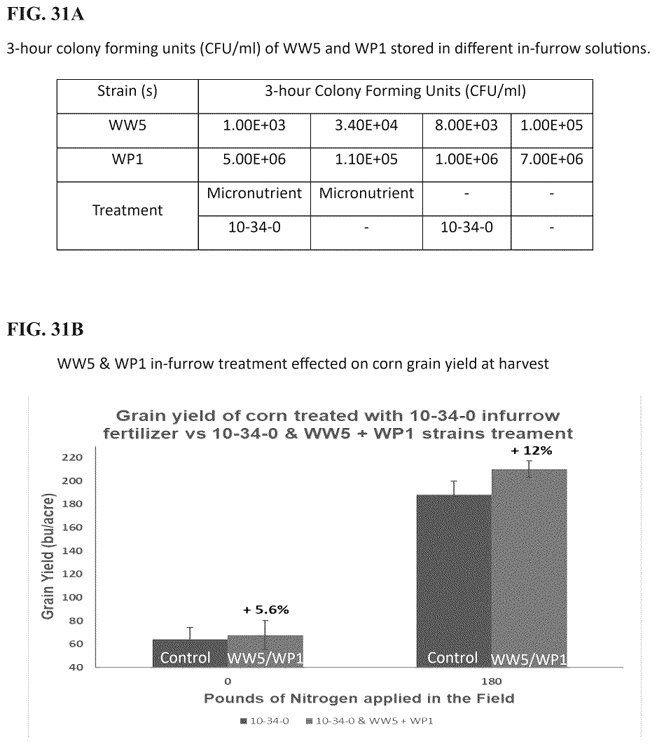

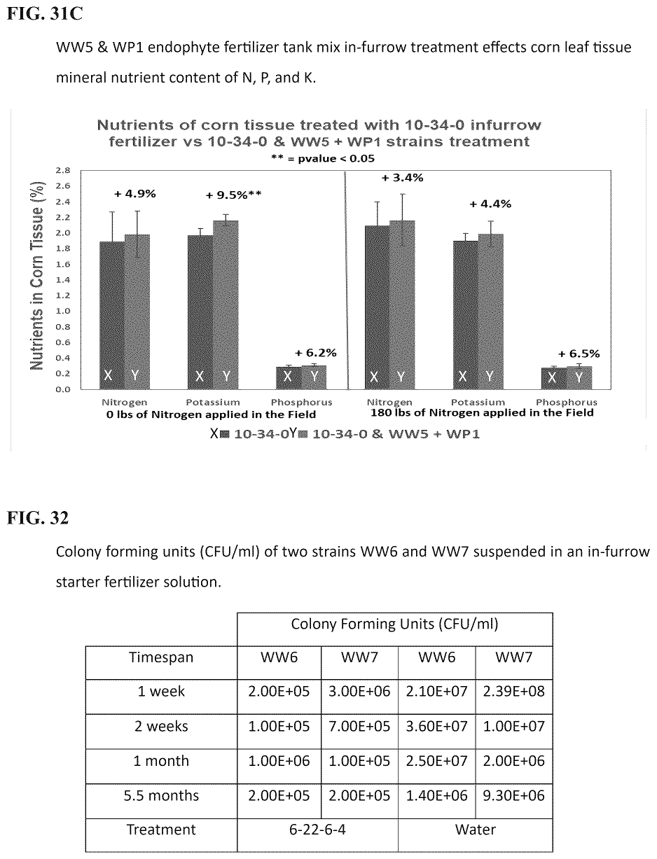

The ability of the endophyte strain(s) to be added with a variety of commonly used tank mix solutions for in-furrow and foliar application was evaluated based on the survivability of the strains over different periods of 3 hours to 1 month. WW5 and WP1 survivability was evaluated in ammonium polyphosphate (10-34-0), a liquid starter fertilizer used for in-furrow nutrient applications and/or a Micronutrient product (4% Ammoniacal Nitrogen, 3% Water Soluble Nitrogen, 9.0% Chelated Zinc). Volumes were scaled down from 5 gallon/acre to 50 ml for experimental purposes. The mixed compositions contained: 32 fl oz/acre of micronutrient, 5 gallon/acre of 10-34-0 fertilizer, 16 fl oz/acre of the strain WW5 inoculant, and water. Three hours after the mixtures were created the colony forming units (CFU/ml) of the two strains were determined by plating on NLM semi-solid medium. The results given in FIG. 31A demonstrate both WW5 and WP1 survived when 10-34-0 was present in the aqueous in-furrow fertilizer+endophyte tank mix.

Following, these results a tank mix of 3 gallon/acre of 10-34-0 fertilizer & 16 fl oz/acre of the strains WW5+WP1 was evaluated in the field in a replicated block trial applied as an in-furrow tank mix for growing corn. The tank mixture was dribbled on top of the seeds in-furrow controls received only the fertilizer tank mix at the same rates and no endophytes. The field was either not fertilized with N or it had a full N rate at 180 lb. N per acre.

The results given in FIG. 31B shows a +12% increase in crop grain yield at commercial harvest when the strains WW5+WP1 were combined with 10-34-0 fertilizer and corn was grown under full NPK fertilizer at conventional midwestern WI USA rates.

Additionally, mineral nutrient content was measured in the leaves of the corn plants at V9 growth stage. FIG. 31C demonstrated a consistent increase of total nitrogen, potassium and phosphorous (NPK) when the WW5 and WP1 strains were applied in-furrow inside the fertilizer tank mix at the time of planting. Importantly, the potassium (K) increases inside leaves taken from the blocks that received no nitrogen was a significant +9.5% increase demonstrating increased K uptake and assimilation into shoots in addition to N and P.

### Example 25

Fertilizer, micronutrient, & herbicide compatibility commonly used during in-furrow and foliar applications.

The effects of endophyte fertilizer, & micronutrient compatibility were tested using WW6 and WW7 strains that were evaluated for their long-term survivability when mixed with 6-22-6-4, a common liquid starter fertilizer often used for in-furrow nutrient applications. The test solution mix was scaled down from 5 gallons/acre to 50 ml for experimental container size purposes. The composition contained: 5 gallon/acre of 6-22-6-4 fertilizer, 40 fl oz/acre of the of WW6+WW7 microbial composition. Colony forming units (CFU/ml) of the two strains were determined by plating on NLM semi-solid medium over time between 1 week to 5.5 months. The results given in FIG. 32 show a slight reduction in survivability of both strains when 6-22-6-4 was present in the solution mix.

### Example 26

### Endophyte Compositions Tested for Foliar Herbicide Compatibility

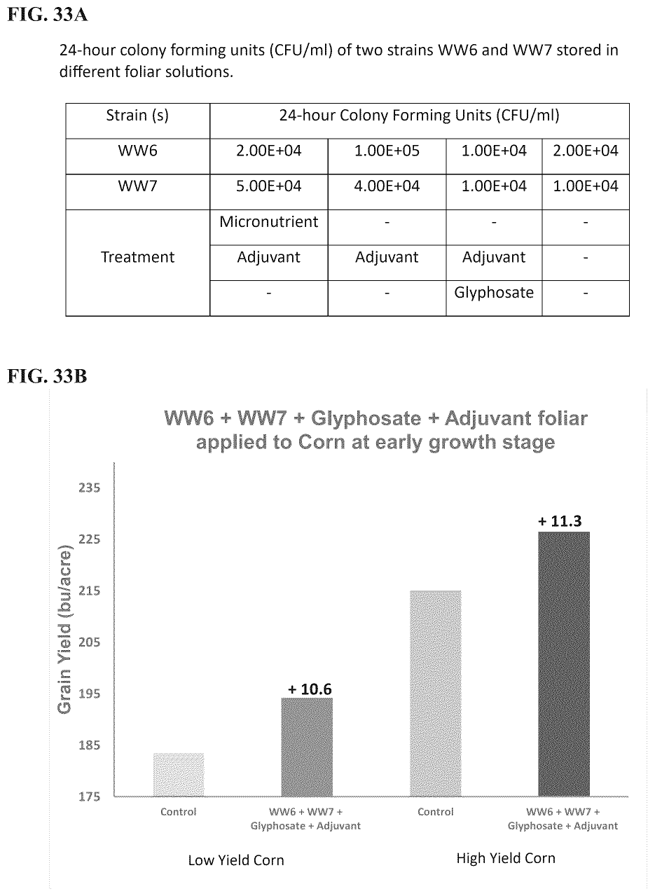

WW6 and WW7 survivability was evaluated in a composition including glyphosate, a broad-spectrum systemic herbicide used in foliar application and/or adjuvant (modified vegetable oil, polyoxyethylene sorbitan fatty ester, vegetable oil, and soybean oil ethoxylated) and/or micronutrient composition (sulfur 3.6%, boron 0.1%, manganese 3.0%, and zinc 4.0%). Solution mixes were scaled down from 10 gallon/acre to 50 ml for experimental purposes. The mixes contained a unique combination of the following products: 32 fl oz/acre of a micronutrient, 16 fl oz/acre of an adjuvant, 24 fl oz/acre of Glyphosate, and 16 fl oz/acre of WW6+WW7 microbial fermentate composition prepared according to the methods described herein, which had a pH of 5.3. Twenty-four hours after the mixtures were created, the colony forming units (CFU/ml) of the two strains WW6+WW7 were determined by plating on NLM semi-solid medium. The results given in FIG. 33A show no reduction in survivability of both strains in the different compositions.

A field study was performed to demonstrate the effects of the WW6 and WW7 composition when applied as a foliar spray to corn with the glyphosate herbicide and adjuvant. A tank mix was prepared by adding 32 oz/acre of the WW6 and WW7 inoculum composition, 32 oz/acre of Cornerstone 5 Plus (a glyphosate herbicide from WinField® United), 32 oz/acre of MasterLock (an adjuvant from WinField® United), and 10 gallon/acre of water. The field study included 27.5 ft.×5 ft. plots of high yielding and low yielding corn varieties. Three plots were treated with the tank mix described above and 3 plots were with a tank mix with the same ingredients excepting the endophyte strains. The results given in FIG. 33B shows a 10.6 bu/acre increase in crop yield for low yielding corn variety whereas the high yielding variety had a 11.3 bu/acre increase with WW6+WW7 endophyte strains compared to controls receiving no endophyte treatment.

### Example 27

### Effects of WW5, WW6, WW7, PTD1 on Enhancing Plant Tolerance to Flooding and Saturated Soils.

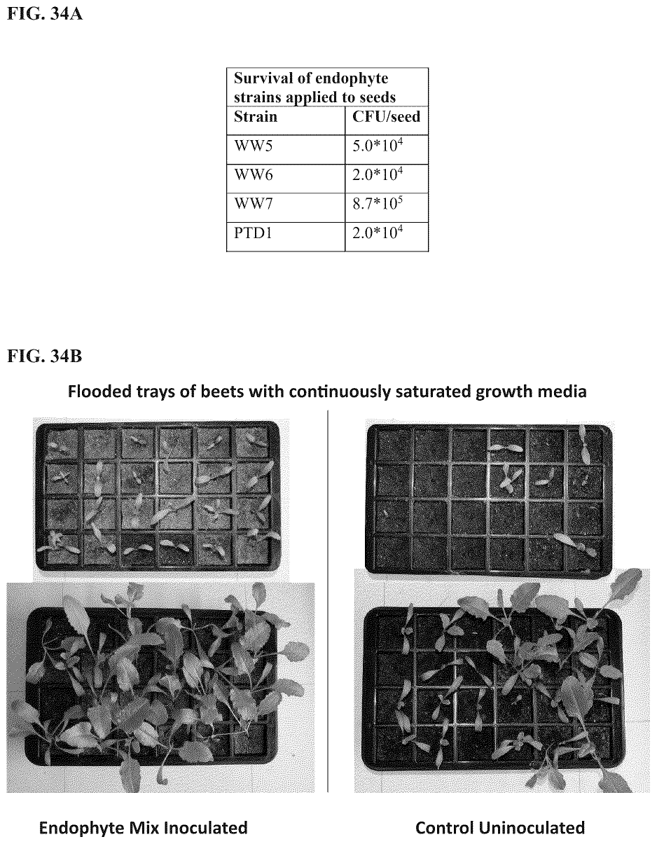

Flooding of agricultural soils is a large problem in US and global food production systems often resulting in plant death and crop loss on a massive scale. To test the ability of the endophytes to provide flood tolerance to crops, beets (Beta vulgaris) were commercially treated using commercial seed treatment methods with and without the application of heterologous endophytes (WW5, WW6, WW7, PTD1). Seed coating was applied to control plants and the seed coating combined with co-fermented endophyte strains WW5, WW6, WW7, and PTD1 was applied to the experimental seeds. The coated seeds were subsequently washed, and the experimental group was tested for the survival of the endophyte strains thereon. The bacterial enumeration was assayed using 10 seeds vortexed and washed in 10 mL of potassium-phosphate (KP) buffer to remove and dissolve the coat followed by dilution plating on NLM agar media. The assay showed clear survival of the endophytes with titers shown as CFU/seed in FIG. 34A.

Twenty-four endophyte treated seeds were each planted into individual cells and twenty-four control seeds were each planted into an individual cell. Seeds were consistently overwatered in fully saturated soil and germinated in standard commercial transplant potting media. The media was overwatered until saturated each day and grown out at 25° C. under natural diurnal light conditions. Once germinated, plants were continually overwatered and subjected to flood like conditions. After this flood exposure, plants were assessed for symptoms of flood stress, germination, and overall growth effects. Plants inoculated with the endophyte strains exhibited faster germination and establishment, less flood damage, and better growth under continually flooded saturated soil conditions when compared with controls, as shown in FIG. 34B.

The foregoing data demonstrate that the co-fermented heterologous endophytes WW5+WW6+WW7+PTD1 successfully improved beet plant response to abiotic stress (flooding), caused by overwatering a saturated media, increasing both early germination rates, and improved biomass growth compared controls. The visual results clearly demonstrate the efficacy of the co-fermented heterologous endophytes and their ability to enhance physiological performance of non-native host crop plants.

### Example 28

### Effects of Combined Endophyte Strains (WW6+WW7) Seed Treatment on Plant Cold Tolerance.

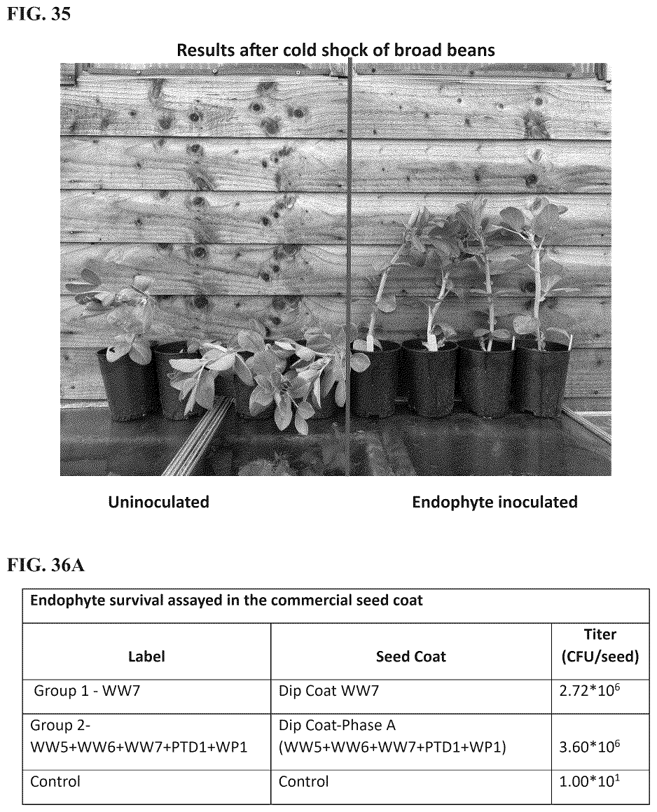

Two sets of broad beans (Vicia faba) were prepared, a control group treated with a commercial seed treatment and an experimental group treated with the commercial seed treatment and co-fermented heterologous endophyte strains WW6 and WW7 and prebiotic UBS 016 (from Unium Bioscience Ltd.). The co-fermented WW6+WW7 endophyte mixture was fermented in low-nitrogen media and then freeze-dried into a powder. Five grams of the freeze-dried fermentate was mixed with 5 grams dried sodium alginate in 500 ml of water. The mixture was then combined with 500 ml of the pre-biotic UBS 016 in a ratio of 1:1 the endophyte sodium alginate mixture to the pre-biotic composition. The combination was then incorporated into a commercial seed treatment slurry applied at a rate of about 4 L to about 6 L per metric ton of broad bean seed. Control group seeds were treated with the pre-biotic alone. Seeds were germinated in standard potting media and grown in 6-inch pots in a greenhouse at a daily minimum temperature in a range of 45° F. to 55° F. and a maximum temperature in a range of 65° F. to 70° F. under natural diurnal light conditions. Once germinated, the plants were fertilized with a 90-day slow-release complete fertilizer (Osmocote 15-9-12 Coated Granule Fertilizer). After 6 weeks post germination, both sets of plants were exposed to a cold shock treatment of 34° F. for 6 hours. After this exposure, plants were photographed and assessed for symptoms of cold stress and damage. As shown in FIG. 35, the experimental plants inoculated with WW6+WW7 exhibited less wilt and cold damage than control plants, which were severely wilted. Additionally, the endophyte treated plants recovered completely from the cold stress whereas the non-endophyte treated plants never fully recovered and showed signs of chlorosis and necrosis.

The foregoing data demonstrate that the co-fermented heterologous endophytes WW6+WW7 successfully improved broad bean plant response to abiotic stress (cold shock) compared controls. The results demonstrate the efficacy of the co-fermented heterologous endophytes to enhance physiological performance of non-native host plants.

### Example 29

### Effects of Individual and Combined Endophyte Strains on Plant Tolerance to Sodic/Saline Soils High in Boron and Chloride.

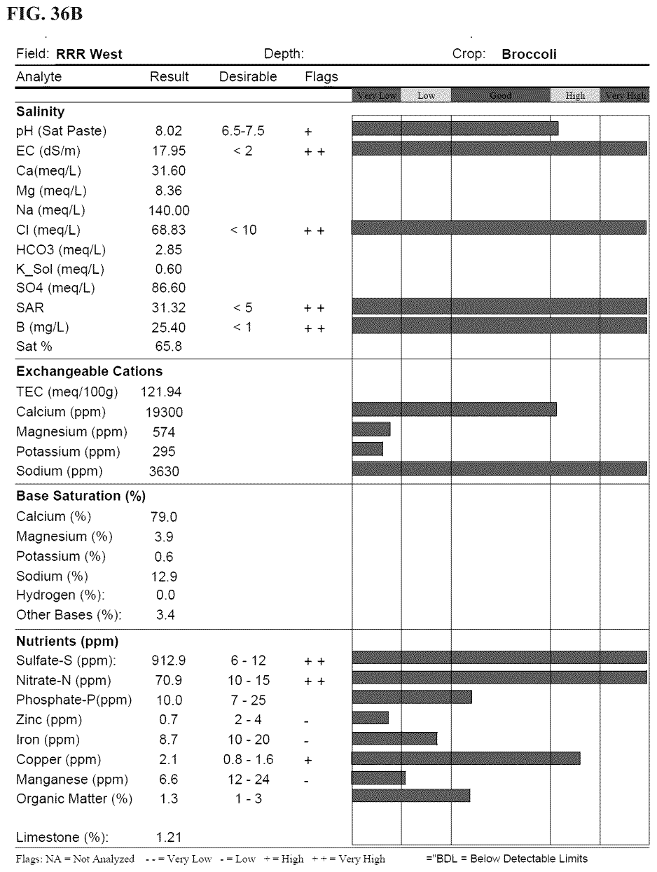

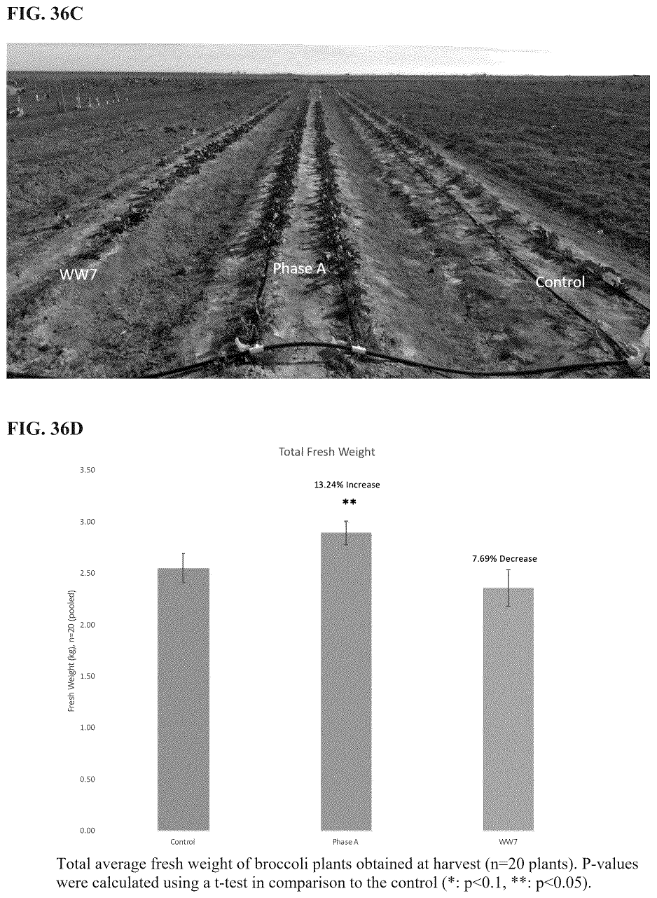

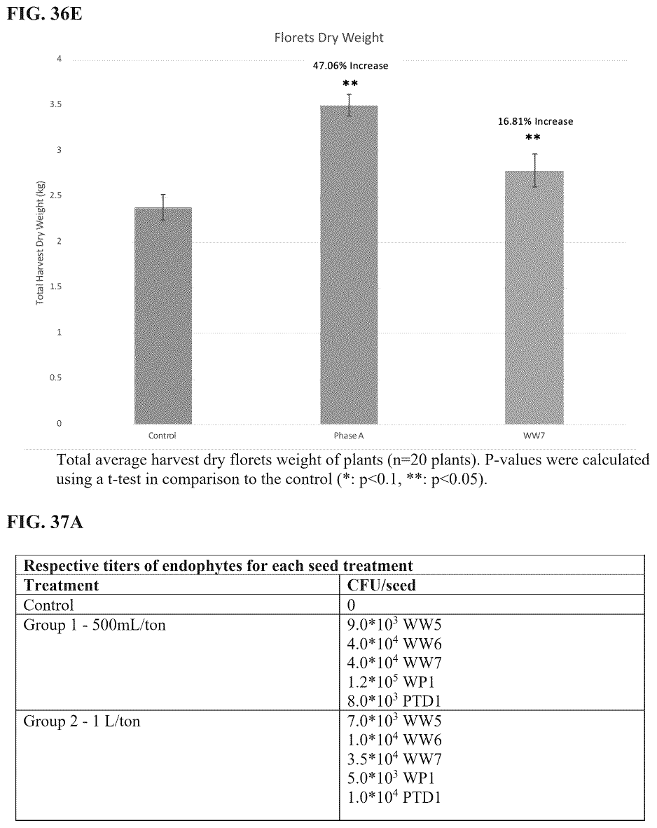

Soils that are characterized as sodic and/or saline limit plant growth and often result in plant death, crop loss and reduced yields. Globally it is estimated by the USDA that 40% of once arable farmland is now unusable for agriculture due to soils being high in salts. The novel endophytes strains disclosed herein were tested for their ability to increase crop plant growth after a seed treatment in sodic and saline soil. Control and experimental groups of Heritage broccoli (Brassica oleracea) seeds were prepared as follows: two experimental groups were coated using a commercial seed treatment method including a polymer dip coat combined with WW7 (Group 1) or endophyte strains WW5+WW6+WW7+PTD1+WP1 (referred to as “Phase A”) and a control group treated with the polymer dip coat and the broccoli protection package. The seeds were then assayed for total endophyte survival in the commercial seed coat and the total enumeration follows in FIG. 36A.

Seeds of the two endophyte treatments (Groups 1 and 2) and the control seeds were then grown in a commercial transplant greenhouse in Santa Monica, California prior to planting into the field trial. A location characterized by the USDA as having high sodic/saline soils and high boron and chloride levels was chosen for the field trial. The location was Five Points, California. An exchangeable sodium percentage (ESP) of more than 6% is considered a sodic soil and an ESP of 15% is considered highly sodic The ESP value indicates the percentage of the soil's cation exchange capacity (CEC) occupied by sodium. The poor-quality soil had the following chemistry profile. As shown in FIG. 36B, the soil used in the trial had an ESP of 12.9. The soil also included 25 ppm of boron, where a level of around 3-5 ppm is detrimental to plants. The soil also has very high chloride levels at 68 ppm. These characteristics indicated very poor soil used in the trial.

Using a rotor tiller, three identical 72-inch beds were created in a homogenous high salinity field site named (RRR west), two drip tape irrigation lines were carefully installed down each bed and tested. Bed size was 100 ft long beds, 6 ft wide 2 rows (2 ft from each bed edge, and 2 ft in between rows). 290 plants were planted from each of the 2 endophyte treatments and untreated control seeds. At the time of harvest, a photograph of the beds was taken 91 days after transplanting, which is provided as FIG. 36C. The salt tolerance of the endophyte-enhanced plants (denoted as WW7 and Phase A in the figure) can easily be visualized in Group 2 (Phase A).

Ninety-one_days after transplanting seven-week-old broccoli plants, they were harvested and weighed. Group 2 plants exhibited a 13.24% statistically significant increase in the fresh weight (p<0.05) in comparison to the control group. The graph provided in FIG. 36D provides the data for the broccoli field trial. The broccoli florets were then dried after harvest in a drying oven. The total dry weight of the Group 2 plants (Phase A) exhibited a statistically significant increase of 47.06% (p<0.05) and the Group 1 plants (WW7) exhibited a statistically significant increase of 16.81% (p<0.05) in comparison to the control group. See FIG. 36E.

The data demonstrate that the co-fermented heterologous endophytes WW5+WW6+WW7+PTD1+WP1 successfully improved broccoli fresh weight and dry weight in comparison to controls and (2) the endophyte strain improved broccoli dry weight in comparison to controls in conditions of abiotic stress (sodic/saline soil conditions). The results demonstrate the efficacy of the heterologous endophytes to enhance physiological performance of non-native host plants.

### Example 30

### Effects of Combined Heterologous Endophyte Strains on Increasing Plant Tolerance and Recovery in Drought Conditions.

Drought stress affects crops, plants, grass and trees in negative ways often resulting in plant death and crop loss. The occurrence of drought is increasing and drought causes many physiological and molecular biochemical changes in plants. Internal processes that help plants tolerate drought stress involve scavenging of reactive oxygen species (ROS), osmotic adjustment (OA), stomatal closure, and synthesis of protective molecules including inducible dehydrins. Recovery of plants after drought stress involves a series of steps occurring over time that can possibly be helped or facilitated by internal beneficial endophytes. To demonstrate the effects of the endophytes on increasing tolerance to drought, a fermentate mixture was prepared including endophyte strains WW5+WW6+WW7+PTD1 together with a fungal yeast endophyte WP1 and termed as “Phase A mix”. Phase A mix fermentate was with applied with a commercial seed treatment that incorporates a polymer with talc powder as a carrier to tall fescue seed (Festuca arundinacea—a forage grass used in livestock animal production) and dried into a shell like natural hard coat. Two treatment groups were used:
Group 1, treated with 0.5 L fermentate per ton of seed, and
Group 2, treated with 1.0 L (0.5 L fermentate+0.5 L 2% alginate) per ton of seed.

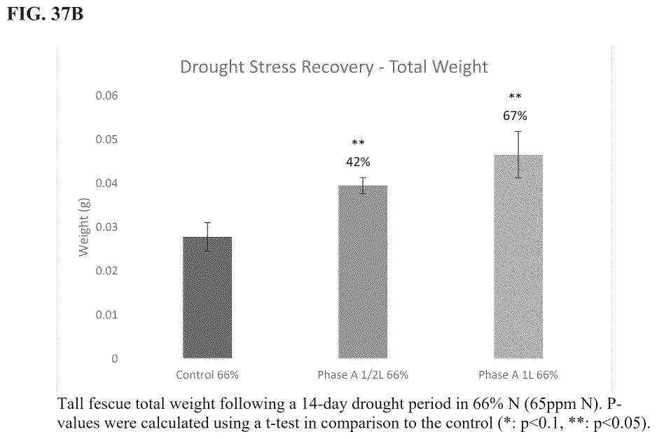

A control group received the commercial seed treatment without any endophyte strains included. These treatments were assayed and resulted in endophyte survival on the seed shown in FIG. 37A.

Control and experimental Groups 1 and 2 were planted at the same density in 3 flats containing a low-carbon growth media consisting of washed play sand, perlite and vermiculite. The seeds were watered three times weekly for 4 weeks with a modified low-nitrogen Hoagland's nutrient solution with 65 ppm nitrogen. The grass was grown at 30° C. and then subjected to a 14-day no watering, drought stress period that resulted in complete drying of the growth media. After stopping the drought stress treatment, the watering of the flats was resumed to allow the grass an opportunity to recover. Each group was then harvested and weighed. Groups 1 and 2 had a statistically significant (p<0.05) increase in dry weight: 42% and 67%, respectively. The total weight results are provided below in the graph of FIG. 37B.

The foregoing data demonstrate that the co-fermented heterologous endophytes WW5+WW6+WW7+PTD1+WP1 successfully improved fescue fresh weight in comparison to controls in conditions of abiotic stress (drought conditions). The results demonstrate the efficacy of the heterologous endophytes to enhance physiological performance of non-native host plants.

### Example 31

### Endophytes Increase Seedling Germination, Seedling Emergence, and Seedling Biomass Weight.

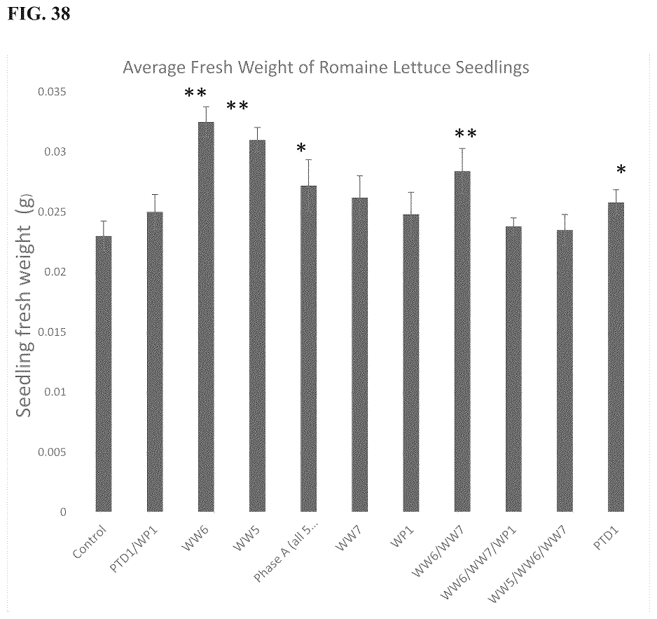

A series of tests were conducted in which the endophyte strains were applied to seeds to determine the effect of the application on increase seedling germination, increase seedling emergence from the seed coat and soil, and increase the seedling biomass weight. A first experiment included the treatment of romaine seed with 10 treatment groups and a control group, as identified in FIG. 38. Each treatment was applied to romaine seeds with a clay seed coat and the limited nitrogen endophyte fermentate mixture in a 1 w/v % alginate solution. The control did not include an endophyte fermentate. The seeds were coated using commercial methods, including clay coats and seed coat polymers including the control seeds. The seeds were germinated on square petri dishes with seed germination paper wetted by deionized sterile water 14 mL per sterile seed gemination container 4 in.×5 in. The seeds were then germinated, and seedlings grown under fluorescent without any nutrients in DI water under light for 14 days prior to being weighed. The results showed that the inoculated seedlings were larger and were able to grow better under limited nitrogen conditions. The results strongly suggest that the inoculated plants are able to fix atmospheric nitrogen and utilize nutrients from the seedling germination paper better than the controls. In relation to nitrogen fixation, it is observed that those strains that appear to fix the most nitrogen and grow the best under nitrogen free bacterial media conditions also resulted in the largest lettuce seedling weights in the following order WW6>WW5>WW6/WW7>endophyte mix (WW5, WW6, WW7, PTD1+WP1)>PTD1>WW7.

### Example 32

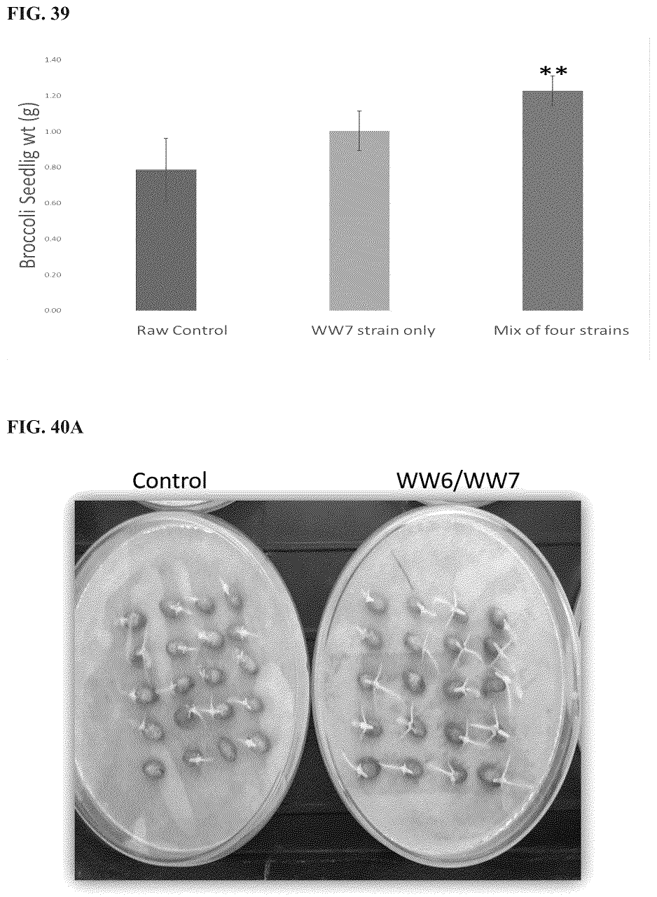

Experiments were conducted in which the endophyte strains were applied to broccoli seeds to determine the effect of the application on seedling biomass weight. The experiment included 4 treatment groups (WW7 strain only and mix of WW5, WW6, WW7 & PTD1) and a control group, as identified in FIG. 39. Each treatment was applied to broccoli seeds with a clay seed coat and the limited nitrogen endophyte fermentate mixture in a 1% w/v alginate solution. The control did not include an endophyte fermentate. The seeds were coated using commercial methods, including clay coats and seed coat polymers including the control seeds. The seeds were germinated on square petri dishes with seed germination paper wetted by deionized sterile water 14 mL per sterile seed gemination container 4 in.×5 in.

The seeds were germinated on square petri dishes with seed germination paper wetted by deionized sterile water 14 ml per sterile seed gemination container 4×5″. The seedlings were then grown under fluorescent without any nutrients under light for 14 days prior being weighed. The results showed that the inoculated seedlings were bigger and were able to grow better under the limited conditions. It is likely these inoculated plants fixed atmospheric nitrogen and utilized nutrients from the seedling germination paper better than the controls, resulting in the largest seedling weights as shown in FIG. 39.

### Example 33

Experiments were conducted in which the endophyte strains were applied to barley seeds to determine the effect of the application on seedling emergence. The experiment included a treatment group treated with the co-fermentate WW6+WW7 and a control group. Each treatment was applied to barley seeds with a clay seed coat and the limited nitrogen endophyte fermentate mixture in a 1 w/v % alginate solution. The control did not include an endophyte fermentate. The seeds were coated using commercial seed treatment containing polymers and crop protectants, including clay coats and seed coat polymers including the control seeds. The seeds were germinated on square petri dishes with seed germination paper wetted by deionized sterile water 14 mL per sterile seed gemination container 4 in.×5 in.

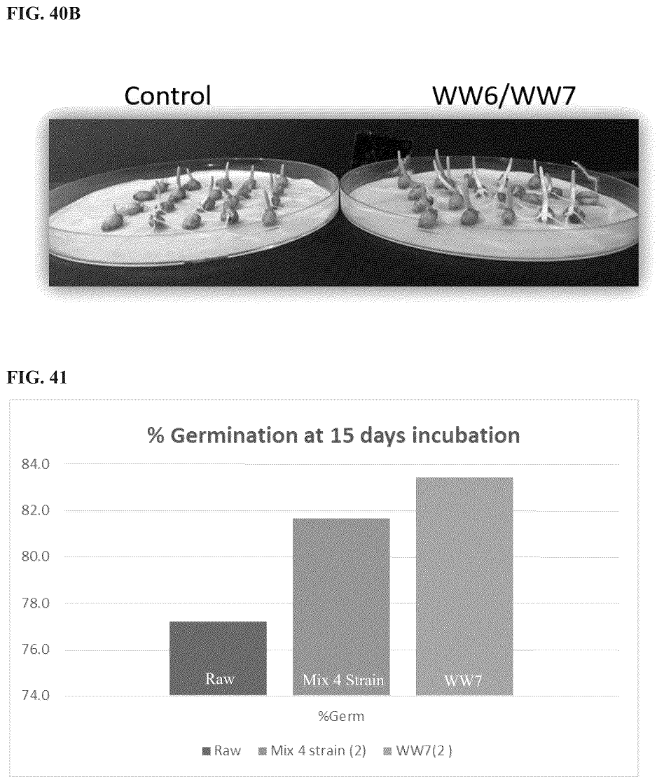

The seedlings were then germinated without any nutrients under white light for four days prior to first being photographed. The results showed that the inoculated seedlings were visibly bigger FIG. 40A and continued to grow bigger over time four days later as depicted in FIG. 40B.

### Example 34

Experiments were conducted in which the endophyte strains were applied to broccoli seeds to determine the effect of the application on seedling emergence. The experiment included two treatment groups one treated with the WW7 fermentate and a second treated with a four-strain mix (14WP), and a control group. Each treatment was applied to broccoli seeds with a clay seed coat and the limited nitrogen endophyte fermentate mixture in a 1% w/v alginate solution. The control did not include an endophyte fermentate. The seeds were coated using commercial seed treatment containing polymers and crop protectants, including clay coats and seed coat polymers including the control seeds.

The treatment groups and control broccoli seedlings were planted in individual cells. Seeds were germinated in standard commercial transplant potting media. The media was watered each day and grown out at 25° C. under natural diurnal light conditions for 15 days. Plants were then assessed for germination. An increase percent germination at 15 days was observed for the endophyte treated groups. Plants inoculated with the endophyte strains exhibited faster germination (see FIG. 41) and establishment and better growth.

### Example 35

Experiments were conducted in which the endophyte strains were applied to sugar beet cultivars seeds (C578 and M5) were treated with a seed inoculant composition. The experiment included two treatment groups one treated with the WW7 fermentate and a second treated with a four-strain mix (14WP), and a control group. The treatment group seeds were commercially treated at a rate of 50 mL of the fermentate and were applied per 1 kg of seed together with the crop protection products Thiram, Metalaxyl, Hymexazol, Penthiopyrad and Poncho Beta (Clothianidin). Control seed of the same variety received the same preparation, but without the fermentate. Seed was then stored for 1 month under normal industry conditions and was commercially planted.

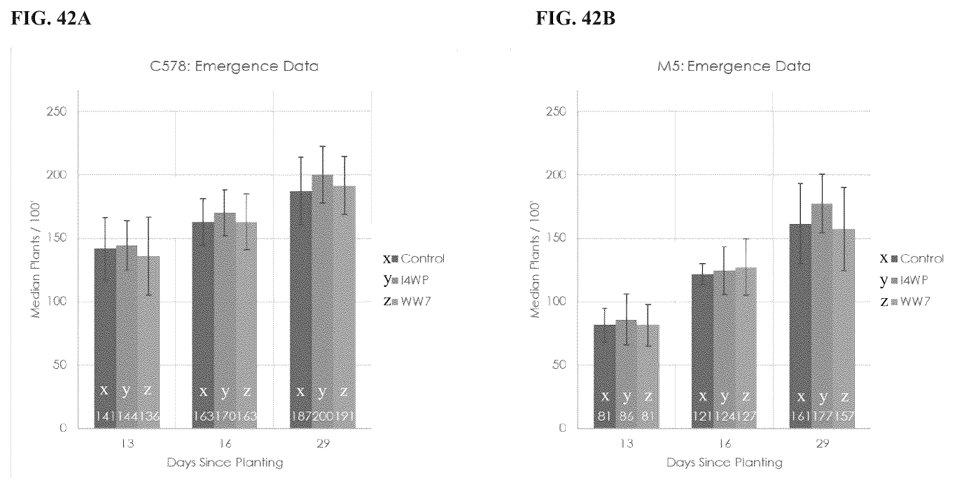

Beet seedling emergence after sewing was measured over time. The endophyte seed treatment composition allowed for the survival of WW5, WW6, WW7, PTD1 of all four strains (14WP) and on average the twenty-nine-day plant emergence of the beet cultivars was improved in comparison to the control group. In this trial, the emergence of 200 plants was treated as equaling a 75% plant stand in sugar beets and endophytes improved average emergence at 13, 16, and 29 days after planting. The C578 cultivar control had a 29-day emergence of 187 plants whereas the four strains (14WP) had 200 plants emerged (13 more) and for the M5 cultivar control emergence after 29 days it had 161 plants whereas the treatment group treated with the 14WP mixture resulted in 177 beet plants emerged (16 more), as shown in FIGS. 42A and 42B.

### Example 36

Experiments were conducted in which the endophyte strains were applied to wheat seeds as a seed inoculant composition. The experiment included a seed treatment group treated with co-fermented WW6/WW7 fermentate and a control group. The treatment group seeds were commercially treated at a rate of 500 mL of the fermentate and 500 mL of 1% alginate were applied per 1 metric ton of seed together with a pre-biotic UBS 016 following manufacturer's instruction. Control seed of the same variety received the same preparation, but without the fermentate. Seed was then stored for 1 month under normal industry conditions and was commercially planted.

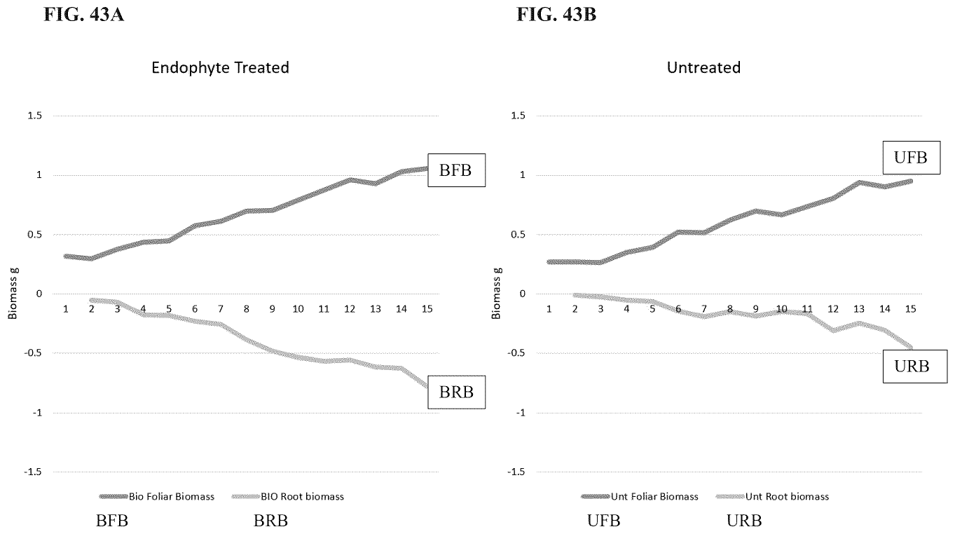

Wheat seedling emergence was measured over time in CRO field trials. The treatment group showed improved the growth of wheat seedlings over untreated controls after emergence that were harvested over time randomly from the appropriate field plots. The final seedling fresh weights after 15 days of tracking weights were for the WW6/WW7 combo was 1.054 g for foliage and 0.781 g for roots, while the control was 0.95 g for foliage and 0.44 g for roots. Fresh weights for new plants pulled on each day are presented below in FIGS. 43A and 43B.

### Example 37

### Effects of WW6 or WW7 Applied as a Seed Treatment on Grain Yield in the Field.

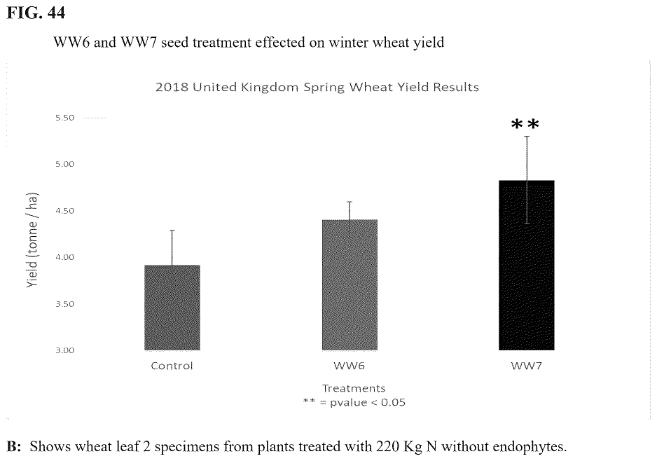

Endophytes were applied as seed treatments to enhance field yields under highly optimized nutrient regimes, spring wheat seeds were treated with WW6 or WW7 endophyte NLM fermentate. Prior to seed treatment, the fermentate was freeze dried into a powder. A seed treatment slurry was prepared by adding five grams of the freeze-dried fermentate along with 5 grams dried sodium alginate per liter of water. A 6 L volume of seed slurry was used to treat 1 metric ton of spring wheat seed variety Tybalt. The results given in FIG. 44 shows a 12% increase in crop yield for spring wheat seed treated with WW6 and seed treated with WW7 showed a statistically significant 23% increase in crop yield compared to controls receiving no endophyte treatment.

### Example 38

Reduction in Wheat Crop Nitrogen Fertilizer Requirements while Maintaining Harvest Yields.

A field study was designed to demonstrate the ability of endophyte seed treatment compositions to provide substantial harvested yields under reduced nitrogen fertilizer application. Bluerock romaine lettuce (Lactuca sativa, Vilmorin-Mikado USA) and corn (Zea mays) seeds were treated with WW6+WW7 fermentate, which was blended with 0.5% sodium alginate (Scogin™ LDH) to be used as a seed coat inoculum.

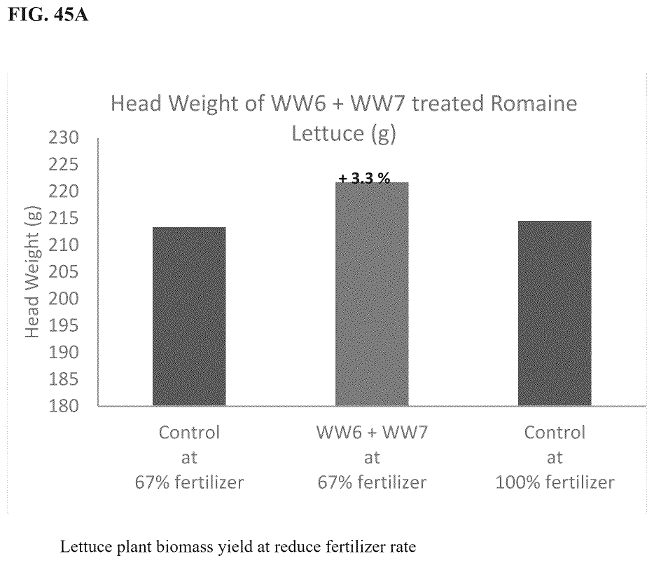

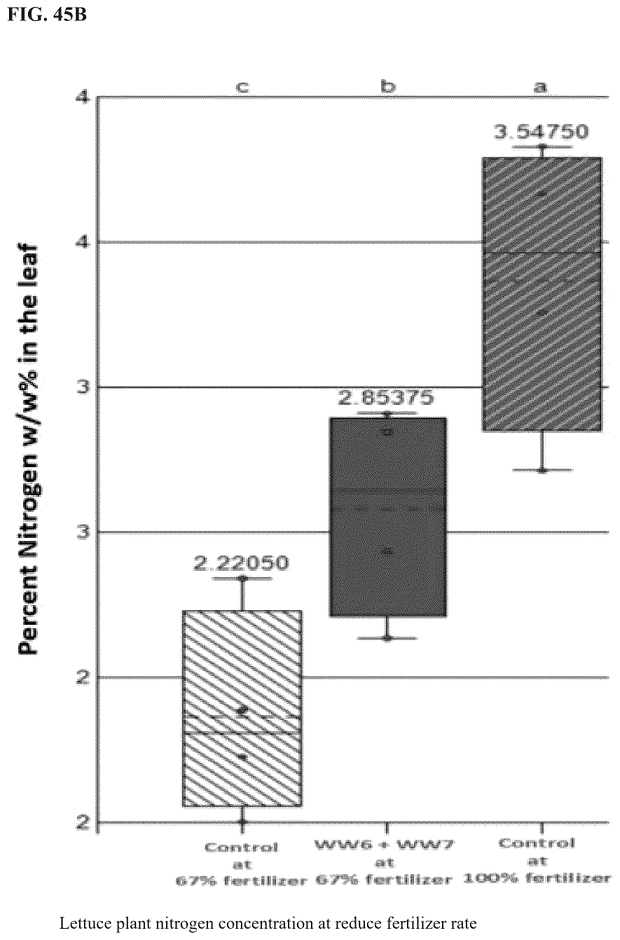

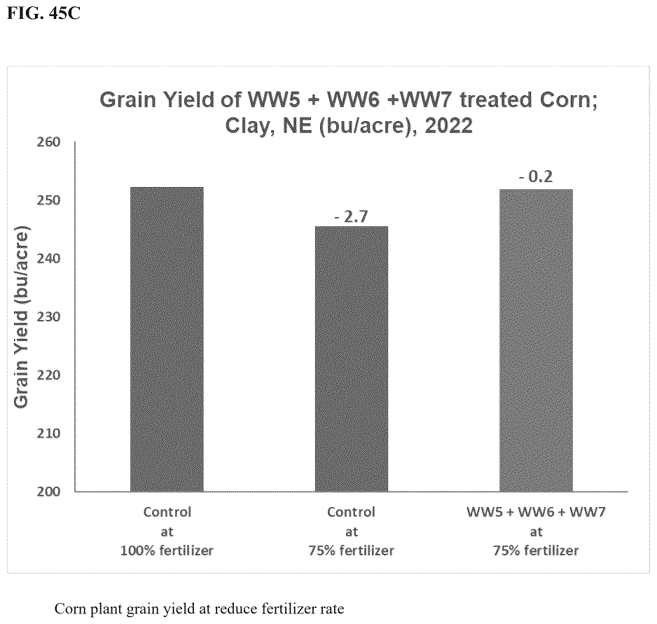

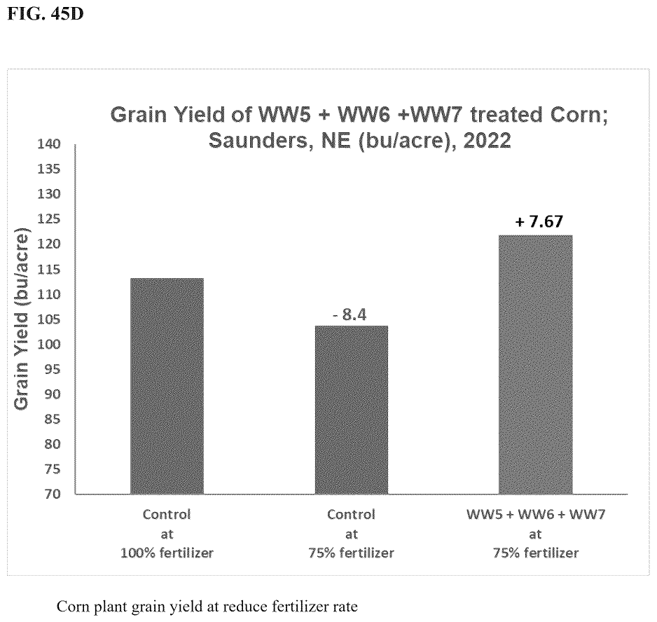

The lettuce seeds were planted in Fresno, CA in the fall using commercial methods in a CRO field trial on a farm fertilized at normal and 33% reduced nitrogen fertilizer rate. Field plots were divided into groups and fertilized at different rates by drip irrigation: (1) 25 lb/acre of calcium ammonium nitrate (17-0-0), (2) 50 lb/acre of 0-0-30 and 50 lb/acre of 0-46-0. The normal fertilized control plots also received 50 lb/acre of UN-32 nitrogen fertilizer. Irrigation was controlled at the discretion of the farm manager following commercial farming standards. At the time of harvest, 10 lettuce heads were collected from each plot and there were 6 plots per treatment. An average head weight was calculated per plot and then a total average was calculated. FIG. 45A shows the WW6+WW7 treatment under 33% reduced nitrogen resulted in 3.3% increase of head weight compared to the full nitrogen control plants. Additionally, plant tissue samples were collected from 4 of the 6 harvest plots for nitrogen tissue concentration assay. FIG. 45B provides the data demonstrating that the WW6+WW7 treated plants grown in reduced nitrogen showed statistically significant increases in leaf nitrogen concentration in comparison to the control plants under the same reduced nitrogen fertilizer regiment.

The WW5+WW6+WW7 fermentate was blended with 0.5% sodium alginate (Scogin™ LDH) and this composition was used to over treat corn (Channel 216-36 STX RIB) seeds previously treated with Prothioconazole, Metalaxyl, Fluoxastrobin, Clothianidin, LCO SP104, and Bacillus firmus 1-1582. The seeds were planted in Clay, Nebraska using commercial methods in a university field trial on a farm fertilized at normal and 25% reduced nitrogen fertilizer rate. The trial was a split-block and split-plot design, and each treatment included six plots that were fertilized at different rates: (1) 165 lbs of nitrogen/acre of anhydrous ammonia as a preplant for the 25% reduce rate and (2) 220 lbs of nitrogen/acre for the normal rate. Once the grain moisture reached approximately 15.5%, the grain yield was collected from the two middle planted rows from each plot. The average grain yield was calculated for the six plots per treatment. FIG. 45C shows the WW5+WW6+WW7 treatment group under 25% reduced nitrogen resulted in 0.2% reduction of average grain yield compared to the full nitrogen control plants whereas the control plants under reduce nitrogen resulted in 2.7% reduction of average grain yield.

Additionally, the WW5+WW6+WW7 fermentate was blended with 0.5% sodium alginate (Scogin™ LDH) and this composition was used to treat corn (Channel 213-19 VT2P RIB) seeds that was previously treated with Prothioconazole, Metalaxyl, Fluoxastrobin, Clothianidin, and LCO SP 104. The seeds were planted in Saunders, NE using commercial methods in a university field trial on a farm fertilized at normal and 25% reduced nitrogen fertilizer rate. The trial was a split-block and split-plot design and each treatment had six plots that were fertilized at different rates: (1) 112.5 lbs of nitrogen/acre of liquid UAN 32-0-0 as a preplant for the 25% reduce rate and (2) 150 lbs of nitrogen/acre for the normal rate. Once the grain moisture reached approximately 15.5%, the grain yield was collected from the two middle planted rows from each plot. The average grain yield was calculated for the six plots per treatment. FIG. 45D shows the WW5+WW6+WW7 treatment under 25% reduced nitrogen resulted in 7.67% increase of average grain yield compared to the full nitrogen control plants whereas the control plants under reduce nitrogen resulted in 8.4% reduction of average grain yield.

### Example 39

### Endophyte Herbicide Compatibility Commonly Used During Agricultural Foliar Applications to Control Weeds in a Wide Range of Monocot and Dicot Crops.

Three liquid foliar herbicide chemistries; Enlist One (Corteva Agriscience, LLC), Impact (AMVAC Chemical Corporation) and Callisto (Syngenta Crop Protection, LLC) were evaluated for their compatibility as a tank mix with the WW6 and WW7 strains. The solution mixes were scaled down from 20 gallon/acre to 10 ml for experimental purposes. The mixes contained 32 fl oz/acre of WW6+WW7 endophyte fermentate composition in combination with the following products: 16 fl oz/acre of Enlist One (2,4-D choline salt 55.7% w/w, Glycerol≥3-≤10% w/w, Dipropylene glycol monomethyl ether>=3-<10% w/w, Balance>20% w/w), 1 fl oz/acre of Impact (Topramezone 29.7% w/v, Inert Ingredients 70.3% w/v), 3 fl oz/acre of Callisto (Ethylene Glycol<15% w/v, Other ingredients>45% w/v, Mesotrione 40% w/v) or water. Four hours after the mixtures were created the colony forming units (CFU/ml) of the two strains were determined by plating on NLM semi-solid medium-FIG. 46.

The results given in FIG. 46 show no significant reduction in survivability of both endophyte strains in the different solution mixes over a prolonged period of time allowing for the use of these endophyte nutrient use efficiency, biomass and stress tolerance enhancement formulations in combination with commercial agricultural herbicide foliar spray applications.

### Example 40

### Example of Fertilizer-Biostimulant Compatibility for Commonly Used Agricultural Foliar Applications in a Wide Range of Monocot and Dicot Crops.

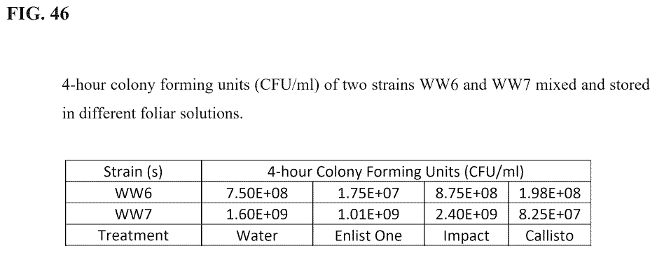

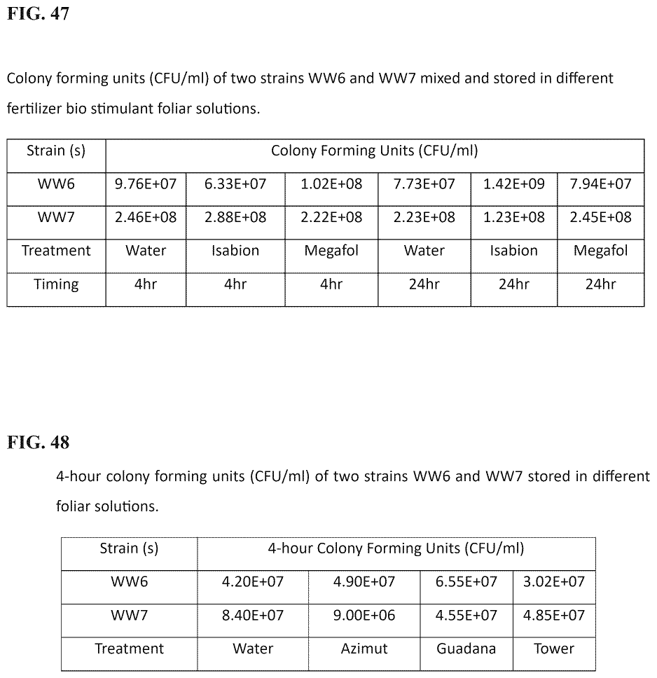

Two foliar fertilizers or bio-stimulants, Isabion (Syngenta Agro AG) and Megafol (Syngenta Crop Protection AG) were tested for their compatibility with the WW6 and WW7 strain compositions as a foliar tank mix. Tank mixes were scaled down from 400 liter/ha to 10 ml for experimental purposes. The mixes contained 2.4 liter/ha of the WW6+WW7 microbial composition in combination with the following products: Isabion at 6 liter/ha, Megafol at 3 liter/ha or water. Colony forming units (CFU/ml) of the two strains were evaluated four and twenty-four hours after the mixtures were created by plating on NLM semi-solid medium. See FIG. 47.

The results given in FIG. 47 show no significant reduction in survivability of both strains in the different solution mixes over a prolonged period of time allowing for the use of this endophyte composition in commercial agricultural fertilizer and bio stimulant foliar spray applications.

### Example 41

Examples of Stable Freeze-Dried Powdered Endophytes Compatibility with Foliar Herbicide Applications Commonly Used in a Wide Range of Agricultural and Monocot Cereals Crops.

Compatibility testing and evaluation was performed for the WW6 and WW7 strains as a freeze-dried composition reconstituted in water as an aqueous solution and mixed in separately with different standard herbicide tank mixes commonly used in cereals crops. Compatibility with the products Azimut (Comercial Quimica Masso, S.A), Guadana (Comercial Quimica Masso, S.A) and Tower (Comercial Quimica Masso, S.A) were all tested separately. Solution mixes were scaled down from 400 liter/ha to 10 ml for experimental purposes. The mixes contained 0.25% of a WW6+WW7 microbial product (10 grams of freeze-dried product mixed with 1 liter) in separate combination with the following products: 0.13% of Azimut (Florasulam 5 g/L (0.5% w/v)+Aminopyralid (potassium salt) 10 g/L (1% w/v)+2,4-D (ester-2-etilhexil) 180 g/L (18% w/v)), 0.15% of Guadana (Flufenacet 40% w/v (400 g/1) (32.4% w/w)+Diflufenican 20% w/v (200 g/1) (16.2% w/w)), 0.50% of Tower (Diflufenican 4%+Chlortoluron 25%+Pendimethalin 30% (SC)) or water as the control. Four hours after the mixtures were created and stored the colony forming units (CFU/ml) of the two strains were determined by plating on NLM semi-solid medium. See FIG. 48.

The results given in FIG. 48 show no significant reduction in survivability of both strains in the different product mixes over a prolonged period of time allowing for the use of this endophyte composition for nutrient use efficiency, biomass and stress tolerance enhancement product formulations in combination with commercial agricultural herbicide foliar spray applications.

### Example 42

The Ability to Enhance Nitrogen Acquisition in Crops after Treatment with an Endophyte Inoculum Seed Coat Suspension.

Tests were conducted to demonstrate the ability of different endophytes to fix atmospheric nitrogen inside crop plants germinated from inoculum coated hybrid corn seed using a seed treatment suspension solution mixed with a nutrient additive sodium alginate carbohydrate suspension at 0.5% w/v. Cultivation of endophyte inoculums as individual strains WW5, WW6, and PTD1 was performed using a tailored fermented inoculum until cultures reached a titer of at least 1.0E⁸ cells per mL and then combined with the nutrient additive carbohydrate. The seed treatment inoculums were QC checked for live cells and the appropriate species and strain colony morphologies were genetically confirmed using specific primers for colony PCR. This endophyte solution seed treatment composition was then mixed together with a widely used chemical seed treatment containing the fungicides Fludioxonil, Mefenoxam, and the neonicotinoid insecticide Thiamethoxam. The endophyte inoculum seed treatment solution was applied at a rate of 2.4 mL/1800 corn seeds and added to the chemical seed treatments following manufacturer suggested rates and application instructions for corn seed. Treated seeds were then dried and the colony enumeration of live microbes were assayed using seed coat washes and a KP buffer enumeration dilution plating on nitrogen limited media NLM plus agar. The enumeration results demonstrated that the on-seed microbe survival 1 month after the mixed slurry seed coat was applied were as follows: WW6˜400 CFU/seed, WW5˜40 CFU/seed and PTD1˜40 CFU/seed.

The seeds were then potted in 2-gallon pots containing 2.5 kg of field soil amended with perlite germinated and grown for 4-5 weeks until the V6 stage with no added fertilizer in the soil at 1,200 ppm total N plus 10 ppm soluble N (NO₃+NH₃). The plants were grown in the green house and analyzed for biological nitrogen fixation (BNF) using the ¹⁵N isotope dilution assay which specifically measures the percentage of nitrogen derived from the air. The proportional dependence of inoculated corn plants on atmospheric and soil nitrogen was estimated by comparing the natural ¹⁵N content of inoculated plant biomass with that of an adjacent reference non-inoculated plant subsisting solely on soil nitrogen. Total N and ¹⁵N isotope concentrations in corn shoot tissue were measured at V6 growth stage 4-5 weeks after planting using an Elementar EA Vario Pyrocube for total N and then 15N was measured using an Elementar IRMS GeoVisION, Isotope Ratio Mass Spectrometer (IRMS). To quantify the percent nitrogen derived from air (NDFA %) in plant shoots the ¹⁵N isotopic nitrogen amount measured by IRMS is subtracted from the total nitrogen concentrations and the difference is the NDFA reported as a percentage of the total N pool.

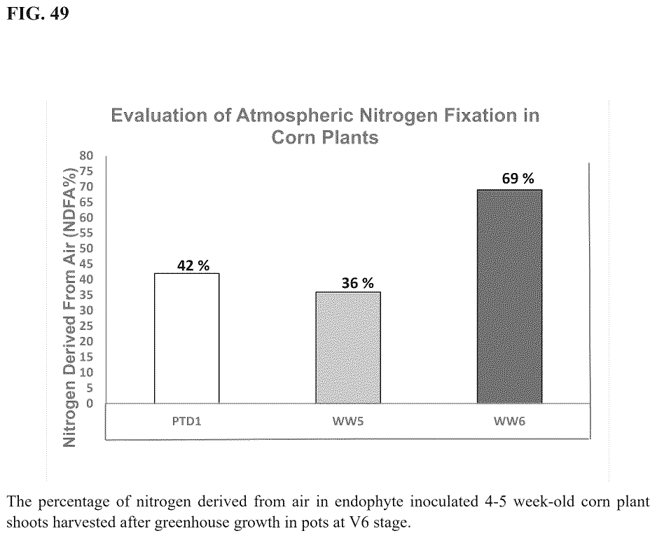

The results of the tests are shown in FIG. 49, demonstrating that seed treatment with the endophyte seed treatment inoculums resulted in major percentages of total nitrogen in corn shoots being derived from the air as follows; PTD1 42% NDFA, WW5 36% NDFA, and WW6 69% NDFA. The results clearly demonstrate that an endophyte seed treatment of corn can be stabilized after drying and used to inoculate seeds and the subsequent crop plants germinated from the seeds has enhanced biological nitrogen fixation during V6 a vegetative growth stage of corn plants.

### Example 43

The Ability to Enhance Atmospheric Nitrogen Fixation after a Formulated Liquid Endophyte Inoculum Treatment was Applied Directly to Young Wheat Plant Roots.

To demonstrate the ability of different endophytes to fix atmospheric nitrogen inside crop plants after a soil or infurrow-type rhizospheric inoculation, cultivations of endophyte liquid inoculums were produced for individual strains of WW5, WW6, PTD1 and for a co-cultured synergistic mixture of WW6+WW7 strains. The culture suspensions were prepared simultaneously using a tailored nitrogen limited NLM media with nutrient additives until cultures reached a titer of at least 1.0E⁸ cells per mL. The nutrient additive inoculums were then analyzed for live cells and colony morphologies. The presence of the WW5, WW6, PTD1 and the co-cultured mixture of WW6+WW7 strains in their respective inoculums were confirmed using specific primers for colony PCR. The endophyte suspension inoculums were then used to inoculate 2-week-old wheat plant roots. Inoculum suspensions were applied using 1 mL per plant applied to roots at plant base after transplanting from flats of potting soil. Transplants were planted into 1-gallon greenhouse pots filled with 1.5 kg of field soil amended with perlite with no added fertilizer in field soil at 1,200 ppm total N plus 10 ppm soluble N (NO₃+NH₃). The plants were grown in the green house until the jointing growth stage about 4-5 weeks after transplanting. The tissue was then harvested and analyzed for biological nitrogen fixation (BNF) measured as nitrogen derived from air using an Elementar EA Vario Pyrocube for total N analysis and 15N was measured using an Elementar IRMS GeoVisION, Isotope Ratio Mass Spectrometer (IRMS).

The results are shown in FIG. 50, demonstrating that seed treatment with the endophyte seed treatment inoculums resulted in substantial percentages of nitrogen being derived from the air in wheat shoots as follows; 43% NDFA for PTD1, 51% NDFA for WW5, 38% NDFA for WW6 and 47% NDFA for the WW6+WW7 synergistic combination. The results clearly demonstrate that an endophyte inoculation of wheat roots in both single strain and co-fermented nutrient additive solutions enhances biological nitrogen fixation abilities that is carried through into the main vegetative growth stages of the wheat plants.

### Example 44

Effects of Endophyte Synergistic Combinations WW6+WW7, Over Single Strains when Applied as a Freeze-Dried Reconstituted Seed Treatment Formulation to Barley.

The purpose of this trial was to determine if a mixed synergistic consortium of endophytes can better increase total barley plant biomass (shoot+root) than when single strains are used alone. Dry weight biomass was measured after 26 days when grown under reduced nitrogen in a controlled environment. Bacterial inoculums that were first freeze dried, stored and then resuspended at their original growth solution water content were made into a formulated seed treatment slurry and used as an endophyte seed inoculum (500 mL resuspended endophyte freeze-dried culture 1.1% w/v plus addition of sterile sodium alginate solution 2% w/v) and a sterile control nitrogen limited media (NLM) in a final alginate 2% w/v was added alone with no endophytes. Both treatments were applied at a rate of 1 L per 1 metric ton of seed. Spring barley was treated with 3 different seed formulation solutions using endophyte strains WW6, WW7, and co-fermented WW6+WW7 and dried overnight in a laminar flow cabinet. Four pots with 1 plant per pot were planted and grown for each of the 4 treatment groups with n=4 plants per treatment. Control plants were given a Hoagland's nitrogen drop out solution made at 65 ppm N (100%) and reduced 32 ppm N (50%). Endophyte inoculated experimental treatment groups were also given reduced 32 ppm N (50%). Hoagland's nutrients were given M, W, F via tray flood and after ½ hr trays were drained. Plants were grown for a period of 26 days after gemination in a plant growth incubator at 25° C., with artificial lighting set for 12 h light/12 h dark and then harvested and dried in paper bags at 45° C. for 48 days.

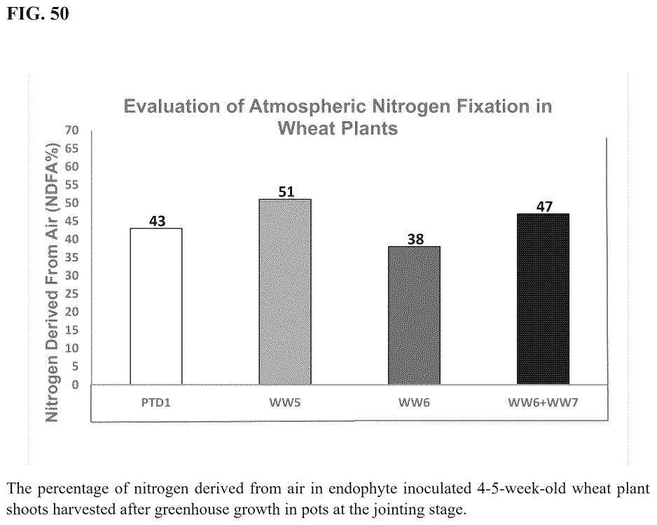

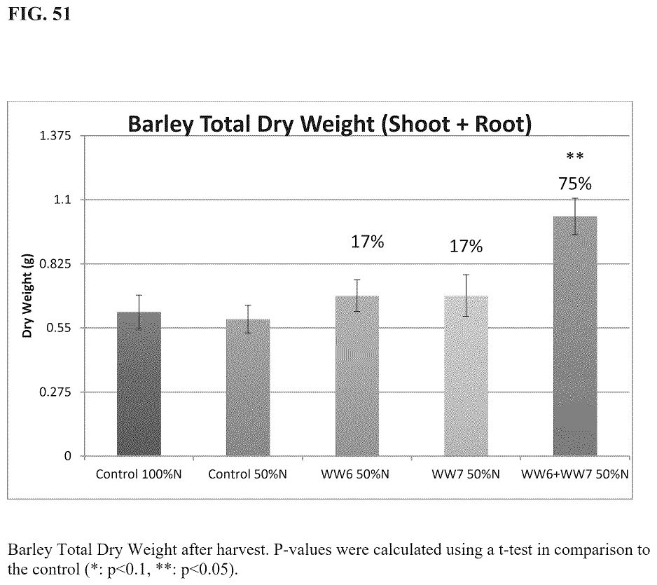

The are results shown in FIG. 51 demonstrate the synergistic effects of the combined treatments WW6+WW7 which increased total barley dry weight biomass under 50% reduced nitrogen the most causing a significant 75% increase in total biomass weight. Whereas WW6 alone had a non-significant 17% total biomass increase, and the WW7 alone had a non-significant 17% increase in total dry weight biomass over the controls when grown at the same reduced nitrogen rate.

### Example 45

### Stacking Endophyte Strains for a Synergistic Application to Increase Grain Yield Biomass Through an Inoculum Seed Treatment of Commercial Field Grown Spring Wheat.

To test the ability of the specific endophyte combinations to provide synergistic benefit to cereal crops, a variety of spring wheat (Sy Ingmar) was commercially treated using seed treatment methods with a co-fermentate of WW6+WW7, and a co-fermentate of WW5+WW6+WW7. No other seed treatments were applied. The seeds were coated by mixing the seed treatment slurries of the formulated solutions (>1.0 E⁶ CFU/mL NLM fermentate plus a carbohydrate solution 1-0.5% w/v) and applying the slurries at a rate of 0.23 mL bacteria per 1 lb of wheat seed using seed treatment equipment. Controls were not inoculated with endophyte fermentate. Wheat was planted in Berthold, ND USA in late May and harvested in September after 117 days of field growth. Plots sizes were: 5 ft×30 ft and contained Williams Silt Loam soil. Four rows of wheat were planted per plot with ten seeds per row spaced evenly. Four replications were conducted per treatment in a randomized complete block design, with 1,500,000 seeds planted per acre. Grain yield data was adjusted to 14% moisture. Fertilizer was applied at planting and consisted of a blend of 15 gal/ac of 10-34-0 & 44.5 gal/ac of 28-0-0. A soil test was conducted on soil at 0 to 24″ of depth, which exhibited the following characteristics: pH: 6.2, N: 16 lb/ac, P: 9 ppm, K: 345 ppm, O.M. 3.7%, CEC=20.83, Ca: 2427 ppm, Mg: 547 ppm, S: 404 lb/ac, and Zn: 1.11 ppm.

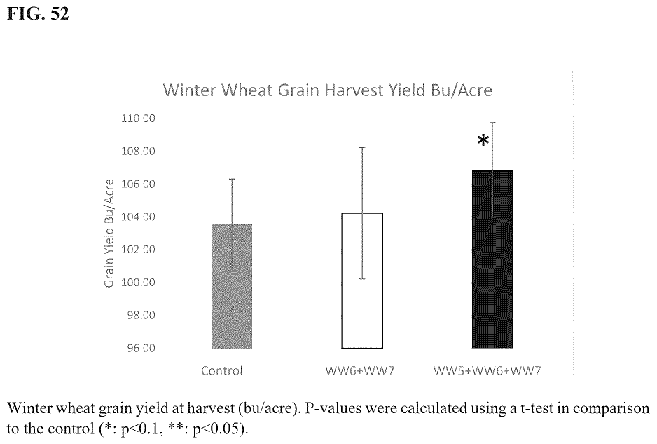

The results of the study are shown in FIG. 52. The two-strain treatment (WW6+WW7) increased average yield by 0.7 bu/ac while the 3-strain stacking of strains treatment was synergistic and further increased the average yield by 3.33 bu/ac over the controls and was statistically significant at a p<0.1.

### Example 46

### Stacking Endophyte Strains for Synergistic Applications to Increase Biomass Yield Though an Inoculum Seed Treatment of Commercial Field Grown Romaine Lettuce.

To test the ability of the specific endophyte combinations applied to provide a yield benefit to lettuce crops, a variety of romaine (River Road CVS) was commercially treated using single bacterial endophyte strains and co-fermented astrain combinations (2, 3, and 4 strains) of the WW5, WW6, WW7, and PTD1 strains with endophytic yeast WP1. All fermentates were mixed into a treatment slurry of the following formulation: CFU/mL>1.0 E⁶ cells in the NLM fermentate plus a carbohydrate solution 1% w/v. The treatment slurries were also mixed using seed treatment equipment into a clay seed coat then applied to seeds using standard commercial techniques. The endophyte solutions were applied at the following rates: 10 mL final volume of endophyte inoculum per ⅓ lb of romaine seed. In the case of co-fermentates including two endophyte strains, 5 mL of each strain were applied. In the case of co-fermentates including five endophyte strains, 2 mL of each strain was co-applied. Commercial standard field planting parameters included 80″ beds planted at 142,000 seeds for approximately one acre near Spreckles, CA.

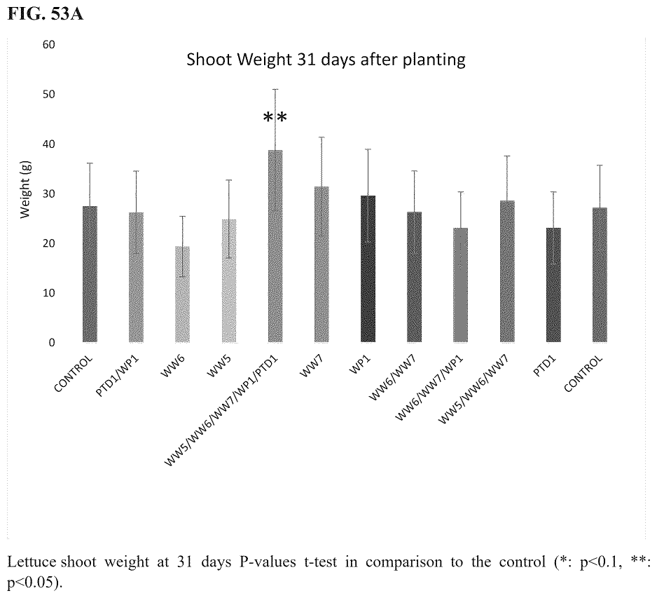

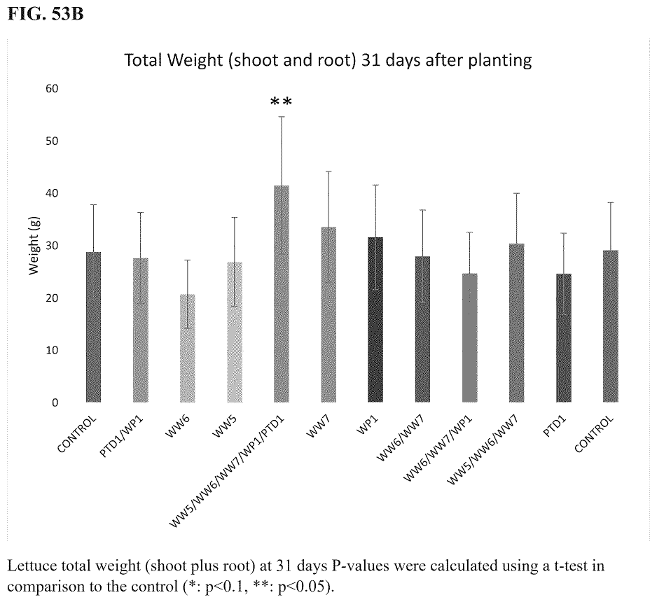

As shown in FIGS. 53A and 53B, single strain treatments improved yield. The combined mixed treatment of strains WW5, WW6, WW7, and PTD1 with the yeast strain WP1 showed a significant 43% increase in shoot biomass weight after commercial field growth.

### Example 47

Effects of Endophyte Synergistic Combinations WW6+WW7 Over Single Strains when Applied as a Seed Treatment to Canola after Mixed Together with a Prebiotic Carrier Composition in the Form of a Compatible Biostimulant.

Tests were run to determine whether a mixed synergistic consortium of endophytes when mixed together with a prebiotic plant microbial booster can better increase total canola plant biomass (shoot+root) in comparison to a single strain used alone with a prebiotic plant microbial booster. Fresh weight biomass was measured after 21 days when grown under reduced nitrogen in a controlled environment. Bacterial inoculums were freeze dried, stored, and then resuspended at their original growth solution water content and then incorporated into a formulated seed treatment slurry. The slurry was used to prepare an endophyte seed inoculum of the following formula: 500 mL of resuspended endophyte freeze dried culture at 1.1% w/v, 500 mL of a prebiotic and microbial biostimulant, and 4% w/v sterile sodium alginate solution made in H₂O. A sterile control nitrogen limited media (NLM) including the prebiotic and microbial bio stimulant, and 4% w/v sterile sodium alginate. The treatments were applied at a rate of 1 L per 1 metric ton of seed. The seeds (Untreated Spring Canola “Atomic TT”) were treated with 3 different seed formulation solutions using endophyte strains WW6, WW7, and WW6+WW7. The seeds were dried overnight in a laminar flow cabinet after application. Three pots with 1 plant per pot were planted and grown for each of the four treatment groups with n=3 plants each treatment. Control plants were given a Hoagland's nitrogen drop out solution made at 65 ppm N (100%) and 32 ppm N (50%). Experimental groups were given 32 ppm N (50%). Nutrients were given M, W, F via tray flood and after trays were drained. Plants were grown for a period of 21 days after gemination in a plant growth incubator at 25° C., with artificial lighting set for 12 h light/12 h dark and then harvested.

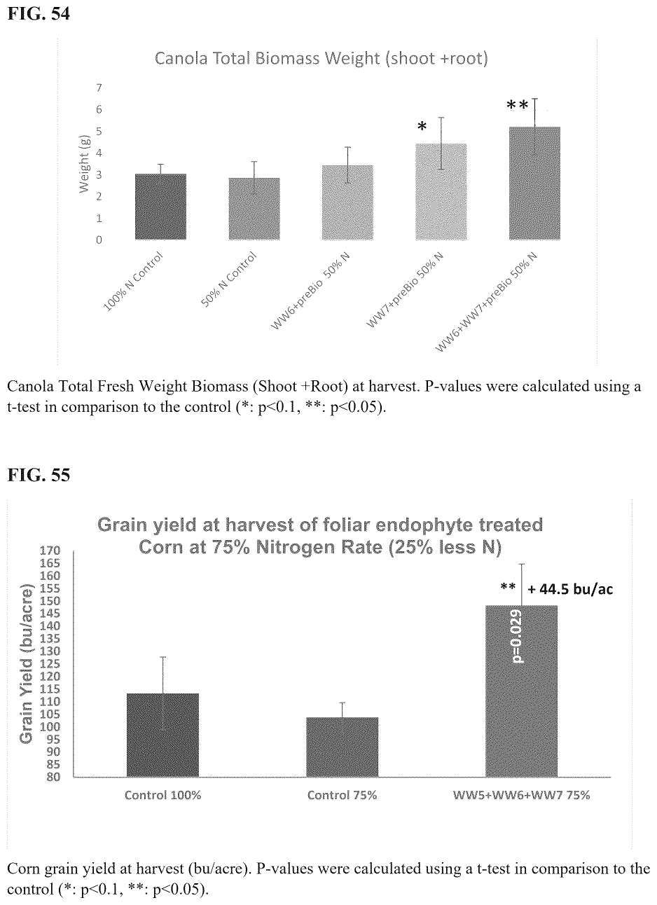

As shown in FIG. 54, the combined synergistic mixed treatment WW6+WW7+prebiotic increased total biomass the most causing a significant 82% increase in total biomass weight. Whereas the WW6+prebiotic increased total biomass 20% and the WW7 increased total biomass 55%.

### Example 48

### Use of Three Synergistic Endophyte Strains for Foliar Applications on Corn Shoots in Order to Reduce Fertilizer Requirements and Increase Harvest Grain Yields.

Tests were conducted to determine the efficacy of a novel endophyte combination (WW5+WW6+WW7) applied as a foliar spray to improve nitrogen fixation in a hybrid corn variety (Channel 113 day213-19VT2PRIB). The corn variety was first commercially treated with the seed chemistry Acceleron following manufactures methods. Corn was planted in early May in Mead, Nebraska, USA. The soil was Tomek Silt Loam, which had pre-planting nutrient levels that included P at 11.1 ppm, K at 344 ppm, and S at 7.4 ppm, with pH 5.8, O.M. at 4.1%, and CEC at 17.6. The corn plants were treated with a foliar spray applied at V6 using a pressurized sprayer deploying a mist spray of either a control or the experimental treatment including endophyte strains WW5, WW6, and WW7. Plots sizes were 10 ft×40 ft with four rows of planted corn per plot. Six replications were performed per treatment in a split block design. Fertilizer was also applied, including soil nitrate pre-fertilizer application applied at 17 lbs per acre, 75 lbs of nitrogen per acre as liquid formulation UAN 32-0-0 at pre-planting; and additional 37.5 lbs of nitrogen per acre was applied for a total of 130 lb N/ac. The N application to the treatment group was 75% of the standard nitrogen application practices for the planting area. A secondary control check treatment was incorporated into the study that received 170 lbs of nitrogen (100% of the standard nitrogen application). Herbicide was also applied. Pre-Acuron+Roundup was applied to the plants on May 13^{th} shortly after planting. The middle two rows were harvested in late October after 163 days of field growth and the grain was weighed and the statistical analyses were performed. Results are summarized in FIG. 55.

The results at 75% N fertilization rate demonstrated that the synergistic three strain endophyte foliar application applied at V6 significantly increased average corn grain yields +44.5 bu/ac (p=0.03). The results of endophyte foliar at V6 increased average yields when compared to the full 100% N control treatment 35 bu/ac.

### Example 49

Stacking Two Endophyte Strains for Synergistic in-Furrow Applications Used to Increase Harvested Grain Yields at Reduced Nitrogen 75%.

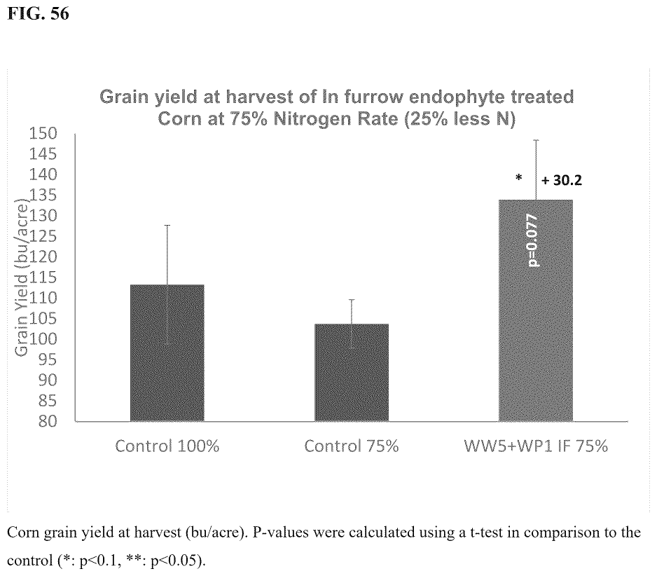

Tests were conducted to determine the efficacy of a specific endophyte combination WW5+WP1 applied as an in-furrow liquid composition to improve nitrogen fixing inoculum and yield in a hybrid corn variety (Channel 113 day213-19VT2PRIB). The corn variety was first commercially treated with the seed chemistry Acceleron following manufactures methods. Corn was planted in early May in Mead, Nebraska, USA. The soil was Tomek Silt Loam, which had pre-planting nutrient levels that included P at 11.1 ppm, K at 344 ppm, and S at 7.4 ppm, with pH 5.8, O.M. at 4.1%, and CEC at 17.6. The corn plants were treated at planting with a WW5+WP1 endophyte solution applied in furrow as an overlay on top of seed in the furrow using a dribble tube. Plots sizes were 10 ft×40 ft with four rows of planted corn per plot. Six replications were performed per treatment in a split block design. Fertilizer was also applied, including soil nitrate pre-fertilizer application applied at 17 lbs per acre, 75 lbs of nitrogen per acre as liquid formulation UAN 32-0-0 at pre-planting; and additional 37.5 lbs of nitrogen per acre was applied for a total of 130 lb N/ac. The N application to the treatment group was 75% of the standard nitrogen application practices for the planting area. A secondary control check treatment was incorporated into the study that received 170 lbs of nitrogen (100% of the standard nitrogen application). Herbicide was also applied. Pre-Acuron+Roundup was applied to the plants on May 13th shortly after planting. The middle two rows were harvested in late October after 163 days of field growth and the grain was weighed and the statistical analyses were performed. Results are summarized in FIG. 56.

The results at 75% N fertilization rate demonstrated that the synergistic two strain endophyte in furrow application applied at planting increased average corn grain yields +30.2 bu/ac (p=0.07). The results of endophyte in-furrow at planting increased average yields when compared to the full 100% N control treatment 20 bu/ac.

### Example 50

### Stacking Endophyte Strains for Synergistic Applications to Increase Biomass Yield in Strawberry Plants Using a Liquid Root Spray of Transplants Prior to Planting in Fields.

Tests were conducted to determine the efficacy of endophyte inoculations applied as liquid root spray to improve biomass yield in Albion strawberry plants at an organic strawberry farm in Salinas CA. Transplant roots were sprayed until covered with a thin mist of different endophyte inoculums. The spray treatment groups included WW5 alone, WW6 alone, WW7 alone, PTD1 alone, and mix of WW5, WW6, WW7, and PTD1. The control group included no endophyte strains. The treatments were applied, and the plants were planted using standard methods in early November 2016. Strawberries were planted in beds, containing two rows of strawberry plants spaced 25-30 cm apart (200-240 plants/row), and 3 beds per treatment were spaced 120 cm apart. Strawberries were fertilized with standard methods under the guidance of a registered CCA and harvested on May 20, 2017 after 28 weeks of growth.

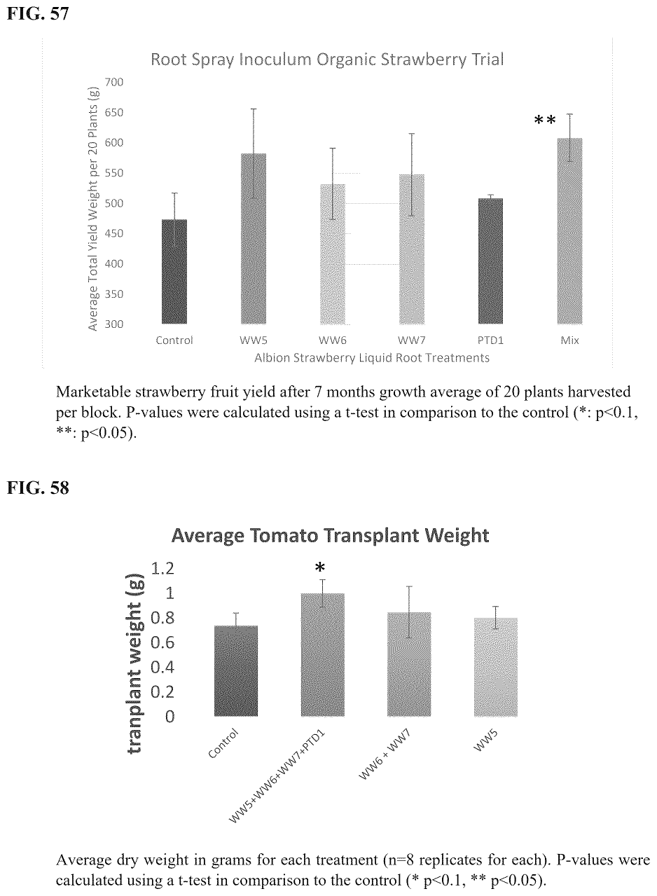

As shown in FIG. 57, the treatment group including mix of all 4 strains WW5, WW6, WW7, and PTD1 performed the best providing a 25% increase in fruit yield measured by fresh weight over the controls. The single strain inoculation treatments resulted in smaller increases over controls.

### Example 51

### Combining Endophyte Strains in Hard Partially Hydrated Beads as a Dry Granular Carrier for Synergistic Applications to Increase Tomato Transplant Biomass.

Tests were conducted to determine the efficacy of WW5 individually, a combination of WW6 and WW7, and a combination of four endophyte strains WW5, WW6, WW7, and PTD1 to increase biomass yield in tomatoes. Fermentate suspensions were blended into a sodium alginate slurry and used to drip into a calcium chloride 100 mM water bath in which a cation exchange reaction occurs and makes fully hydrated but hard calcium alginate beads prior to being dried to a final moisture content of about 4% to about 6% moisture content at a 2 mm final bead size. Quality 47 (Q47) hybrid tomato seeds were then placed on top of or adjacent to a single bead containing an endophyte treatment. Controls were treated with beads containing no endophyte strain. The plants are then germinated and grown in a commercial transplant potting mix with high organic matter composed of primarily peat and perlite inside a 125-cell transplant planter tray under normal commercial greenhouse fertilization rates and normal lighting at 25° C. for three weeks. After 21 days, 8 replicated plants per treatment were weighed and the total plant biomass dry weight was collected. Mix inoculated Q47 tomato plants had a 100% survival rate, compared to un-inoculated controls which had a less than optimal 88% germination rate. The endophyte enhanced biomass (shoot+root) results of the transplants growth are presented in FIG. 58.

The results clearly demonstrated a synergistic effect of growth in tomato plants inoculated with the alginate beads containing the combination of four endophyte strains WW5, WW6, WW7, and PTD1 compared to control. The mix of endophytes caused a +35% statistically significant increase in total plant weight (shoot and root). The single endophyte treatment WW5 and the combination of WW6 and WW7 provided smaller increases.

### Example 52

### Combining Endophyte Strains in Hard Partially Hydrated Beads as a Dry Granular Carrier for Synergistic Inoculum Applications to Loose-Leaf Lettuce Grown Under Deficient Bioavailable N and P.

Tests were conducted to determine the efficacy of endophyte strains in dried calcium alginate beads to reduce both nitrate and phosphate applications in a loose leaf lettuce (Lactuca sativa, variety Refugio) while increasing edible harvest yields. In the experiments, the bioavailable forms of N and P we reduced to deficiency and the experiments were run in a greenhouse using a specific soil-less media with the composition of a Terragreen which is a baked calcined clay gravel mix consisting of 9 kg Terragreen, 0.66 kg peat moss, 40 g of 8-3-5 organic fertilizer resulting in a nutrient concentration, and a well-drained organic rich soil profile that includes nitrate at 12 ppm, ammonia at 5 ppm, phosphate at 11 ppm, potassium at 328 ppm, sulfate at 690 ppm, SAR of 2.71, pH 7.28, EC of 2.89 dS/m, TEC of 18.76 meq/100 g, and total nitrogen of 1831 ppm mostly constituted as amino acids. The greenhouse trial was harvested 106 days after planting. Each seed was planted in a pint size starter pot with 1 bead per seed both placed ½ inch in the growth medium and watered. The alginate bead was applied adjacent or nearby germinating seeds in the soil. Plants were then carefully transplanted at 3 weeks using a hand trowel to remove all roots with bead and the surrounding soil intact. The roots, bead, and soil were placed in a same sized hole into 2-gallon felt smart pots. All plants were automatically watered with the same amount of water via a controlled drip system every 12 hours. Greenhouse lights (high pressure sodium halide lamps) were used to supplement low evening sunlight starting at 4:15 pm until 7:15 pm to allow for a full 12-hour growth cycle. Lettuce plants were inoculated with beads containing WW7 or PTD1 individually, or a mix of all four bacterial endophytes (WW5+WW6+WW7+PTD1). Controls received an alginate bead containing no endophytes. The treatments were all replicated six times per treatment n=6 pots each.

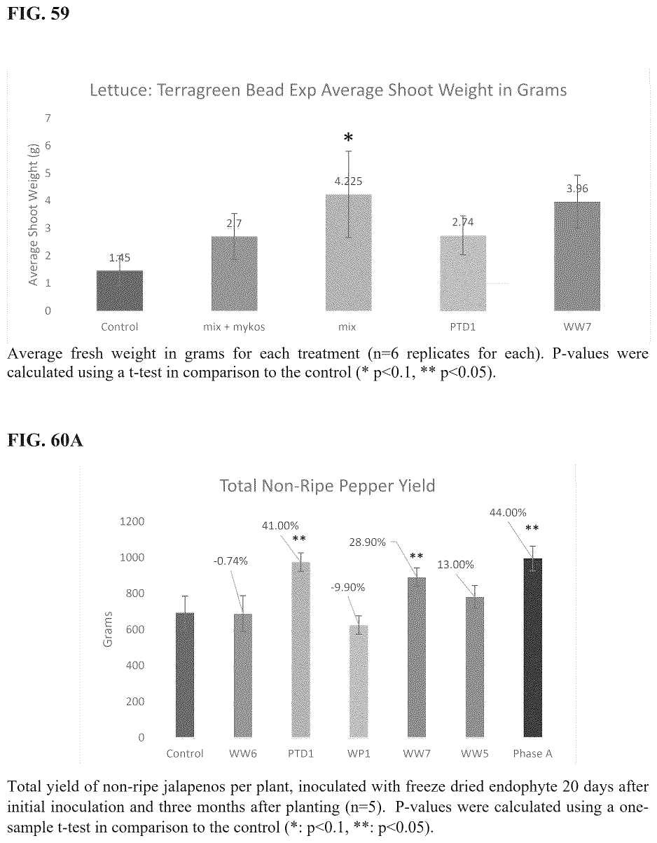

FIG. 59 shows the results of the fresh weight shoot biomass analyses. The treatment including a mix of all four endophytes performed the best with a shoot weight averaging 4.23 g per plant increasing the shoot weight over the control uninoculated plants by 191%, a statistically significant result at p<0.1. WW7 beads yielded an average of 3.96 g per plant, increasing the average shoot weight by 173%. PTD1 bead inoculation enhanced shoot biomass an average of 2.74 g per plant, increasing the average yield by 89%.

### Example 53

### Combining Endophyte Strains in Freeze Dried Powders for Reconstitution and a Foliar Spray Applied to Jalapeno Peppers in the Field.

To determine the efficacy of endophyte inoculum from a freeze-dried endophyte mix on growth and biomass jalapeño plants (variety RPP 7042). Approximately 420 jalapeno plants were inoculated in the late spring by a foliar spray applied at early bloom. Five individual endophytes strains (WW5, WW6, WW7, PTD1, and WP1) resuspended from freeze-dried powders were used. A mixture of the five resuspended endophyte strains was also prepared. One gram of freeze-dried endophytes was added per one liter of DI water to rehydrate the freeze-dried endophytes, and then placed into a foliar sprayer. The jalapeno seeds were planted in May to early June. Harvest dates were mid-September to early October. The reconstituted mixture was applied to foliage along each 50-foot treatment block. A 50-foot control treatment separated each treatment, three treatments per row. Fifty feet equaled approximately 75 plants. A series of growth and biomass analyses were conducted at the time of harvest 2 months later.

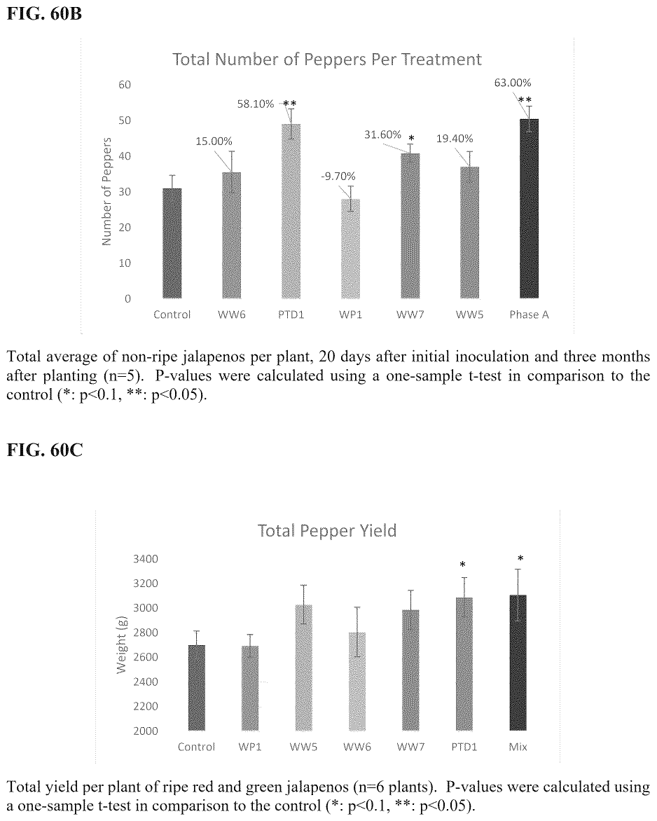

As shown in FIG. 60A, all inoculation treatments using a reconstituted freeze-dried powder at first blossom increased on average non-ripe pepper yield per plant over control, except for the treatment with the WP1 strain. The mix of all 5 strains (phase A) was found to be the best performing inoculum and yielded a synergistic effect. Phase A increased total non-ripe pepper yields +44% and had a positive impact on RPP 7042 jalapeño plants.

Additionally, the ability to increase the total number of peppers per treatment was assayed and the results follow in FIG. 60B. Phase A inoculation also demonstrated the best results with an initial 66% increase on average pepper number per each plant over control, except for WP1. The mix or Phase A, PTD1, and WW7 all showed to be statistically significant indicating freeze dried endophytes have an early positive impact on the jalapeño plants.

The total ripe pepper yield was also assayed three months after initial inoculation and four months after planting and reported in FIG. 60C. The results showed that endophyte inoculation using a reconstituted freeze-dried powder applied as a foliar spray at first flower break can be used to increase average jalapeño pepper yields per plant in comparison to controls. Phase A inoculum increased pepper biomass yields more than the other treatments. All of the tested bacterial endophyte strains increased yield versus controls, while Phase A showed an increased synergistic effect. The results are supportive that freeze dried endophytes once reconstituted into a solution and sprayed on flowers and foliage have an early positive impact on total jalapeño plant yield.

### Example 54

### Effects of Different Endophyte Seed Treatment Compositions Using WW5, WW6, WW7, PTD1 and WP1 on Leaf Chlorophyll.

A mixed synergistic composition of endophytes was freeze dried and resuspended in water and used to treat canola seeds to determine whether the resuspended endophyte fermentate can increase canola leaf chlorophyll after 36 days when grown under reduced nitrogen. The endophyte seed inoculum included resuspended endophyte freeze dried powder in 500 mL water at a concentration of 1.1% w/v mixed with 500 ml of a 4% sterile aqueous sodium alginate solution and applied at a rate of 1 L per metric ton of seed. Endophyte inoculum compositions were prepared for the following strain and strain combinations: WP1, GWW6+WW7, and WW5+WW6+WW7+WP1+PTD1). The seed variety used in the study was Spring Canola “Atomic TT”. The seeds were treated with the prepared endophyte treatment compositions: WP1, WW6+WW7, WW5+WW6+WW7+WP1+PTD1. A control group was treated with the NLM alone without endophytes. After the seeds were treated, all groups were dried at room temperature overnight in a laminar flow cabinet using. Four pots with 2 plants per pot were planted for each of the 5 treatment groups (n=8 plants/treatment). Control plants were given a Hoagland's nitrogen drop out solution made at 65 ppm N (100% N) and 32 ppm N (50% N). Experimental groups were given 32 ppm N (50% N). Nutrients were given three days per week (M, W, F) via tray flood and after 30 minutes trays were drained. Plants were grown for a period of 36 days after gemination in a plant growth incubator at 25° C., with artificial lighting set for 12 h light/12 h dark and then harvested.

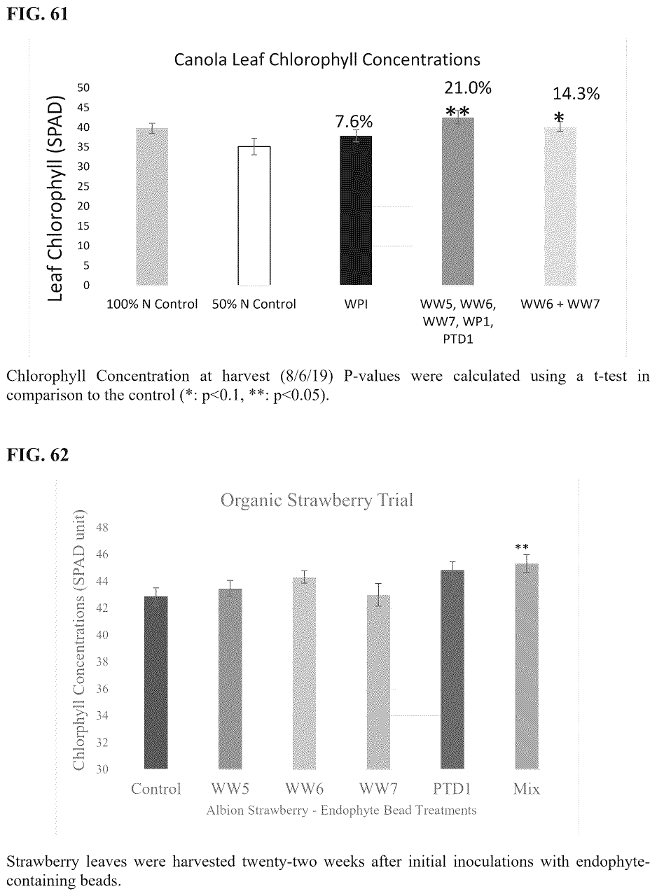

The results demonstrated that an endophyte composition used as a seed treatment of canola had a synergistic effect on leaf chlorophyll and the two strain WW6+WW7 increased leaf chlorophyl by 14.3% p<0.1 while the synergistic 5 strain endophyte consortia composition increased leaf chlorophyll by 21.0% p<0.05. See FIG. 61.

### Example 55

### Stacking Endophyte Strains in Alginate Beads for Synergistic Applications to Increase Leaf Chlorophyll in Strawberry Plants.

Tests were conducted to determine the efficacy of endophyte inoculum applied as a composition to increase leaf chlorophyll in Albion strawberry plants. Tests were conducted at an organic strawberry farm in Salinas CA. Strawberry plants were transplanted over five 2 mm calcium alginate beads placed in holes prior to planting. Calcium alginate beads were prepared with one of the following treatment groups: endophyte inoculum including WW5 alone, endophyte inoculum including WW6 alone, endophyte inoculum including WW7 alone, endophyte inoculum including PTD1 alone, endophyte inoculum including WW5, WW6, WW7, and PTD1, or a control with no endophyte strains. The plants were planted using standard methods in early November 2016. Strawberries were planted in beds containing two rows of strawberry plants spaced 25-30 cm apart (200-240 plants/row), and 3 beds per treatment were spaced 120 cm apart. Strawberries were fertilized with standard methods under the guidance of a registered CCA and harvested on May 20, 2017 after 28 weeks of growth.

As shown in FIG. 62, the treatment group including mix of all 4 strains WW5, WW6, WW7, and PTD1 performed the best providing a greater leaf chlorophyll and significantly increased the leaf chlorophyll by 5.5% p<0.05.

### Example 56

Effects of Different Endophyte Seed Treatment Compositions Using WW6+WW7, Vs. The Market Leading Biological and a Biostimulant on Winter Wheat Leaf Total Chlorophyll.

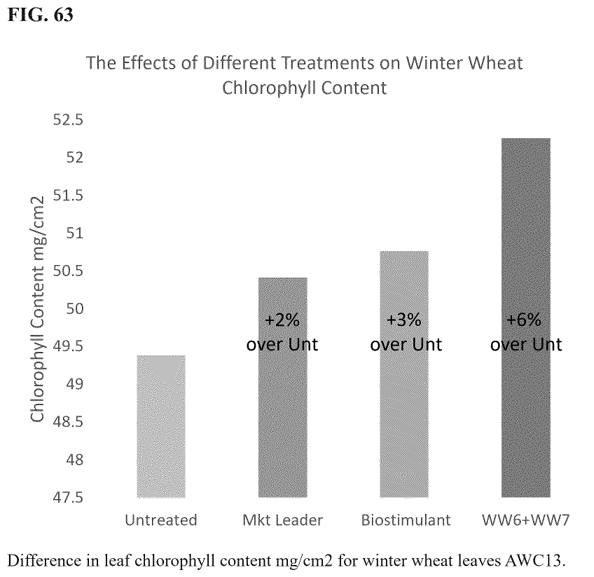

Experiments were conducted to determine whether a composition of endophytes that were freeze-dried and resuspended can increase leaf chlorophyll in treated host plants when applied to seeds. The endophytes were applied to seeds of winter wheat var. AWC13. Freeze-dried endophyte seed inoculum compositions were prepared by resuspending freeze-dried WW6+WW7 endophyte strains in 500 mL of water at concentration of 1.1% w/v and mixing the resuspension with 500 mL of a 4% sterile aqueous sodium alginate solution. A control was prepared with the sodium alginate solution and 500 mL of water with no endophytes. The treatment compositions were applied at a rate of 1 L per 1 metric ton of seed. The winter wheat plants were grown under optimum nitrogen in a field trial and then harvested in the vegetative phase. Chlorophyl was extracted from leaf sections that were 1 cm² in size and extracted using acetone. The results in FIG. 63 showed an average increase of 6% over the untreated control.

### Example 57

Enhancing Glutamate/Glutamine Glx in Corn Leaves after Treatment with an Endophyte Inoculum Seed Coat Composition.

Tests were conducted to determine whether a seed coat containing a heterologous endophyte composition can fix N₂ atmospheric nitrogen gas, produce ammonium, and then convert it into the first amino acid end products glutamate and glutamine via the GOGAT GS and GDH ammonium assimilation pathways in a host plant. A seed treatment including WW6 fermentate and sodium alginate 0.5% w/v was prepared. The seed treatment was applied to corn seeds and the seeds were then dried and stored for 1 month. The treated seeds and control untreated seeds were then potted in 1-gallon pots containing 1.5 kg sand, vermiculite and perlite, and germinated and grown for 3 weeks. During the growth period, the seeds and resulting plants were watered with a Hoagland's nitrogen drop out nutrient solution supplemented with 50 ppm N. The plants were grown in the green house, harvested, freeze dried and analyzed by AAA labs Inc USA for common amino acids using a Shimadzu HPLC with post column ninhydrin derivatization. The results presented in FIG. 64 demonstrate that the WW6 inoculated corn seeds grew into plants that had significantly (p=0.6)+46% more Glutamate and Glutamine amino acid content in their leaves when compared to the controls. Furthermore, when the genome of WW6 was analyzed for the presence of the genes encoding for the Glutamine Synthase GS enzyme, a total of 6 different GS enzyme gene copies were discovered in locations adjacent other genes associated with nitrogen assimilation, quorum sensing, and motility. These results further support the other findings involved with atmospheric N₂ fixation, and enhanced N concentrations in shoots of WW6 treated corn. These results also demonstrate the mechanistic basis for the efficacy of WW6 as a biological inoculum for increasing nitrogen assimilation directly from the atmosphere into amino acids glutamate and glutamine in crop plants.

### Example 58

### Detection and Quantification of Glutamine Synthetase (GS) Activity in Plant Tissue

Glutamine Synthetase (GS) is a key enzyme for bacterial atmospheric nitrogen assimilation via the GOGAT pathway from N₂ through ammonia/ammonium and into synthesis of the amino acid glutamine. The in-planta effect of the endophyte strain(s) WW6 and WW7 on this process was evaluated in the shoots of wheat plants (Triticum aestivum). The Zenda variety of the wheat seed was first treated with (1) Cruiser Maxx Vibrance Cereals (Syngenta Crop Protection, LLC) at 5 fl oz. per 100 pounds of seed, and (2) Cruiser 5FS at 0.75 fl oz. per 100 pounds. The wheat seed was coated with the WW6 and WW7 fermented inoculum blended with 0.5% sodium alginate by weight, which was applied at a rate of 500 ml per 2000 lb of seed and the treated seeds were air dried at room temperature and stored for a month. A control group was prepared with a seed coating solution having only alginate and growth media without the endophyte bacteria.

Five seeds were planted into 5 different 3.5-inch pots for each treatment group. The pots contained a mixture of washed play sand, vermiculite, and perlite potting mixture. The pots were maintained at 4° C. for 24 hours to induce vernalization and were then transferred to a growing room held at 25° C. under LED light with a 14 hr light/10 hr dark lighting growth cycle. Seven days later the number of seedlings per pot was thinned and reduced to three. Additionally, the plants were watered and fertilized with a Hoagland's dropout hydroponics solution with a reduced nitrogen concentration (25 ppm N) applied in trays 2-3 times per week as needed to maintain moist soil. Thirty-one days after transplanting, the plants were removed from the soil. The shoots and roots were separated and then flash-frozen with liquid nitrogen before storing the tissue in the −80° C. freezer. The plant material from each pot was individually ground into a fine powder with a mortar and pestle using liquid nitrogen. Approximately 100 mg samples of the ground tissue from each treatment were then transferred to five separate 1.5 ml tubes and frozen with liquid nitrogen before being stored in the −80° C. freezer for later use.

A glutamine synthetase microplate assay kit (MyBioSource, Inc) was used to detect and quantify the glutamine synthetase (GS) enzyme activity in the prepared samples. Shoot tissue samples from three pots for the control group plants and three of the experimental group plants (treated with WW6+WW7) were used for the GS enzyme assay. Three technical replications for each sample was prepared and then the average reading of all the replications was used to calculate GS synthetase activity U/g.

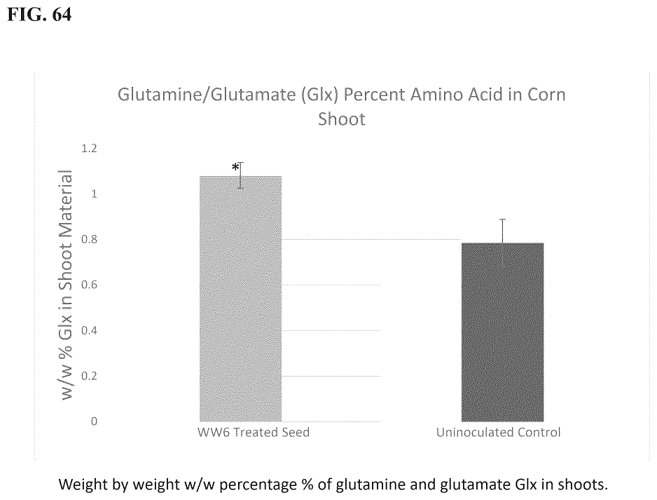

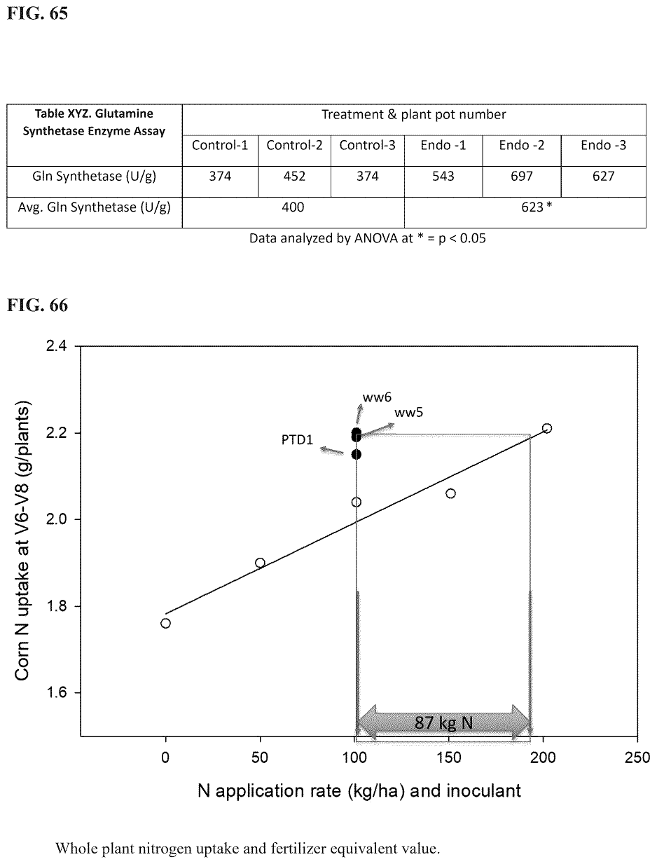

FIG. 65, demonstrated the quantification of GS (U/g—the U unit is the amount of enzyme that catalyzes the reaction of 1 μmol of substrate per minute), which was adjusted according to the amount of tissue tested. ANOVA analysis with a post hoc Tukey test was used to analyze the data. The results demonstrated that the endophyte inoculum composition used as a seed treatment resulted in plants that had on average a 55% higher glutamine synthetase GS enzyme activity then the un-inoculated control plants and the increased activity was statistically significant at the p<0.05.

In summary, WW6 and WW7 treated seed had a profound effect on increasing the glutamine synthetase activity in wheat shoots. This GS enzyme GOGAT relevant result correlates with data from the field studies of Example 62 herein showing nitrogen accumulation in endophyte inoculated wheat shoots and the greenhouse studies of Example 57 herein showing nitrogen assimilation derived from air increased in inoculated wheat.

### Example 59

Reduction in Corn Crop Nitrogen Fertilizer Requirements while Maintaining Harvest Yields.

A field study was designed to demonstrate the ability of endophyte seed treatment compositions to provide substantial harvested yields under reduced nitrogen fertilizer application. Corn seeds were treated with WW6 endophyte inoculant combined with 0.5% w/v sodium alginate to coat the seed. The treated seeds were planted and grown in Kansas USA field soil using urea as fertilizer. Field plots were divided into groups and fertilized at different rates by broadcasting: (1) a normal rate was 201.75 kg/ha of Urea (100% rate), (2) the reduced rate of 141.22 kg/ha of Urea (70% rate), (3) a second reduced rate of 100.875 kg/ha of Urea (50% rate), and (4) a no Urea group (0% rate) was applied by broadcasting.

The grain was collected from each plot once the grain moisture reaches approximately 15.5% and there were 4 plots per treatment. An average grain yield was calculated for all the plots representing each treatment. FIG. 66 shows the average shoot nitrogen uptake for the endophyte inoculated corn plants in comparison to the controls. The graph demonstrates the endophyte reduction in corn fertilizer requirements or how much less fertilizer is needed to maintain the same grain yield due to the endophytes versus the un-inoculated control corn plants. The results demonstrate that due to either WW6 or WW5 endophyte inoculation, 87 Kg less nitrogen fertilizer is required per hectare.

### Example 60

### Enhanced Nitrogen Uptake by Single and Double Strain Seed Treatments of Wheat.

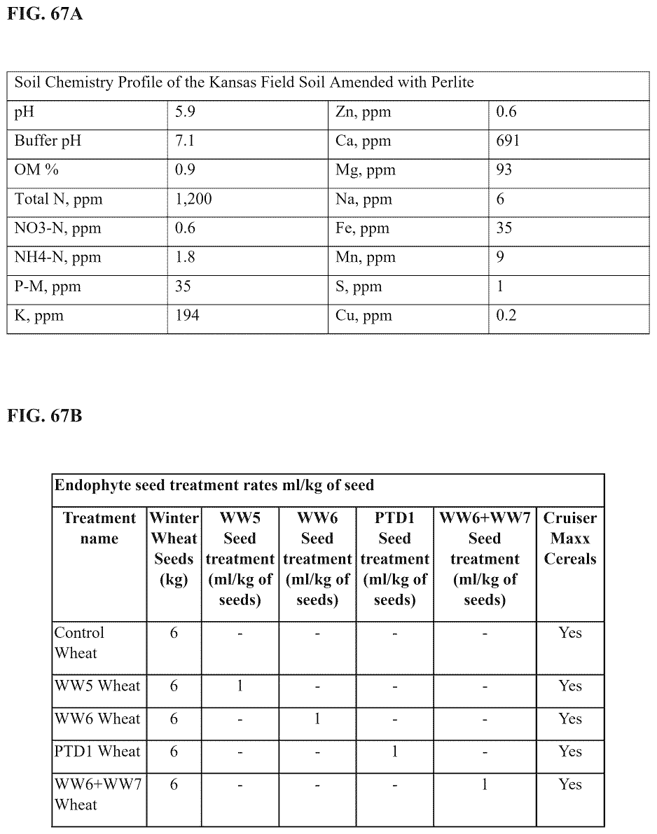

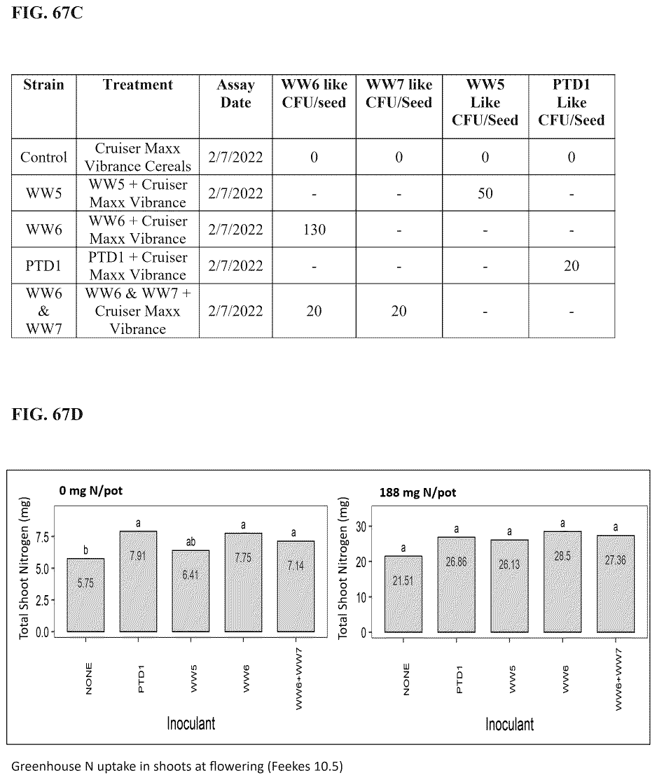

In order to demonstrate the ability of endophyte seed treatment compositions to increase nitrogen uptake in crop plants, Winter Wheat seeds (Everest) were treated with endophyte inoculum and were then grown in pots under three different conditions: (1) field soil (from Kansas, USA) amended with perlite alone; (2) nitrogen sufficient field soil amended with perlite; and (3) field soil fertilized with urea. The field soil chemistry profile is provided in FIG. 67A.

The seeds were treated with the endophyte seed treatment inoculum composition consisting of the bacterial fermentate combined with 0.5% w/v sodium alginate solution applied at a rate of 1 ml/kg of seeds and a standard seed treatment chemical (Cruiser Maxx Vibrance™ seed treatment) at a rate of 5.7 fl oz/100 lbs, as shown in FIG. 67B.

Endophyte survival after dehydration and storage for 1 month on the treated seed was enumerated and the results demonstrated bacterial endophyte survival on the seed for each strain, as shown in FIG. 67C. The seeds were washed and were sampled for enumeration of the seed wash. The wash sample of the winter wheat control seeds had no microbes with morphology similar to the different endophyte strains. All treated seed had a clear presence of the correct strains demonstrating survival and compatibility after treatment and storage with Cruiser Maxx Vibrance. The WW6 alone treatment resulted in the highest CFU survival per wheat seed. The CFU levels detected on seed are possibly under representative of the actual microbial loading rates due to limited detection using this seed coat wash and enumeration plating method.

In the controlled greenhouse maintained a temperature between range of 60° F. to 75° F., a wheat plant pot growth trial was conducted in which five winter wheat seedlings were planted in pots and later thinned to three plants. One ml of the WW5, WW6, PTD1, and WW6+WW5 solution was applied to the seedling and the plants were harvested after 3-months at flowering seed set (Feekes 10.5). The results demonstrate that the endophyte inoculum seed treatment composition caused a substantial increase in N uptake in shoots in both the unfertilized zero nitrogen pots and in the 188 mg N (urea) pots, as shown in the results provided in FIG. 67D.

Under the 188 mg N urea/pot fertilized conditions, the double strain WW6+WW7 and the single strain WW6 resulted in increased nitrogen content. In the urea treated and unfertilized conditions, the single strains WW6 and PTD1 were most effective at increasing shoot nitrogen content. Inoculation with the two strain WW6+WW7 combination also resulted in significant increases in nitrogen content in the shoots of the treated plants in the urea treated and unfertilized conditions.

### Example 61

### Enhanced Nitrogen Uptake by Single and Double Strain Seed Treatments of Corn.

In order to demonstrate the ability of endophyte seed treatment compositions to increase nitrogen uptake in crop plants, a greenhouse study was designed to first treat corn seed and then grow wheat plants in Kansas USA field soil in pots amended with perlite alone (see table XYZ below) and in nitrogen sufficient field soil amended with perlite and urea in the fertilized field soil.

Corn seed was treated with each of the individual endophyte (WW5, WW6, PTD1) seed treatment inoculum compositions mixed together at a 0.5% w/v sodium alginate solution and applied at a rate of 2.4 ml/1800 seeds and a standard seed treatment chemical (Cruiser Maxx™ seed treatment-Syngenta) at a rate of 5.7 fl oz/100 lbs. The seeds were air dried at room temperature and stored for one month prior to planting.

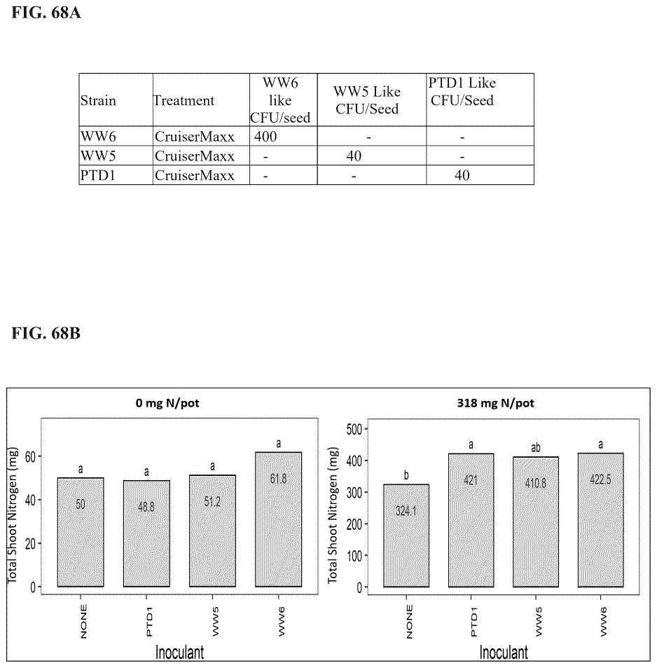

Endophyte survival after dehydration and storage for 1 month on the treated seed was enumerated and the results demonstrated bacterial endophyte survival on the seed for each strain, as shown in FIG. 68A. The seeds were washed and were sampled for enumeration of the seed wash. The wash sample of the corn control seeds had no microbes with morphology similar to the different endophyte strains. All treated seed had a clear presence of the correct strains demonstrating survival and compatibility after treatment and storage with Cruiser Maxx. The WW6 alone treatment resulted in the highest CFU survival per corn seed. Both WW5 and PTD1 seed had 40 CFU/seed with a colony morphology exactly matching and the WW6 treated seeds had the highest at 400 WW6 CFU/seed. The CFU levels detected on seed are possibly under representative of the actual microbial loading rates due to limited detection using this seed coat wash and enumeration plating method.

In the controlled greenhouse that maintained a temperature between range of 60° F. to 75° F. F., a corn plant pot growth trial was conducted in which three corn seeds were planted per pot and later thinned to one seedling after emergence. Corn seed was treated with each of the individual endophyte (WW5, WW6, PTD1) seed treatment inoculum compositions mixed together at a 0.5% w/v sodium alginate solution and applied at a rate of 2.4 ml/1800 seeds and a standard seed treatment chemical (Cruiser Maxx™ seed treatment-Syngenta) at a rate of 5.7 fl oz/100 lbs. The plants were harvested after approximately 1.5-months at vegetative stage eight. The results demonstrate that the endophyte inoculum seed treatment composition caused a substantial increase in N uptake in shoots in both the unfertilized zero nitrogen pots and in the 318 mg N (urea) pots, as shown in the results provided in FIG. 68B. WW6 treatment increased total shoot nitrogen uptake under unfertilized field soil conditions by ˜12 mg per plant shoot over the controls. PTD1 and WW5 did not increase shoot nitrogen in the greenhouse without added fertilizer.

Under the 318 mg N urea/pot fertilized conditions, treatment with the individual PTD1, WW5, and WW6 strains resulted in increased nitrogen content. In the unfertilized conditions, the single strain WW6 treatment increased shoot nitrogen content. The results from endophyte inoculation of seeds via seed coat under fertilized conditions resulted in ˜100 mg more nitrogen per shoot for all three treatments with WW6 performing the best followed by PTD1 and these increases were statistically significant at a p<0.05 level. WW5 seed treatment also increased the nitrogen content in corn shoots ˜85 mg over the controls.

### Example 62

### Use of Two and Three Synergistic Endophyte Strain Compositions for Seed Coat Applications on Corn Shoots in Order to Increase Harvest Grain Yields.

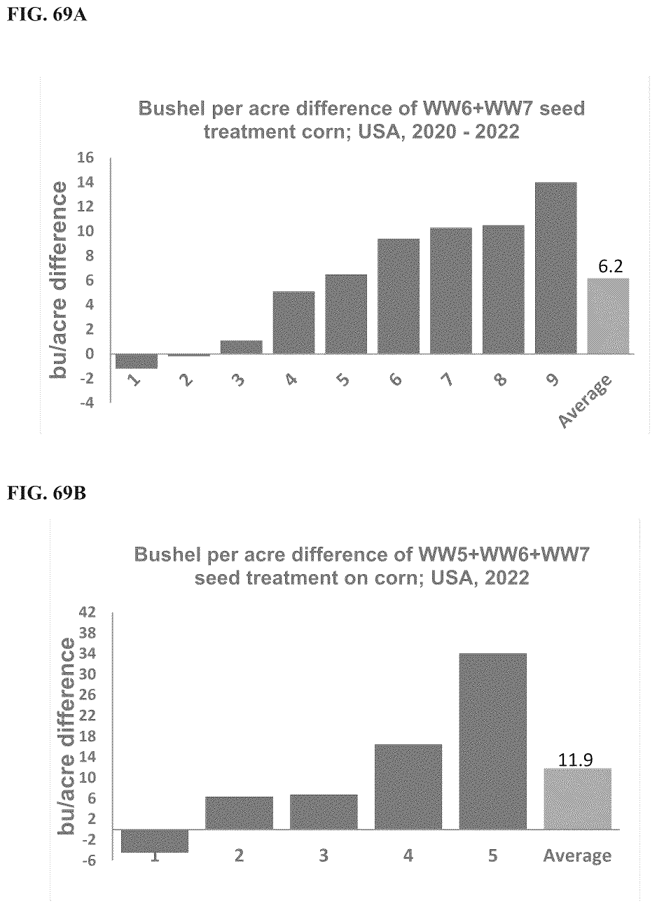

Specific endophyte combinations (WW6+WW7) and (WW5+WW6+WW7) were applied as seed coats to test whether the endophyte strain combinations resulted in synergistic effects on nitrogen fixation in treated hybrid corn varieties. Corn was planted in a series of different states in the midwest USA by a variety of CRO field trial testing organizations in early May 2020-2022. The plot sizes were 10 ft×40 ft with 4 rows of planted corn in each plot and thirty evenly spaced plants per row. The study included six block replications per treatment. Standard fertilizer applications were utilized. The residual soil nitrate pre-fertilizer application was 17 lbs/acre and 170 lbs of additional nitrogen was applied. The herbicide Roundup was applied to the plots on vegetative stage three to six depending on environmental conditions. Soil types varied by location and organic matter content ranged from 2.1-4.1%. The middle two rows were harvested in late October and the grain was weighed. Yield results for the WW6+WW7 treatment are summarized in average bushel/acre in comparison to the uninoculated controls and are shown in FIG. 69A. Yield results for the WW5+WW6+WW7 treatment are summarized in average bushel/acre in comparison to the uninoculated controls and are shown in FIG. 69B. Each numbered bar in FIG. 69A and FIG. 69B is a separate field trial, showing the bushel per acre difference from that trial's untreated control, and the average across all trials is shown with the bar on the far right. The results demonstrated an average yield increase for the WW6+WW7 treated seeds of 6.2 bushels/acre and for the WW5+WW6+WW7 treated seeds of 11.9 bushels/acre. These results demonstrate the synergistic beneficial effects of stacking multiple strain compositions together for use as a seed treatment.

### Example 63

### Effects of Endophyte Seed Treatment Compositions Using WW6+WW7 on Leaf Total Chlorophyll, Over a Variety of Trials Testing Different Grower Programs in UK Winter Wheat.

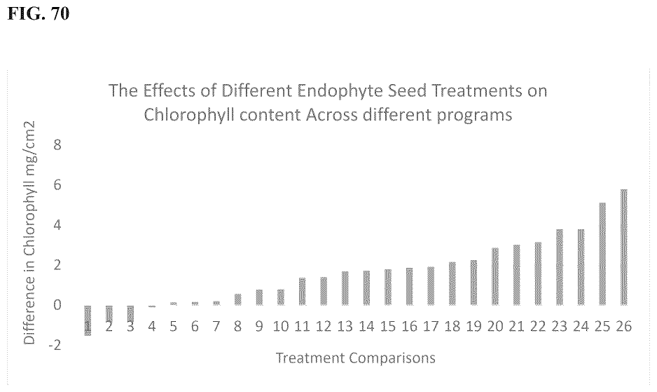

A composition of endophytes that were freeze dried and resuspended in water was used to treat winter wheat var GS59 seeds to test whether the endophyte strains can increase leaf chlorophyll. Freeze dried endophyte seed inoculum composition consisting of WW6+WW7 500 ml water resuspended endophyte freeze dried powder 1.1% w/v with 500 mL of a 4% sterile sodium alginate solution made in H₂O and applied at a rate of 1 L per metric ton of seed. A field trial was conducted in which the wheat plants were grown under optimum nitrogen and harvested at the vegetative phase. Chlorophyll was extracted from leaf sections that were one cm² in size and extracted using acetone. Difference in leaf chlorophyll content mg/cm² for winter wheat leaves GS59 under different programs. The endophyte application increased leaf chlorophyll in the experimental groups compared to controls in 21 out of 26 field trials conducted using different industry standard growers programs over the untreated control, as shown in FIG. 70.

### Example 64

### Nitrogen Limited Biomass Increase in Maize and Teosinte Inbred Genetic Lines.

The effect of application of the WW5 endophyte in a coating on Zeasyn corn seed on shoot growth. Inbred seeds from the Zeasyn population used in the study are from a synthetic population that includes both maize alleles and alleles from Teosinte. The Zeasyn population was made via several generations of random mating of the nested-association mapping (NAM) founders and 11 geographically distinct teosinte individuals, and the final genomic percentage make up is ˜38% B73 (maize parental breeding line), ˜2% of NAM parents+Mo17 (maize parental breeding line), and ˜1% of teosinte. The seeds were treated with a WW5 solution that includes 1.0 E⁸ CFU WW5 per mL plus a 1% w/w sodium alginate. The process of seed treatment was as follows: cooled WW5 single strain culture was mixed well and carefully pipetted onto seed in a Ziplock™ bag at a rate of 3.4 mL per lb of seed and dispersed in drops in 1 mL additions in a sterile laminar flow hood. The seed was manually tumbled and massaged carefully in the bag after each 1 mL addition. Once the addition of the 3.4 mL was complete, the seed was massaged/shaken/tumbled for 2-3 minutes until all corn seed appeared visibly wet in the bag. The bag was then opened and turned inside out for drying with air flow in a sterile laminar flow hood at room temperature overnight. The dried seed was stored for 1 month prior to being washed and then bacterial survival was enumerated by plating. The plating assay showed that there was variable endophyte coating (WW5 CFU/seed) on the treated seeds. This variable endophyte concentration allowed for a wide range of CFU seed representatives: 0˜1000 WW5 CFU/seed.

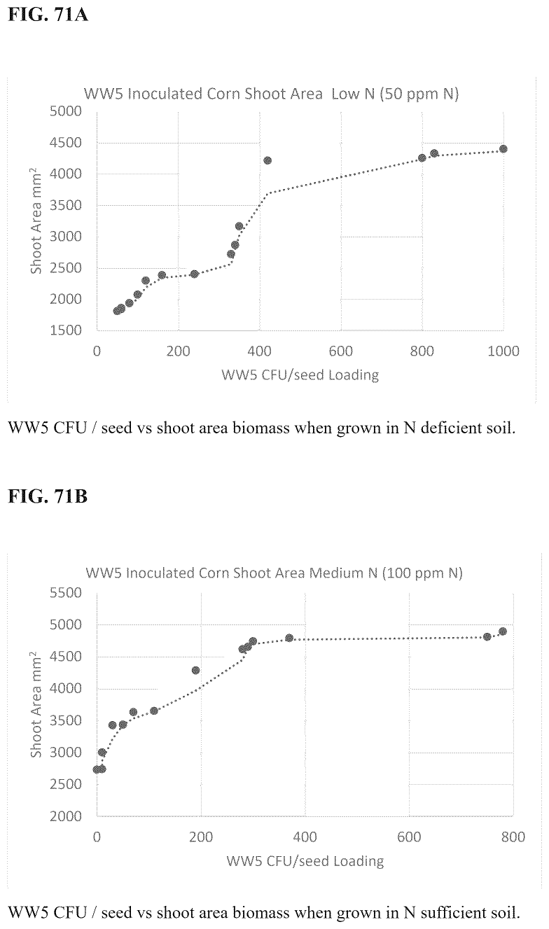

The seeds were tested in greenhouse trials that maintained a temperature in a range of 70° F. to 80° F. and a 12 to 14-hour photoperiod in two groups: low nitrogen soil (50 ppm N) and sufficient nitrogen soil (100 ppm N). The seeds were grown for three weeks prior to leaf biomass analysis. A physiological camera was used to image the plant shoot area of treated plants as a measure of biomass. The imaging was performed after three weeks of growth by phenotype screening equipment on the treated plants. The results from the biomass phenotype screen show that there is a clear correlation between WW5 CFU/seed concentration and corn biomass shoot production irrespective of the inbred line genetic traits, as shown in FIGS. 71A-71B. A correlation between increased biomass and endophyte concentration was evident from the collected data. The optimum CFU/seed appears to be around ˜400 WW5 CFU/seed when plants were grown under nitrogen limited soils and around ˜300 WW5 CFU/seed when plants were grown under nitrogen sufficient soils. Seed treatment using WW5 increased the shoot biomass ˜2.3 times in nitrogen deficient soil and increased the shoot biomass ˜1.7 times in nitrogen sufficient soil growth conditions.

### Example 65

Reduction in Wheat Crop Nitrogen Fertilizer Requirements while Maintaining Harvest Yields.

A field study was designed to demonstrate the ability of endophyte seed treatment compositions to provide substantial harvested yields under reduced nitrogen fertilizer application. Wheat seeds were treated with WW6 endophyte fermentate combined with 0.5% w/v sodium alginate as a seed coating. The treated seeds were planted and grown in Kansas USA field soil using urea as fertilizer. Field plots were divided into groups (four per treatment) and fertilized at different rates by broadcasting: (1) a rate of 112 kg/ha of Urea (100% rate), (2) a reduced rate of 78.4 kg/ha of Urea (70% rate), (3) a second reduced rate of 56 kg/ha of Urea (50% rate), and (4) a no Urea group (0% rate).

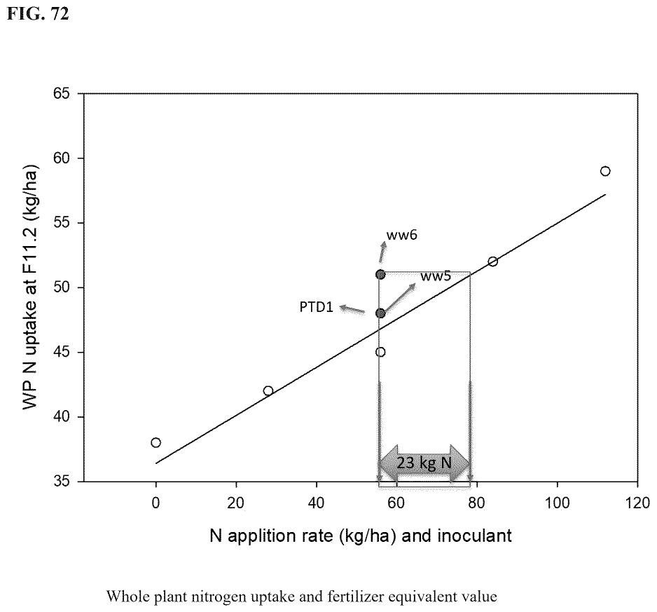

The wheat grain was harvested from each plot once it was fully ripened and mature. An average grain yield was calculated for all the plots representing each treatment. FIG. 72 shows the average shoot nitrogen uptake for the endophyte inoculated wheat plants in comparison to the controls. The graph demonstrates the endophyte reduction in wheat fertilizer requirements or how much less fertilizer is needed due to the endophytes versus the un-inoculated control wheat. The results demonstrate that due to WW6 endophyte inoculation a reduction in the wheat fertilizer requirements was 23 kg less nitrogen per hectare.

### Example 66

Enhanced Root and Shoot Growth of Grape Cuttings in Production and Endophyte Propagated Traits Exist Over the Long-Term after Inoculation with Endophyte Beads.

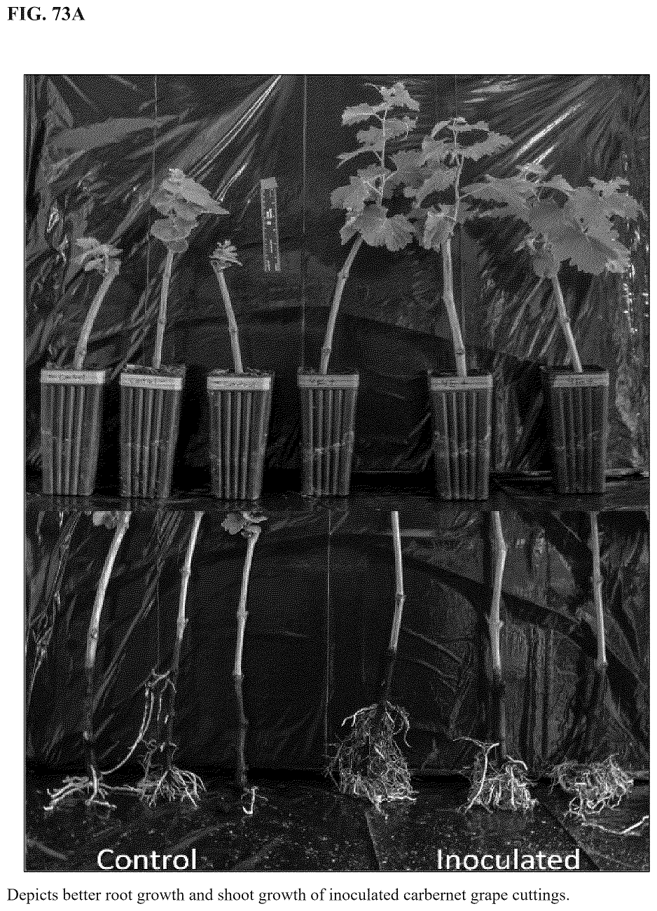

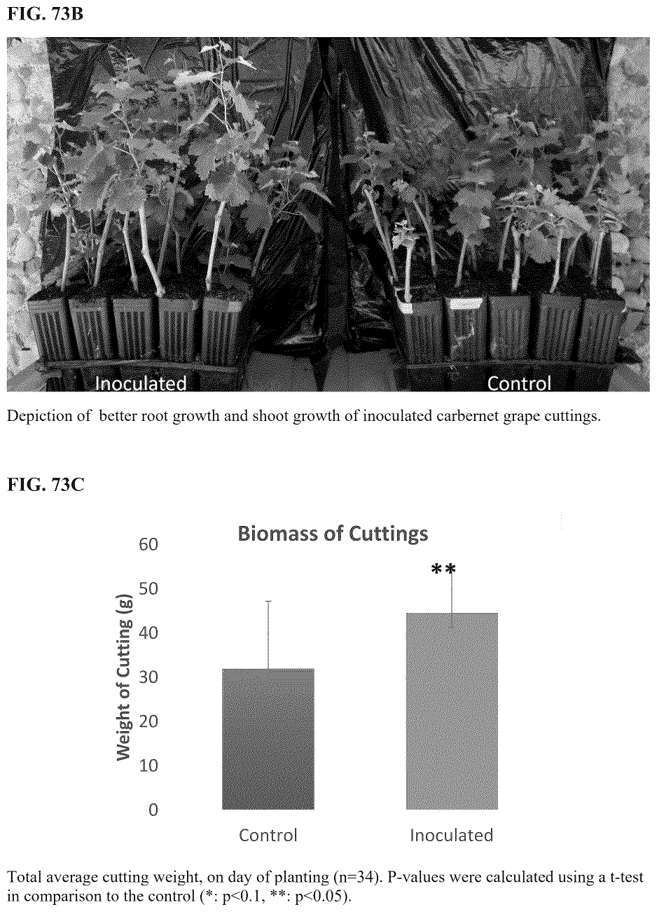

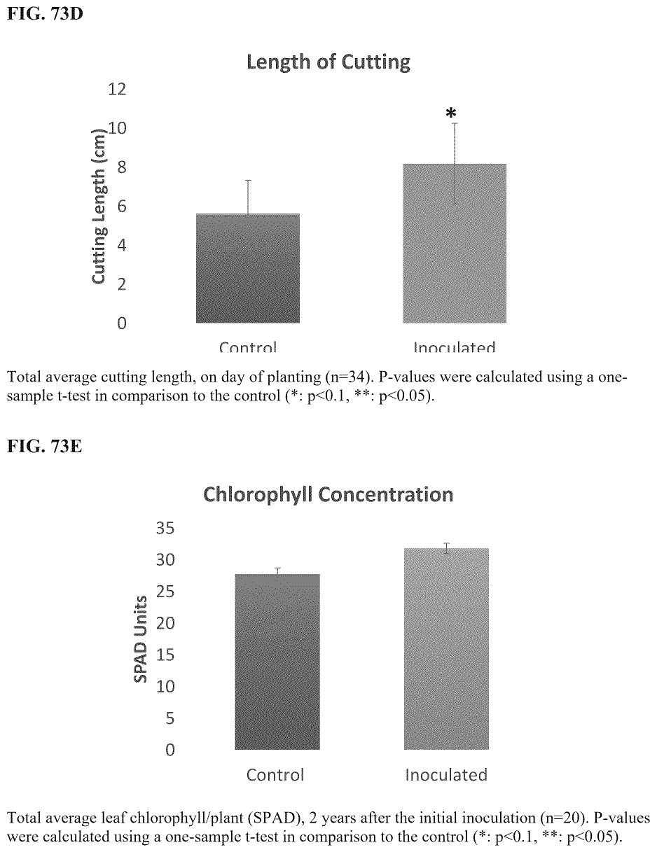

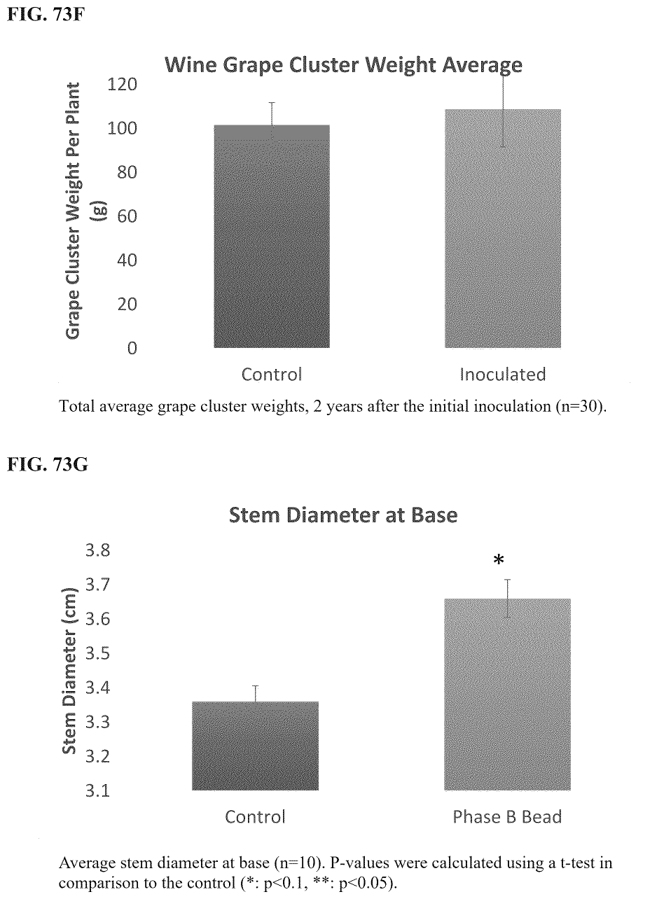

In new varietal grape production scenarios endophyte propagated traits that exist over the long-term after inoculation are advantageous for creating stooling beds for clonal propagation. A long-term test was designed for cuttings inoculated with endophyte beads that were planted in the field and their physiology was tracked. Approximately 20 beads of the endophyte containing calcium alginate beads were placed adjacent to commercial cabernet wine grape cuttings pushed 5-7 cm deep in potting soil (sunshine mix #4). The endophyte beads contained an endophyte consortium inoculum including WW5, WW6, WW7 and PTD1 strains (mix referred to as “Phase B”) in calcium alginate beads. The results after 2 weeks of rooting in pots in the greenhouse showed that the cuttings inoculated with the endophyte beads had a greater root system and bigger shoots compared to controls, as shown in FIG. 73A. Plant growth was continued for two more weeks in a greenhouse maintained at a temperature between range of 70° F. to 80° F. and a 12 hour to 14-hour photoperiod. The plants showed persistence of enhanced shoot and root growth phenotype during the further growth period, as shown in FIG. 73B.

The control un-inoculated group of cabernet wine grapes is shown at the right of FIG. 73B, and the inoculated cuttings are shown at left. Biomass and cutting length measurements were taken at the stage shown in FIG. 73B, and the plants were then transplanted to field plots. The collected biomass data collected showed that the total biomass of the inoculated cuttings had grown 40% larger than significantly more than the control group, as shown in FIG. 73C. Also, the endophyte inoculated cabernet cuttings had a 31% height increase over un-inoculated controls, as shown in FIG. 73D.

Two years after field planting the endophyte-inoculated cuttings, the cuttings had matured into vines and the increased chlorophyll phenotype was observed as well. FIG. 73E provides data showing that the inoculated cuttings had greater average leaf chlorophyll.

Additionally, inoculated vines had an average 7% increase of average grape cluster weights on two-year old wine grapes versus controls. FIG. 73F provides the data demonstrating increased grape cluster weight in the inoculated cuttings.

In a separate field trial experiment conducted in the same location approximately 20 beads of the endophyte WW5, WW6, WW7 and PTD1 strains (phase B) in calcium alginate were pushed 5-7 cm deep into potting soil (sunshine mix #4) adjacent to Grignolino wine grape cuttings. The field trial results one year and three months after planting showed similar benefits in that the endophyte inoculated (Phase B) cuttings had a 9% average increase in stem base diameter compared to non-inoculated control after two years. The results are provided in FIG. 73G.

### Example 67

### Greenhouse Cutting Clonal Production of Cabernet Wine Grapes Endophyte Rooting Effects.

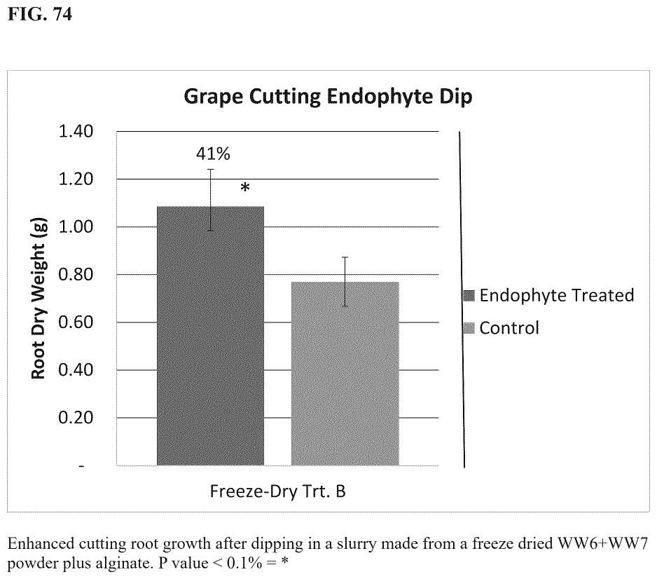

The root production of cuttings is a key factor for clonal propagation in greenhouse settings. The effect of treatment of cuttings with endophyte strains on root production was tested. Commercial cabernet wine grape cuttings were inoculated with slurry dip prepared from freeze dried WW6+WW7 powder combined with sodium alginate that was then reconstituted in well water. The cuttings were dipped in the endophyte slurry immediately prior to being placed on a mister bench to facilitate root growth. The results of the root growth are shown in FIG. 74, which demonstrate that the clonal propagation of cuttings was greatly increased or improved from a production with a statistically significant 41% increase in root growth in the cuttings treated with the WW6+WW7 endophyte inoculation.

Taken together the results from the grape cutting propagation experiments and field transplant of the vines of Example 65 demonstrated an improvement in the clonal propagation of cuttings and continued phenotypic enhancements after transplant. The results support the ability to make new varietals with enhancements from endophyte inoculation and clonal, or vegetative propagation of the traits internally to the plants for production purposes.

### Example 68

### Methods for Preparing Inoculant Formulations.

Starter cultures for fermentation of the endophyte strains of the present technology may be prepared by aseptically pipetting 1 to 2 ml of stock samples of the endophyte strains in glycerol into a flask or bioreactor (e.g., 500 ml to 10-liter volume) with nitrogen limited media (NLM), nitrogen free media (NFM), or rich liquid media and grown as an aerobic culture for 2 to 4 days at 23° C. to 30° C. to reach a final concentration of about 1×10⁶ CFU to about 1×10¹⁰ CFU. Optionally, the starter culture may be scaled up to an intermediate volume of 50 to 200 liters of the endophyte strains. The starter culture volume for each strain should be 0.01% to 1% of the total volume for the subsequent fermentation step.

Large-scale fermentation may be initiated with the starter preparation by adding the starter culture of the endophyte strain (e.g., WW5, WW6, WW7, or PTD1) into a tank with 50 to 10,000 liters of pasteurized NLM, NFM, or rich liquid media and growing the culture aerobically via agitation with sparging air flow at a rate of 0.1 volume of air per unit volume of growth medium per minute (VVM) to 0.5 VVM at 23° C. to 30° C. for 6 to 24 hours.

A second strain for a co-fermentation process may be added to the initial fermentation following 10 to 24 hours of initial endophyte strain growth. The starter culture of the second endophyte strain (e.g., prepared as described above in this example) is added to the fermentation media and the growth of the co-fermenting, two-strain batch may be fermented for additional 24 to 72 hours or until a total CFU of 1×10⁷ to 1×10¹².

### Example 69

### Methods for Preparing Co-Fermented Inoculant Formulations.

Separate starter cultures for each of the endophyte strains of the present technology (e.g., WW5, WW6, WW7, or PTD1) may be prepared by aseptically pipetting 1 to 2 ml of stock samples of the endophyte strains in glycerol into separate flasks or bioreactors (e.g., 500 ml to 10-liter volume) with nitrogen limited media (NLM), nitrogen free media (NFM), or rich liquid media and grown as an aerobic culture for 2 to 4 days at 23° C. to 30° C. to reach a final concentration of about 1×10⁶ CFU to about 1×10¹⁰ CFU. Optionally, the starter culture may be scaled up to an intermediate volume of 50 to 200 liters of the endophyte strains. The starter culture volume for each strain should be 0.01% to 1% of the total volume for the subsequent fermentation step.

Large-scale fermentation may be initiated with a starter preparation of one of the endophyte strains (e.g., WW5, WW6, WW7, and/or PTD1) into a tank with 50 to 10,000 liters of pasteurized NLM, NFM, or rich liquid media and growing the culture aerobically via agitation with sparging air flow at a rate of 0.1 VVM to 0.5 VVM at 23° C. to 30° C. for 6 to 24 hours.

Starter culture for one or more additional endophyte strains (e.g., WW5 and/or WW6) may be added to the initial fermentation for a co-fermentation process following 10 to 24 hours of initial endophyte strain fermentation. The starter culture of the additional endophyte strains (e.g., prepared as described above in this example) is added to the fermentation media and the growth of the co-fermenting, multi-strain batch may be fermented for additional 24 to 72 hours or until a total CFU of 1×10⁷ to 1×10¹².

### Example 70

### Methods for Preparing Co-Fermented Inoculant Formulations.

Separate starter cultures for each of the endophyte strains of the present technology (e.g., WW5, WW6, WW7, etc.) may be prepared by aseptically pipetting 1 to 2 ml of stock samples of the endophyte strains in glycerol into separate flasks or bioreactors (e.g., 500 ml to 10-liter volume) with nitrogen limited media (NLM), nitrogen free media (NFM), or rich liquid media and grown as an aerobic culture for 2 to 4 days at 23° C. to 30° C. to reach a final concentration of about 1×10⁶ CFU to about 1×10¹⁰ CFU. Optionally, the starter culture may be scaled up to an intermediate volume of 50 to 200 liters of the endophyte strains. The starter culture volume for each strain should be 0.01% to 1% of the total volume for the subsequent fermentation step.

Large-scale fermentation may be initiated with adding starter preparations for multiple endophyte strains (e.g., WW5, WW6, WW7, and/or PTD1) into a tank with 50 to 10,000 liters of pasteurized NLM, NFM, or rich liquid media and growing the culture aerobically via agitation with sparging air flow at a rate of 0.1 VVM to 0.5 VVM at 23° C. to 30° C. for 6 to 24 hours or until a total CFU of 1×10⁷ to 1×10¹².

### Example 71

Liquid Formula Microbial Stabilization Inside Packaging Immediately after Fermentation.

After fermentation of an inoculant according to the methods disclosed herein, the inoculant microbial population may be stabilized. Once each of the inoculated strains has reached the minimum CFU of about 1×10⁷ to about 1×10⁹ then sterile DI water is added at a 1:1, 1:2, 1:3, 2:1, or 3:1 to ratio. For example, 380 liter DI water may be added 380 liter of fermentate for a total volume of 760 L (˜200 gallons) to create a final shelf stable inoculum mixture.

As an alternative or additional stabilizing step, the fermentate may be combined with additional liquid stabilizing osmoprotectant or carbohydrate solutions (e.g., sterile alginate solution having an alginate concentration of about 0.05% to about 1.5% by weight). For example, the alginate solution may be created by mixing the alginate in water, sterilizing the mixture, and cooling to 25° C. The fermentate may then be mixed with the alginate solution at a variety of ratios: 1:1, 1:2, 1:3, 2:1, or 3:1.

The alginate solution may then be transferred to a bladder bag (e.g., up to 275-gallon bag) to prepare the endophyte inoculum for shelf stability. The bladder bags may be partially filled to leave 15-25% available head space for expansion.

### Example 72

### Liquid Formulation Monitoring and Quantification by PCR Checked Growth Enumeration Plating.

The liquid inoculant formulation may be tested for active endophyte presence after fermentation, after storage, or other circumstances. The inoculant may be sampled aseptically and tested in triplicate sterile dilution series using a buffer solution diluent at a neutral pH of about 7.0. The serial dilutions of 10⁻⁴, 10⁻⁵, 10⁻⁶, 10⁻⁷, 10⁻⁸ and/or 10⁻⁹ may be plated using the standard microbiology spread plate method. The media spread plates use includes NFM (Nitrogen free media), NLM (Nitrogen limited free media), TSA (Tryptic Soy Agar) PDA (Potato Dextrose Agar), and/other media. The colonies are grown for 48-72 hours at about 75° F. to about 86° F. depending on plate type and strains. The colonies on the plates are then counted based on the morphology of each colony to arrive at a CFU/mL measurement, which is converted to CFU/g.

Colony PCR and 16S—The colonies may be further verified using two molecular confirmation steps. First, two to five colonies of each morphology are used for colony PCR, which is done with specific primers for each strain. Second, one to three colonies of each morphology are selected for 16S PCR. The selected samples are sent to a DNA sequencing vendor for sequencing. The sequences may then be compared to the known 16S sequences for each of the endophyte strains expected in the inoculant composition.

Digital CFU—DNA may be isolated from the liquid formula or a dilution of the formula. The DNA is then used for quantitative PCR, such as qPCR or DDPCR. Digital PCR enables the direct calculation of CFU for the endophyte strain. The quantitative digital PCR data is used in conjunction with a mathematical formula of the expected live colonies via microbiology plating on media providing a digital CFU value.

Propidium Monoazide (PMA) Dye—PMA dye is a photo-reactive dye that binds to exposed DNA. PMA may be added to a sample of the inoculant composition or a dilution thereof and can then be exposed to light. The DNA that is not currently inside a cell is bounded by the PMA which makes the DNA unavailable for PCR, thereby eliminating DNA from dead bacteria from contaminating the PCR results. The DNA is isolated from this sample and then used for quantitative PCR e.g., qPCR or DDPCR. The values from this PCR can be considered as digital CFU.

### Example 73

### Methods for Freeze Dried Powder Inoculum Formulations.

To enable the creation of a stable, freeze dried (FD) microbial formulation, the fermentate may be exposed to an initial cold shock of the liquid inoculum bacteria at 44° F. in a cold room or refrigerator for a period of at least 24 hours. In order to preserve the endophyte bacteria in ice prior to freeze-drying, the cooled fermentate may be flash frozen as solid blocks in evaporator trays at −40° F. Alternatively, the cooled fermentate formulation may be first flash frozen using liquid N₂ to form 0.13-in² pellets. Alternatively, the endophyte fermentate may be first concentrated using microbial filters or bulk centrifugation to make a semi-solid bacterial paste that may be flash frozen and then freeze dried.

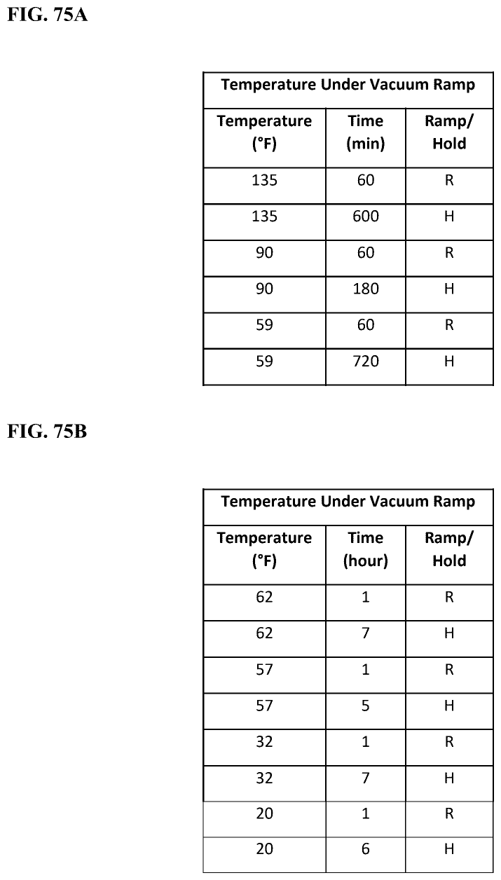

The frozen fermentate may subsequently be freeze-dried using varying equipment and techniques. For example, the frozen fermentate formulation may be dried in a vacuum chamber under the following 30-hour drying cycle, as shown in FIG. 75A

As a further example, the frozen fermentate may be dried in a vacuum chamber under the following 30-hour drying cycle, as shown in FIG. 75B. At end of the cycle, the pressure is slowly released for over an hour to prevent formula disturbance.

### Example 74

### Methods for Spray Drying Powdered Inoculums.

An endophyte liquid fermentate as described herein (e.g., those described in Examples 65-67) may be used to generate a spray-dried powder inoculum formulation. The liquid fermentate may be added in a low to high-shear mixer with about 0.1% to about 10% w/v (e.g., about 0.5 to about 6%, and other values therein) of additional carbohydrate, sugar, and/or protein solids in about equal amounts to produce a slurry. For example, sodium alginate, maltodextrin, whey protein, mannitol, starch, sucrose, PEG, talc or other compatible solid may be added as constituents at 0.1% to 10% (0.5-6%) w/v. The slurry may be spray-dried with a target outlet of about 50° C. to about 120° C. and depending on strains used and a final target moisture of 4% to 10% water. The spray-dried composition provides a dry stable endophyte microbe inoculum composition that can be easily reconstituted.

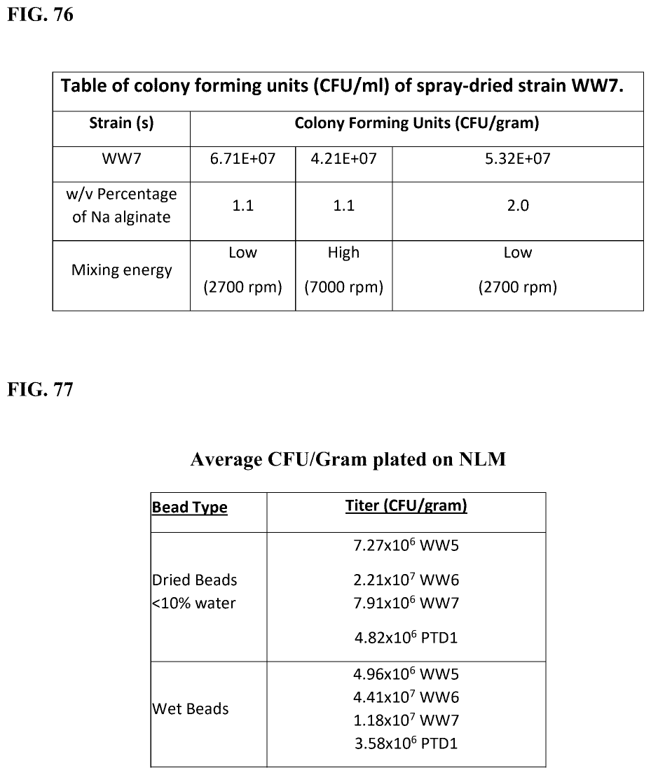

As an example, a liquid fermentate inoculum composition that contains the WW7 endophyte strain was used to generate a new composition comprising of a spray-dried, powdered combined with sodium alginate (DuPont Nutrition USA, In). The spray-dried composition was made by adding 9.46 liters of the liquid composition containing WW7 and 1.1% to 2.2% w/v of sodium alginate mixed with a rotor stator mixer under low energy (2700 rpm) or high energy (7000 rpm) mixing. Then the mixture was then spray-dried with a target outlet of about 80° C. The colony forming units (CFU/gram) of the spray-dried product was quantified by plating the product on NLM semi-solid medium provided in the table in FIG. 76. The results given in the table show a survivability of the WW7 endophyte strain when mixed with different percentages of alginate and mixing energy. The data demonstrates the endophyte composition can be made and used for blending, coating, and delivery into commercial agricultural practices.

### Example 75

### Methods for Manufacturing the Capsulated Bead Formula

The following process may be used to make a variety of different sizes of partially hydrated, capsulated calcium alginate beads ranging in sizes from about 200 micrometers up to about 5 mm in diameter. The endophytes encapsulated in this way are semi-dormant and have a final bacterial titer of about 10⁴ CFU/gram to about 10¹⁰ CFU/gram of bead. The endophyte strains of the present technology may be incorporated into the beads either as a single strain or with multiple strains (e.g., two strains, three strains, or 4 strains of WW5, WW6, WW7, PTD1) and may further include the yeast strain WP1.

Alginate beads containing endophyte strains may be manufactured by the following method. Deionized water, corn starch, and high purity low viscosity sodium alginate extracted from seaweed may be mechanically blended. Liquid endophyte strain cultures (e.g., comprising one or more of WW5, WW6, WW7, and PTD1, and optionally adding WP1) are mechanically mixed into a final slurry in the following weight percentages: water 70-90%, starch 1-8% w/v, bacterial fermentate mixture 2-10% v/v, and alginate 1-4% w/v. The slurry is then agitated constantly and dripped using a peristaltic pump and a large bank of 6-gauge stainless steel dispensing needles into a 1.1% w/v CaCl₂) cationic saltwater bath (MgCl₂ or other appropriate salt may be used) from a 6-inch height in order to create individual beads. The bath may be recirculated and replenished with salt according to calcium depletion rates. The slurry volume may vary from 300 gallons to 5000 gallons. After the beads are finished dripping, and the cation exchange cross linkage has occurred, the beads are mechanically hoisted from the bath using the collection tote and drained well. Subsequently, the beads are weighed out into 100-lb. aliquots and may be dried in a tray or fluid bed at 50° C. The beads are dried to a moisture content <10%, measured with a Karl Fischer Titrator for precise moisture measurements.

The foregoing method was used to produce alginate beads containing a fermentate containing a combination of the WW5, WW6, WW7 and PTD1 endophyte strains. The method yielded about 138,800 beads/kg. The average size and weight of the beads produced for a set of large needle gauges was as follows: 5-gauge needles produced beads of a 2.6 mm diameter and 7.7 mg, 6-gauge needles produced beads of a 2.1 mm diameter and 7.7 mg, and 9 gauge needles produced beads of a 1.8 mm diameter and 7.2 mg. The CFU/g titers of the 6-gauge 2 mm beads of the four-strain mixture of WW5, WW6, WW7 and PTD1 are provided in the table shown in FIG. 77.

Post manufacturing, the beads were stored at temperatures below 110° F. with recommended storage temperatures ranging from 40° F. to 80° F. Under such conditions, the endophyte strains will remain viable in the partially hydrated encapsulated alginate bead shell for up to 3 years.

### Example 76

### Dry Carrier Powder Inoculum Mixtures and Fertilizer Coating Methods.

Dry powder formulations on various carriers may be produced by spraying a liquid endophyte formulation or by mixing the liquid endophyte formulation in a dried powder formulation. The carriers may be inert powdered carriers or mineral fertilizers. The carriers may include sodium alginate, powdered or coarse biochar, Dolomite Lime, triple superphosphate, maltodextrin, whey protein, cellulose/hemicellulose, tapioca flour, sucrose, or other dry carriers. In other implementations, the endophyte formulation or a dry powder formulation comprising the endophyte formulation may be used to coat other fertilizers e.g., triple superphosphate (calcium dihydrogen phosphate+monocalcium phosphate), tricalcium phosphate, urea, DAP, KNO₃, NH₃—NO₃, and/or other appropriate fertilizer compositions. The dry powdered carrier mixture formulations may be manufactured by spraying liquid endophyte formulations onto powders moving down a conveyor belt or by blending in a mechanical mixer. In other implementations, the powdered freeze-dried or spray-dried endophyte inoculums (e.g., as described in Examples 68 and 69) being mixed with carrier(s) and/or fertilizer(s).

### Example 77

Methods and Protocols for Seed Treatment with Endophyte Inoculums.

Seed coating and nutrient formulation formulas may include one or more of the endophyte strain inoculums (e.g., WW5, WW6, WW7, and/or PTD1), a carbohydrate such as sodium alginate at about 0.01% to about 2% w/v, nitrogen-limited media, amino acids, mineral nutrients, sucrose, and mannitol. The seed coating and nutrient formulation may be applied to a seed as a first step. Subsequently, one or more of the following specific seed chemistries may be applied: Mefenoxam, Fludioxonil, Azoxystrobin, Sedaxane, Thiabendazole Thiram, Metalaxyl, Hymexazol, Penthiopyrad, Clothianidin, beta-Cyfluthrin, Thiamethoxam, Tebuconazole, Prothioconazole, Imidacloprid, Clothianidin, Acibenzolar-S-Methyl, Difenoconazole, Cyantraniliprole, Spinosada, Oxathiapiprolin, Pydiflumetofen, Cyromazine, Picarbutrazox, Mandipropamid, Mancozeb, Thiodicarb, Flupyradifurone, Penflufen, Prothioconazole, Trifloxystrobin, Beta-Cyfluthrin, Triadimenol, Pyraclostrobin, Boscalid, Abamectin, Captan, and Fluopyram. Seed coat polymers may be applied with the seed chemistry(ies) until the seed is well coated with both formulas and then dried under lowest possible forced air temperatures. In other implementations, the endophyte strain inoculum can be applied after the seed has been coated with the seed chemistry and/or seed polymers.

In some implementations, the seed chemistry(ies) and the seed coat polymers may be applied simultaneously to the seed with the endophyte strain inoculum in a seed treater using separated formula streams. The formula streams may be thoroughly mixed in a seed treatment bowl or container until seed is well coated with both formulas, and then dried under lowest possible forced air temperatures.

In further implementations, the seed chemistry(ies), the seed coat polymers, and the endophyte strain inoculum may be pre-mixed prior to application to the seed in a slurry until the mixture is homogeneous. The mixture may be applied to the seed within 24 hours in a seed treater. Allow thorough mixing to occur in the bowl or container until seed is well coated with both formulas and dried under lowest possible forced air temperatures.

### Example 78

Use of a Powdered Endophyte Formulation Applied Directly Mixed into a Powdered Cellulose Hemicellulose Seed Coat, and a Liquid Endophyte Formulation Applied onto a Dry Carrier Seed Coat.

Endophyte encapsulated beads were produced using the endophyte liquid inoculum nutrient formulation comprising Sphingomonas sp. (WW5) mixed with sodium alginate using the methods described herein (e.g., Example 70) to produce 2 mm-diameter, 10% water calcium alginate beads. 200 g of the beads were ground to a fine powder and then mixed into a cellulose and hemi-cellulose carrier mixture and applied as a natural seed coat followed by a white seed polymer and used to coat 250,000 tomato seeds (Syngenta Qual-47). The powdered carrier and inoculum mixture was built up on the seeds in the mixing bowl and resulted in WW5 being present on treated seed at 3.68E+07 CFU/seed.

As a separate treatment group 490 mL of WW5 liquid seed treatment nutrient inoculant formula was mixed into the same hemicellulose powdered carrier and together with a white seed coat polymer was used to treat 250,000 tomato seeds (Syngenta Qual-47). The liquid seed treatment nutrient inoculant plus powder carrier formula resulted in a similar seed CFU of 8.33E+07 CFU/seed.

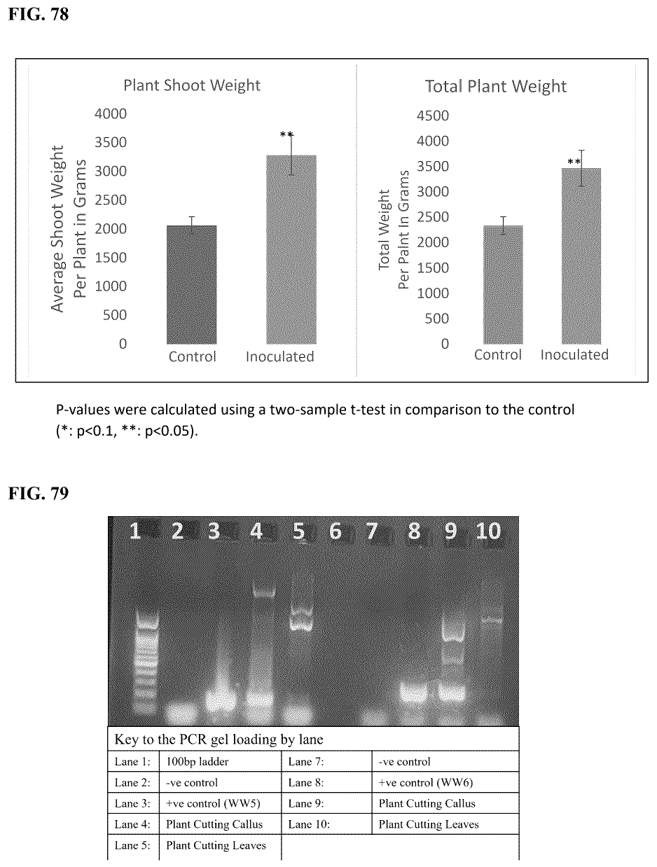

Because the CFU/seed was similar, the two WW5 endophyte treated tomato seed groups were then pooled together and labeled as (inoculated). A control seed group treated with hemicellulose plus polymer without endophyte was also prepared. The seed groups were planted in a California tomato transplant house for 40 days and then a commercial grower planted these in a field trial near Huron, California. At harvest the data demonstrated a clear increased plant biomass of WW5 over uninoculated controls in a large-scale field trial, as demonstrated in FIG. 78.

### Example 79

### Methods for Using Endophyte Inoculum Solutions for Making Stable Endophyte Inoculated New Crop Cultivars.

Peach bud wood cuttings were prepared and incubated overnight in an endophyte inoculum solution including WW5 and WW6 strains. The bud wood cuttings were then grown out and maintained for production purposes. Two weeks after inoculation, the root callus had formed, and new leaves emerged from peach branch cuttings. The tissue was carefully harvested, surface sterilized tissues using 2% bleach, and then rinsed with sterile DI. PCR investigation of the undifferentiated root callus and leaf tissues was performed. The results showed that endophytes were present in the root callus and leaf tissues.

FIG. 79 provides PCR gel data showing that the endophyte strains WW5 and WW6 were present in both tissues and were more prevalent inside the undifferentiated root callus.

### Example 80

WW5 is a Novel Species Based on dDDH Analysis.

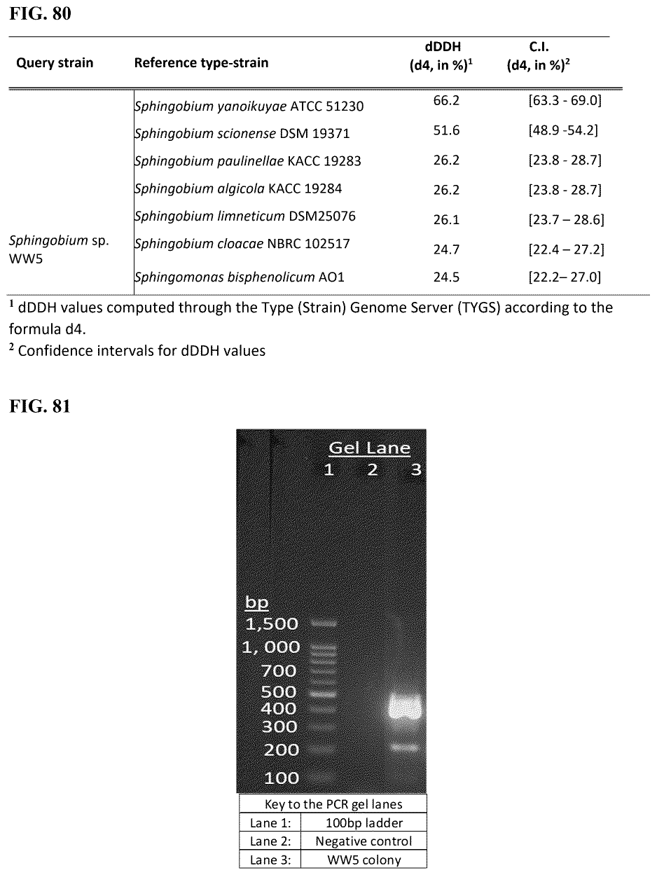

To evaluate the affiliation of the WW5 strain to Sphingobium type strains, pairwise digital DNA-DNA hybridization values (dDDH) were calculated for the WW5 strain to determine its interspecies relatedness with the representatives (type-strains) of the Sphingobium genus. Pairwise dDDH percent values between WW5 and Sphingobium type-strains were all lower than 70%, indicating that the WW5 strain is representative of a novel Sphingobium species, as shown in FIG. 80.

### Example 81

### PCR Analysis Demonstrating Presence of Nif D Gene in WW5.

The nif D gene encodes for the a subunit of nitrogenase, which contains the catalytic site for the reduction of nitrogen gas to ammonia and it binds the FeMo-cofactor, required for nitrogen fixation and participates in transfer of electrons from ferredoxin to nitrogenase, causing the reduction of N₂ to NH₃. Colony PCR and DNA sequence analyses of the WW5 strain was performed on the WW5 endophyte strain to determine whether nif D was present in the genome. The PCR demonstrated nif D was also present in WW5. Further WW5 sequence analyses of the amplicon using primers made from a degenerate sequence demonstrated that the WW5 nif D sequence is a 91% match to the nif D gene in Pseudacidovorax intermedius (NCBI Sequence ID: KM103913). The PCR analysis was conducted in accordance with the method disclosed by Darcy L. McRose, Xinning Zhang, Anne M. L. Kraepiel, and François M. M. Morel, Diversity and Activity of Alternative Nitrogenases in Sequenced Genomes and Coastal Environments, Frontiers in Microbiology, 8, 2017.

FIG. 81 provides gel data regarding the presence of the nif D in the WW5 strain. The presence of the 300 bp and 500 bp bands indicates the presence of the nif D gene according to the method of Darcy et al.

### Example 82

### Additional Nitrogen Assimilation Pathway Genes in WW6.

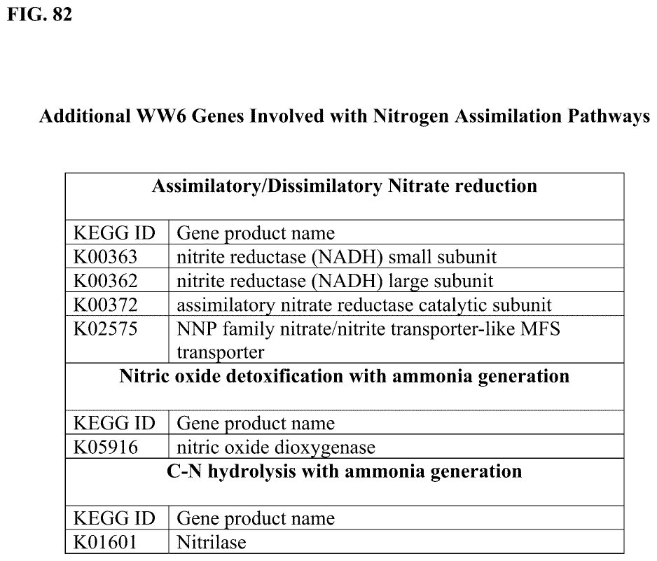

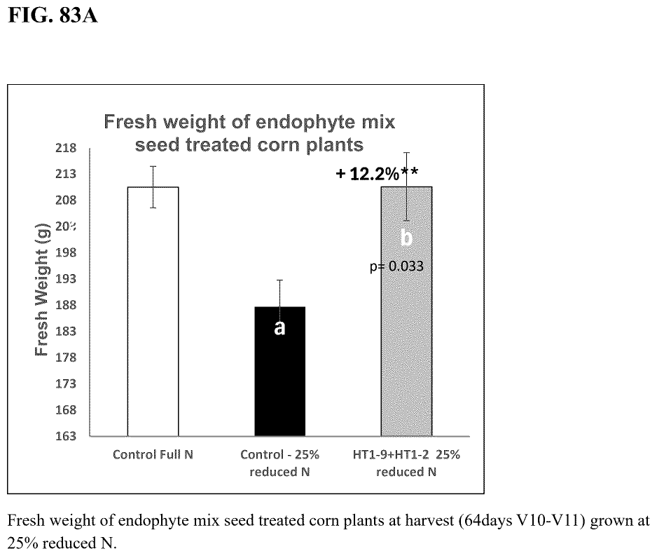

It has been experimentally demonstrated that WW6 strain possesses in its genome the following genes involved in the following nitrogen metabolism and assimilation processes. As listed in the table of FIG. 82, the WW6 genome includes genes involved with assimilatory and dissimilatory nitrate reduction pathways, including NasA (assimilatory nitrate reductase catalytic subunit), which catalyzes the reduction of nitrate into nitrite:

Nitrite+Acceptor+H₂O<=>Nitrate+Reduced acceptor

The WW6 genome also includes Nir (nitrite reductase) involved in the reduction of nitrite into ammonium using the following reaction:

Nitrite+3 NADH+5H⁺→ammonia+3 NAD⁺+2 H₂O

The WW6 genome also includes a nitric oxide dioxygenase which means WW6 has the capacity to also couple NO detoxification with ammonia generation by Nas-Nir enzymes.

2NO+2O₂+NAD(P)H→2 Nitrate+NAD(P)⁺+H₊

Finally, the WW6 nitrilase protein can also catalyze the hydrolysis of C—N organic compounds in the environment to the corresponding carboxylic acids and ammonia, thereby mobilizing ammonia for nitrogen uptake by a host plant. The table provided in FIG. 82 provides KEGG database identifications for genes present in WW6 that are involved in nitrogen assimilation pathways.

### Examples 83-87

### Procedures Use of Co-Fermented Endophyte Mixtures in Novel Foliar or Seed Treatments Under Reduced Nitrogen Fertilizer.

Examples 83-87 evaluated the effects of beneficial endophyte inoculations on hybrid corn growth under reduced nitrogen (N) fertilization. A co-fermented endophytic bacterial mixture was applied either as a foliar spray or as a seed coating, and plant performance was compared under low versus standard agricultural nitrogen conditions. Key response variables included aboveground biomass, leaf chlorophyll content, and shoot mineral nutrient uptake, as indicators of improved nitrogen use efficiency (NUE). To obtain agriculturally relevant results, the experiment was conducted in controlled growth chambers simulating field conditions. Real field soil (European Cambisol, a medium-to-fine loamy soil ideal for corn) was used as the growth medium, and chamber environments were programmed to mimic an entire growing season-starting with cool spring temperatures and short days and gradually transitioning to warmer summer conditions with longer daylength. This approach ensured that corn plants experienced realistic field-like conditions of soil, temperature, and light throughout the experiment.

### Soil Preparation and Potting

Field soil (Cambisol from Lower Austria) known for low native N content was used as the base. To standardize and further limit nutrients, the Cambisol was mixed with a nutrient-poor peat and perlite blend in a 1:3:1 ratio (1 part soil:3 parts steamed low-nutrient peat substrate, Einheitserde Type 0:1 part perlite). This soil blend provided a consistent, low-fertility growing medium. Each treatment group consisted of 8 replicate pots (3 L volume each) filled to the top with the soil mixture.

### Planting and Growth Conditions

Commercial hybrid corn seeds were used for planting. Two seeds were sown per 3 L pot (to ensure germination; later thinned to one). Pots were placed in growth chambers with controlled temperature, light, and daylength settings. Illumination in the chambers was provided at up to ˜1000 μmol/m²/s at canopy level. The environmental conditions were adjusted weekly to simulate natural seasonal progression (see Table 1). In the first week, temperatures were kept cooler (around 11.5° C. at night, 19° C. during day) with a 15-hour photoperiod to resemble spring. Each subsequent week, day and night temperatures were gradually increased and photoperiod slightly extended, reaching about 22° C. night, 32° C. day, and ˜15.5 hours of light by week 10. Corn seedlings emerged approximately 7 days after sowing, at which point the temperature and light conditions were following the schedule in Table 1.

### Foliar Endophyte Application

Foliar treatments with the endophytic bacterial mixture were applied at the V3-V4 growth stage (17 days after sowing, roughly 10 days after seedling emergence). The endophyte formulation (a co-fermented consortium of beneficial strains) was diluted 1:40 (v/v) in a 10 mM MgCl₂ solution for spraying. Control plants were sprayed with 10 mM MgCl₂ alone (no endophytes). The application rate was calibrated to an agriculturally relevant field rate: 32 oz/acre (0.946 L/acre) of the endophyte mixture in approximately 10 gallons of water per acre (37.8 L/acre). In practice, each corn plant was misted using a small hand spray bottle (2 oz capacity) with a fine nozzle. The bottle was held ˜5 cm above the plant and about 10 quick sprays were applied while moving around the plant, ensuring all leaves were evenly coated to the point of run-off. Each treated plant received the same number of sprays (10) to standardize the foliar dose.

### Seed Treatment Application

For seed-applied inoculation, the endophyte mixture was formulated in a 0.5% (w/v) sodium alginate solution to create a gel coating. Hybrid corn seeds (100 seeds total) were coated with this formulation at a commercially relevant rate: 2.4 mL of the endophyte-alginate mixture per 1 lb of seed. The coated seeds were then dried and stored. This polymer-based coating was intended to protect the bacteria and attach them to the seed surface until sowing.

### Endophyte Viability on Coated Seeds

To verify that the endophytic bacteria survived the seed coating and storage process, a viability assay was performed one month after treatment. Coated seeds had been stored dry at ambient room temperature for 4 weeks. Twenty of these treated seeds were taken for testing. Each seed was placed in 10 mL of sterile 1×PBS buffer and vortexed vigorously to wash off the bacterial coating. The resulting wash solution (containing bacteria dislodged from the seed surface) was serially diluted and plated on appropriate growth media. After incubation, colony counts were performed to enumerate surviving bacteria. The colonies' identities were confirmed by their characteristic morphology and by PCR, ensuring they were the inoculated endophyte strains. Dilution plating results confirmed clear endophyte survival on the seeds: all introduced strains were recoverable even after drying and storage. The bacterial load on the seeds was expressed as colony forming units per seed (CFU/seed) for each strain. (Separate example data sets indicated the specific CFU/seed values for various strains, demonstrating that the formulation successfully preserved viable endophytes in a dormant state on the seed coat.)

### Fertilization Regime

Nitrogen fertilization treatments were applied to create “full N” and “low N” conditions. The full N treatment represented 100% of the recommended nitrogen rate for corn, while the low N treatment received only 25% of that rate (to stress the plants and test N-use efficiency improvements). Granular calcium ammonium nitrate (CAN, 27% N) was used as the fertilizer source, reflecting common field practice. Twenty days after sowing (DAS), corresponding to about 13 days after germination, thinning and fertilization took place. In each pot, if both seeds had germinated, the smaller seedling was removed to leave one plant per pot. Then, CAN fertilizer was applied to the soil surface at the appropriate dose for each treatment. The full N pots received 2.6 g CAN per pot (equivalent to 172.5 kg N/ha), and the low N pots received 1.7 g CAN per pot (approx. 115 kg N/ha, which was intended as 25% of the normal field N rate). This single application provided the main nitrogen supply for the experiment.

In addition to the solid CAN fertilizer, all pots were given a dilute nutrient solution regularly to supply other essential nutrients (without adding nitrogen). Starting at 20 DAS, a 1/10-strength Hoagland's No. 2 basal salt solution lacking nitrogen (e.g., HiMedia TS1117-5L formulation) was applied via top-watering to each pot. This maintenance feeding ensured plants had access to micronutrients and other macronutrients (except N) throughout growth. The watering schedule was as follows: 200 mL of the Hoagland minus-N solution was added per pot on days 20, 27, 34, and 38 DAS; then the volume was increased to 300 mL per pot on days 45, 49, 52, 55, 57, 59, and 62 DAS. Normal water was used as needed in between to maintain soil moisture, but care was taken to prevent leaching.

### Harvest and Sample Analysis

At 64 days after sowing, all plants were at the V10-V11 vegetative stage (ten to eleven fully expanded leaves). The above-ground part of each plant was harvested by cutting at soil level. Several growth parameters were measured in situ just before and at harvest:
a. Leaf chlorophyll content: Measured on a fully expanded leaf (mid-leaf, avoiding the midrib) using an MC-100 Chlorophyll Concentration Meter. Three readings per plant were taken and averaged to assess relative chlorophyll, which correlates with leaf nitrogen status.
b. Stalk diameter: Measured 10 cm above the soil surface on each plant's stalk. A digital caliper was used to record the diameters of the major and minor axes of the stalk cross-section, indicating stem thickness and strength.
c. Fresh weight: Immediately after cutting, the shoot of each plant was weighed to get the wet biomass.

After these measurements, the plant material was dried for biomass and tissue nutrient analyses. Shoots were oven-dried at 85° C. until constant weight and then weighed to determine dry biomass per plant. This dry weight is used in calculating total nutrient uptake by multiplying dry weight by nutrient concentration to give the total nutrient mass per plant.

The dried shoot tissue of each plant was first coarsely chopped using an IKA MultiDrive basic crusher. A 2-gram subsample of this chopped material was then finely ground in a Retsch MM400 mill to a uniform powder. To eliminate any residual moisture, the ground samples were further dried at 105° C. for at least 4 hours and cooled in a desiccator containing silica gel. The fully dried, ground plant powder was used for elemental analyses of carbon, nitrogen, and mineral nutrients.

For total carbon (% C) and nitrogen (% N) content, three replicates (3 plants) per treatment group were randomly selected. An aliquot of the dried, ground tissue from each of these plants was weighed and sealed in a tin foil capsule. These samples would be analyzed using a C/N analyzer (via combustion method) to determine the percentage of C and N in the shoot tissue.

The same three representative plants per treatment were used for detailed mineral uptake analysis. Approximately 150 mg of each ground plant sample was subjected to acid digestion for inductively coupled plasma optical emission spectrometry (ICP-OES). The digestion was carried out by mixing the 150 mg sample with 2 mL of deionized water and 4 mL of concentrated nitric acid (65% HNO₃) in a digestion tube. This mixture was heated to 150° C. for 10 minutes to completely break down organic matter, then allowed to cool to ˜70° C. After cooling, the digest was diluted with deionized water to a final volume of 15 mL. The resulting solution, containing the dissolved minerals from the plant, was analyzed using a PerkinElmer Optima 8300 ICP-OES instrument at BOKU University. This provided concentrations of macro- and micronutrients, including P, K, Ca, Mg, and Fe in the shoot tissue. Finally, by multiplying these concentrations by the plant's dry mass, the total uptake of each nutrient per plant (in mg/plant) could be calculated for comparison across treatments.

### Example 83

Seed Treatment with HT1-9+HT1-2 Endophyte Mixture

Corn seeds were coated with a formulation containing the endophytic bacterial strains HT1-9 and HT1-2, using 0.5% (w/v) sodium alginate as a binder, as described above. The coated seeds were dried and stored for one month prior to planting, resulting in viable on-seed endophyte populations of approximately 5,100 CFU/seed (HT1-9) and 3,833 CFU/seed (HT1-2) at the time of sowing. Plants grown from these inoculated seeds were subjected to a low-nitrogen regime (25% reduced N relative to the optimal level) to test effects on nitrogen use efficiency. Uninoculated seeds grown under the same low-N conditions served as controls, while a full-N treatment (100% optimal N) was included as a reference for normal growth response of the corn hybrid.

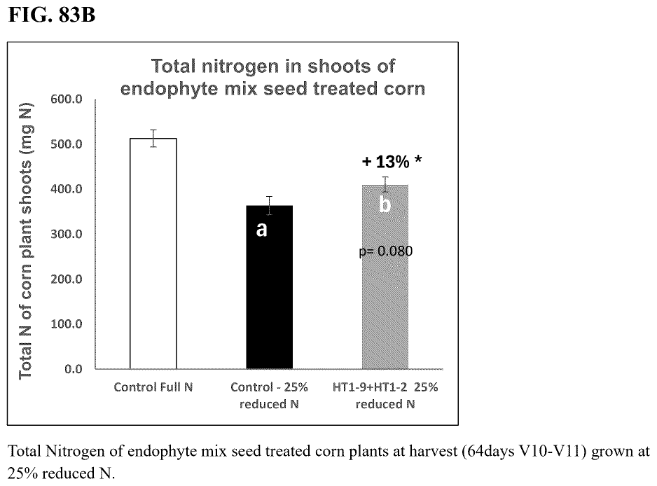

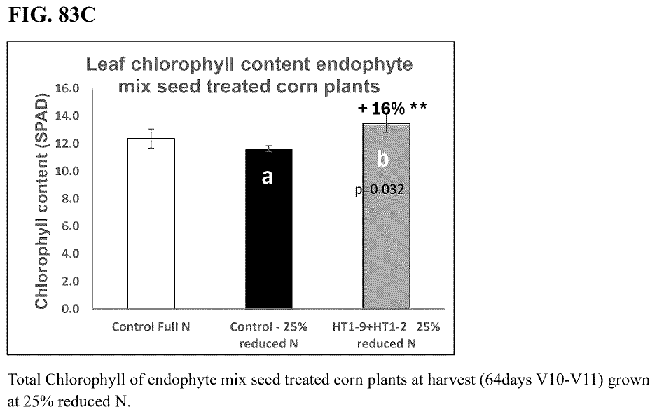

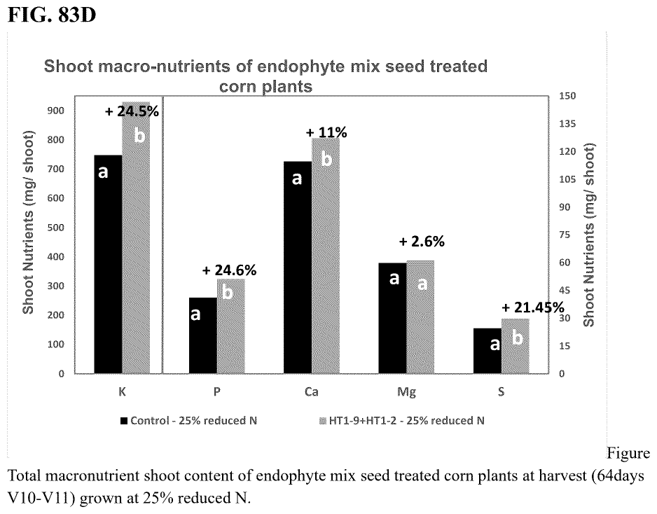

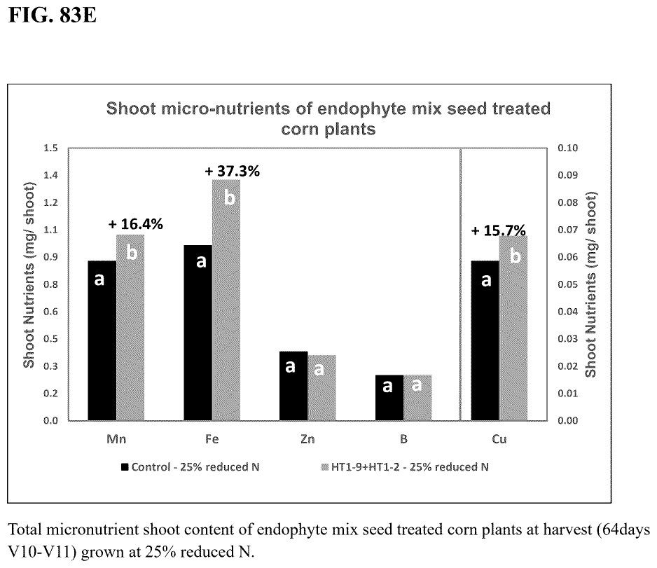

The plants were harvested at 64 days after planting at the V10-V11 growth stage. Corn plants from HT1-9+HT1-2 treated seeds showed improved growth and nutrient content compared to low-N controls, which were treated with 25% reduced N and no endophyte inoculum. Inoculated plants had a 12.2% higher fresh shoot biomass than uninoculated low-N controls (p≤0.05). See FIG. 83A. Inoculated plants accumulated 13% more total nitrogen in shoots than controls (p≤0.05). See FIG. 83B. Foliar chlorophyll content was 16% higher in the inoculated plants, indicating greener leaves under N stress than in controls (p≤0.05). See FIG. 83C. Major nutrient levels in shoots were elevated in treated plants: potassium (K) by +24.5%, phosphorus (P) by +24.6%, calcium (Ca) by +11%, and sulfur(S) by +21.45%. See FIG. 83D. These increased nutrient levels are all statistically significant improvements (p≤0.1). Magnesium (Mg) also showed a modest ˜2.6% increase in inoculated plants. Treated plants also had higher micronutrient accumulation: manganese (Mn) increased by +16.4%, iron (Fe) by +37.3%, and copper (Cu) by +15.7%. These increases were significantly higher than controls at p≤0.1. See FIG. 83E.

### Example 84

Seed Treatment with HT1-9+11R-BC Endophyte Mixture

Corn seeds were coated with a mixed inoculum of HT1-9 and 11R-BC endophytic strains with 0.5% w/v sodium alginate as binder, following the seed treatment protocol described above. After drying (˜1 month) and just before planting, HT1-9 viable count on seeds was about 5,917 CFU/seed, and 11R-BC was about 13,967 CFU/seed. Inoculated and control seeds were grown under low-nitrogen conditions (25% less N than optimal) to evaluate growth promotion under N limitation. An uninoculated low-N group served as the control, and a full-N (100% N) group was grown as a reference for normal growth of the hybrid.

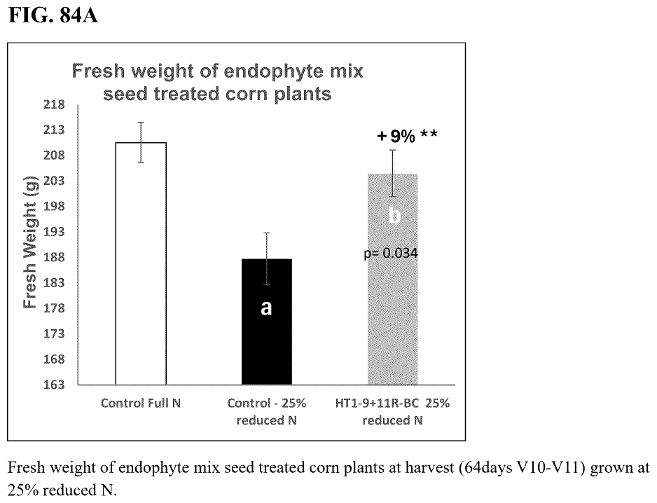

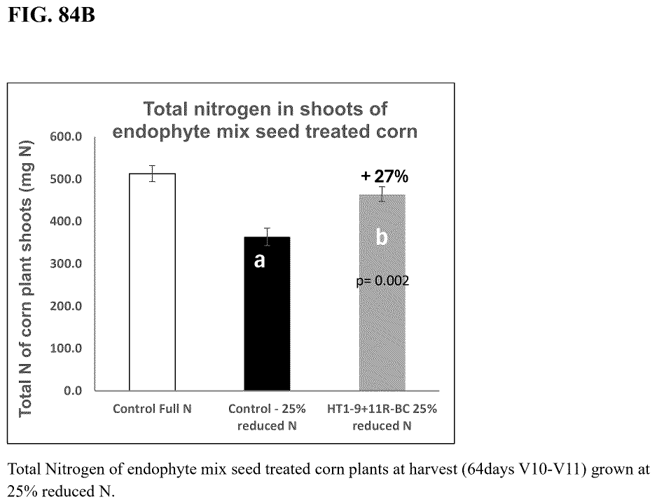

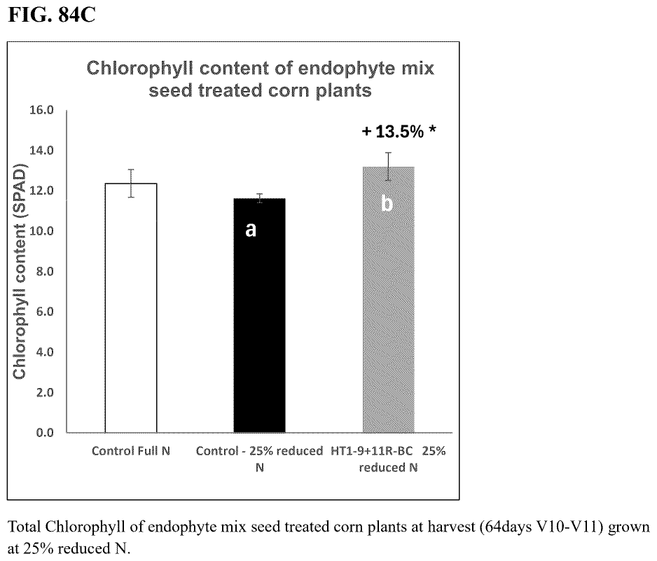

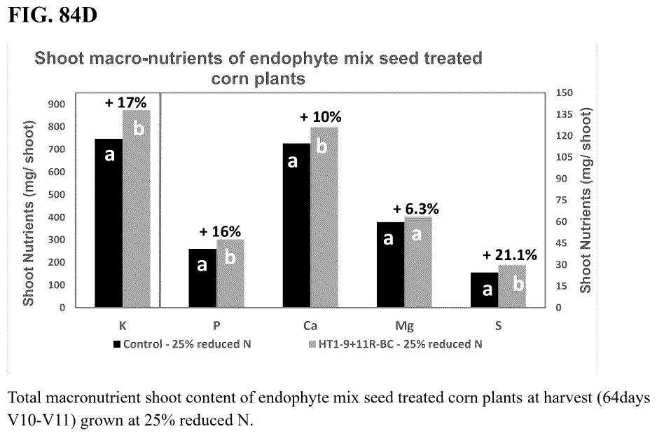

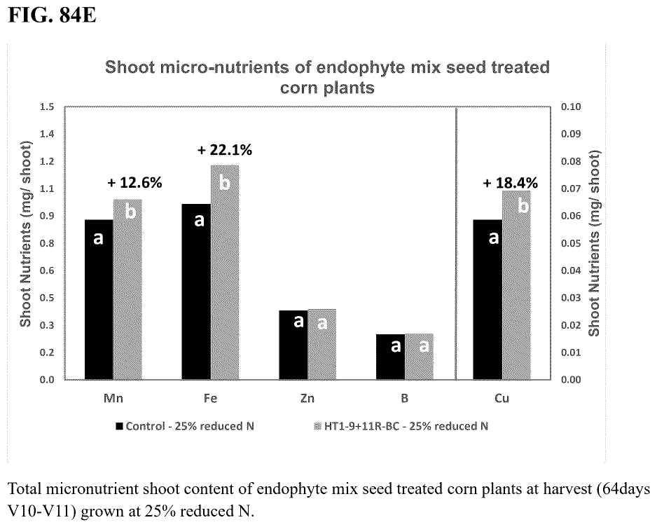

The plants were harvested at 64 days after planting at the V10-V11 growth stage. The HT1-9+11R-BC seed inoculation resulted in substantial improvements in plant performance under low-N stress compared to controls. Endophyte-inoculated plants showed a 9% increase in fresh shoot biomass over low-N controls (p≤0.05). See FIG. 84A. Shoot nitrogen content was 27% higher in inoculated plants relative to controls, a significant enhancement in N uptake/storage (p≤0.05). See FIG. 84B. Inoculated corn had 13.5% greater leaf chlorophyll content than control plants, indicating improved leaf N status (p<0.05). See FIG. 84C. Key macronutrients in shoots were elevated with inoculation: K by +17%, P by +16%, Ca by +10%, and S by +21.1%. See FIG. 84D. The increases in macronutrients were all statistically significant increases (p≤0.1). Magnesium (Mg) also rose by about +6.3% on average in treated plants. The endophyte treatment also boosted certain micronutrients: Mn increased by +12.6%, Fe by +22.1%, and Cu by +18.4% in shoots. The increases in macronutrients were all statistically significant increases (p≤0.1). See FIG. 84E.

### Example 85

Foliar Treatment with HT1-9+11R-BB Endophyte Mixture

Corn plants were treated with a foliar spray containing a consortium of HT1-9 and 11R-BB endophyte strains, applied at an early vegetative stage, using the same foliar application procedure described above. The spray formulation had a bacterial titer of approximately 7.6×10⁷ CFU/mL for HT1-9 and 1.44×10⁸ CFU/mL for 11R-BB at the time of application. Treated plants were grown under low-nitrogen conditions (25% reduced N), with untreated low-N plants as controls. A full-N (100% optimal nitrogen) group was maintained as a reference for normal growth but was not inoculated.

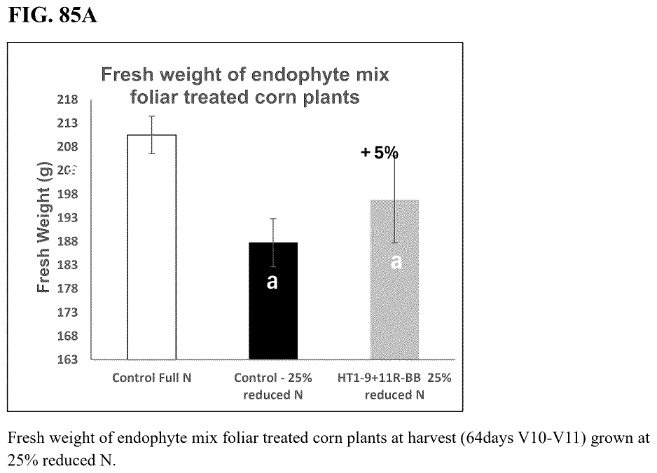

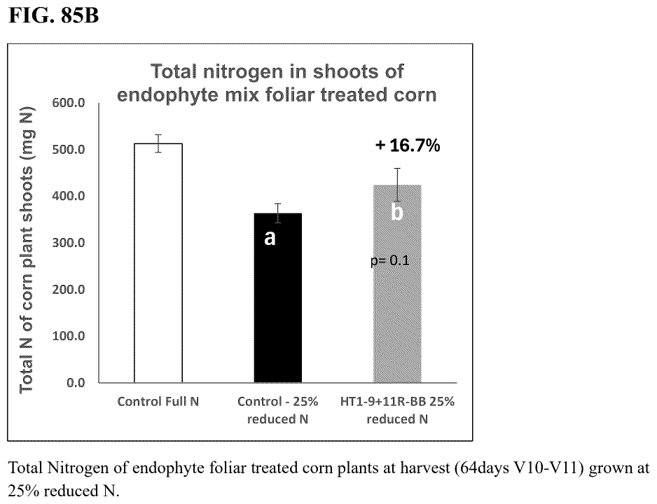

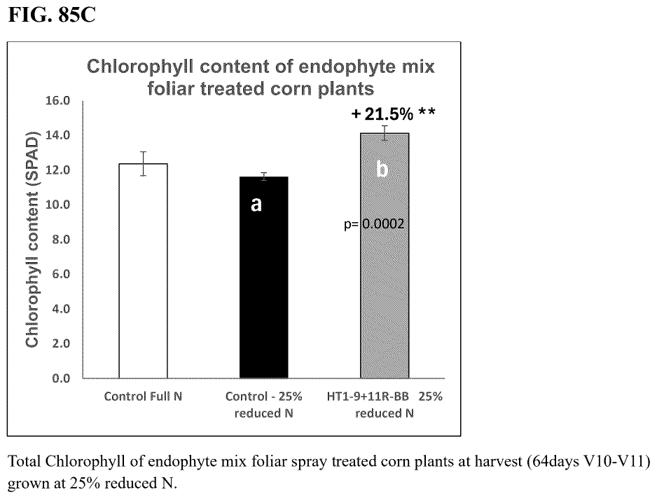

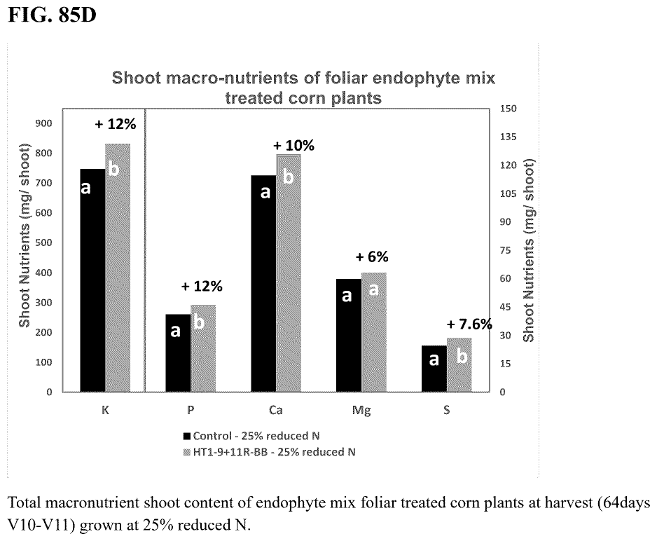

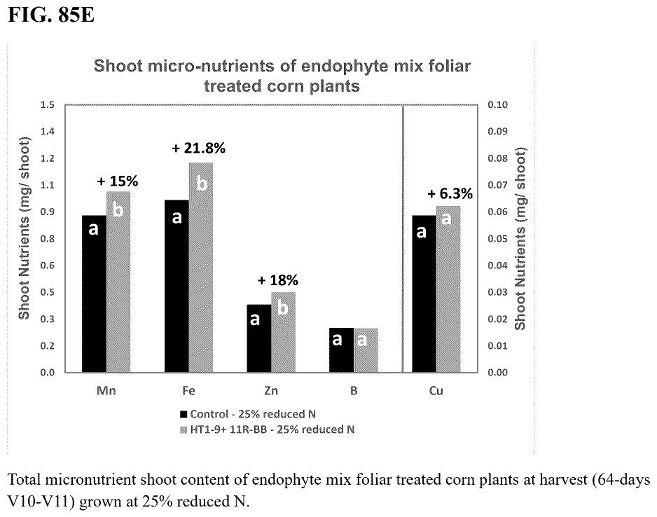

The plants were harvested at 64 days after planting at the V10-V11 growth stage. The HT1-9+11R-BB foliar inoculation had improved several growth and nutrient parameters in low-N corn plants. Inoculated plants tended toward higher biomass, with about a 5% increase in fresh shoot weight over low-N controls. See FIG. 85A. Treated plants had a significantly higher shoot N content, about 16.7% above the control levels (p≤0.05). See FIG. 85B. Foliar chlorophyll content was 21.5% greater in endophyte-sprayed plants compared to controls, a significant increase (p≤0.05). See FIG. 85C. Several macronutrient levels rose in inoculated plant shoots: K by +12%, P by +12%, Ca by +10%, and S by +7.6%. The increases in macronutrients were all statistically significant increases (p≤0.1). See FIG. 85D. Magnesium also increased +6.0% in treated vs. control. Several micronutrient levels rose in inoculated plant shoots: Mn increased by +15%, Fe by +21.8%, Zn by +18%, and Cu by +6.3% in shoots of treated plants. The increases in micronutrients were all statistically significant increases (p≤0.1). See FIG. 85E.

### Example 86

Foliar Treatment with HT1-9+PTD1 Endophyte Mixture

Corn plants were treated with a foliar spray containing a consortium of HT1-9 and PTD1 endophyte strains, applied at an early vegetative stage, using the same foliar application procedure described above. The mixed formulation had approximate cell densities of 1.27×10⁸ CFU/mL for HT1-9 and 1.38×10⁷ CFU/mL for PTD1 at the time of application. Untreated low-N treated plants (25% reduced N) served as controls, and a full-N uninoculated group was grown in parallel as a normal-growth reference.

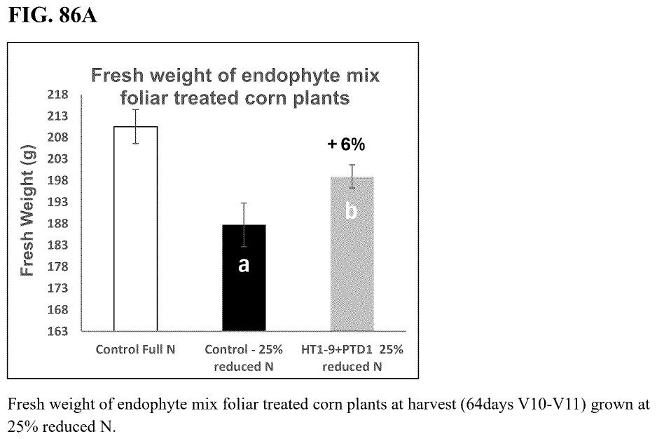

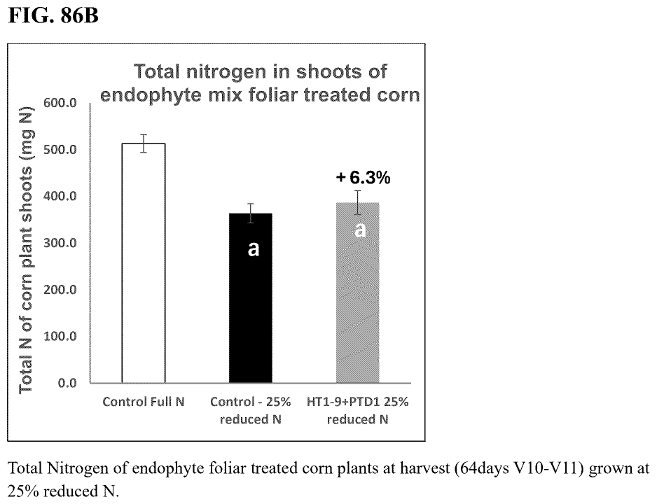

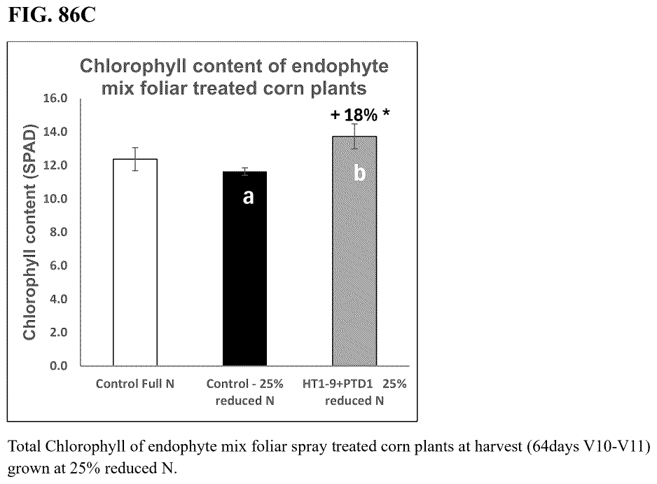

The plants were harvested at 64 days after planting at the V10-V11 growth stage. Corn plants treated with the HT1-9+PTD1 foliar spray showed notable improvements in growth metrics under N limitation (25% reduced N). Inoculated plants yielded about 6% greater fresh shoot weight compared to low-N controls, a marginal increase (p≤0.1). See FIG. 86A. Shoot nitrogen content was ˜6.3% higher in HT1-9+PTD1 treated plants relative to controls. See FIG. 86B. Treated plants exhibited a significant 18% increase in leaf chlorophyll content over control plants (p≤0.05), indicating better leaf nitrogen status or photosynthetic capacity under low-N conditions. See FIG. 86C.

### Example 87

Foliar Treatment with WW5+WW6+WW7 Endophyte Mixture

Corn plants were treated with a three-strain endophyte consortium (WW5, WW6, and WW7) as a foliar spray at an early vegetative stage in the low-N treatment (25% reduced N), using the same protocol as above. The foliar formulation contained approximately 1.22×10⁷ CFU/mL of WW5, 7.85×10⁷ CFU/mL of WW6, and 4.52×10⁷ CFU/mL of WW7 at application time. Uninoculated plants under low-N served as controls, and a full-N uninoculated group was included for reference to normal growth outcomes.

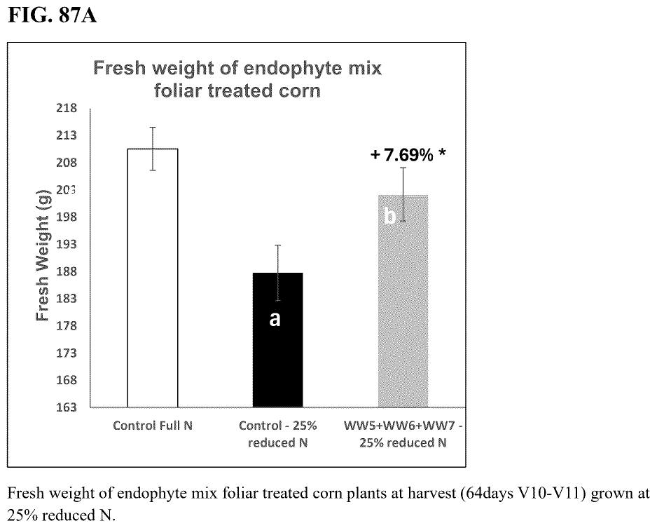

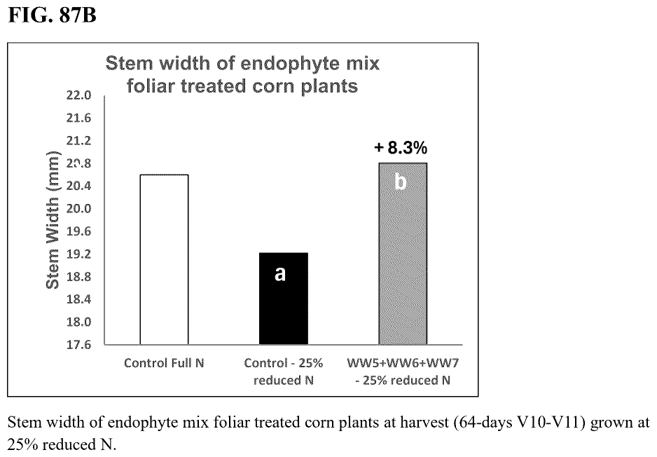

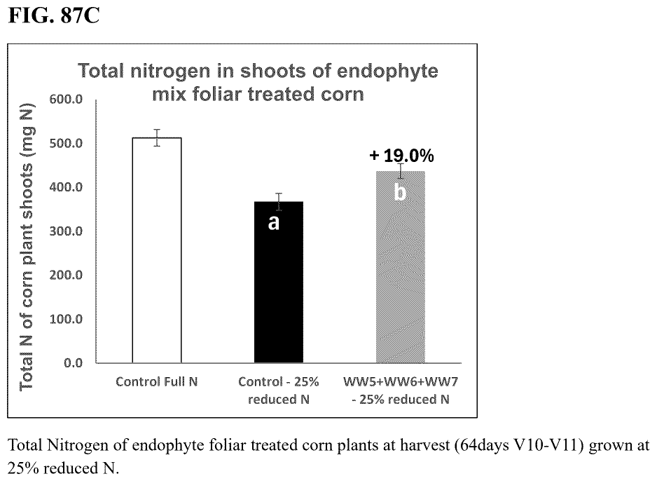

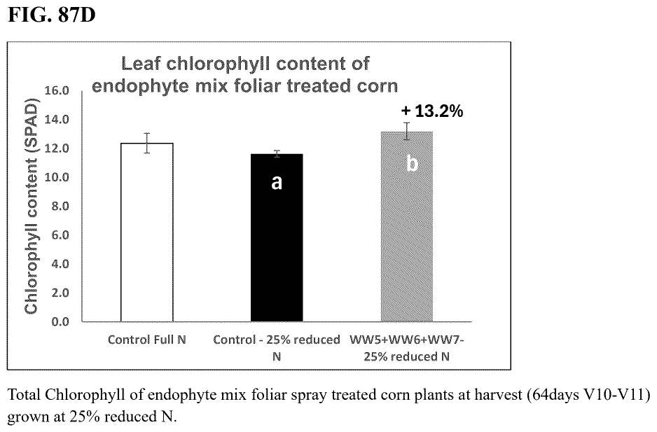

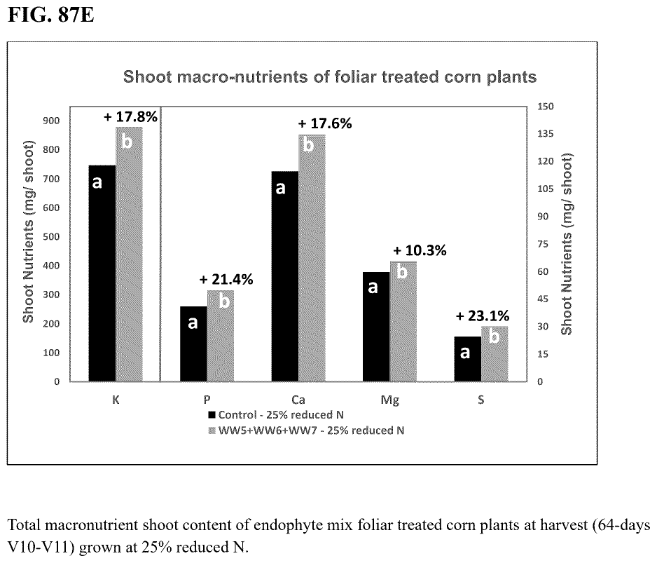

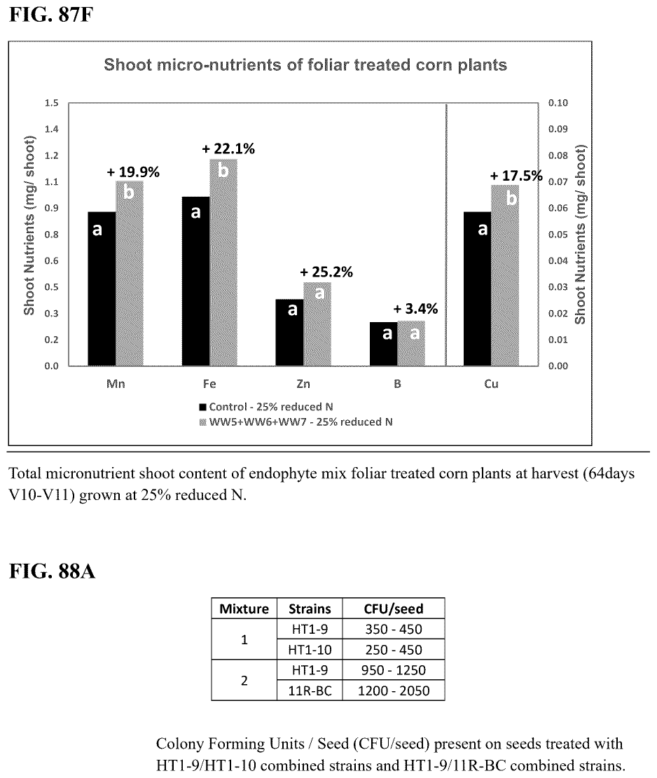

The plants were harvested at 64 days after planting at the V10-V11 growth stage. The WW5+WW6+WW7 foliar treatment produced significant positive effects on corn growth and nutrient accumulation under nitrogen limitation. Treated plants had a 7.7% higher fresh biomass yield compared to low-N control plants, a noticeable improvement (p≤0.1). See FIG. 87A. The lower stalk (stem) thickness of inoculated plants was 8.3% greater than that of control plants (p≤0.1), suggesting enhanced structural growth. See FIG. 87B. Endophyte-treated plants showed a 19.0% increase in total shoot N content relative to controls, a substantial and significant boost in nitrogen uptake/utilization (p≤0.05). See FIG. 87C. Foliar chlorophyll levels were 13.2% higher in the WW5+WW6+WW7 treated plants versus controls, reflecting improved leaf N status (p≤0.05). See FIG. 87D. Treated plants accumulated more macronutrients in shoots than controls: −K by +17.8%, P by +21.4%, Ca by +17.6%, Mg by +10.3%, and S by +23.1%. See FIG. 87E. The increases in macronutrients were all statistically significant increases relative to controls (p≤0.1). Similarly, shoot micronutrient concentrations were elevated with the WW5+WW6+WW7 consortium: Mn increased by +19.9%, Fe by +22.1%, Zn by +25.2%, and Cu by +17.5% in treated plants (significant at p≤0.1). See FIG. 87F. Boron (B) also showed a minor average increase (˜+3.4%), but this change was not statistically significant relative to controls.

### Example 88

Field Seed Treatment in Wheat with Strains HT1-9, HT1-10, and 11R-BC

Wheat seeds were treated with endophyte strains HT1-9, HT1-10, and 11R-BC to evaluate improvements in wheat grain yield under field conditions. The objective was to determine whether inoculating wheat seeds with these specific endophyte strains increases crop yield without adversely affecting seed viability or agronomic performance.

In order to characterize the harvest yield effects of seed-applied endophytic strains HT1-9, HT1-10, and 11R-BC under normal nitrogen fertilizer rates in field trials, an experiment was conducted using winter wheat as the test crop. Wheat seeds (cultivar Stingray CL+) were treated with two different endophyte mixtures formulated with fermented endophyte broth plus 0.5% (w/v) sodium alginate as a carrier. The endophyte formulation was applied to the seed at a rate of 1 fluid ounce per hundredweight (1 fl oz/cwt). Prior to endophyte application, all seeds were pre-treated with a conventional seed treatment package of fungicides (DIFENOCONAZOLE, MEFENOXAM, THIABENDAZOLE, and TOLCLOFOS). Control seeds of the same wheat variety received the same chemical seed treatment package minus the endophytes. After treatment, seeds were allowed to air-dry completely and then stored at room temperature for 3 months.

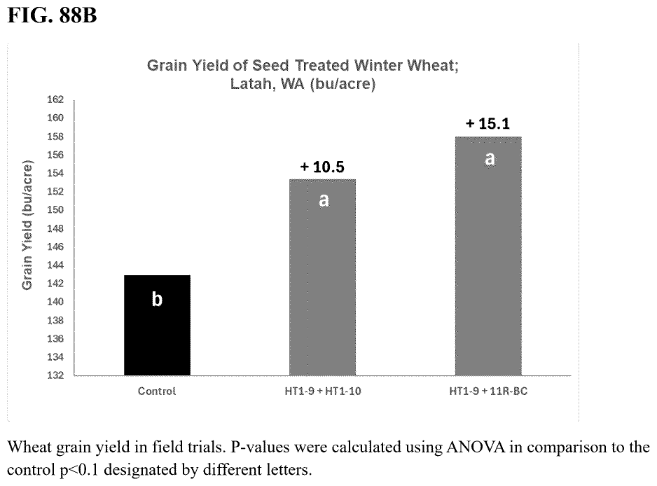

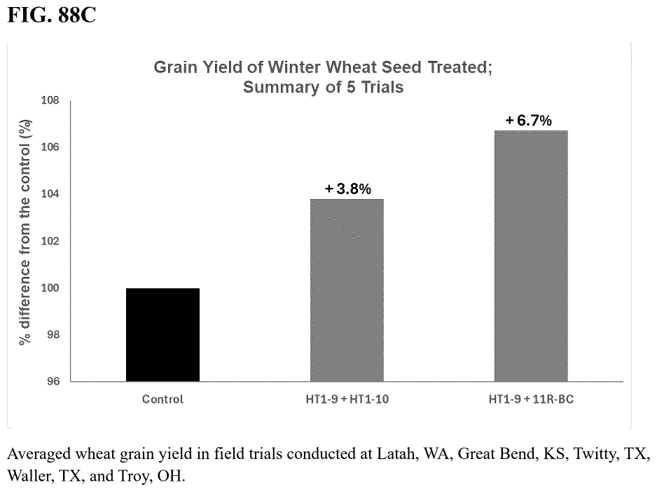

Endophyte viability on the seed after the 3-month storage period was confirmed by microbial enumeration. Twenty seeds from each treatment were washed in 10 mL of sterile 1× phosphate-buffered saline (PBS) to dislodge microbes from the seed surface, and the wash solution was serially dilution-plated to quantify surviving bacteria. The results, expressed as colony forming units per seed (CFU/seed), demonstrated that each of the applied strains remained alive and dormant on the seed coat after storage. FIG. 88A summarizes the enumeration results. For the seed treatment containing the combination of HT1-9+HT1-10, approximately 350-450 CFU/seed of strain HT1-9 and 250-450 CFU/seed of strain HT1-10 were recovered. For the seed treatment containing the combination of HT1-9+11R-BC, approximately 950-1250 CFU/seed of strain HT1-9 and 1200-2050 CFU/seed of strain 11R-BC were recovered.

Treated and untreated (control) seeds were planted in a field trial at Latah, Washington, USA in the fall season, using standard commercial planting methods. The experimental design consisted of small plots of approximately 10 ft×30 ft each, with six replicated plots per treatment, including six control plots. The field was fertilized according to typical wheat agronomic practice with an N—P—K formulation of 100-10⁻¹⁰ lbs/acre of nitrogen-phosphorus-potassium, and rainfall served as the water source with no additional irrigation provided. During the trial, there were no prolonged drought conditions, and no symptoms of crop disease or pest damage were observed in any of the plots. The wheat crop was grown to maturity and harvested 294 days after planting.

Grain yield data from the Latah trial showed that both endophyte-treated groups produced higher yields than the control group. Statistical analysis by one-way ANOVA indicated that the yield increases with endophyte treatment were significant at p<0.1. In particular, wheat plants grown from seeds treated with the HT1-9+HT1-10 endophyte mixture yielded on average about 10.5 bushels per acre (bu/acre) more grain than the control, and those from seeds treated with the HT1-9+11R-BC mixture yielded about 15.1 bushels per acre more than the control. FIG. 88B illustrates the wheat grain yield results at Latah; treatments labeled with different letters in FIG. 88B differ significantly from each other (p<0.1 by ANOVA).

To evaluate the consistency of the yield improvements across different environments, the same seed treatments (HT1-9+HT1-10, HT1-9+11R-BC, and an untreated control) were tested in a series of field trials at five distinct locations. These trial sites were located in five different states across the United States: Latah, WA; Great Bend, KS; Twitty, TX; Waller, TX; and Troy, OH. Each site utilized a locally adapted wheat variety suited to that region, and followed local standard agronomic practices, including irrigation or rain-fed management, fertilization, and harvest timing. At each site, the crop yield from each endophyte-treated plot was compared to the yield from the control plots, and the percentage increase in yield relative to the control was calculated.

The yield improvement data from all five sites were compiled and analyzed using the ARM Summary Across Trials (ST) software program (Agricultural Research Manager, GDM Solutions Inc., Brookings, SD). The multi-site analysis showed that the endophyte seed treatments consistently increased wheat grain yield compared to the untreated control across all trial locations. As shown in FIG. 88C, the seed treatment containing HT1-9+HT1-10 produced an average yield increase of approximately +3.8% over the control, and the seed treatment containing HT1-9+11R-BC produced an average yield increase of approximately +6.7% over the control, when averaged across the five trial sites. These results demonstrate a positive yield effect of the endophyte treatments under normal fertilizer input conditions in multiple field environments.

### Example 89

### Effects of Endophyte Formulation Foliar Application on Corn Growth Under Reduced Nitrogen

To demonstrate the nutrient use efficiency effects caused by the use of nitrogen fixing endophyte formulations applied as a foliar spray on corn plants, seeds were first germinated in a soil mixture of equal parts 1:1:1 vermiculite, perlite and peatmoss soil mixture and plants were grown in a tightly controlled environment plant growth room. Endophyte formulations of HT1-9, PTD1, 11R-BB, and HT1-10, were prepared as described in Example 2A. The endophyte strains were mixed in different formulations as follows: HT1-9+PTD1, HT1-9+11R-BB, HT1-9+HT1-10 and were applied as a foliar spray to corn one week after planting. The spray was applied at a rate of 32 ounces per acre, diluted in a 20-gallon solution to ensure even distribution across the treated plants. Plants were harvested, dried, and weighed after 30 days.

The trial design included 9 four-inch standard plastic green pots for each treatment were exposed to LED lights at ˜700 uEinsteins at 25° C. in a controlled grow room. Plants were watered 3 times per week as needed to maintain moist-slightly dry soil. They were fertilized once a week using a modified Hoagland's solution with reduced nitrogen at 75 ppm N. Controls were raised under the same conditions with no endophyte formulation foliar treatments. After 30 days growth, plants were dried and weighed.

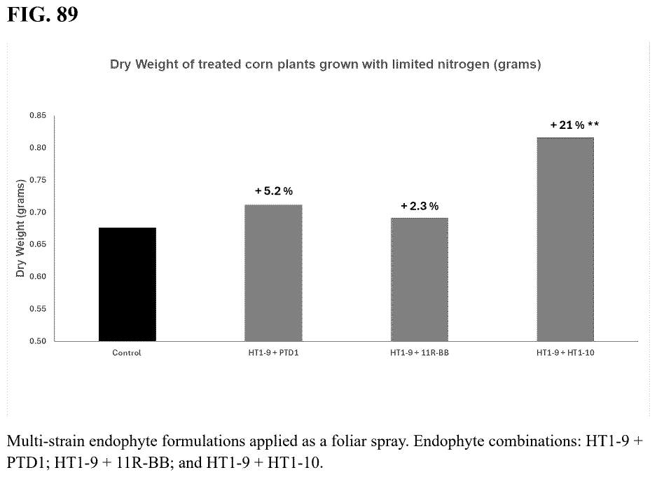

The results provided in FIG. 89 demonstrate endophyte mix formulations applied as a foliar spray caused average corn plant shoot increase in dry weight under moderate N deficiency 30 days after germination. The strains tested demonstrated varying positive growth effects under low N on the dry weight of the corn compared to the control group. The HT1-9+HT1-10 treatment group showed a +21% increase in dry weight and was statistically significant at p<0.05. The HT1-9+PTD1-treated plants resulted in a +5.2% increase in dry weight and the HT1-9+11R-BB treated plants caused a positive increase of +2.3%.

### Example 90

### Effects of Different Endophyte Formulation Combinations on Corn Growth Under Reduced Nitrogen

To demonstrate the nutrient use efficiency effects caused by the use of a different set of nitrogen fixing endophyte formulations applied as a foliar spray on corn plants, seeds were first germinated in a soil mixture of equal parts 1:1:1 vermiculite, perlite and peatmoss soil mixture and plants were grown in a tightly controlled environment plant growth room. Foliar spray strain mix combinations of HT1-9+11R-BB, HT1-9+HT1-2, HT1-9+R10, and PTD1+WP5 were applied separately to corn at a rate of 32 ounces per acre, diluted in a 20-gallon solution to ensure even distribution across the treated plants. Plants were harvested, dried, and weighed after 30 days. The trial design and plant growth was the same as above.

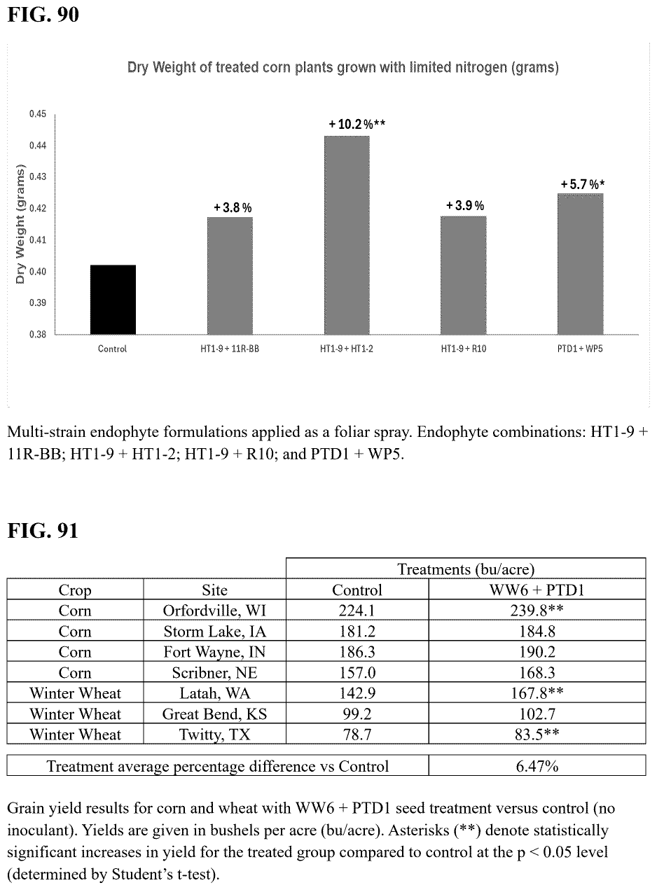

The results provided in FIG. 90 demonstrate multiple strain endophyte formulations applied as a foliar spray caused a positive biomass increase as follows: the HT1-9+HT1-2 treatment group corn showed a +10.2% increase in dry weight p<0.05, the PTD1+WP5 treated group had a +5.7% increase in dry weight p<0.1, the HT1-9+R10 treated plants had a +3.9% increase in dry weight, and the HT1-9+11R-BB treated plants showed an increase of +3.8% in biomass.

### Example 91

### Effects of (WW6+PTD1) Applied as a Seed Treatment on Harvested Yield Under Normal Nitrogen Fertilizer Rates in Wheat and Corn Agricultural Fields.

An inoculant formulation containing the endophyte fermentates of strains WW6 and PTD1 was prepared for seed treatment with 0.5% w/v sodium alginate as a carrier. This WW6+PTD1 inoculant was applied as a seed coating at a rate of 1 fl oz per cwt (hundredweight) of wheat seed and 4 fl oz per 80,000 corn seeds. Prior to inoculation, all seeds had been treated with standard commercially available crop protection packages (fungicide/insecticide seed treatments), ensuring that both treated and control seeds received identical conventional seed protections aside from the endophyte inoculant. The treated seeds were allowed to dry thoroughly before planting.

Field trials were conducted to evaluate the effect of the WW6+PTD1 seed treatment on harvested grain yield in winter wheat and corn under normal (optimum) nitrogen fertilizer rates and typical agronomic conditions. Treated and untreated control seeds were planted in small plot trials with six replications per treatment at multiple geographically distinct sites:

Winter wheat was planted in the fall at three sites were used: Latah, Washington; Great Bend, Kansas; and Twitty, Texas. Wheat plots measured approximately 10 ft×30 ft each. Local winter wheat varieties adapted to each region were used. Corn seed were planted in the spring at four our sites were used: Orfordville, Wisconsin; Storm Lake, Idaho; Fort Wayne, Indiana; and Scribner, Nebraska. Corn plots measured approximately 10 ft×50 ft each, using locally adapted corn hybrids at each location.

All trials were conducted using standard commercial farming practices for that region. Each site received the normal recommended nitrogen fertilizer rate along with other necessary nutrients for the crop, and irrigation and other crop management practices (e.g. planting density, pest control, and harvest timing) were in accordance with local agronomic practices. Each field trial location utilized a variety or hybrid specifically suited to the local growing conditions. Grain yield was measured in bushels per acre at maturity for each plot and averaged per treatment for comparison.

FIG. 91 provides a table summarizing the harvested grain yields for corn and wheat with and without the WW6+PTD1 seed treatment. Across all seven field trial sites, the WW6+PTD1 endophyte treatment consistently increased grain yield compared to the untreated control. In the combined data, treated plots yielded on average about 6.47% higher grain yield than controls under equivalent fertilizer and management conditions.

As shown in FIG. 91, every trial site exhibited a positive yield response to the WW6+PTD1 inoculant treatment. Notably, at the Orfordville, WI corn site and the Latah, WA winter wheat site, the inoculated seeds produced a markedly higher yield than the control, with increases from 224.1 to 239.8 bu/acre for corn and 142.9 to 167.8 bu/acre for wheat, respectively. These differences were statistically significant (p<0.05). The Twitty, TX wheat trial also showed a significant improvement, with yields increasing from 78.7 to 83.5 bu/acre. Other locations demonstrated yield increases with the WW6+PTD1 treatment as well at lower levels.

Overall, the inclusion of the WW6+PTD1 endophyte seed treatment under normal nitrogen fertilization conditions resulted in improved grain yields in both wheat and corn. This consistent trend across multiple states and growing conditions suggests that the WW6+PTD1 inoculant can enhance crop yield when used as a seed treatment alongside standard agricultural practices. The average 6.47% yield increase observed with the treated seeds (vs. control seeds) underscores the potential agronomic benefit of this endophytic inoculant formulation in corn and wheat production.

### Example 92

### Effects of (WW6+PTD1 and PTD1+WP1) Applied as a Foliar Treatment on Harvested Yield Under Normal Nitrogen Fertilizer Rates in Wheat and Corn Agricultural Fields.

Field trials were conducted to evaluate the impact of foliar applications of endophyte fermentate mixtures WW6+PTD1 and PTD1+WP1 on grain yield in wheat and corn under standard nitrogen fertilization rates: optimum nitrogen supply with no intentional N limitation. The endophyte fermentate treatments were applied at a rate of 16 fl oz/acre as a foliar spray. For winter wheat, the application was made at the spring green-up growth stage, and for corn, the application was made between the V3 and V6 growth stages.

The trials were performed in small-plot research settings using commercial agronomic practices. Winter wheat was planted in the fall using plots of approximately 10 ft×30 ft at three different sites: Latah, Washington; Great Bend, Kansas; and Waller, Texas. Corn was planted in the spring at two sites in plots of about 10 ft×50 ft: Richland, Iowa and Ellendale, Minnesota. Each treatment was replicated six (6) times per site in a randomized trial layout. Each field site utilized a locally adapted crop variety seed cultivar appropriate for that region. Irrigation, fertilizer applications, and harvest timing at each location were done according to local standard practices to ensure that crop management was optimal and representative of normal farming conditions. There were no extraordinary environmental stresses or pest/disease outbreaks reported during the trials beyond typical conditions for each locale.

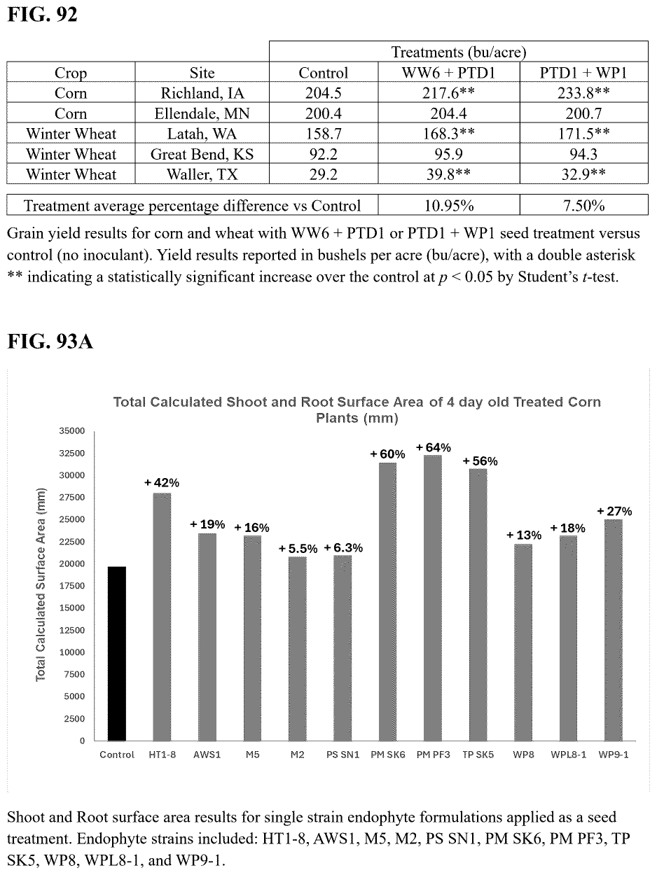

Across all five field trial sites, foliar treatment with the WW6+PTD1 and PTD1+WP1 fermentate mixtures resulted in increased grain yield of both wheat and corn compared to the untreated control plots. The yield results for each site are reported in bushels per acre (bu/acre) in FIG. 92. FIG. 92 illustrates the grain yield outcomes for each treatment at each site for both corn and wheat, with the statistical significance of differences noted by ** symbols. The WW6+PTD1 and PTD1+WP1 foliar treatments consistently improved yield in both crops relative to the untreated control. In particular, the yield increases in corn at the Richland, and in wheat at the Latah and Waller sites were pronounced and statistically significant (p<0.05) for both treatment mixtures. For example, for corn at the Richland, IA site, the WW6+PTD1 treatment increased yield by about 13.1 bu/acre above the control, and the PTD1+WP1 treatment by about 29.3 bu/acre above the control. At Latah, WA, a similar trend was observed in wheat: WW6+PTD1 increased wheat yield by 9.6 bu/acre, and PTD1+WP1 by 12.8 bu/acre. At the Waller, TX site, WW6+PTD1 boosted wheat yield by +10.6 bu/acre over control, while PTD1+WP1 increased yield by +3.7 bu/acre. It should be noted that the Waller, TX site had overall lower yields due to environmental factors typical of that region. All these noted increases were significant at the 95% confidence level.

Results from the other trial locations for corn (Ellendale, MN) and wheat (Great Bend, KS) also showed positive yield trends with the treatments. In Ellendale, MN, both treated corn groups yielded approximately the same as the control (around 200 bu/acre). In Great Bend, KS, treated wheat yields were higher than control (increases of ˜2-4 bu/acre).

When averaged across all five field trials, the WW6+PTD1 foliar treatment resulted in an overall yield increase of about +10.95% relative to the control, while the PTD1+WP1 treatment produced an average increase of approximately +7.50% versus control. These percentage improvements calculated from the yield differences at each site underscore the beneficial effect of the endophytic foliar applications under standard nitrogen-fertilized conditions. The consistency of the yield enhancement indicates that the endophyte inoculum mixtures provided an additive growth-promoting effect.

The data in FIG. 92 demonstrate that foliar application of the WW6+PTD1 and PTD1+WP1 endophyte compositions can advantageously increase crop yield in wheat and corn farming systems. These results confirm that, under typical field conditions with sufficient fertilizer, the introduction of beneficial endophytic microbes via foliar spray can further boost grain production.

### Example 93

### Effects of Endophytes Application on Germinating Seedling Growth.

To demonstrate the effect of endophyte inoculants on early crop seedling biomass, corn seeds were treated with various endophytic bacterial formulations. Endophyte formulations of HT1-8, AWS1, M5, M2, PS SN1, PM SK6, PM PF3, TP SK5, WP8, WPL8-1, and WP9-1 were prepared as described in Example 2A herein. One milliliter of each endophyte strain formulation was mixed with nine milliliters of distilled water (dH₂O). Each mixture was added to a Petri dish lined with two Whatman filter papers, and five corn seeds were placed in each dish. The corn seedlings were incubated for 4 days after treatment, then evaluated for growth. Images of the 4-day-old seedlings were analyzed using ImageJ software to quantify total seedling biomass. A known distance (mm) scale was set in ImageJ to convert pixel measurements to actual length, and color threshold adjustments were applied to isolate and highlight root and shoot seedling tissues from the background, ensuring accurate boundary detection. The total combined root and shoot seedling area was measured for each seedling as an indicator of biomass, and values from three replicate dishes per treatment were combined. From these measurements, the percent change in total biomass relative to a water-only control was calculated for each endophyte treatment. This image-based approach enabled a rapid and standardized assessment of early seedling vigor across all endophyte formulations tested under identical conditions.

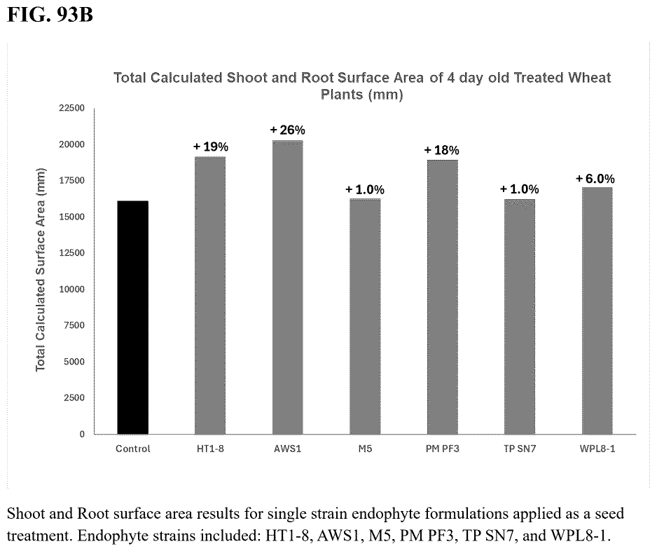

The endophyte strains tested on corn demonstrated varying positive effects on seedling biomass compared to the control group. All treated corn seeds showed increased total seedling biomass relative to the water-treated control. Specifically, the observed increases in total biomass relative to controls were approximately +42% for strain HT1-8, +19% for AWS1, +16% for M5, +5.5% for M2, +6.3% for PS SN1, +60% for PM SK6, +64% for PM PF3, +56% for TP SK5, +13% for WP8, +18% for WPL8-1, and +27% for WP9-1. The results are provided in FIG. 93A, and demonstrate that endophyte formulations applied to corn seeds can substantially increase total root and shoot biomass of germinating seedlings compared to untreated controls.

In a further study, the effects of similar endophyte inoculations on wheat seedling biomass were evaluated using the same methodology. Endophyte formulations of HT1-8, AWS1, M5, PM PF3, TP SN7, and WPL8-1 were prepared as described in Example 2A herein. One milliliter of each formulation was mixed with nine milliliters of dH₂O, and five wheat seeds were placed in Petri dishes with the treated filter papers. Wheat seedlings were grown for 4 days after treatment and then imaged for analysis. The 4-day-old wheat seedling images were analyzed in ImageJ to quantify total seedling biomass following the same procedure used for corn, including setting a scale, thresholding to isolate seedlings, and measuring combined root and shoot area for three replicates per treatment.

The endophyte strains tested on wheat also showed positive effects on seedling biomass compared to the control group, though the magnitude of improvement varied by strain. The treated wheat seeds exhibited increased total biomass relative to water-treated control seeds by approximately +19% for HT1-8, +26% for AWS1, +1% for M5, +18% for PM PF3, +1.0% for TP SN7, and +6.0% for WPL8-1. The results are provided in FIG. 93B, and demonstrate that endophyte formulations applied to wheat seeds can increase seedling root and shoot biomass relative to untreated controls. Each of the endophyte inoculants thus conferred some improvement in early seedling growth, with certain strains yielding higher biomass increases in wheat seedlings after 4 days of growth.

### Example 94

### Effects of Endophytes Application on Germinating Seedling Growth.

A field study was designed to demonstrate the ability of endophyte foliar treatment compositions to provide substantial harvested yields under normal and reduced nitrogen fertilizer application. WW6+WW7 and PTD1+WP1 fermentates were evaluated as foliar applications for harvested yield effects on corn under optimum rates of nitrogen and 20% reduction of nitrogen.

Field trial had small plots (10′×40′) and 6 replications per treatment. UAN 32 fertilizer was applied as a preplant at 200 lb/acre for the optimal fertilizer rate (100% rate) and 160 lb/acre for the reduced 80% application rate. The endophyte fermentate mixtures were foliar applied at 16 fl oz/acre on corn plants at the V4 stage. The grain was collected from each plot once the grain moisture reached approximately 15.5%.

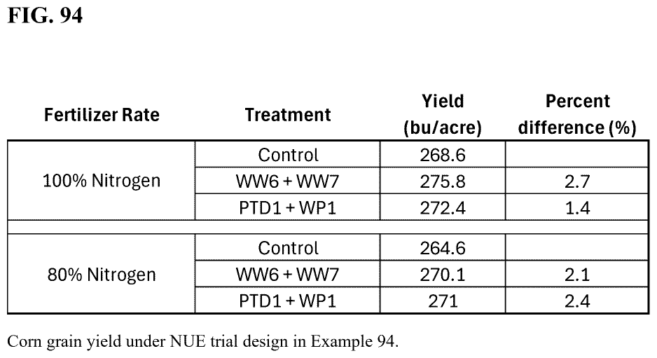

The results were evaluated via a factorial analysis and showed there was a statistical difference in grain yield between the 100% fertilizer treatments and the 80% fertilizer treatments indicated proper NUE trial design. The WW6+WW7 treatment with 100% fertilizer application increased grain yield 2.7% compared to the control treatment. The WW6+WW7 treatment in combination with 80% fertilizer application increased grain yield 2.1% compared to the control treatment. The PTD1+WP1 treatment with 100% fertilizer application increased grain yield 1.4% compared to the control treatment. Whereas the PTD1+WP1 treatment with 80% fertilizer application increased grain yield 2.4% compared to the control treatment. The WW6+WW7 and PTD1+WP1 endophyte treatments consistently increased corn grain crop yields when compared to controls. The results are presented in FIG. 94 and demonstrate the endophyte treatments reduce the corn fertilizer requirements needed to maintain the same grain yield as control corn plants.

### Example 95

### Use of Co-Fermented Endophyte Mixtures Applied as Novel Wheat Seed Treatment Under Reduced Nitrogen Fertilizer.

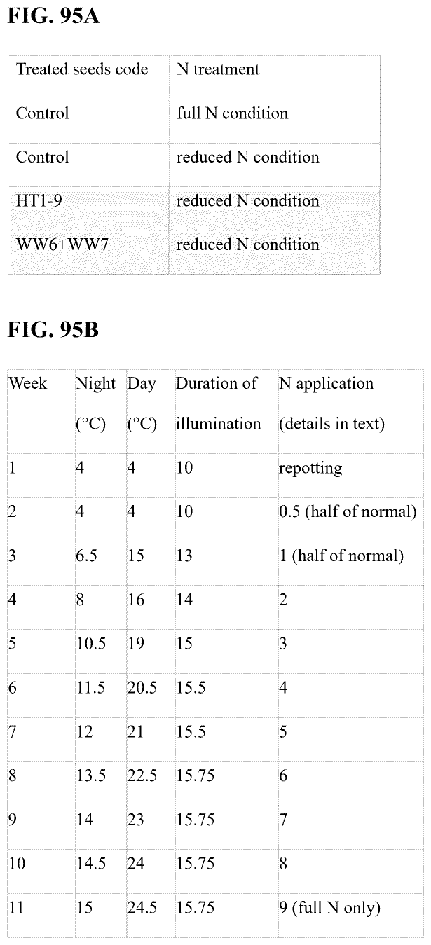

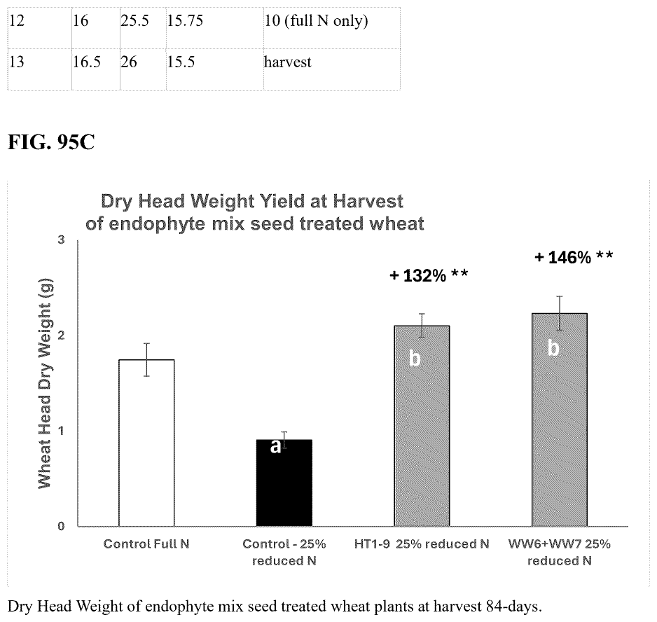

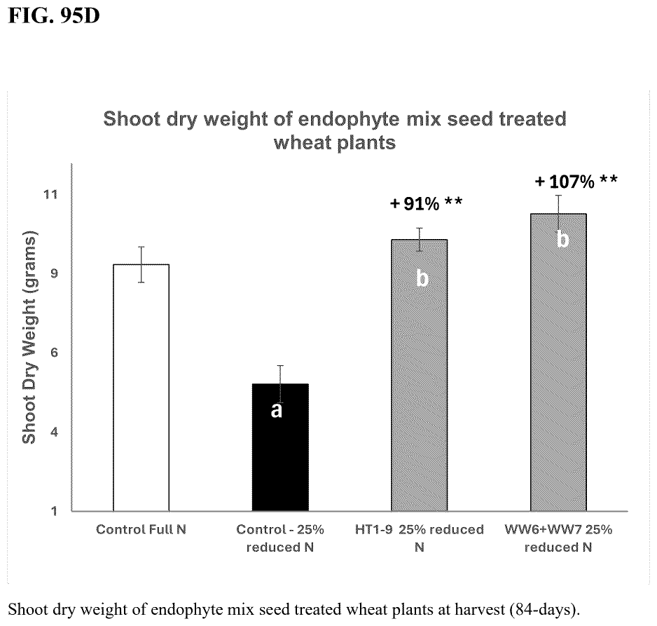

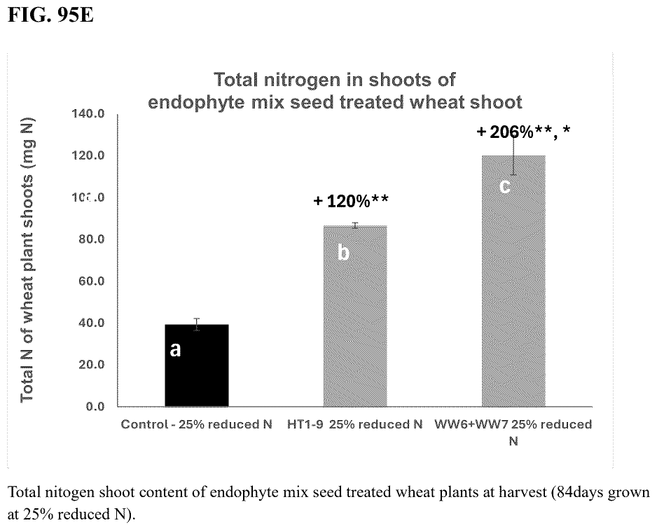

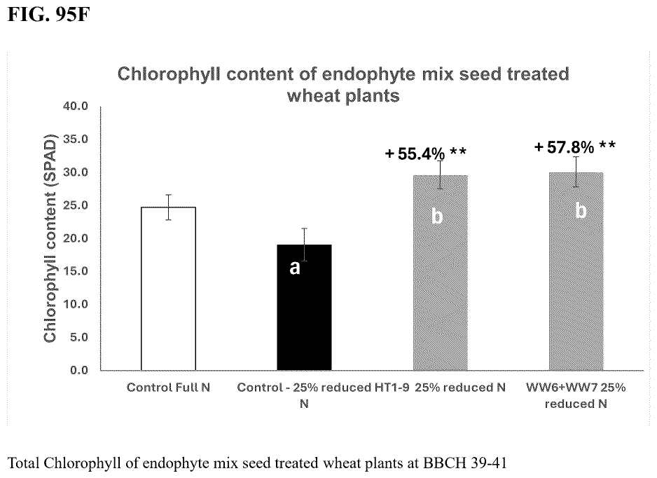

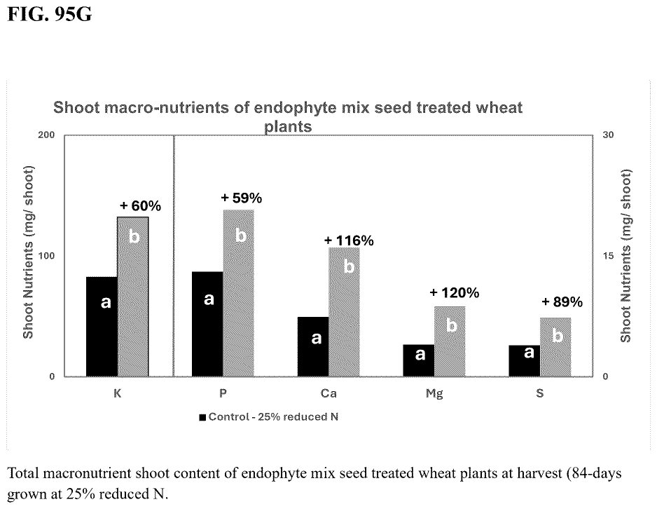

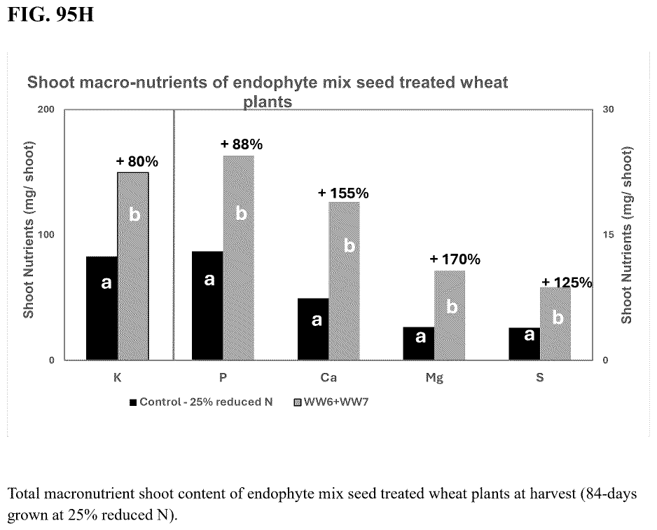

The effects of endophyte treatment on winter wheat plant biomass growth, wheat head grain yield at harvest, chlorophyll and mineral nutrient shoot uptake were tested in a reduced nitrogen conditions after endophyte inoculation. In the experimental groups, HT1-9 strain was applied as a seed treatment for one experimental group and the combination of the strains WW6+WW7 were applied as a seed treatment for a second experimental group. The experimental endophyte seed treatment inoculums were prepared as described in Example 2A herein. Wheat seeds were treated with the inoculum with added 0.5% w/v sodium alginate and applied on seed as described in above methods. The on-seed endophyte survival rates at the time of planting (1 month after drying) were 25 CFU/seed for the HT1-9 strain, 200 CFU/seed for the WW6 strain, and 3,000 CFU/seed for the WW7 strain. Endophytes were applied to wheat seeds using commercial rates germinated and grown with low-N (25% N reduction) in comparison to untreated low-N uninoculated control seeds (25% N reduction). The full-N (100% N optimal) control plant set was included as a positive comparison and reference to show a normal nitrogen biomass growth response of the common winter wheat variety used in conventional agriculture. The experimental groups for the different seed treatments are summarized in FIG. 95A.

An environmental simulated field study was conducted to replicate field seasonal winter wheat environmental and soil conditions. Agricultural EU Cambisol soil from Lower Austria was investigated for commonality and a low nitrogen content then diluted using nutrient-limited peat substrate in the ratio 1:3:1. 1 part cambisol soil, 3 parts steamed standardized nutrient-poor peat substrate (Einheitserde Type-0), and 1 part perlite. The fertilization recommendation for this agronomically relevant winter wheat field harvest simulation was 136 N kg/ha, which was used for the full N condition based on the Austrian Agency of health and Food Safety (AGES) nutrient supply calculator for winter wheat production (https://agesnahrstoffe.agrarcommander.at/). The growth was conducted inside controlled plant growth chambers mimicking winter wheat annual field growth conditions, including, atmospheric temperatures and light intensity duration. The conditions were transitioned through all seasonal conditions starting with fall conditions and ending with elevated summer day/night warmer conditions that track the conditions of the applicable latitude. The environmental growth conditions are provided in FIG. 95B.

For reduced N condition, the fertilization corresponded to 84 N kg/ha (it was planned to use 106 N kg/ha, but we stopped the fertilization of the reduced N conditions earlier to accentuate the difference between full N and reduced N conditions. Liquid ammonium nitrate was applied as the N fertilizer. The addition for reduced N condition per pot corresponded to 36 mg ammonium nitrate per week and 288 mg in total per pot (8 times applied). This corresponded to 84 N kg/ha fertilization. The addition of full N conditions (136 N kg/ha) per pot corresponded to 47 mg ammonium nitrate per week and 470 mg in total per pot (10 times applied). A 1/10 Hoagland No. 2 basal salt without nitrogen solution (https://www.neolab.de/en/h-hi-media-hoagland-no.2-basal-salt-mixturew-o-nitrogen-5-1-ts1117-5l) was used to dilute the ammonium nitrate stock. Thus, Hoagland's nutrient solution was added weekly, even if no N was applied in case of reduced N conditions. In addition, watering was done based on optical assessment of soil. The plants were incubated for 84 days in a growth capsule at defined temperature, duration of illumination and humidity (60%) conditions.

Seeds were placed in pots filled with soil with one seed per pot. In total, 20 seeds per conditions were sown, of which 8 were finally repotted. They were incubated at room temperature and natural light/dark cycles until successful germination was visible. Then the seedlings were covered with a thin layer of soil and vernalized for 4 weeks at 4° C. in the dark. After vernalization, the plants and all material from the small pots were placed in 11 cm×11 cm×12 cm pots. For N fertilization, the addition started one week after repotting. The plant developmental stage was determined weekly using the BBCH scale starting 2 weeks after repotting. In addition, the chlorophyll content of the full flag leaf was measured at BBCH 39-41 using a chlorophyll concentration meter (MC-100 apogee) in technical triplicates per plant.

Twelve weeks after planting and germination, the above-ground part of every plant was harvested, dry weight (shoot and heads), number of heads and number of spikelets per heads were measured. The shoot material was dried at 85° C. before measuring the dry weight. For CN and ICP-OES mineral nutrient analyses, the whole aboveground shoot material per plant was ground using a grinding mill (Retsch, MM400). Subsequently, the ground material was dried for at least 4 h at 105° C. and the samples were then cooled down in a desiccator with silica gel. For CN analysis, three randomly chosen replicates per treatment were used and for each an aliquot of the dried and ground material was transferred into tin foil. The CN analysis was performed using standard methods and an Elementar Vario MACRO Cube CNHS analyzer by BOKU University. For ICP-OES mineral nutrient analysis, the same three replicates per treatment as for CN analysis were chosen and 150 mg of dried and ground material were mixed with 2 ml of MilliQ purified water and 4 ml of 65% nitric acid (HNO₃) for acidification. This suspension was heated to 150° C. for 10 min and then cooled down to 70° C. before filling it up to 15 ml using MilliQ purified water. The extract was then measured using ICP-OES Optima 8300 (Perkin Elmer) by BOKU University.

At mature harvest (84-days post germination) the endophyte HT1-9 treated plants and the WW6+WW7 treated plants showed increased dry head weight yield compared to controls. The plants treated with HT1-9 showed a 132% (p≤0.05) increase in dry head weight yield relative to controls. The plants treated with WW6+WW7 showed a 146% (p≤0.05) increase in biomass relative to controls. FIG. 95C provides the dry head weight yield data.

At mature harvest (84-days post germination) the endophyte HT1-9 treated plants and the WW6+WW7 treated plants showed increased shoot dry weight compared to controls. The plants treated with HT1-9 showed a 91% (p≤0.05) increase in shoot dry weight relative to controls. The plants treated with WW6+WW7 showed a 107% (p≤0.05) increase in biomass relative to controls. FIG. 95D provides the shoot dry weight data.

At mature harvest (84-days post germination) the endophyte HT1-9 treated plants and the WW6+WW7 treated plants showed increased nitrogen content in shoot tissue compared to controls. The plants treated with HT1-9 showed a 120% (p≤0.05) increase in nitrogen content in shoot tissue relative to controls. The plants treated with WW6+WW7 showed a 206% (p≤0.05) increase in biomass relative to controls. FIG. 95E provides the shoot nitrogen content data.

The endophyte HT1-9 treated plants and the WW6+WW7 treated plants showed increased leaf chlorophyll content compared to controls. The plants treated with HT1-9 showed a 55.4% (p ≤0.05) increase in leaf chlorophyll content relative to controls. The plants treated with WW6+WW7 showed a 57.8% (p≤0.05) increase in biomass relative to controls. FIG. 95F provides the leaf chlorophyll content data.

At mature harvest (84-days post germination) the endophyte HT1-9 treated plants showed increased macronutrient content in shoot tissue compared to controls. The plants treated with HT1-9 showed increases in macronutrients compared to controls as follows: 60% higher potassium, P 59% higher phosphorus, 116% higher calcium, 120% higher magnesium, and 89% higher sulfur. All results were statistically significant at the p≤0.05 level. FIG. 95G provides the macronutrient content data for the HT1-9-treated strains.

At mature harvest (84-days post germination) the endophyte WW6+WW7 treated plants showed increased macronutrient content in shoot tissue compared to controls. The plants treated with WW6+WW7 showed increases in macronutrients compared to controls as follows: 80% higher potassium, 88% higher phosphorus, 155% higher calcium, 170% higher magnesium, and 125% higher sulfur. All results were statistically significant at the p≤0.05 level. FIG. 95H provides the macronutrient content data for the WW6+WW7-treated strains.

### EXEMPLARY EMBODIMENTS AND IMPLEMENTATIONS

### Composition Embodiments

In some embodiments, the present invention relates to a plant inoculant composition for treating a host plant comprising at least one heterologous endophyte bacterial strain. In some implementations, the at least one heterologous endophyte bacterial strain is selected for an ability to fix atmospheric nitrogen, increase nitrogen uptake in plants, and a media composition containing at least one nutrient additive operable to enhance survival and colonization of the at least one heterologous endophyte bacterial strain in the host plant. In some implementations, the at least one heterologous endophyte bacterial strain is one or more of the following strains HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, TP SK3, TP SK5, TP SN7, PM PF3, PM SK6, PS SN1, M2, M3, M5, WP8, or WPL8-1. In some implementations, the at least one heterologous endophyte bacterial strain is a plurality of heterologous endophyte bacterial strains that includes at least two of HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1. In some implementations, the at least one heterologous endophyte bacterial strain comprises (i) at least two, (iii) at least three, (iii) at least four, (iv) at least five, or (v) at least six independently selected heterologous endophyte bacterial strains from the following strains: HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1. In some implementations, the at least one heterologous endophyte bacterial strain is selected for an ability to fix atmospheric nitrogen. In some implementations, the at least one heterologous endophyte bacterial strain is selected for an ability to solubilize phosphate salts. In some implementations, the at least one heterologous endophyte bacterial strain is selected for its ability to solubilize phosphate compounds present in soil. In some implementations, the at least one heterologous endophyte bacterial strain is selected for the ability to increase solubilization of multiple forms of insoluble phosphorus. In some implementations, the at least one of the plurality of endophyte bacterial strains is selected to solubilize insoluble forms of phosphorus. In some implementations, the at least one heterologous endophyte bacterial strain has a nitrogenase gene subunit Nif H, Nif D, or Nif K. In some implementations, the at least one heterologous endophyte bacterial strain reduces acetylene. In some implementations, the at least one heterologous endophyte bacterial strain comprises a plurality of endophyte bacterial strains selected to fix atmospheric nitrogen. In some implementations, the at least one heterologous endophyte bacterial strain exhibits fixation of atmospheric nitrogen in the host plant treated with the composition when the host plant is planted in nitrogen depleted soil or when the host plant is planted in nitrogen rich soil. In some implementations, the at least one heterologous endophyte bacterial strain produces Fe siderophores. In some implementations, the at least one heterologous endophyte bacterial strain produces Fe siderophores. In some implementations, the at least one heterologous endophyte bacterial strain is tolerant to biocides, wherein the biocides include fungicides, insecticides, nematocides, and herbicides. In some implementations, the at least one heterologous endophyte bacterial strain is tolerant to plant hormones, plant elicitors, mineral micronutrients, and mineral macronutrients. In some implementations, the at least one heterologous endophyte bacterial strain is encapsulated in microbeads incorporated into the inoculant composition. In some implementations, the at least one heterologous endophyte bacterial strain is selected for and used in conjunction with plant-microbial boosters or prebiotics comprising one or more of an osmoprotectants, an amino acid, an antioxidant, a mineral nutrient, a plant hormone or plant growth elicitor, a carbohydrate, and a mucilage gel used either as separate applications or as a mixture applied to the host plant. In some implementations, the at least one heterologous endophyte bacterial strain has genetic machinery to make Fe siderophores. In some implementations, the at least one heterologous endophyte bacterial strain provides antifungal activity in the host plant. In some implementations, the at least one heterologous endophyte bacterial strain increases glutamine synthetase (GS) enzyme activity in planta.

In some implementations, the inoculant composition includes at least one nutrient additive operable to enhance survival and colonization of the at least one heterologous endophyte bacterial strain in the host plant. In some implementations, the inoculant composition increases nitrogen uptake in the host plant. In some implementations, the inoculant composition increases total plant carbon in the host plant colonized thereby. In some implementations, the inoculant composition increases biomass in the host plant treated with the composition. In some implementations, the inoculant composition increases biomass in the host plant treated with the composition. In some implementations, the inoculant composition increases amino acid production in the host plant. In some implementations, the inoculant composition increases acquisition of essential macronutrients and micro-nutrients from the soil and air without substantial disruption of plant nutrient ion stoichiometry of the host plant. In some implementations, the inoculant composition increases leaf chlorophyll in the host plant treated with the inoculant composition. In some implementations, the inoculant composition increases tolerance to abiotic stress in the host plant treated with the inoculant composition. In some implementations, the inoculant composition increases the host plant's tolerance to plant disease. In some implementations, the inoculant composition increases seedling germination, seedling emergence, and seedling biomass weight in the host plant treated with the inoculant composition. In some implementations, the inoculant composition increases total nitrogen accumulation in plant shoots in the host plant. In some implementations, the inoculant composition increases seedling germination, seedling emergence, and seedling biomass weight in the host plant treated with the inoculant composition. In some implementations, the inoculant composition increases root biomass, root branching, root hairs, and/or any root ultra-structure changes in the host plant. In some implementations, the inoculant composition further comprises one or more Rhizobium species. In some implementations, the inoculant composition further comprises one or more Mycorrhizae species. In some implementations, the inoculant composition further comprises the endophytic yeast strain WP1. In some implementations, the inoculant composition further comprises one or more of a fungicide, an insecticide, an herbicide, a biostimulant, a plant growth regulator, a prebiotic, an adjuvant and a fertilizer used either as separate applications or as a mixture applied to the host plant. In some implementations, the inoculant composition further comprises a carrier composition enabling the plant inoculant composition to be applied to seeds. In some implementations, the inoculant composition further comprises a carrier composition enabling the plant inoculant composition to be applied to foliar portions of the host plant. In some implementations, the inoculant composition further comprises a carrier composition enabling the plant inoculant composition to be applied to soil adjacent to the host plant. In some implementations, the inoculant composition is a suspension composition. In some implementations, the inoculant composition is a dry composition. In some implementations, the inoculant composition comprises dry granules composition. In some implementations, the inoculant composition comprises dry granules composition. In some implementations, the inoculant composition comprises a liquid carrier and is operable to be applied as a foliar spray.

### Method Embodiments

In some embodiments, the present invention relates to a method of enhancing a host plant comprising applying an inoculant composition to a tissue or seed of the host plant, wherein the inoculant composition comprises at least one heterologous endophyte bacterial strain. In some implementations, the at least one heterologous endophyte bacterial strain comprises one or more of the following strains HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, TP SK3, TP SK5, TP SN7, PM PF3, PM SK6, PS SN1, M2, M3, M5, WP8, or WPL8-1. In some implementations, the at least one heterologous endophyte bacterial strain comprises a plurality of heterologous endophyte bacterial strains that includes at least two of HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1. In some implementations, the plurality of heterologous endophyte bacterial strains comprises (i) at least two, (iii) at least three, (iii) at least four, (iv) at least five, or (v) at least six independently selected heterologous endophyte bacterial strains from the following strains: HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1. In some implementations, the at least one heterologous endophyte bacterial strain is selected for an ability to fix atmospheric nitrogen. In some implementations, the at least one heterologous endophyte bacterial strain is selected for an ability to solubilize phosphate salts. In some implementations, the at least one heterologous endophyte bacterial strain is selected for its ability to solubilize phosphate compounds present in soil. In some implementations, the at least one heterologous endophyte bacterial strain is selected for the ability to increase solubilization of multiple forms of insoluble phosphorus. In some implementations, the at least one of the plurality of endophyte bacterial strains is selected to solubilize insoluble forms of phosphorus. In some implementations, the at least one heterologous endophyte bacterial strain has a nitrogenase gene subunit Nif H, Nif D, or Nif K. In some implementations, the at least one heterologous endophyte bacterial strain reduces acetylene. In some implementations, the at least one heterologous endophyte bacterial strain comprises a plurality of endophyte bacterial strains selected to fix atmospheric nitrogen. In some implementations, the at least one heterologous endophyte bacterial strain exhibits fixation of atmospheric nitrogen in the host plant treated with the composition when the host plant is planted in nitrogen depleted soil or when the host plant is planted in nitrogen rich soil. In some implementations, the at least one heterologous endophyte bacterial strain produces Fe siderophores. In some implementations, the at least one heterologous endophyte bacterial strain produces Fe siderophores. In some implementations, the at least one heterologous endophyte bacterial strain is tolerant to biocides, wherein the biocides include fungicides, insecticides, nematocides, and herbicides. In some implementations, the at least one heterologous endophyte bacterial strain is tolerant to plant hormones, plant elicitors, mineral micronutrients, and mineral macronutrients. In some implementations, the at least one heterologous endophyte bacterial strain is encapsulated in microbeads incorporated into the inoculant composition. In some implementations, the at least one heterologous endophyte bacterial strain is selected for and used in conjunction with plant-microbial boosters or prebiotics comprising one or more of an osmoprotectants, an amino acid, an antioxidant, a mineral nutrient, a plant hormone or plant growth elicitor, a carbohydrate, and a mucilage gel used either as separate applications or as a mixture applied to the host plant. In some implementations, the at least one heterologous endophyte bacterial strain has genetic machinery to make Fe siderophores. In some implementations, the at least one heterologous endophyte bacterial strain provides antifungal activity in the host plant. In some implementations, the at least one heterologous endophyte bacterial strain increases glutamine synthetase (GS) enzyme activity in planta.

In some implementations, the inoculant composition includes at least one nutrient additive operable to enhance survival and colonization of the at least one heterologous endophyte bacterial strain in the host plant. In some implementations, the inoculant composition increases nitrogen uptake in the host plant. In some implementations, the inoculant composition increases total plant carbon in the host plant colonized thereby. In some implementations, the inoculant composition increases biomass in the host plant treated with the composition. In some implementations, the inoculant composition increases biomass in the host plant treated with the composition. In some implementations, the inoculant composition increases amino acid production in the host plant. In some implementations, the inoculant composition increases acquisition of essential macronutrients and micro-nutrients from the soil and air without substantial disruption of plant nutrient ion stoichiometry of the host plant. In some implementations, the inoculant composition increases leaf chlorophyll in the host plant treated with the inoculant composition. In some implementations, the inoculant composition increases tolerance to abiotic stress in the host plant treated with the inoculant composition. In some implementations, the inoculant composition increases the host plant's tolerance to plant disease. In some implementations, the inoculant composition increases seedling germination, seedling emergence, and seedling biomass weight in the host plant treated with the inoculant composition. In some implementations, the inoculant composition increases total nitrogen accumulation in plant shoots in the host plant. In some implementations, the inoculant composition increases seedling germination, seedling emergence, and seedling biomass weight in the host plant treated with the inoculant composition. In some implementations, the inoculant composition increases root biomass, root branching, root hairs, and/or any root ultra-structure changes in the host plant. In some implementations, the inoculant composition further comprises one or more Rhizobium species. In some implementations, the inoculant composition further comprises one or more Mycorrhizae species. In some implementations, the inoculant composition further comprises the endophytic yeast strain WP1. In some implementations, the inoculant composition further comprises one or more of a fungicide, an insecticide, an herbicide, a biostimulant, a plant growth regulator, a prebiotic, an adjuvant and a fertilizer used either as separate applications or as a mixture applied to the host plant. In some implementations, the inoculant composition further comprises a carrier composition enabling the plant inoculant composition to be applied to seeds. In some implementations, the inoculant composition further comprises a carrier composition enabling the plant inoculant composition to be applied to foliar portions of the host plant. In some implementations, the inoculant composition further comprises a carrier composition enabling the plant inoculant composition to be applied to soil adjacent to the host plant. In some implementations, the inoculant composition is a suspension composition. In some implementations, the inoculant composition is a dry composition. In some implementations, the inoculant composition comprises dry granules composition. In some implementations, the inoculant composition comprises dry granules composition. In some implementations, the inoculant composition comprises a liquid carrier and is operable to be applied as a foliar spray.

In some implementations, the method further comprises selecting the at least one heterologous endophyte bacterial strain for its ability to produce exogenous ammonium (NH₄) in nitrogen limited growth media. In some implementations, the method further comprises selecting the at least one heterologous endophyte bacterial strain for its ability to increase solubilization of multiple forms of insoluble phosphorus in liquid bacterial growth cultures. In some implementations, the method further comprises mixing the composition with an organic or synthesized nutrient or chemical pre-biotic for the purpose of a combined seed treatment, combined foliar or combined in-furrow application. In some implementations, applying the inoculant composition to the tissue or seed of the host plant includes inoculating a dormant seed embryo for a short or long term. In some implementations, at least two of the plurality of heterologous endophyte bacterial strains are co-fermented prior to application to the foliar portions of the host plant and the application of the at least two co-fermented heterologous endophytes provides a greater increase in one of the following characteristics relative to treatment with a single one of the heterologous endophytes: nitrogen uptake, total leaf chlorophyll, glutamine synthetase (GS) enzyme activity in planta, total biomass, total plant carbon, amino acid production, tolerance to abiotic stress in the host plant treated with the inoculant composition, tolerance to plant disease, total nitrogen accumulation in plant shoots, increase root biomass, root branching, root hairs, seedling germination, seedling emergence, and seedling biomass weight.

### Plant Embodiments

In some embodiments, the invention relates to a plant treated with an inoculant composition comprising at least one heterologous endophyte bacterial strain selected for an ability to fix atmospheric nitrogen, wherein the at least one heterologous endophyte bacterial strain is incorporated into tissue of the plant. In some implementations, the at least one heterologous endophyte bacterial strain comprises at least one of HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, TP SK3, TP SK5, TP SN7, PM PF3, PM SK6, PS SN1, M2, M3, M5, WP8, or WPL8-1. In some implementations, the at least one heterologous endophyte bacterial strain comprises is a plurality of heterologous endophyte bacterial strains comprising at least two of HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1. In some implementations, the at least one heterologous endophyte bacterial strain reduces acetylene. In some implementations, the colonization of the at least one heterologous endophyte bacterial strain in the plant results in increased biomass in the plant. In some implementations, at least one heterologous endophyte bacterial strain comprises a plurality of heterologous endophyte bacterial strains selected for the ability to fix atmospheric nitrogen. In some implementations, the at least one heterologous endophyte bacterial strain is selected for an ability to solubilize phosphate salts. In some implementations, the at least one heterologous endophyte bacterial strain is selected for its ability to solubilize phosphate compounds present in soil. In some implementations, the at least one heterologous endophyte bacterial strain is selected for the ability to increase solubilization of multiple forms of insoluble phosphorus. In some implementations, the at least one of the plurality of endophyte bacterial strains is selected to solubilize insoluble forms of phosphorus. In some implementations, the at least one heterologous endophyte bacterial strain has elevated expression of nitrogenase gene subunit Nif H, Nif D, or Nif K. In some implementations, the at least one heterologous endophyte bacterial strain has elevated expression of nitrogenase gene subunit Nif F. In some implementations, the colonization of the at least one heterologous endophyte bacterial strain in the plant results in increased amino acid production in the plant. In some implementations, the at least one heterologous endophyte bacterial strain exhibits fixation of atmospheric nitrogen in the plant when the plant is grown in nitrogen depleted soil or when the plant is grown in nitrogen rich soil. In some implementations, the at least one heterologous endophyte bacterial strain provides antifungal activity in the host plant.

The foregoing descriptions of specific embodiments of the present invention have been presented for purposes of illustration and description. They are not intended to be exhaustive or to limit the invention to the precise forms disclosed, and many modifications and variations are possible considering the above teaching. The embodiments were chosen and described to best explain the principles of the invention and its practical application, to thereby enable others skilled in the art to best utilize the invention and various embodiments with various modifications as are suited to the particular use contemplated.

What is claimed is:

1. A plant inoculant composition for treating a host plant comprising at least one heterologous endophyte bacterial strain, wherein said at least one heterologous endophyte bacterial strain is selected from the group consisting of HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, TP SK3, TP SK5, TP SN7, PM PF3, PM SK6, PS SN1, M2, M3, M5, WP8 and WPL8-1.

2. The composition of claim 1, comprising a plurality of heterologous endophyte bacterial strains, each independently selected from the group consisting of: HT1-2, HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, WW5, WW6, WW7, PTD1, TP SK3, TP SK5, TP SN7, PM PF3, PM SK6, PS SN1, M2, M3, M5, WP1, WP8 and WPL8-1.

3. The composition of claim 2, wherein said plurality of heterologous endophyte bacterial strains comprises (i) at least two, (iii) at least three, (iii) at least four, (iv) at least five, or (v) at least six independently selected heterologous endophyte bacterial strains from the group of strains defined in claim 2.

4. The composition of claim 2, wherein said plurality of heterologous endophyte bacterial strains comprises one of the following combinations of strains:
a. HT1-9+WP1+PTD1
b. SD1+HT1-10+PTD1
c. SD2+11RB1+11RB2
d. HT1-9+HT1-2
e. HT1-9+11R-BB
f. HT1-9+11R-BC
g. HT1-9+11R-B1+11R-B2
h. SD2+HT1-2
i. HT1-9+HT1-10
j. HT1-9+SD1
k. PTD1+WP5
l. HT1-9+WP5
m. HT1-9+PTD1
n. HT1-9+R10
o. WW5+WP5
p. PTD1+HT1-10
q. PTD1+HT1-9
r. SD2+11RBC
S. SD2+11RBB
t. SD2+HT1-9
u. SD1+SD2
v. SD1+PTD1,
w. WW7+TPSK5
x. HT1-10+HT1-8
y. HT1-9+HT1-8
z. R10+WP5
aa. WP5+WP1
bb. AWS1+WW5.
cc. 11R-B1+11R-B2

5. The composition of claim 2, wherein the plurality of heterologous endophyte bacterial strains comprises at least two independently selected heterologous endophyte bacterial strains and said two strains provide for a further enhancement of nutrient uptake and/or yield in host plants.

6. The composition claim 1, wherein at least one heterologous endophyte bacterial strain has the ability to fix and incorporate atmospheric nitrogen

7. The composition claim 1, wherein at least one heterologous endophyte bacterial strain has the ability to produce exogenous ammonium (NH₄⁺).

8. The composition of claim 1, wherein at least one heterologous endophyte bacterial strain has the ability to mobilize insoluble phosphate salts in soil.

9. The composition of claim 1, further comprising one or more additional constituent selected from the group consisting of a fungicide, an insecticide, an herbicide, a biostimulant, a plant growth regulator, an adjuvant, or a fertilizer.

10. The composition of claim 1, additionally comprising an agrochemically acceptable diluent or carrier.

11. The composition of claim 1, wherein at least one heterologous endophyte bacterial strain is encapsulated.

12. The composition of claim 1, wherein composition is in liquid form.

13. The composition of claim 12, wherein the liquid formulation comprises fermentation broth from the manufacture of said at least one heterologous endophyte bacterial strain, or from the manufacture of each of said heterologous endophyte strain(s).

14. The composition of claim 1, wherein said inoculant composition comprises said at least one heterologous endophyte bacterial strain at a concentration from 10³ CFU/mL to 10¹⁰ CFU/mL, from 10⁴ CFU/mL to 10⁹ CFU/mL, from 10⁵ CFU/mL to 10⁸ CFU/mL. from 10⁶ CFU/mL to 10⁹ CFU/mL. from 10⁷ CFU/mL to 10⁹ CFU/mL. from 10⁸ CFU/mL to 10⁹ CFU/mL, 10⁷ CFU/mL, 10⁸ CFU/mL, or 10⁹ CFU/mL.

15. The composition of claim 2, wherein each heterologous endophyte strain in said inoculant is present at a concentration of about 10³ CFU/mL to 10¹⁰ CFU/mL, from 10⁴ CFU/mL to 10⁹ CFU/mL, from 10⁵ CFU/mL to 10⁸ CFU/mL. from 10⁶ CFU/mL to 10⁹ CFU/mL. from 10⁷ CFU/mL to 10⁹ CFU/mL. from 10⁸ CFU/mL to 10⁹ CFU/mL, 10⁷ CFU/mL, 10⁸ CFU/mL, or 10⁹ CFU/mL.

16. The composition of claim 1, further comprising at least one nutrient additive that enhances survival and colonization of the at least one heterologous endophyte bacterial strain, comprising one or more of an organic or synthesized bio-stimulant, a fatty acid, a carbohydrate, an amino acid, or a mineral nutrient.

17. A method of enhancing nutrient uptake in a host plant, said method comprising applying to the foliage or seed of said host plant, or to the locus of said plant, or to the locus of said seed is to be sown, a composition as defined in claim 1.

18. A method of increasing biomass in a host plant, said method comprising applying to the foliage or seed of said host plant, or to the locus of said plant, or to the locus of said seed is to be sown, a composition as defined in claim 1.

19. A plant treated with a composition as defined in claim 1, wherein at least one heterologous endophyte bacterial strain from said composition is incorporated into tissue of said plant.

20. A method of manufacturing a plant inoculant composition for enhancing a host plant, comprising formulating at least one heterologous endophyte bacterial strain as defined in claim 1 with a carrier.

21. The method of claim 20, comprising fermenting at least one heterologous endophyte bacterial strain.

22. The method of claim 20, comprising co-fermenting (i) at least two, (iii) at least three, (iii) at least four, (iv) at least five, or (v) at least six independently selected heterologous endophyte bacterial strains selected from HT1-4, HT1-7, HT1-8, HT1-9, HT1-10, SD1, SD2, R10, 11R-B1, 11R-B2, 11R-BB, 11R-BC, AWS1, WP5, WP4-10-4, WP4-4-2, WP4-5-3, WP4-3-3, WP4-4-6, WP4-3-1, TP SK3, TP SK5, TP SN7, PM PF3, PM SK6, PS SN1, M2, M3, M5, WP8 and WPL8-1.


SEQUENCE LISTING (37 sequences)

SEQ ID NO: 1 - RNA, 816 nt, Azotobacter beijerinckii
  source 1..816: mol_type = rRNA
accgtccccccgaaggttagactagctacttctggagcaacccactcccatggtgtgacgggcggtgtgtacaaggcccgggaacgtattcaccgcgacattctgattcgcgattactagcgattccgacttcacgcagtcgagttgcagactgcgatccggactacgatcggttttctgggattagctccgcctcgcgacttggcaaccctctgtaccgaccattgtagcacgtgtgtagccctggccgtaagggccatgatgacttgacgtcatccccaccttcctccggtttgtcaccggcagtctccttagagtgcccaccatgacgtgctggtaactaaggacaagggttgcgctcgttacgggacttaacccaacatctcacgacacgagctgacgacagccatgcagcacctgtctctgcgctcccgaaggcacccaggtatctctacccagttcgcaggatgtcaaggccaggtaaggttcttcgcgttgcttcgaattaaaccacatgctccaccgcttgtgcgggcccccgtcaattcatttgagttttaaccttgcggccgtactccccaggcggtcgacttaatgcgttagctgcgccactaagctctcaaggagcccaacggctagtcgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccgtactctagtc

SEQ ID NO: 2 - RNA, 808 nt, Azospirillum palustre
  source 1..808: mol_type = rRNA
tgcaagtcgaacgaaggcttcggccttagtggcgcacgggtgagtaacacgtgggaacctgcctttcggttcggaataacgtttggaaacgaacgctaacaccggatacgccctttgggggaaagtttacgccgagagaggggcccgcgtcggattaggtagttggtgaggtaatggctcaccaagccttcgatccgtagctggtctgagaggatgatcagccacactgggactgagacacggcccagactcctacgggaggcagcagtggggaatattggacaatgggcgcaagcctgatccagcaatgccgcgtgagtgatgaaggccttagggttgtaaagctctttcgcacgcgacgatgatgacggtagcgtgagaagaagccccggctaacttcgtgccagcagccgcggtaatacgaagggggctagcgttgttcggaattactgggcgtaaagggcgcgtaggcggcctgtttagtcagaagtgaaagccccgggctcaacctgggaatagcttttgatactggcaggcttgagttccggagaggatggtggaattcccagtgtagaggtgaaattcgtagatattgggaagaacaccggtggcgaangcggccatctggacggacactgacgctgaggcgcgaaagcgtggggagcaaacaggattagataccctggtagtccacgccgtaaacgatgaatgctagacgtcggggtgcatgcacttcggtgtcgccgctaacgcattaagcattccgcctggggagtacggccgcaaggttaaaactcaaaggaattga

SEQ ID NO: 3 - RNA, 820 nt, Herbiconiux solani
  source 1..820: mol_type = rRNA
gctttcatgattgncgccgntgtgtanaagccccggcnntattcactggggcgttgtgatccantttaatagcgnncngtttcagggggtgnnttggaagtctaaatgggacaggngcggntttttgnttgctnntactttaggnattggancttttggacggatgttttgnttnnngagacaactaatannntcatgangaatnatgtcncaccacantcgtccgngttgaccccggcgtntttttatttcttcccccataatgtgctgnaacataggngangnntgcgctngttgatnnattaanccacnatctcacganacgantgagggcaaccatgcnccacctggttaccanaaacaaaganncgaccntttctgtccngttctcgtttangtcaancttggtatnttcttcgcgttggcattgaattaatccgcctggccccgtcggtttgggcccnnccccccgttcaatttcctttttgagtttttagcccttggcgncccctacctccccccagggcggggtaactttaattgtctttttaccctgccgacaccgaagacccgtggnnnngattcccccaacaatctaannnccccaaacggtttacgncgtagnaccaaccaaannaaaattcttaatccctgnttcgcctccccnnacgcnttttccggttccntaaaggtagtagagntaaggggcccaaggacactccnggcctcttgcggcccatccggtngntgactattncttgggnntantttggncgccattttcccaaccgggngaacaacnncaaagnaaattnntcca

SEQ ID NO: 4 - RNA, 1331 nt, Sphingobium sp.
  source 1..1331: mol_type = rRNA
ggtcgcctgcctccttacggttagctcaacgccttcgagtgaatccaactcccatggtgtgacgggcggtgtgtacaaggcctgggaacgtattcaccgcggcatgctgatccgcgattactagcgattccgccttcacgctctcgagttgcagagaacgatccgaactgagacgacttttggagattagctccctctcgcgaggtggctgcccactgtagtcgccattgtagcacgtgtgtagcccaacgcgtaagggccatgaggacttgacgtcatccccaccttcctccggcttatcaccggcggttcctttagagtacccaactaaatgctggcaactaaaggcgagggttgcgctcgttgcgggacttaacccaacatctcacgacacgagctgacgacagccatgcagcacctgtcacctatccagccgaactgaaggaaagtgtctccacgatccgcgatagggatgtcaaacgttggtaaggttctgcgcgttgcttcgaattaaaccacatgctccaccgcttgtgcaggcccccgtcaattcctttgagttttaatcttgcgaccgtactccccaggcggataacttaatgcgttagctgcgccaccaaaacaccatgtgccctgacagctagttatcatcgtttacggcgtggactaccagggtatctaatcctgtttgctccccacgctttcgcacctcagcgtcaataccagtccagtgagccgccttcgccactggtgttcttccgaatatctacgaatttcacctctacactcggaattccactcacctctcctggattcaagctatctagtttcaaaggcagttccggggttgagccccgggctttcacctctgacttgaatagccgcctacgtgcgctttacgcccagtaattccgaacaacgctagctccctccgtattaccgcggctgctggcacggagttagccggagcttattctcccggtactgtcattatcatcccgggtaaaagagctttacaaccctaaggccttcatcactcacgcggcattgctggatcaggctttcgcccattgtccaatattccctactgctgcctcccgtaggagtctgggccgtgtctcagtcccagtgtggctgatcatcctctcagaccagctaaggatcgtcgccttggtgagcctttaccccaccaactagctaatcctacgcgggctcatccttgggcgataaatctttggacttacgtcatcatccggtattagcttccgtttccagaagttattccgaacccaagggcagattcccacgcgttacgcacccgtgcgccacta

SEQ ID NO: 5 - RNA, 1362 nt, Curtobacterium oceanosedimentum
  source 1..1362: mol_type = rRNA
atgatcaggagcttgctcctgtgattagtggcgaacgggtgagtaacacgtgagtaacctgcccctgactctgggataagcgttggaaacgacgtctaatactggatatgatcactggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaaccct

SEQ ID NO: 6 - RNA, 1393 nt, Curtobacterium sp.
  source 1..1393: mol_type = rRNA
gcaagtcgaacgatgatgcccagcttgctgggtggattagtggcgaacgggtgagtaacacgtgagtaacctgcccctgactctgggataagcgttggaaacgacgtctaatactggatacgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgnggaaggagccgtcgaa

SEQ ID NO: 7 - RNA, 1350 nt, Rhizobium wenxiniae
  source 1..1350: mol_type = rRNA
tgcaagtcgagcgccccgcaaggggagcggcagacgggtgagtaacacgtgggaatctacccatccctgcggaacaactccgggaaactggagctaataccgcatacgcccttcgggggaaagatttatcggggatggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccttagggttgtaaagctctttcaccgatgaagataatgacggtagtcggagaagaagccccggctaacttcgtgccagcagccgcggtaatacgaagggggctagcgttgttcggaattactgggcgtaaagcgcacgtaggcggatatttaagtcaggggtgaaatcccgcagctcaactgcggaactgcctttgatactgggtatcttgagtatggaagaggttagtggaattgcgagtgtagaggtgaaattcgtagatattcgcaggaacaccagtggcgaaggcggctaactggtccataactgacgctgaggtgcgaaagcgtggggagcaaacaggattagataccctggtagtccacgccgtaaacgatgaatgttagccgtcggcaagtttacttgtcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatgtccggctcaccacagagatgtggttttcccttcggggaccggaacacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgaaggagcgatcccgagctaatctccaaaagccatctcagttcggattgcactctgcaactcgagtgcatgaagttggaatcgctagtaatcgcggatcagcacgccgcggtgaatacgttcccgggccttgtacacaccgcccgtcacaccatgggagttggttttacccgaaggtagtgcgctaaccgcaaggaggcagctaac

SEQ ID NO: 8 - RNA, 1349 nt, Azorhizobium sp.
  source 1..1349: mol_type = rRNA
aagtcgagcgcccagcaatgggagcggcagacgggtgagtaacacgtggggatgtgcccaaaggtacggaatagctccgggaaactggaattaataccgtatgtgcccttcgggggaaagatttatcgcctttggatcaacccgcgtctgattagctagttggtgaggtaaaggctcaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctctttcgccggtgaagataatgacggtaaccggagaagaagccccggctaacttcgtgccagcagccgcggtaatacgaagggggctagcgttgctcggaatcactgggcgtaaagcgcacgtaggcgggtcgttaagtcaggggtgaaatcctggagctcaactccagaactgcccttgatactggcgatcttgagttcgagagaggttggtggaactgcgagtgtagaggtgaaattcgtagatattcgcaagaacaccagtggcgaaggcggccaactggctcgatactgacgctgaggtgcgaaagcgtggggagcaaacaggattagataccctggtagtccacgccgtaaacgatggatgctagccgttaggcagcttgctgcttagtggcgcagctaacgcattaagcatcccgcctggggagtacggtcgcaagattaaaactcaaaggaattgacgggggcccgcacaagcggtggagcatgtggtttaattcgaagcaacgcgcagaaccttaccagcctttgacatgtcaggacgacttccggagacggatttcttccagcaatggacctgaacacaggtgctgcatggctgtcgtcagctcgtgtcgtgagatgttgggttaagtcccgcaacgagcgcaaccctcgcctttagttgccatcattcagttgggcactctaaagggactgccggtgataagccgcgaggaaggtggggatgacgtcaagtcctcatggcccttacgggctgggctacacacgtgctacaatggtggtgacagtgggacgcaagctcgcgagagcaagcaaatctccaaaagccatctcagttcggattgcactctgcaactcgagtgcatgaagttggaatcgctagtaatcgtggatcagcacgccacggtgaatacgttcccgggccttgtacacaccgcccgtcacaccatgggagttggttttacccgaaggcgttgcgctaacccgcaagggaggcaggcg

SEQ ID NO: 9 - RNA, 1346 nt, Rhizobium sp.
  source 1..1346: mol_type = rRNA
tgcaagtcgaacgccccgcaaggggagtggcagacgggtgagtaacgcgtgggaatctaccctttactacggaataacgcatggaaacgtgtgctaataccgtatgtgtccttcgggagaaagatttatcggtaaaggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggagaagataatgacggtatccggagaagaagccccggctaacttcgtgccagcagccgcggtaatacgaagggggctagcgttgttcggatttactgggcgtaaagcgcacgtaggcggatcgatcagtcaggggtgaaatcccagagctcaactctggaactgcctttgatactgtcgatctggagtatggaagaggtgagtggaattccgagtgtagaggtgaaattcgtagatattcggaggaacaccagtggcgaaaggcggctcactggtccattactgacgctgaggtgcgaaagcgtggggagcaaacaggattagataccctggtagtccacgccgtaacgatgaatgttagccgtcgggcagtatactgttcggtggcgcagctaacgcattaaacattccgcctggggagtacgntcgcaagattaaaactcaaaggaattgacgggggcccgcacaagcggtggagcatgtggtttaattngaagcaacgcgcaganccttaccagcccttgacatgtccggctatccagagagatttggagttcccttcggggaccggagcacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgaacgggcgaccgtgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggttttacccgaaggtagtgcgctaaccgcaaggaggcagc

SEQ ID NO: 10 - RNA, 1016 nt, Rahnella aceris
  source 1..1016: mol_type = rRNA
tgcagtcgagcggcagcggaaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgangcgtggcttccggagctaacgcgttaagtcgaccgcctggggagtacggccgcaaggttaaaactcaaatgaattgacgggggcccgcacaagcggtggagcatgtggtttaattcgatgcaacgcgaagaancttacctactcttgacatccacgggaattcgccaganatggcttagtgccttcgggaaccgtgagacaggngctgcatggctgtcgtcagctc

SEQ ID NO: 11 - RNA, 1381 nt, Priestia sp.
  source 1..1381: mol_type = rRNA
gcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacaagagtaactgcttgtaccttgacggtacctaaccagaaagccacggctaactacgtgccagcagccgcggtaatacgtaggtggcaagcgttatccggaattattgggcgtaaagcgcgcgcaggcggtttcttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgt

SEQ ID NO: 12 - RNA, 1353 nt, Pseudomonas sp.
  source 1..1353: mol_type = rRNA
gcttgctcctcgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtggggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggccgttacctaatacgtgatggttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtagggcagagggtggtggaatttcctgtgtagcggtgaaatgcgtagatataggaaggaacaccagtggcgaaggcgaccacctgggctcatactgacactgaggtgcgaaagcgtggggagcaaacaggattagattaccctggtagtccacgccgtaaacgatgtcaactagccgtttggaatccttgagattttagtggcgcagctaacgcattaagttgaccgcctggggagtacggccgcaaggtttaaaactcaaatganttgacgggggcccgcacaagcggtggagcatgtggtttaattcgaagcaacgcgaagaaccttaccaggccttgacatgcagagaactttccagagatggataggtgccttcgggaactctgacacaggtgctgcatggctgtcgtcagctcgtgtcgtgagatgttgggttaagtcccgtaacgagcgcaacccttgtccttagttaccagcacgtgatggtgggcactctaaggagactgccggtgacaaaccggaggaaggtggggatgacgtcaagtcatcatggcccttacggcctgggctacacacgtgctacaatggtcggtacagagggtcgccaagccgcgaggtggagctaatctcacaaaaccgatcgtagtccggatcgcagtctgcaactcgactgcgtgaagtcggaatcgctagtaatcgcgaatcagaacgtcgcggtgaatacgttcccgggccttgtacacaccgcccgtcacaccatgggagtgggttgcaccaga

SEQ ID NO: 13 - RNA, 1384 nt, Erwinia sp.
  source 1..1384: mol_type = rRNA
acggtagcacagaggagcttgctccttgggtgacgagtggcggacgggtgagtaatgtctggggatctgcccgatggagggggataaccactggaaacggtggctaataccgcataacgtcgcaagaccaaagtgggggaccttcgggcctcacaccatcggatgaacccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggggacgaggttaataacctcgttcattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggctgtgagcatgactcgtgggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctgctcttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagcagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaac

SEQ ID NO: 14 - RNA, 1385 nt, Pseudomonas sp.
  source 1..1385: mol_type = rRNA
tgcagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtcta

SEQ ID NO: 15 - RNA, 1395 nt, Paraburkholderia sp.
  source 1..1395: mol_type = rRNA
gtcctccttgcnggtatagactagccacttctggtaaaacccactcccatggtgtgacgggcggtgtgtacaagacccgggaacgtattcaccgcggcatgctgatccgcgattactagcgattccagcttcacgcactcgagttgcagagtgcgatccggactacgatcggttttctgggattggctccccctcgcgggttggcgaccctctgttccgaccattgtatgacgtgtgaagccctacccataagggccatgaggacttgacgtcatccccaccttcctccggtttgtcaccggcagtctccctggagtgctcttgcgtagcaactagggacaagggttgcgctcgttgcgggacttaacccaacatctcacgacacgagctgacgacagccatgcagcacctgtgttatggctccctttcgggcacgtccacctctcagcggacttccatacatgtcaagggtaggtaaggtttttcgcgttgcatcgaattaatccacatcatccaccgcttgtgcgggtccccgtcaattcctttgagttttaatcttgcgaccgtactccccaggcggtcaacttcacgcgttagctacgttaccaagtcaatgaagacccgacaactagttgacatcgtttagggcgtggactaccagggtatctaatcctgtttgctccccacgctttcgtgcatgagcgtcagtattggcccagggggctgccttcgccatcggtattcctccacatctctacgcatttcactgctacacgtggaattctacccccctctgccatactctagcccgccagtcacaaatgcagttcccaggttaagcccggggatttcacatctgtcttagcgaaccgcctgcgcacgctttacgcccagtaattccgattaacgcttgcaccctacgtattaccgcggctgctggcacgtagttagccggtgcttattcttccggtaccgtcatccccccgccatattagggcagaggatttctttccggacaaaagtgctttacaacccgaaggccttcttcacacacgcggcattgctggatcagggttgcccccattgtccaaaattccccactgctgcctcccgtaggagtctgggccgtgtctcagtcccagtgtggctggtcgtcctctcagaccagctacagatcgtcgccttggtaggcctttaccccaccaactagctaatctgccatcggccgcccctgtagcgcgaggtcctaagatcccccgctttcctccgcagagcgtatgcggtattaatccggctttcgccgggctatcccccactacaggacacgttccgatgtattactcacccgttcgccactcgccaccagggttgcccccgtgctgccgttcgactgca

SEQ ID NO: 16 - RNA, 1396 nt, Herbaspirillum sp.
  source 1..1396: mol_type = rRNA
tgcagtcgnncggcagcataggagcttgctcctgatggcgagtggcgaacgggtgagtaatatatcggaacgtgccctagagtgggggataactagtcgaaagactagctaataccgcatacgatctacggatgaaagtgggggatcgcaagacctcatgctcctggagcggccgatatctgattagctagttggtggggtaaaagctcaccaaggcgacgatcagtagctggtctgagaggacgaccagccacactgggactgagacacggcccagactcctacgggaggcagcagtggggaattttggacaatgggggcaaccctgatccagcaatgccgcgtgagtgaagaaggccttcgggttgtaaagctcttttgtcagggaagaaacggttctggataatacctggagctaatgacggtacctgaagaataagcaccggctaactacgtgccagcagccgcggtaatacgtagggtgcaagcgttaatcggaattactgggcgtaaagcgtgcgcaggcggttgtgtaagtcagatgtgaaatccccgggctcaacctgggaattgcatttgagactgcacggctagagtgtgtcagaggggggtagaattccacgtgtagcagtgaaatgcgtagatatgtggaggaataccgatggcgaaggcagccccctgggataacactgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatggatggaatcccgaagagatttgggagtgctcgaaagagaaccatcacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggattggagtctgcaactcgactccatgaagttggaatcgctagtaatcgcggatcagcatgtcgcggtgaatacgttcccgggtcttgtacacaccgcccgtcacaccatgggagcgggttttaccagaagtgggtagcctaaccgcaaggagggcg

SEQ ID NO: 17 - RNA, 1383 nt, Paraburkholderia sp.
  source 1..1383: mol_type = rRNA
tgcagtcgaacggcagcacgggggcaaccctggtggcgagtggcgaacgggtgagtaatacatcggaacgtgtcctgtagtgggggatagcccggcgaaagccggattaataccgcatacgctctgcggaggaaagcgggggatcttaggacctcgcgctacaggggcggccgatggcagattagctagttggtggggtaaaggcctaccaaggcgacgatctgtagctggtctgagaggacgaccagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatcctctgccctaatatggcggggggatgacggtaccggaagaataagcaccggctaactacgtgccagcagccgcggtaatacgtagggtgcaagcgttaatcggaattactgggcgtaaagcgtgcgcaggcggttcgctaagacagatgtgaaatccccgggcttaacctgggaactgcatttgtgactggcgggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgtcgggtcttcattgacttggtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtatggaagtccgctgagaggtggatgtgcccgaaagggagccataacacaggtgctgcatggctgtcgtcagctcgtgtcgtgagatgttgggttaagtcccgcaacgagcgcaacccttgtccctagttgctacgcaagagcactccagggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggatcgcactctgcaactcgagtgcgtgaagctggaatcgctagtaatcgcggatcagcatgccgcggtgaatacgttcccgggtcttgtacacaccgcccgtcacaccatgggagtgggttttaccagaagtggctagtctatacc

SEQ ID NO: 18 - RNA, 1391 nt, Rahnella sp.
  source 1..1391: mol_type = rRNA
tgcagtcgagcggtagcacanggagagcttgctctctgggtgacgagcggcggacgggtgagtaatgtctgggaaactgcctgatggagggggataactactggaaacggtagctaataccgcatgacctcgcaagagcaaagtgggggaccttcgggcctcacgccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaagggttcagtgttaatagcactgtacattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcaaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagctt

SEQ ID NO: 19 - RNA, 1402 nt, Rahnella sp.
  source 1..1402: mol_type = rRNA
gcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacatactttatgaggtccgcttgctctcgcgagtttgcttctctttgtatatgccattgtagcacgtgtgtagccctactcgtaagggccatgatgacttgacgtcatccccaccttcctccggtttatcaccggcagtctcctttgagttcccaccattacgtgctggcaacaaaggataagggttgcgctcgttgcgggacttaacccaacatttcacaacacgagctgacgacagccatgcagcacctgtctcacggttcccgaaggcactaagctatctctagcgaattccgtggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacagcgtggactaccagggtatctaatcctgtttgctccccacgctttcgcacctgagcgtcagtctttgtccagggggccgccttcgccaccggtattcctccagatctctacgcatttcaccgctacacctggaattctacccccctctacaagactctagcttgccagtttcaaatgcagttcccaagttaagctcggggatttcacatctgacttaacaaaccgcctgcgtgcgctttacgcccagtaattccgattaacgcttgcaccctccgtattaccgcggctgctggcacggagttagccggtgcttcttctgcgagtaacgtcaatcgctgcagctattaactacaacgccttcctcctcgctgaaagtgctttacaaccctaaggccttcttcacacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattaccccacctactagctaatcccatctgggcacatccgatggcgtgaggcccgaaggtcccccactttgctcttgcgaggtcatgcggtattagctaccgtttccagtagttatccccctccatcaggcagtttcccagacattactcacccgtccgccgctcgtcacccagagagcaagctcttcccgtgctaccgctcg

SEQ ID NO: 20 - RNA, 1349 nt, Paraburkholderia sp.
  source 1..1349: mol_type = rRNA
cctggtggcgagtggcgaacgggtgagtaatacatcggaacgtgtcctgtagtgggggatagcccggcgaaagccggattaataccgcatacgctctgcggaggaaagcgggggatccttcgggacctcgcgctacaggggcggccgatggcagattagctagttggtggggtaaaggcctaccaaggcgacgatctgtagctggtctgagaggacgaccagccacactgggactgagacacggcccagactcctacgggaggcagcagtggggaattttggacaatgggcgcaagcctgatccagcaatgccgcgtgtgtgaagaaggccttcgggttgtaaagcacttttgtccggaaagaaaacctcgtggttaatacccgtgggggatgacggtaccggaagaataagcaccggctaactacgtgccagcagccgcggtaatacgtagggtgcaagcgttaatcggaattactgggcgtaaagcgtgcgcaggcggttcgctaagacagatgtgaaatccccgggcttaacctgggaactgcatttgtgactggcgggctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactagttgtcgggtcttcattgacttggtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtatggaaccctgctgagaggtgggggtgcccgaaagggagccataacacaggtgctgcatggctgtcgtcagctcgtgtcgtgagatgttgggttaagtcccgcaacgagcgcaacccttgtccctagttgctacgcaagagcactccagggagactgccggtgacaaaccggaggaaggtggggatgacgtcaagtcctcatggcccttatgggtagggcttcacacgtcatacaatggtcggaacagagggtcgccaacccgcgagggggagccaatcccagaaaaccgatcgtagtccggatcgcactctgcaactcgagtgcgtgaagctggaatcgctagtaatcgcggatcagcatgccgcggtgaatacgttcccgggtcttgtacacaccgcccgtcacaccatgggagtgggtttcaccagaagtaggtat

SEQ ID NO: 21 - RNA, 965 nt, Rahnella sp.
  source 1..965: mol_type = rRNA
tgcagtcgagcggcagccggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaatcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcccagagatggctta

SEQ ID NO: 22 - RNA, 1076 nt, Rahnella aquatilis
  source 1..1076: mol_type = rRNA
tgcagtcgagcggcagcggaaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtgagacaggtgctgcatggctgtcgtcagctcgtgttgtgaaatgttgggttaagtcccgcaacgagcgcaacccttatcctttgttgccagc

SEQ ID NO: 23 - RNA, 962 nt, Curtobacterium sp.
  source 1..962: mol_type = rRNA
gacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcatggggagcgaacaggattagataccctggtagtccatgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggt

SEQ ID NO: 24 - RNA, 1032 nt, Pseudomonas lutea
  source 1..1032: mol_type = rRNA
gtcgagcggatgaagagagcttgctctctgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgttacctaatacgtgacaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcatccaaaactggcaagctagagtagggcagagggtggtggaatttcctgtgtagcggtgaaatgcgtagatataggaaggaacaccagtggcgaaggcgaccacctgggctcatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccagtgaacttaccagagatggtttggtgccttcgggaacactgagacaggtgctgcatggctgtcgtcagctcgtgtcgtgagatgttgggttaagtccc

SEQ ID NO: 25 - RNA, 554 nt, Rhodotorula graminis
  source 1..554: mol_type = rRNA
gcggcgagcgaagcgggaagagctcaaatttataatctggcaccttcggtgtccgagttgtaatctctagaagtgttttccgcgttggaccgcacacaagtctgttggaatacagcggcatagtggtgaaacccccgtatatggtgcggacgcccagcgctttgtgatacactttcaatgagtcgagttgtttgggaatgcagctcaaattgggtggtaaattccatctaaagctaaatattggcgagagaccgatagcgaacaagtaccgtgagggaaagatgaaaagcactttggaaagagagttaacagtacgtgaaattgttggaagggaaacgcttgaagtcagacttgcttgccggagcttgcttcggtttgcaggccagcatcagttttccggggtggataatgacggtttgaaggtagcagtctcggctgtgttatagctttccgttggatacatcctgggggactgaggaacgcagcgtgctttttgcgaaagactcgtctttttcacgcttaggatgctggtggaatggctttaaacgacccgt

SEQ ID NO: 26 - RNA, 859 nt, Curtobacterium sp.
  source 1..859: mol_type = rRNA
gggcagactagagtgcggtaggggagattggaattcctggtgtagcggtggaatgcgcagatatcaggaggaacaccgatggcgaaggcagatctctgggccgtaactgacgctgaggagcgaaagcatggggagcgaacaggattagataccctggtagtccatgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgggttatgccggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgta

SEQ ID NO: 27 - RNA, 1062 nt, Pseudomonas siliginis
  source 1..1062: mol_type = rRNA
aagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagttcggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggacggttaccacggtgtgattcatgactggggtgaagtcgtaacaaggtaaccg

SEQ ID NO: 28 - RNA, 926 nt, Curtobacterium salicaceae
  source 1..926: mol_type = rRNA
gcccagcttgctgggttgattagtggcgaacgggtgagtaacacgtgagtaacctgcccctgactctgggataagcgttggaaacgacgtctaatactggatatgatcactggtcgcatggcctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcatggggagcgaacaggattagataccctggtagtccatgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatgg

SEQ ID NO: 29 - RNA, 1302 nt, Rhizobium populi
  source 1..1302: mol_type = rRNA
cgagcgccncgcaaggggagcggcagaccggtgagtaacgcgtgggaatctaccttttgctacggaataacgcagggaaacttgtgctaataccgtatgtgtccttcgggagaaagatttatcggcaagagatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggagaagataatgacggtatccggagaagaagccccggctaacttcgtgccagcagccgcggtaatacgaagggggctagcgttgttcggaattactgggcgtaaagcgcacgtaggcggatcgatcagtcaggggtgaaatcccagggctcaaccctggaactgcctttgatactgtcgatctggagtatggaagaggtgagtggaattccgagtgtagaggtgaaattcgtagatattcggaggaacaccagtggcgaaggcggctcactggtccattactgacgctgaggtgcgaaagcgtggggagcaaacaggattagataccctggtagtccacgccgtaaacgatgaatgttagccgtcgggcagtatactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcctgtgttacctctagagatagggggtccacttcggtggcgcagagacaggtgctgcatggctgtcgtcagctcgtgtcgtgagatgttgggttaagtcccgcaacgagcgcaaccctcgcccttagttgccagcatttagttgggcactctaaggggactgccggtgataagccgagaggaaggtggggatgacgtcaagtcctcatggcccttacgggctgggctacacacgtgctacaatggtggtgacagtgggcagcgagcacgcgagtgtgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggtt

SEQ ID NO: 30 - RNA, 1417 nt, Sphingobium sp.
  source 1..1417: mol_type = rRNA
tagattttgatcctggctcagaacgaacgctggcggcatgcctaatacatgcaagtcgaacgagatcttcggatctagtggcgcacgggtgcgtaacgcgtgggaatctgcccttgggttcggaataacttctggaaacggaagctaataccggatgatgacgtaagtccaaagatttatcgcccaaggatgagcccgcgtaggattagctagttggtggggtaaaggctcaccaaggcgacgatccttagctggtctgagaggatgatcagccacactgggactgagacacggcccagactcctacgggaggcagcagtagggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttgttcggaattactgggcgtaaagcgcacgtaggcggctattcaagtcagaggtgaaagcccggggctcaaccccggaactgcctttgaaactagatagcttgaatccaggagaggtgagtggaattccgagtgtagaggtgaaattcgtagatattcggaagaacaccagtggcgaaggcggctcactggactggtattgacgctgaggtgcgaaagcgtggggagcaaacaggattagataccctggtagtccacgccgtaaacgatgataactagctgtcagggcacatggtgttttggtggcgcagctaacgcattaagttatccgcctggggagtacggtcgcaagattaaaactcaaaggaattgacgggggcctgcacaagcggtggagcatgtggtttaattcgaagcaacgcgcagaaccttaccaacgtttgacatccctatcgcggatcgtggagacactttccttcagttcggctggataggtgacaggtgctgcatggctgtcgtcagctcgtgtcgtgagatgttgggttaagtcccgcaacgagcgcaaccctcgcctttagttgccagcatttagttgggtactctaaaggaaccgccggtgataagccggaggaaggtggggatgacgtcaagtcctcatggcccttacgcgttgggctacacacgtgctacaatggcgactacagtgggcagccacctcgcgagagggagctaatctccaaaagtcgtctcagttcggatcgttctctgcaactcgagagcgtgaaggcggaatcgctagtaatcgcggatcagcatgccgcggtgaatacgttcccaggccttgtacacaccgcccgtcacaccatgggagttggattcactcgaaggcgttgagctaaccgtaaggaggcaggcgaccacagtgtggtg

SEQ ID NO: 31 - RNA, 617 nt, Rhodotorula graminis
  source 1..617: mol_type = rRNA
gcatatcaataagcggaggaaaagaaactaacaaggattcccctagtagcggcgagcgaagcgggaagagctcaaatttataatctggcaccttcggtgtccgagttgtaatctctagaagtgttttccgcgttggaccgcacacaagtctgttggaatacagcggcatagtggtgaaacccccgtatatggtgcggacgcccagcgctttgtgatacactttcaatgagtcgagttgtttgggaatgcagctcaaattgggtggtaaattccatctaaagctaaatattggcgagagaccgatagcgaacaagtaccgtgagggaaagatgaaaagcactttggaaagagagttaacagtacgtgaaattgttggaagggaaacgcttgaagtcagacttgcttgccggagcttgcttcggtttgcaggccagcatcagttttccggggtggataatgacggtttgaaggtagcagtctcggctgtgttatagctttccgttggatacatcctgggggactgaggaacgcagcgtgctttttgcgaaagactcgtctttttcacgcttaggatgctggtggaatggctttaaacgacccgtcttgaaacacggacc

SEQ ID NO: 32 - RNA, 1528 nt, Herbiconiux sp.
  source 1..1528: mol_type = rRNA
tcatttacggagagtttgatcctggctcaggacgaacgctggcggcgtgcttaacacatgcaagtcgaacggtgaagcagagcttgctctgtggatcagtggcgaacgggtgagtaacacgtgagtaacctgcccttgactctgggataagcgttggaaacgacgtctaataccggatacgaccctcaaccgcatggttagggggtggaaagattttttggtcaaggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaaactggaggctcaacctccagcctgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggaactagatgtggggaccattccacggtctccgtgtcgcagctaacgcattaagttccccgcctggggagtacggccgcaaggctaaaactcaaaggaattgacgggggcccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgttatggtgggaactcataggagactgccggggtcaactcggaggaaggtggggatgacgtcaaatcatcatgccccttatgtcttgggcttcacgcatgctacaatggccggtacaaagggctgcaataccgtaaggtggagcgaatcccaaaaagccggtctcagttcggattgaggtctgcaactcgacctcatgaagtcggagtcgctagtaatcgtggatcagcaacgccacggtgaatacgttcccgggccttgtacacaccgcccgtcaagtcatgaaagtcggtaacacccgaagccggtggcccaaccttttggagggagccgtcgaaggtgggatcggtgattaggactaagtcgtaacaaggtagccgtaccggaaggtgcggctggatcacctcctttcta

SEQ ID NO: 33 - RNA, 1493 nt, Sphingobium sp.
  source 1..1493: mol_type = rRNA
atcaaacttgagagtttgatcctggctcagaacgaacgctggcggcatgcctaatacatgcaagtcgaacgagatcttcggatctagtggcgcacgggtgcgtaacgcgtgggaatctgcccttgggttcggaataacttctggaaacggaagctaataccggatgatgacgtaagtccaaagatttatcgcccaaggatgagcccgcgtaggattagctagttggtggggtaaaggctcaccaaggcgacgatccttagctggtctgagaggatgatcagccacactgggactgagacacggcccagactcctacgggaggcagcagtagggaatattggacaatgggcgaaagcctgatccagcaatgccgcgtgagtgatgaaggccttagggttgtaaagctcttttacccgggatgataatgacagtaccgggagaataagctccggctaactccgtgccagcagccgcggtaatacggagggagctagcgttgttcggaattactgggcgtaaagcgcacgtaggcggctattcaagtcagaggtgaaagcccggggctcaaccccggaactgcctttgaaactagatagcttgaatccaggagaggtgagtggaattccgagtgtagaggtgaaattcgtagatattcggaagaacaccagtggcgaaggcggctcactggactggtattgacgctgaggtgcgaaagcgtggggagcaaacaggattagataccctggtagtccacgccgtaaacgatgataactagctgtcagggcacatggtgttttggtggcgcagctaacgcattaagttatccgcctggggagtacggtcgcaagattaaaactcaaaggaattgacgggggcctgcacaagcggtggagcatgtggtttaattcgaagcaacgcgcagaaccttaccaacgtttgacatccctatcgcggatcgtggagacactttccttcagttcggctggataggtgacaggtgctgcatggctgtcgtcagctcgtgtcgtgagatgttgggttaagtcccgcaacgagcgcaaccctcgcctttagttgccagcatttagttgggtactctaaaggaaccgccggtgataagccggaggaaggtggggatgacgtcaagtcctcatggcccttacgcgttgggctacacacgtgctacaatggcgactacagtgggcagccacctcgcgagagggagctaatctccaaaagtcgtctcagttcggatcgttctctgcaactcgagagcgtgaaggcggaatcgctagtaatcgcggatcagcatgccgcggtgaatacgttcccaggccttgtacacaccgcccgtcacaccatgggagttggattcactcgaaggcgttgagctaaccgtaaggaggcaggcgaccacagtgggtttagcgactggggtgaagtcgtaacaaggtagccgtaggggaacctgcggctggatcacctcctttcta

SEQ ID NO: 34 - RNA, 1528 nt, Herbiconiux sp.; identical to SEQ ID NO: 32
  source 1..1528: mol_type = rRNA

SEQ ID NO: 35 - RNA, 1479 nt, Rahnella aceris
  source 1..1479: mol_type = rRNA
tggctcagattgaacgctggcggcaggcctaacacatgcaagtcgagcggcagcgggaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcacacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttagggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcg

SEQ ID NO: 36 - RNA, 1388 nt, Pseudomonas graminis
  source 1..1388: mol_type = rRNA
gctcagattgaacgctggcggcaggcctaacacatgcaagtcgagcggatgaagagagcttgctctctgattcagcggcggacgggtgagtaatgcctaggaatctgcctggtagtgggggacaacgtctcgaaagggacgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagcgaataccttgctgttttgacgttaccgacagaataagcaccggctaactctgtgccagcagccgcggtaatacagagggtgcaagcgttaatcggaattactgggcgtaaagcgcgcgtaggtggtttgttaagttgaatgtgaaatccccgggctcaacctgggaactgcatccaaaactggcaagctagagtagggcagagggtggtggaatttcctgtgtagcggtgaaatgcgtagatataggaaggaacaccagtggcgaaggcgaccacctgggctcatactgacactgaggtgcgaaagcgtggggagcaaacaggattagataccctggtagtccacgccgtaaacgatgtcaactagccgttggaagccttgagcttttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacagagggttgccaagccgtgaggtggagctaatcccagaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccat

SEQ ID NO: 37 - RNA, 1548 nt, Rahnella aceris
  source 1..1548: mol_type = rRNA
ttttaattgaagagtttgatcatggctcagattgaacgctggcggcaggcctaacacatgcaagtcgagcggcagcggaaagtagcttgctactttgccggcgagcggcggacgggtgagtaatgtctgggaaactgcctgatggagggggataactactggaaacggtagctaataccgcatgacctcgaaagagcaaagtgggggatcttcggacctcacgccatcggatgtgcccagatgggattagctagtaggtgaggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttagggttgtaaagcactttcagcgaggaggaaggcatcatacttaatacgtgtggtgattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaattcgccagagatggcttagtgccttcgggaaccgtgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttacc